(19) United States
(12) Patent Application Publication
(10) Pub. No.: US 2026/0098026 A1
(43) Pub. Date: Apr. 9, 2026

(54) M4 ACTIVATORS/MODULATORS AND USES THEREOF

(71) Applicant: Cerevel Therapeutics, LLC, Boston, MA (US)

(72) Inventors: Hanh Nguyen, Acton, MA (US); Shea Johnson, Medford, MA (US); Xiaofeng Zhang, Malden, MA (US); Sokhom Pin, Lunenburg, MA (US); Jongwon Lim, Lexington, MA (US); Hongjun Zhang, West Roxbury, MA (US)

(21) Appl. No.: 19/112,200

(22) PCT Filed: Sep. 15, 2023
(86) PCT No.: PCT/US2023/032844

§ 371 Date: Mar. 14, 2025

Related U.S. Application Data
(60) Provisional application No. 63/407,358, filed on Sep. 16, 2022.

(51) Int. Cl. C07D 401/14; A61K 31/4545; A61K 31/496; A61K 31/497; A61K 31/4995; A61K 31/501; A61K 31/506; A61K 31/513; A61K 31/519; A61K 31/5365; A61K 31/5377; A61K 31/538; A61K 31/695; C07B 59/00; C07D 405/14; C07D 409/14; C07D 413/14; C07D 417/14; C07D 471/04; C07D 487/04; C07D 487/08; C07D 491/107; C07D 495/10; C07D 498/04; C07F 7/08

(52) U.S. Cl. CPC C07D 401/14; A61K 31/4545; A61K 31/496; A61K 31/497; A61K 31/4995; A61K 31/501; A61K 31/506; A61K 31/513; A61K 31/519; A61K 31/5365; A61K 31/5377; A61K 31/538; A61K 31/695; C07B 59/002; C07D 405/14; C07D 409/14; C07D 413/14; C07D 417/14; C07D 471/04; C07D 487/04; C07D 487/08; C07D 491/107; C07D 495/10; C07D 498/04; C07F 7/0812; C07B 2200/05

(57) ABSTRACT

The present disclosure provides compounds of Formula I: (I), or an N-oxide thereof, or a pharmaceutically acceptable salt of the compound or the N-oxide, wherein: A, Y, m, n, p, R1, R2, R3, R3a, R4, R5, R6, R7, and Z are as described herein; processes for the preparation of; intermediates used in the preparation of; and compositions containing such compounds, N-oxides, or salts, and their uses for treating M4-mediated (or M4-associated) disorders including, e.g., Alzheimer's Disease, Parkinson's Disease, schizophrenia (e.g., its cognitive and negative symptoms), pain, addiction, and a sleep disorder.

### FIELD

The present disclosure generally relates to novel pyridine azaspiro compounds which are agonists/activators/modulators of the muscarinic M4 receptor and are useful in the treatment of M4-mediated diseases and disorders, but not limited to schizophrenia, Alzheimer's disease, dementia related psychosis, dementia with Lewy Bodies, Parkinson's disease and related memory and executive dysfunction, bipolar disorder, agitation, and psychosis associated therewith.

### BACKGROUND

Patients with Schizophrenia, Alzheimer's Disease, Parkinson's Disease, Huntington's Disease, depression and various other neurological/neurodegenerative diseases frequently suffer from behavioral and cognitive impairments resulting in debilitating disruption to their daily lives. Over the years, many pharmacological treatments have been discovered that provide some improvement in behavior and cognitive function. However, the improvement is modest at best, and as is often the case, the underlying dose-limiting adverse effects associated with these treatments, including extrapyramidal and metabolic side-effects, lead to partial responsiveness, and non-compliance.

The muscarinic acetylcholine receptor (mAChR) is a viable mechanism for treating such diseases.

There are five mAChR subtypes (M1-M5) that have been identified and are part of the G protein-coupled receptor (GPCR) superfamily. These subtypes are distributed widely throughout the periphery and the central nervous system (CNS), with the M1 and M4 subtypes being predominantly expressed in the CNS. The M4 agonist HTL0016878 being developed for the treatment of major symptoms of Alzheimer's Disease entered into a Phase 2 clinical study.

Thus, a need exists for agonists of the muscarinic M4 receptors f to treat M4-mediated diseases and disorders such as Parkinson's Disease, Schizophrenia, Alzheimer's Disease and others described herein.

### SUMMARY

Provided herein are compounds having a structure of Formula (I), or an N-oxide thereof, or a pharmaceutically acceptable salt of the compound or the N-oxide thereof:

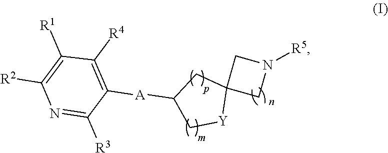

wherein A is a 6-8 membered heterocycle comprising 1 or 2 ring nitrogen atoms and optionally substituted with 1 to 3 C₁₋₃alkyl groups; Y is a bond, S, CH₂, CHF, CF₂, or C(OH)H; m is 1 or 2; n is 1 or 2; p is 1 or 2; R¹ is H, halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, —[O]₀₋₁-5-10 membered heteroaryl, —NH—C₃₋₆cycloalkyl, —NH—C₆₋₁₀ aryl, —NH-4-8 membered heterocycle, —NH-5-10 membered heteroaryl, —N(C₁₋₆alkyl)-C₃₋₆cycloalkyl, —N(C₁₋₆alkyl)-C₆₋₁₀aryl, —N(C₁₋₆alkyl)-4-8 membered heterocycle, —N(C₁₋₆alkyl)-5-10 membered heteroaryl, wherein the heterocycle and heteroaryl each comprises 1, 2, or 3 ring heteroatoms selected from N, O, and S, and when R¹ is C₁₋₆alkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle or —[O]₀₋₁-5-10 membered heteroaryl, R¹ is optionally substituted with 1, 2, or 3 substituents independently selected from halogen, CN, OH, ═O, SO₂, and C₁₋₃alkyl; R² is H, halogen, CN, OH, —N(R⁶)(R⁷), C₁₋₆ alkyl, C₁₋₆haloalkyl, C₁₋₆alkoxy, C₁₋₆haloalkoxy, C₂₋₆heteroalkyl, C₃₋₆cycloalkyl, or 4-8 membered heterocycle comprising 1, 2, or 3 ring heteroatoms selected from N, O, and S; R³ is halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, —[O]₀₋₁-5-10 membered heteroaryl, —NH—C₃₋₆cycloalkyl, —NH—C₆₋₁₀aryl, —NH-4-8 membered heterocycle, —NH-5-10 membered heteroaryl, —N(C₁₋₆alkyl)-C₃₋₆cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, wherein the heterocycle and heteroaryl each comprises 1, 2, or 3 ring heteroatoms selected from N, O, and S, and when R³ is —[O]₀₋₁—C₃₋₆cycloalkyl, —[O]₀₋₁—C₆₋₁₀aryl, —[O]₀₋₁-4-8 membered heterocycle, —[O]₀₋₁-5-10 membered heteroaryl, —NH—C₃₋₆cycloalkyl, —NH—C₆₋₁₀aryl, —NH-4-8 membered heterocycle, —NH-5-10 membered heteroaryl, —N(C₁₋₆alkyl)-C₃₋₆cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, it is optionally substituted with 1, 2, or 3 R^{3a} substituents; each R^{3a} is independently selected from halogen, CN, OH, ═O, SO₂, C₁₋₆alkyl, C₂₋₁₀alkene, C₁₋₆hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-NH₂, C₀₋₆alkylene-NH(C₁₋₆alkyl), C₀₋₆alkylene-N(C₁₋₆alkyl)₂, —S—C₁₋₆alkyl, C₀₋₆alkylene-SO₂C₁₋₆alkyl, C₀₋₆alkylene-C(O)NH₂, C₀₋₆alkylene-C(O)NH(C₁₋₆alkyl), C₀₋₆alkylene-C(O)N(C₁₋₆alkyl)₂, C₀₋₆alkylene-NHC(O)C₁₋₆alkyl, C₀₋₆alkylene-COOH, and C₀₋₆alkylene-3-6 membered heterocycle comprising 1, 2, or 3 heteroatoms selected from N, O, and S; R⁴ is H, halogen, CN, or OH; R⁵ is —CO₂—Z, or a bioisostere thereof; each R⁶ and R⁷ is independently H, C₁₋₆alkyl, C(O)—C₁₋₆alkyl, spiro or bicyclic C₈₋₁₄cycloalkyl, 8-14 membered heterocycle comprising 1, 2, or 3 ring heteroatoms selected from N, O, and S, and when R⁶ or R⁷ is other than H, it can be optionally substituted with 1, 2, or 3 substituents independently selected from the group consisting of halogen, CN, OH, ═O, SO₂, C₀₋₆alkylene-NH₂, C₀₋₆alkylene-NH(C₁₋₆alkyl), C₀₋₆alkylene-N(C₁₋₆alkyl)₂, C₀₋₆alkyene-SO₂C₁₋₆alkyl, C₁₋₆alkyl and C₁₋₆alkoxy, or R⁶ and R⁷, together with the nitrogen to which they are attached, form a 4-10 membered heterocycle comprising 0-2 additional ring heteroatoms independently selected from N, O, and S; and Z is C₁₋₇alkyl, C₁₋₇haloalkyl, C₃₋₆cycloalkyl, or C₂₋₆alkynyl, and Z is optionally substituted with C₁₋₆alkoxy or C₃₋₆ cycloalkyl; with the proviso that when R¹, R², and R⁴ are each H, Y is CH₂, m, n, and p are each 1, A is

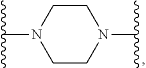 [SMILES: *N1CCN(*)CC1]

and R³ is trifluoroethoxy, trifluoromethoxy, difluoromethoxy, methoxy, or

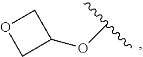 [SMILES: *OC1COC1]

then R⁵ is not CO₂CH₂CH₃.

Further provided herein are pharmaceutical compositions comprising the compounds as disclosed herein. Also provided are methods of treating an M4-mediated (or M4-associated) disease or disorder associated with aberrant M4 receptor activity in a subject, comprising administering to the subject a therapeutically effective amount of a compound as disclosed herein.

Further aspects and advantages will be apparent to those of ordinary skill in the art from a review of the following detailed description, taken in conjunction with the drawings. While the compounds and methods disclosed herein are susceptible of cases in various forms, the description hereafter includes specific cases with the understanding that the disclosure is illustrative, and is not intended to limit the invention to the specific cases described herein.

### DETAILED DESCRIPTION

Provided herein are compounds that have a structure of Formula (I):

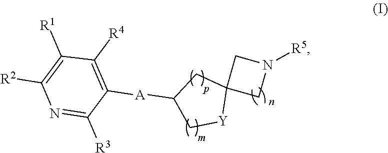

or an N-oxide thereof, or a pharmaceutically acceptable salt of the compound or the N-oxide thereof. Also provided herein are compounds that act on M4 receptors. The compounds described herein can be used to treat M4-mediated or M4-associated diseases.

### Chemical Definitions

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₆alkyl refers to an alkyl group that has 6 carbon atoms. C₁₋₇alkyl refers to an alkyl group having a number of carbon atoms encompassing the entire range (i.e., 1 to 7 carbon atoms), as well as all subgroups (e.g., 2-6, 2-5, 3-6, 2, 3, 4, 5, 6, and 7 carbon atoms).

Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “alkylene” refers to a bivalent saturated aliphatic radical. The term Cₙ means the alkylene group has “n” carbon atoms, e.g., a C₁alkylene is CH₂. For example, C₁₋₆alkylene refers to an alkylene group having a number of carbon atoms encompassing the entire range, as well as all subgroups, as previously described for “alkyl” groups.

As used herein, the term “alkene” or “alkenyl” is defined identically as “alkyl” except for containing at least one carbon-carbon double bond, and having two to thirty carbon atoms, for example, two to twenty carbon atoms, or two to ten carbon atoms. The term Cₙ means the alkenyl group has “n” carbon atoms. For example, C₄alkenyl refers to an alkenyl group that has 4 carbon atoms. C₂₋₇alkenyl refers to an alkenyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Specifically contemplated alkenyl groups include ethenyl, 1-propenyl, 2-propenyl, and butenyl. Unless otherwise indicated, an alkenyl group can be an unsubstituted alkenyl group or a substituted alkenyl group. Unless otherwise indicated, an alkenyl group can be a cis-alkenyl or trans-alkenyl.

As used herein, the term “alkyne” or “alkynyl” is defined identically as “alkyl” except for containing at least one carbon-carbon triple bond, and having two to thirty carbon atoms, for example, two to twenty carbon atoms, or two to ten carbon atoms. The term Cₙ means the alkynyl group has “n” carbon atoms. For example, C₄alkynyl refers to an alkynyl group that has 4 carbon atoms. C₂₋₇alkynyl refers to an alkynyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Specifically contemplated alkynyl groups include ethynyl, 1-propynyl, 2-propynyl, and butynyl. Unless otherwise indicated, an alkynyl group can be an unsubstituted alkynyl group or a substituted alkynyl group.

As used herein, the term “cycloalkyl” specifically refers to a non-aromatic ring in which each atom of the ring is carbon, i.e., a carbocycle, and can be monocyclic, bicyclic, bridged, fused or spirocyclic. The term Cₙ means the cycloalkyl group has “n” ring carbon atoms. For example, C₅ cycloalkyl refers to a cycloalkyl group that has 5 ring carbon atoms in the ring. C₃₋₈ cycloalkyl refers to cycloalkyl groups having a number of ring carbon atoms encompassing the entire range (i.e., 3 to 8 carbon atoms), as well as all subgroups (e.g., 4-8, 3-7, 4-7, 3-6, 4-6, 3-5, 4-5, 3, 4, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. Non limiting examples of bridged cycloalkyl groups include,

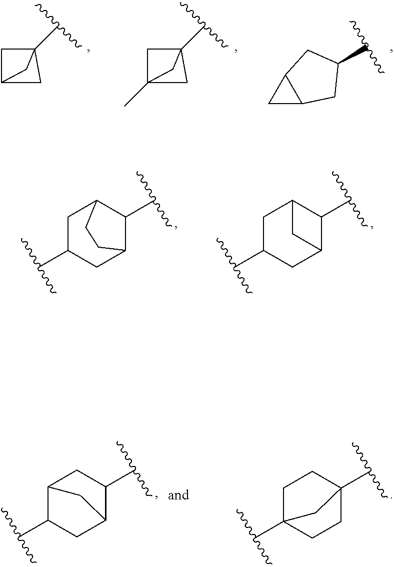

Non limiting examples of spirocycloalkyl groups include

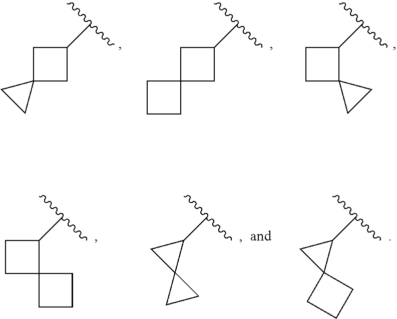 [SMILES: *C1CC2(CC2)C1 ; *C1CC2(CCC2)C1 ; *C1CCC12CC2 ; *C1CCC12CCC2 ; *C1CC12CC2 ; *C1CC12CCC2]

Unless otherwise indicated, a cycloalkyl group can be an unsubstituted cycloalkyl group or a substituted cycloalkyl group.

As used herein, the term “heterocycle” is defined similarly as cycloalkyl, except the ring contains one to four heteroatoms independently selected from oxygen, nitrogen, and sulfur. Additionally, heterocycles of the disclosure can be monocyclic, bicyclic, bridged, fused or spirocyclic. For example, a heterocycle can be a monocyclic, bicyclic, bridged, fused, or spirocyclic 4-8 membered ring having 1 or 2 or 3 heteroatoms selected from N, O, and S. As another example, a heterocycle can be a 8-10 membered bicyclic, bridged, fused, or spirocyclic group having 1 or 2 or 3 ring heteroatoms selected from N, O, and S in the bicyclic ring. Nonlimiting examples of heterocycle groups include azepane, aziridine, piperidine, piperazine, tetrahydrofuran, tetrahydropyran, tetrahydropyridine, dihydrofuran, dihydropyran, morpholine, oxazepane, thiazole, pyrrole, pyridinone, and pyridine. Non limiting examples of heterocycle groups include

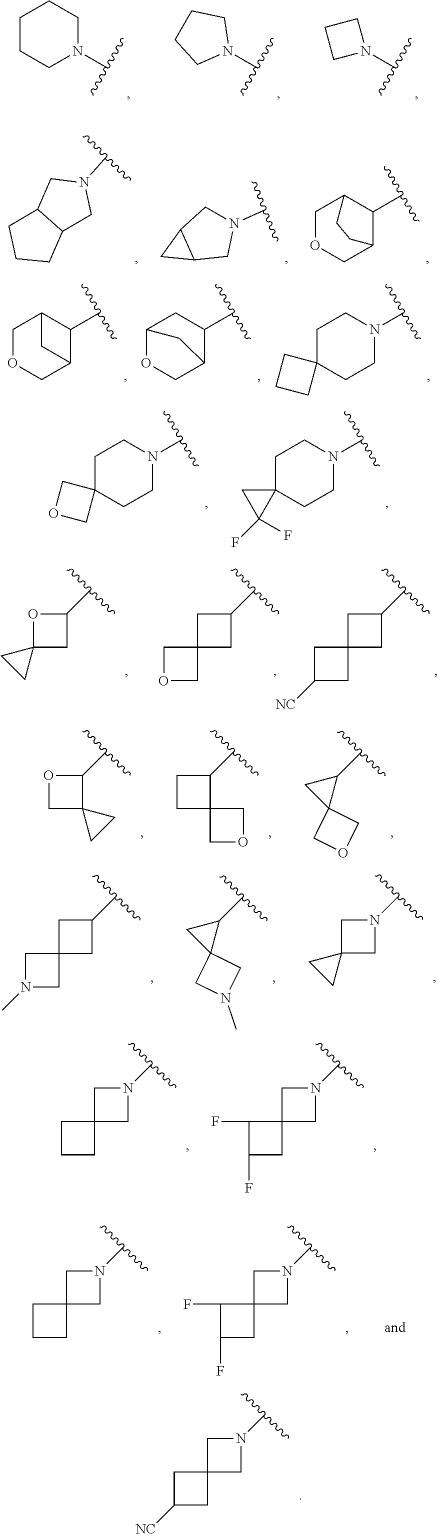

Cycloalkyl and heterocycle groups can be saturated or partially unsaturated ring systems (e.g., having double or triple bonds), but the groups are not aromatic. Cycloalkyl and heterocycle groups can be optionally substituted with, for example, one to three groups, independently selected alkyl, alkoxy, alkylene, OH, C₀₋₆alkylene-C(O)NH₂, NH₂, =O, SO₂, aryl, haloalkyl, haloalkoxy, C₀₋₆alkylene-C(O)-alkyl, C₀₋₆alkylene-SO₂alkyl, halogen, OH, NHC₁₋₃alkylene-aryl, OC₁₋₃alkylene-aryl, C₁₋₃alkylene-aryl, and C₀₋₆alkylene-C₃₋₆heterocycle having 1-3 heteroatoms selected from N, O, and S. Specific substitutions for these groups is described elsewhere in this disclosure.

As used herein, the term “aryl” refers to an aromatic ring in which each atom of the ring is carbon, and can be monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, fluorenyl, tetralinyl. Unless otherwise indicated, an aryl group can be an unsubstituted aryl group or a substituted aryl group.

As used herein, the term “heteroaryl” refers to an aromatic heterocycle, and can be monocyclic or polycyclic (e.g., fused bicyclic and fused tricyclic) aromatic ring systems, wherein one to four (e.g., one to three) ring atoms are selected from oxygen, nitrogen, and sulfur, and the remaining ring atoms are carbon, said ring system being joined to the remainder of the molecule by any of the ring atoms. Nonlimiting examples of heteroaryl groups include, but are not limited to, pyridyl, pyridazinyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, thienyl, quinolinyl, isoquinolinyl, benzoxazolyl, benzimidazolyl, benzofuranyl, benzothiazolyl, triazinyl, triazolyl, purinyl, pyrazinyl, purinyl, indolinyl, phthalzinyl, indazolyl, quinolinyl, isoquinolinyl, cinnolinyl, quinazolinyl, naphthyridinyl, pyridopyridinyl, indolyl, 3H-indolyl, pteridinyl, and quinooxalinyl. Nonlimiting examples of heteroaryl groups include

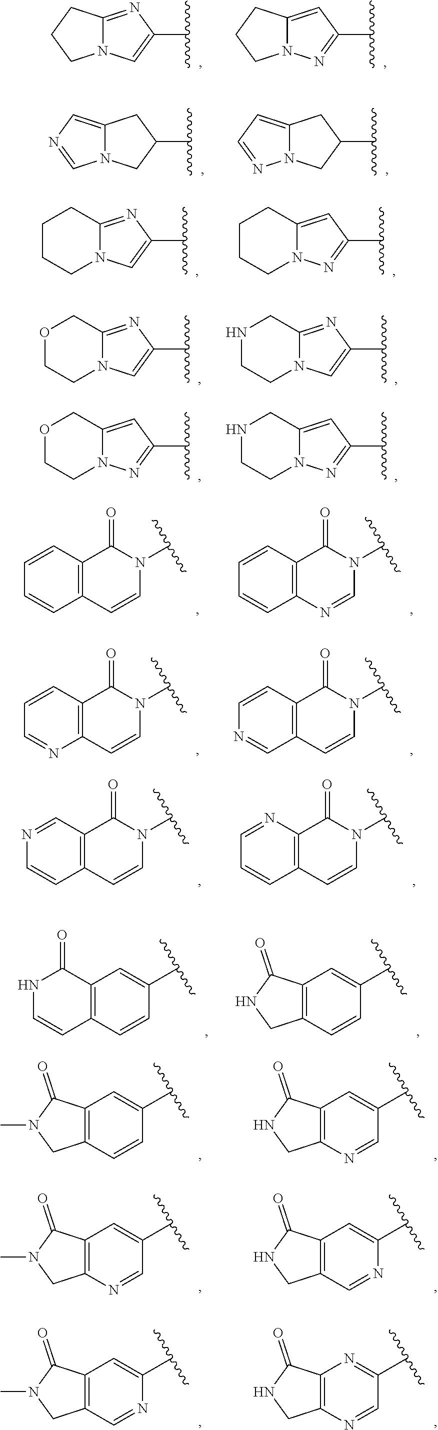

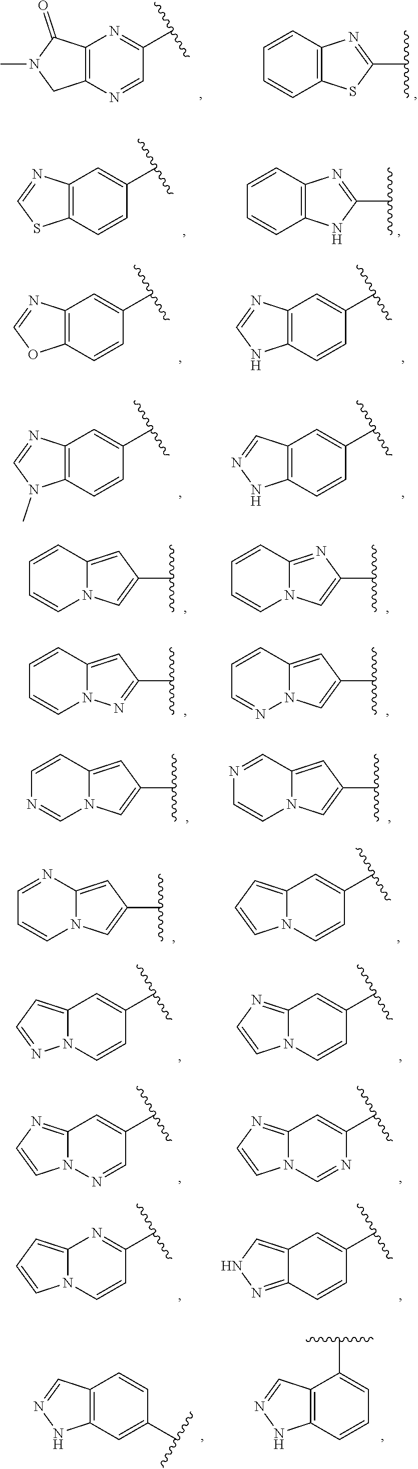

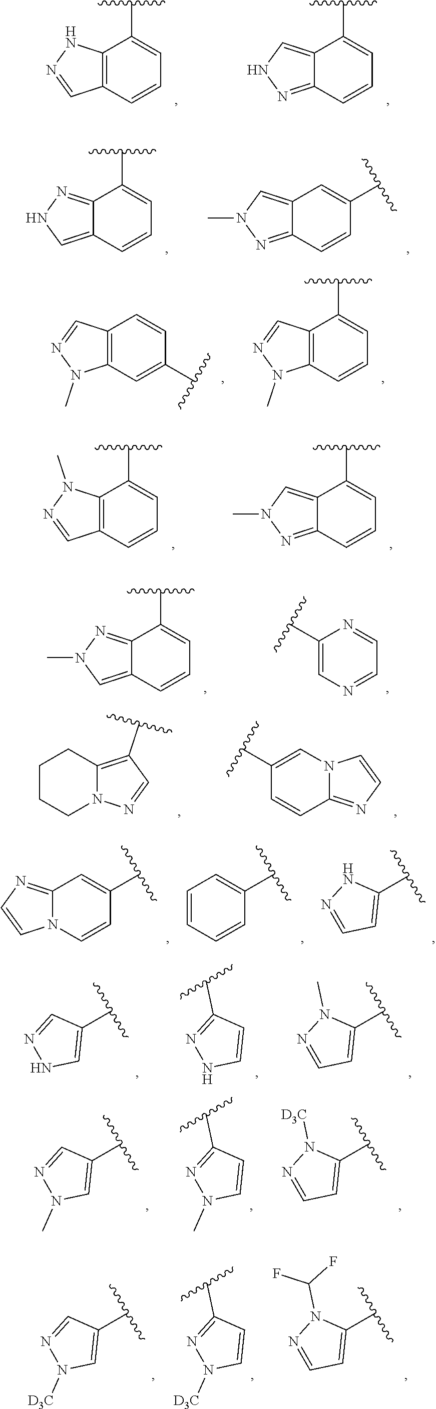

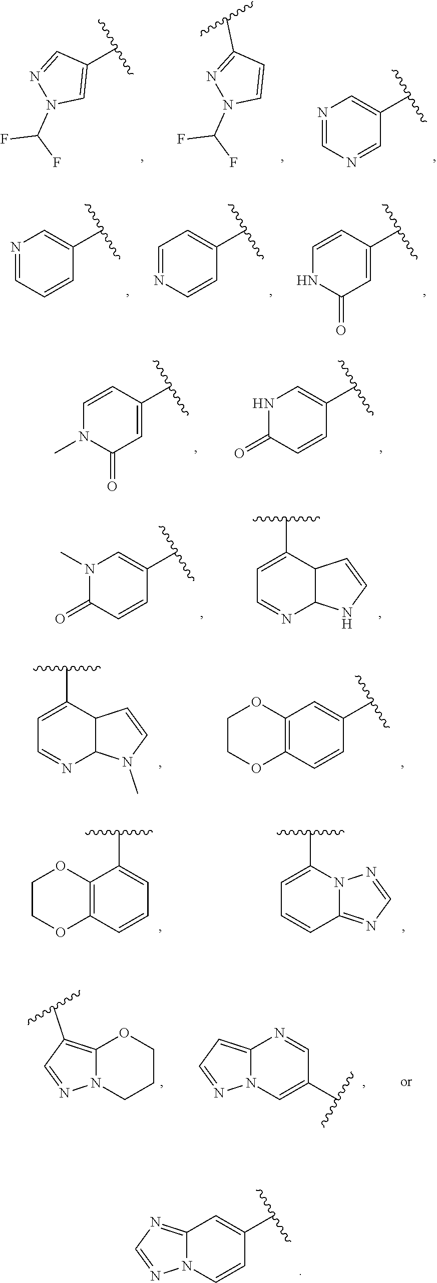

Unless otherwise indicated, a heteroaryl group can be an unsubstituted heteroaryl group or a substituted heteroaryl group.

As used herein, the term “hydroxy” or “hydroxyl” refers to an “—OH” group. Accordingly, a “hydroxyalkyl” refers to an alkyl group substituted with one or more —OH groups.

As used herein, “haloalkyl” refers to an alkyl group where one or more of the hydrogen atoms have been substituted with one or more halogen.

As used herein, the term “alkoxy” or “alkoxyl” refers to a “—O-alkyl” group.

As used herein, the term “halogen” is defined as fluoro, chloro, bromo, and iodo. Accordingly, a “haloalkyl” refers to an alkyl group substituted with one or more halogen atoms. In some cases, the haloalkyl group is a perhaloalkyl, i.e., all hydrogen atoms of the alkyl group have been substituted with a halogen. Some non-limiting examples of haloalkyl groups include CF₃, CHF₂, CH₂F, CCl₃, Cl₃, and CH₂CF₃. Similarly, a “haloalkoxy” refers to an alkoxy group that is substituted with one or more halogen atoms, e.g., OCF₃.

As used herein, the term “heteroalkyl” refers to an alkyl chain interrupted with one or more heteroatoms selected from N, O, and S and having two to thirty carbon atoms, for example, two to twenty carbon atoms, or two to ten carbon atoms. The term Cₙ means the heteroalkyl group has “n” carbon atoms. For example, C₆heteroalkyl refers to an alkyl group that has 6 carbon atoms and the carbon chain is interrupted with one or more heteroatoms. C₂₋₆heteroalkyl refers to an alkyl group having a number of carbon atoms encompassing the entire range (i.e., 2 to 6 carbon atoms), as well as all subgroups (e.g., 2-5, 3-6, 3-5, 4-6, 2, 3, 4, 5, and 6 carbon atoms).

As used herein, the term “bioisostere” refers to a molecule resulting from the exchange of an atom or of a group of atoms with an alternative, broadly similar, atom or group of atoms. For example, an ester group can be replaced by one of the following bioisosteres for the ester, including, without limitation, acylsulfonamides (CONR—SO₂R), hydroxamic acids (CONROH), hydroxamates (CONROR), tetrazoles, hydroxyisoxazoles, isoxazol-3-ones, and sulfonamides (SO₂NR), where each R may independently represent hydrogen, alkyl, fluoroalkyl, carbocyclyl, carbocyclylalkyl, aryl, aralkyl, heterocycloalkyl, heterocycloalkylaikyl, heteroaryl or heteroarylalkyl.

A “substituted” functional group (e.g., a substituted alkyl, cycloalkyl, aryl, or heteroaryl) is a functional group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkenyl, alkynyl, ether, aryl, O-alkylene aryl, N-alkylene aryl, alkylene aryl, heteroaryl, heterocycle, hydroxy, hydroxyalkyl, haloalkoxy, amido, =O, SO₂, alkoxy, ester, thioester, acyl, carboxyl, cyano, nitro, amino, sulfhydryl, and halogen. When a substituted alkyl group includes more than one non-hydrogen radical, the substituents can be bound to the same carbon or two or more different carbon atoms.

### COMPOUNDS OF THE DISCLOSURE

Disclosed herein are compounds having a structure of Formula (I):

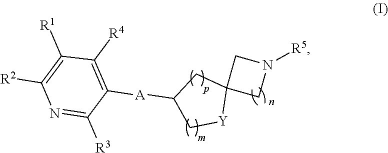

or an N-oxide thereof, or a pharmaceutically acceptable salt of the compound or the N-oxide thereof, wherein:
A is a 6-8 membered heterocycle comprising 1 or 2 ring nitrogen atoms and optionally substituted with 1 to 3 substituents independently selected from halogen, OH, and C₁₋₃alkyl;
Y is a bond, S, O, CH₂, CHF, CF₂, or C(OH)H;
m is 1 or 2;
n is 1 or 2;
p is 1 or 2;
R¹ is H, halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆ cycloalkyl, —[O]₀₋₁—C₆₋₁₀aryl, —[O]₀₋₁-4-8 membered heterocycle, —[O]₀₋₁-5-10 membered heteroaryl, —NH—C₃₋₆cycloalkyl, —NH—C₆₋₁₀aryl, —NH-4-8 membered heterocycle, —NH-5-10 membered heteroaryl, —N(C₁₋₆ alkyl)-C₃₋₆cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, wherein the heterocycle and heteroaryl each comprises 1, 2, or 3 ring heteroatoms selected from N, O, and S, and when R¹ is C₁₋₆alkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle or —[O]₀₋₁-5-10 membered heteroaryl, R¹ is optionally substituted with 1, 2, or 3 substituents independently selected from halogen, CN, OH, ═O, SO₂, and C₁₋₃alkyl,
R² is H, halogen, CN, OH, —N(R⁶)(R⁷), C₁₋₆alkyl, C₂₋₆haloalkyl, C₁₋₆alkoxy, C₁₋₆haloalkoxy, C₂₋₆heteroalkyl, C₃₋₆ cycloalkyl, or 4-8 membered heterocycle comprising 1, 2, or 3 ring heteroatoms selected from N, O, and S;
R³ is halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆ cycloalkyl, —[O]₀₋₁—C₆₋₁₀aryl, —[O]₀₋₁-4-8 membered heterocycle, or —[O]₀₋₁-5-10 membered heteroaryl, wherein the heterocycle and heteroaryl each comprises 1, 2, or 3 ring heteroatoms selected from N, O, and S, and the C₃₋₆cycloalkyl, C₆₋₁₀aryl, 4-8 membered heterocycle, or 5-10 membered heteroaryl is substituted with 0, 1, 2, or 3 R^{3a} substituents;
each R^{3a} is independently selected from halogen, CN, OH, ═O, ═N(C₁₋₃alkyl), C₁₋₆alkyl, C₂₋₁₀alkene, C₁₋₆ hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-NH₂, C₀₋₆ alkylene-NH(C₁₋₆alkyl), C₀₋₆alkylene-N(C₁₋₆alkyl)₂, —S—C₁₋₆alkyl, C₀₋₆alkylene-SO₂C₁₋₆alkyl, C₀₋₆alkylene-C(O)NH₂, C₀₋₆alkylene-C(O)NH(C₁₋₆alkyl), C₀₋₆alkylene-C(O)N(C₁₋₆alkyl)₂, C₀₋₆alkylene-NHC(O)C₁₋₆alkyl, C₀₋₆alkylene-COOH, C₀₋₆alkylene-C₃₋₆cycloalkyl, C₁₋₆alkylene-O—C₁₋₆alklyeneSi(C₁₋₃alkyl)₃, and C₀₋₆ alkylene-3-6 membered heterocycle comprising 1, 2, or 3 heteroatoms selected from N, O, and S;
R⁴ is H, halogen, CN, or OH;
R⁵ is —CO₂—Z, or a bioisostere thereof;
each R⁶ and R⁷ is independently H, C₁₋₆alkyl, C(O)—C₁₋₆alkyl, spiro or bicyclic C₈₋₁₄cycloalkyl, 8-14 membered heterocycle comprising 1, 2, or 3 ring heteroatoms selected from N, O, and S, and when R⁶ or R⁷ is other than H, it can be optionally substituted with 1, 2, or 3 substituents independently selected from the group consisting of halogen, CN, ═O, SO₂, OH, C₀₋₆alkylene-NH₂, C₀₋₆alkylene-NH(C₁₋₆alkyl), C₀₋₆alkylene-N(C₁₋₆alkyl)₂, C₀₋₆alkylene-SO₂C₁₋₆alkyl, C₁₋₆alkyl and C₁₋₆alkoxy, or
R⁶ and R⁷, together with the nitrogen to which they are attached, form a 4-10 membered heterocycle comprising 0-2 additional ring heteroatoms independently selected from N, O, and S; and
Z is C₁₋₇alkyl, C₁₋₇haloalkyl, C₃₋₆cycloalkyl, or C₂₋₆alkyne, and Z is optionally substituted with C₁₋₆alkoxy or C₃₋₆cycloalkyl;
with the proviso that when R¹, R², and R⁴ are each H, Y is CH₂, m, n, and p are each 1, A

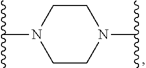 [SMILES: *N1CCN(*)CC1]

and R³ is trifluoroethoxy, trifluoromethoxy, difluoromethoxy, methoxy, or

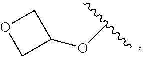 [SMILES: *OC1COC1]

then R⁵ is not CO₂CH₂CH₃.

In compounds of Formula (I), A can be a 6-8 membered heterocycle comprising 1 or 2 ring nitrogen atoms and optionally substituted with 1 to 3 C₁₋₃alkyl groups. In various cases, A is

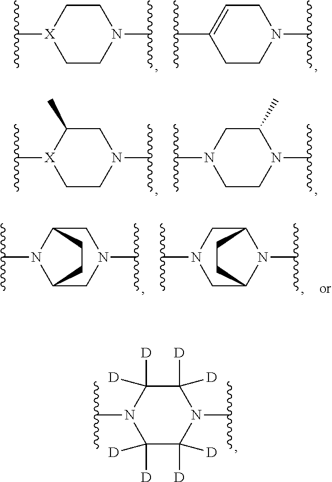

where X is N, CH, CF, or C(OH). In various cases, A is

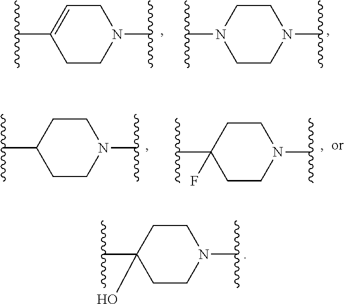

In compounds of Formula (I), X can be N or CH. In various cases, X is N. In various cases, X is CH.

In some cases, the compound has a structure of Formula (Ia):

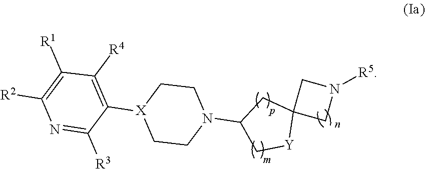

In some cases, the compound has a structure of Formula (Ib):

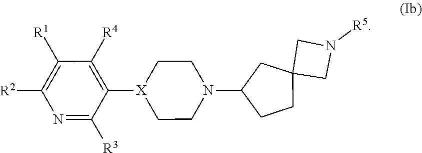

In various cases, Y can be a bond, S, CH₂, CHF, CF₂, or C(OH)H. In various cases, Y is CH₂, CHF, CF₂, or C(OH)H. In some cases, Y is CH₂.

In various cases, m can be 1 or 2. In various cases, m is 1.

In various cases, n can be 1 or 2. In various cases, n is 1.

In various cases, p can be 1 or 2. In various cases, p is 1.

In some cases, the compound has a structure of Formula (Ic):

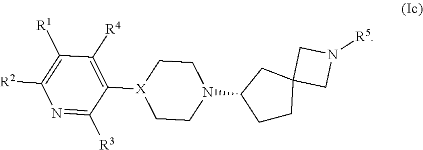

In some cases, the compound has a structure of Formula (Id):

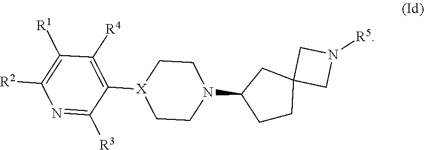

In various cases, R⁵ is —CO₂—Z, or a bioisostere thereof. In various cases, R⁵ is a CO₂Z bioisostere selected from

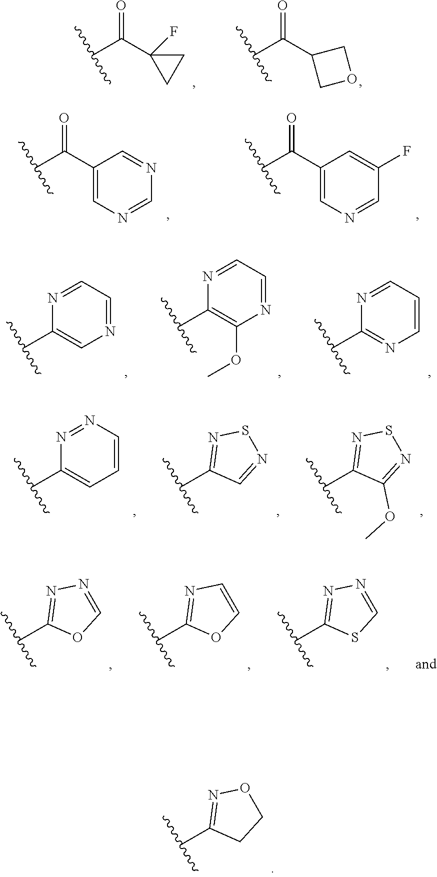

In various cases, R⁵ is selected from CO₂—C₁₋₇alkyl,

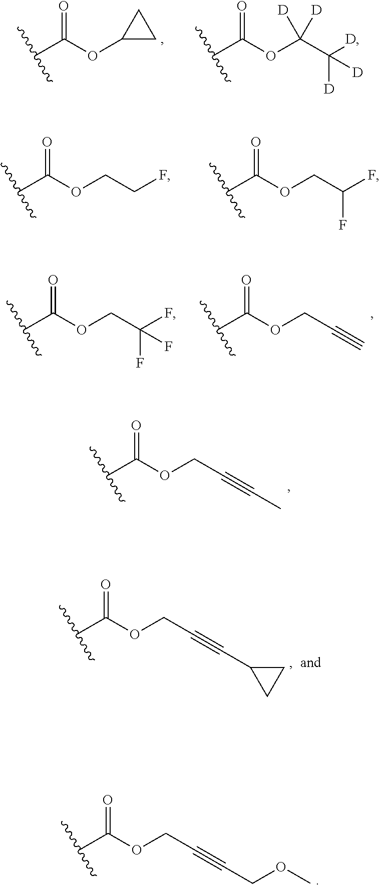

In some cases, R⁵ is CO₂—C₁₋₇alkyl. In some cases, R⁵ is CO₂CH₂CH₃.

In some cases, the compound has a structure of Formula (Ie):

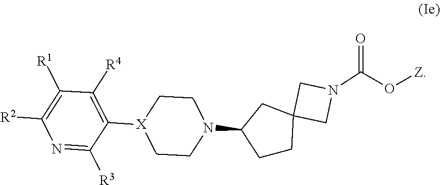

In various cases, R⁴ can be H, halogen, CN, or OH. In various cases, R⁴ is H or halogen. In some cases, R⁴ is H.

In various cases, R¹ can be H, halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle or —[O]₀₋₁-5-10 membered heteroaryl, wherein the heterocycle and heteroaryl each comprises 1, 2, or 3 ring heteroatoms selected from N, O, and S, and when R¹ is C₁₋₆alkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle or —[O]₀₋₁-5-10 membered heteroaryl, and R¹ can be optionally substituted with 1, 2, or 3 substituents independently selected from halogen, CN, OH, ═O, SO₂, and C₁₋₃alkyl. In various cases, R¹ is R¹ is H, halogen, CN, OH, —N(R⁶)(R⁷), C₁₋₆alkyl, or C₁₋₆alkoxy. In some cases, R¹ is H or halogen.

In various cases, R² can be H, halogen, CN, OH, —N(R⁶)(R⁷), C₁₋₆alkyl, C₁₋₆haloalkyl, C₁₋₆alkoxy, C₁₋₆ haloalkoxy, C₂₋₆heteroalkyl, C₃₋₆cycloalkyl, or 4-8 membered heterocycle comprising 1, 2, or 3 ring heteroatoms selected from N, O, and S. In various cases, R² is H, halogen, CN, OH, —N(R⁶)(R⁷), C₁₋₆alkyl, C₁₋₆haloalkyl, C₁₋₆alkoxy, or C₁₋₆haloalkoxy. In some cases, R² is H, halogen, C₁₋₆alkyl, C₁₋₆alkoxy, C₁₋₆haloalkyl, or C₁₋₆haloalkoxy. In some cases, R² is H or halogen. In some cases, R² is H. In some cases, each of R² and R⁴ is H.

In various cases, each R⁶ and R⁷ can independently be H, C₁₋₆alkyl, C(O)—C₁₋₆alkyl, spiro or bicyclic C₈₋₁₄cycloalkyl, 8-14 membered heterocycle comprising 1, 2, or 3 ring heteroatoms selected from N, O, and S, and when R⁶ or R⁷ is other than H, it can be optionally substituted with 1, 2, or 3 substituents independently selected from the group consisting of halogen, CN, OH, ═O, SO₂, C₀₋₆alkylene-NH₂, C₀₋₆alkylene-NH(C₁₋₆alkyl), C₀₋₆alkylene-N(C₁₋₆alkyl)₂, C₀₋₆alkylene-SO₂C₁₋₆alkyl, C₁₋₆alkyl and C₁₋₆alkoxy, or R⁶ and R⁷, together with the nitrogen to which they are attached, form a 4-10 membered heterocycle comprising 0-2 additional ring heteroatoms independently selected from N, O, and S. In various cases, each R⁶ and R⁷ are independently H, C₁₋₆alkyl, or C(O)—C₁₋₆alkyl. In some cases, each R⁶ and R⁷ are independently H or C₁₋₆alkyl. In various cases, at least one R⁶ and R⁷, together with the nitrogen to which they are attached, form a 4-10 membered heterocycle comprising 0-2 additional ring heteroatoms independently selected from N and O.

In various cases, R³ can be halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, wherein the heterocycle and heteroaryl each comprises 1, 2, or 3 ring heteroatoms selected from N, O, and S, and when R³ is —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, it is optionally substituted with 1, 2, or 3 R^{3a} substituents. In various cases, R³ is —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, and is optionally substituted with 1, 2, or 3 R^{3a}. In many cases, at least one of R¹, R², R³, and R⁴ is halogen. In some cases, at least one of R¹, R², R³, and R⁴ is F. In various cases, R³ is

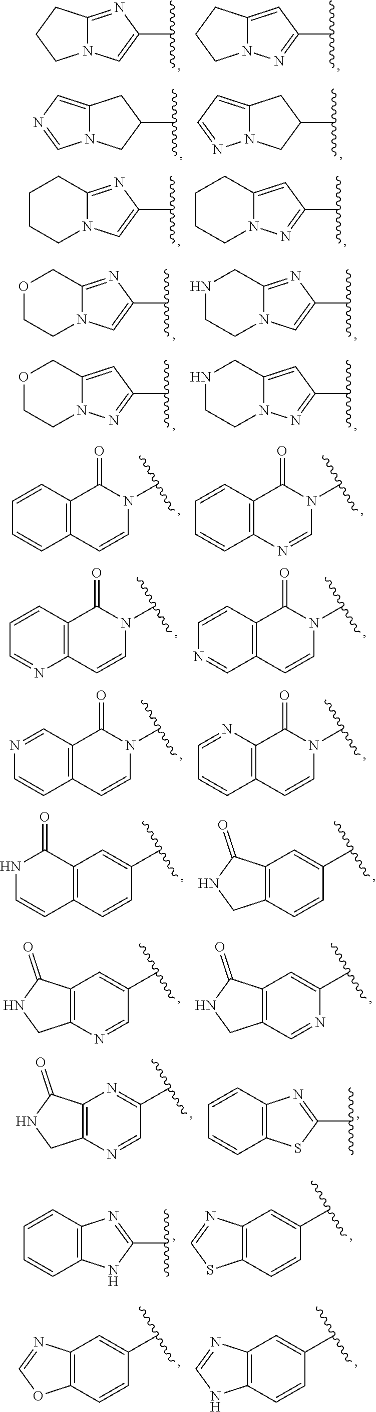

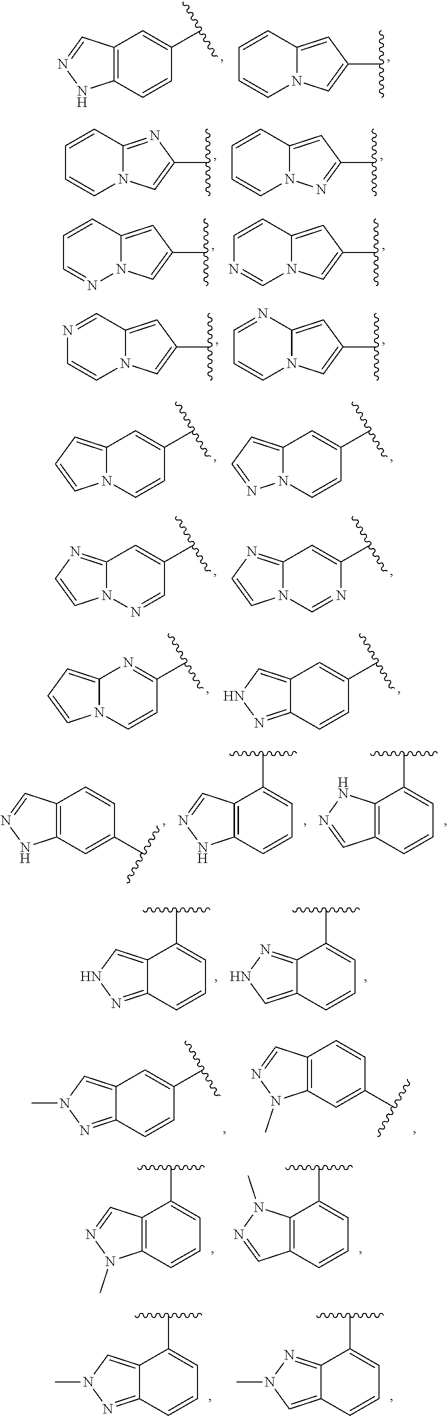

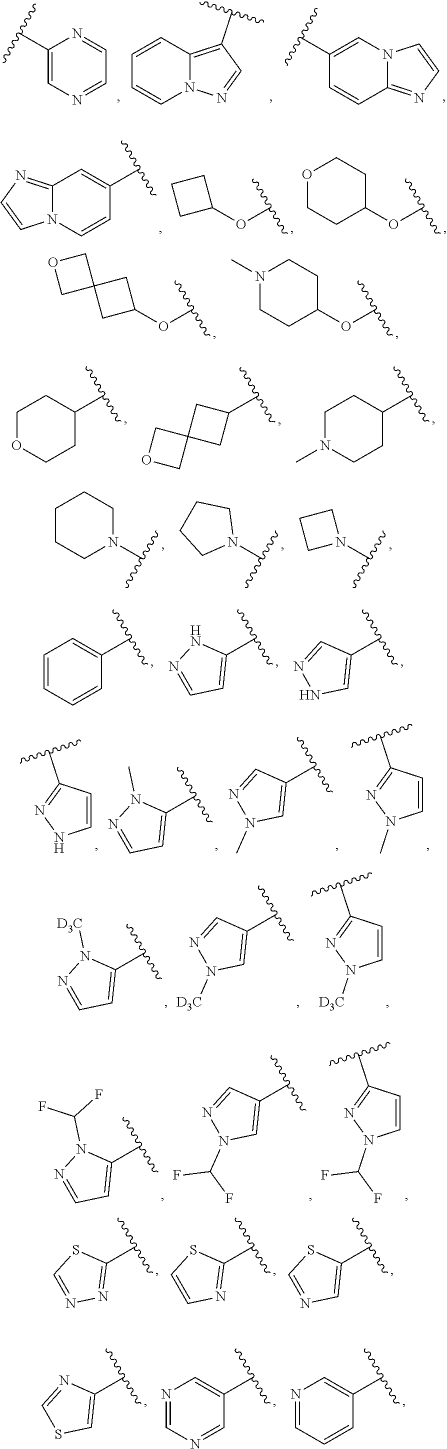

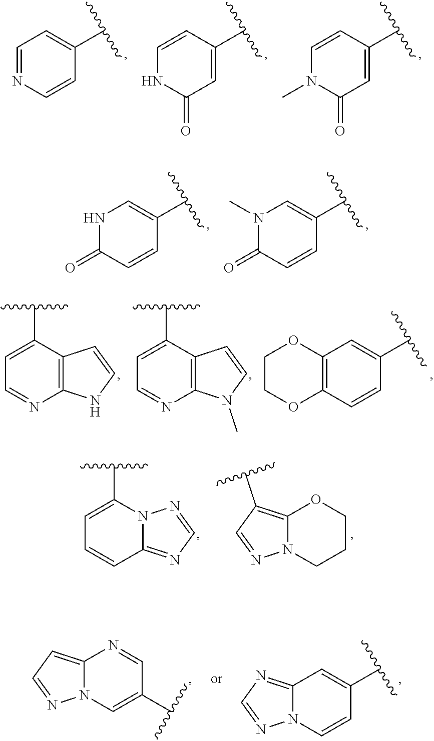

and is optionally substituted with 1, 2, or 3 R^{3a}. In some cases, R³ is

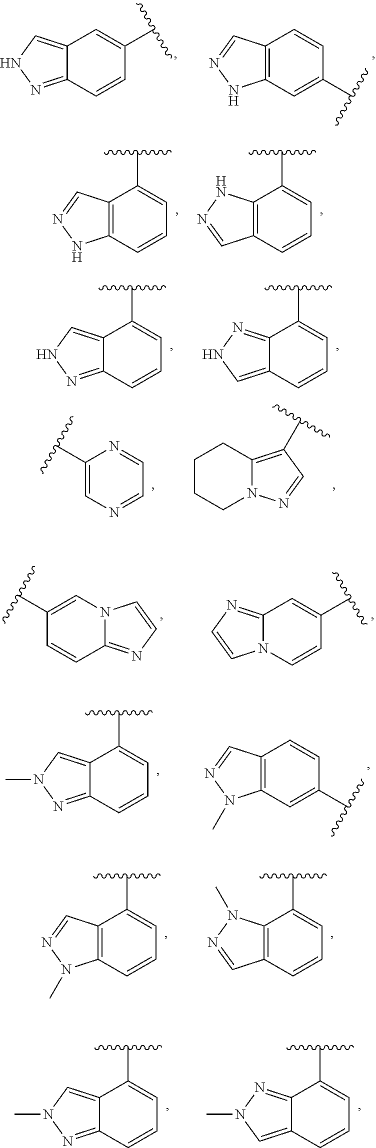

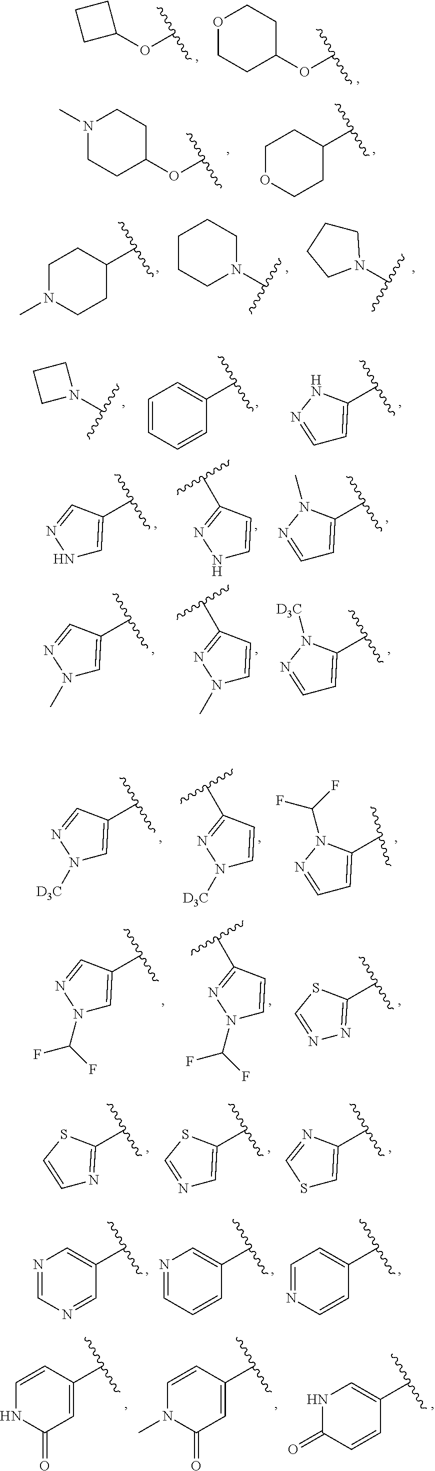

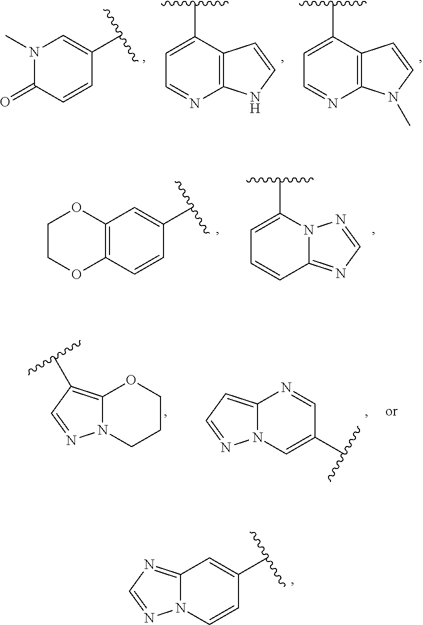

and is optionally substituted with 1, 2, or 3 R^{3a}. In some cases, R³ is C₃₋₆ cycloalkyl, 5-10 membered heteroaryl, or 4-8 membered heterocycle, optionally substituted with 1, 2, or 3 R^{3a}. In some cases, R³ is 5-6-membered heterocycle comprising 1 ring heteroatom selected from S and O, or 5-6-membered heteroaryl comprising 2 or 3 ring heteroatoms independently selected from N and S, and is optionally substituted with 1 or 2 R^{3a} substituents independently selected from halogen, CN, OH, and C₁₋₆alkyl.

In compounds of Formula (I), R³ can be optionally substituted with 1, 2, or 3 R^{3a} groups. In many cases, R³ is unsubstituted. In many cases, R³ is substituted with 1 or 2 R^{3a}. In some cases, R³ is substituted with 1 R^{3a}. In compounds of Formula (I), each R^{3a} can independently be selected from halogen, CN, OH, ═O, SO₂, C₁₋₆alkyl, C₂₋₁₀alkene, C₁₋₆hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-NH₂, C₀₋₆alkylene-NH(C₁₋₆alkyl), C₀₋₆alkylene-N(C₁₋₆alkyl)₂, —S—C₁₋₆alkyl, C₀₋₆alkylene-SO₂C₁₋₆alkyl, C₀₋₆alkylene-C(O)NH₂, C₀₋₆alkylene-C(O)NH(C₁₋₆alkyl), C₀₋₆alkylene-C(O)N(C₁₋₆alkyl)₂, C₀₋₆alkylene-NHC(O)C₁₋₆alkyl, C₀₋₆alkylene-COOH, and C₀₋₆alkylene-3-6 membered heterocycle comprising 1, 2, or 3 heteroatoms selected from N, O, and S. In various cases, at least one R^{3a} is halogen, CN, OH, ═O, SO₂, C₁₋₆alkyl, C₂₋₁₀alkene, C₁₋₆ hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-N(C₁₋₆alkyl)₂, —S—C₁₋₆ alkyl, C₀₋₆alkylene-NHC(O)C₁₋₆alkyl, or C₀₋₆alkylene-3-6 membered heterocycle comprising 1, 2, or 3 heteroatoms selected from N, O, and S. In many cases, at least one R^{3a} is CH₃, CH₂CH₃, CH(CH₃)₂, CH₂CH(CH₃)₂, CF₃, CH₂CH₂F, CH₂CHF₂, CH₂OH, C(CH₃)₂OH, CH₂OCH₃, CH₂CH₂OCH₃, CH₂OCH₂CH₃, F, CN, ═O, SO₂, OH, OCH₃, OCH₂CH₃, OCH(CH₃)₂, OCHF₂, SCH₃, N(CH₃)₂, NHCOCH₃, CD₃,

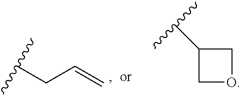

In some cases, at least one R^{3a} is CH₃, CH₂CH₃, F, CN, OH, OCH₃, CF₃, CH₂OH, or OCHF₂.

In some cases, the compound has a structure of Formula (If):

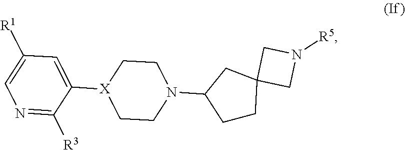

wherein R¹ is halogen; R³ is —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, wherein the heterocycle and heteroaryl each comprises 1, 2, or 3 ring heteroatoms selected from N, O, and S, and R³ is substituted with 0, 1, 2, or 3 R^{3a} substituents; R⁵ is —CO₂—Z, or a bioisostere thereof, and Z is C₁₋₇alkyl, C₁₋₇haloalkyl, C₃₋₆cycloalkyl, or C₂₋₆alkyne, and is optionally substituted with C₁₋₆alkoxy or C₃₋₆cycloalkyl. In some cases, R¹ is Cl or F. In some cases, R³ is 5-6-membered heterocycle comprising 1 ring heteroatom selected from S and O, or 5-6-membered heteroaryl comprising 2 or 3 ring heteroatoms independently selected from N and S, and is substituted with 0, 1 or 2 R^{3a} substituents independently selected from halogen, CN, OH, and C₁₋₆alkyl. In various cases, R⁵ is CO₂C₁₋₇alkyl.

Examples of compounds according to Formulae (I), (Ia), (Ib), (Ic), (Id), (Ie), and (If) of the disclosure are shown in Table A.

TABLE A

| | Structure |
|---|---|
| A1 | 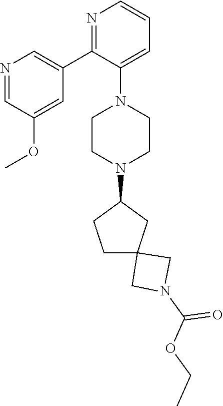 [SMILES: CCOC(=O)N1CC2(CC[C@@H](N3CCN(c4cccnc4-c4cncc(OC)c4)CC3)C2)C1] |
| A2 | 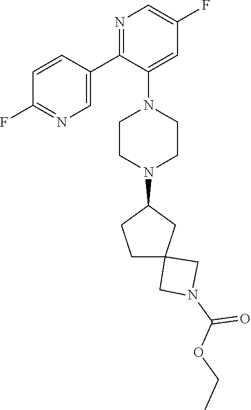 [SMILES: CCOC(=O)N1CC2(CC[C@@H](N3CCN(c4cc(F)cnc4-c4ccc(F)nc4)CC3)C2)C1] |
| A3 | 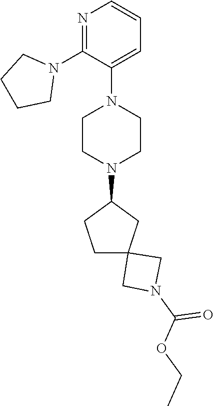 [SMILES: CCOC(=O)N1CC2(CC[C@@H](N3CCN(c4cccnc4N4CCCC4)CC3)C2)C1] |
| A4 | 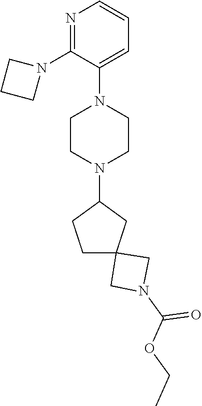 [SMILES: CCOC(=O)N1CC2(CCC(N3CCN(c4cccnc4N4CCC4)CC3)C2)C1] |
| A5 | 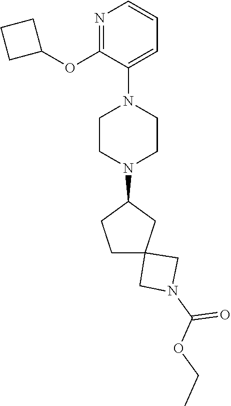 [SMILES: CCOC(=O)N1CC2(CC[C@@H](N3CCN(c4cccnc4OC4CCC4)CC3)C2)C1] |
| A6 | 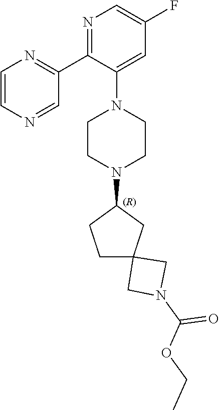 |
| A7 | 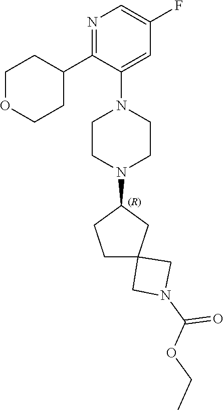 |
| A8 | 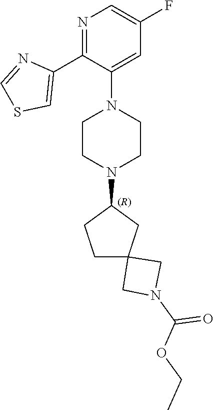 |
| A9 | 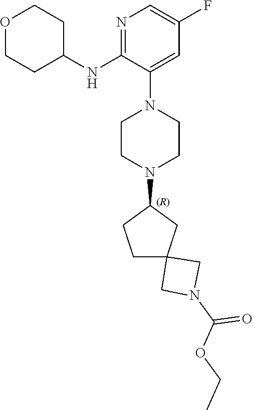 |
| A10 | 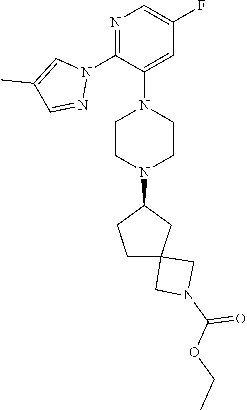 [SMILES: CCOC(=O)N1CC2(CC[C@@H](N3CCN(c4cc(F)cnc4-n4cc(C)cn4)CC3)C2)C1] |
| A11 | 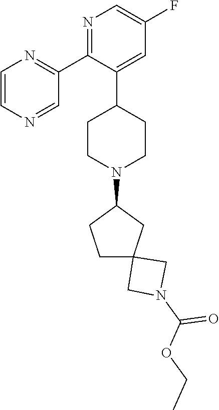 [SMILES: CCOC(=O)N1CC2(CC[C@@H](N3CCC(c4cc(F)cnc4-c4cnccn4)CC3)C2)C1] |
| A12 | 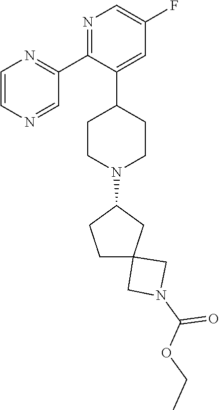 [SMILES: CCOC(=O)N1CC2(CC[C@H](N3CCC(c4cc(F)cnc4-c4cnccn4)CC3)C2)C1] |
| A13 | 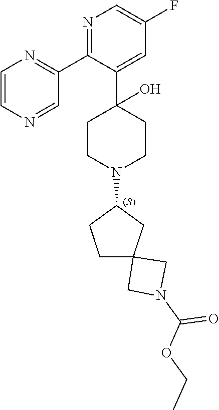 |
| A14 | 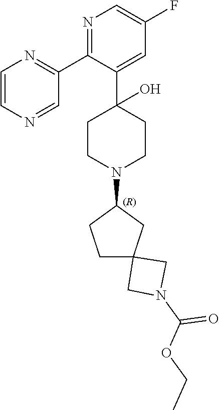 |
| A15 | 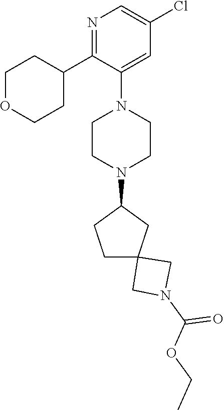 [SMILES: CCOC(=O)N1CC2(CC[C@@H](N3CCN(c4cc(Cl)cnc4C4CCOCC4)CC3)C2)C1] |
| A16 | 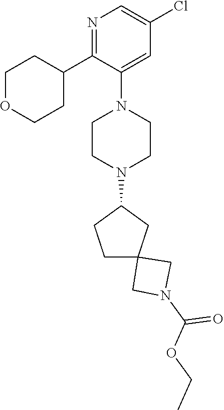 [SMILES: CCOC(=O)N1CC2(CC[C@H](N3CCN(c4cc(Cl)cnc4C4CCOCC4)CC3)C2)C1] |
| A17 | 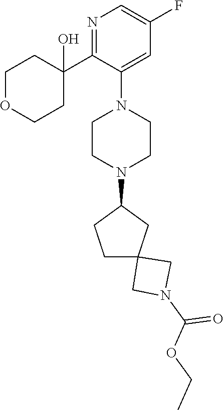 [SMILES: CCOC(=O)N1CC2(CC[C@@H](N3CCN(c4cc(F)cnc4C4(O)CCOCC4)CC3)C2)C1] |
| A18 | 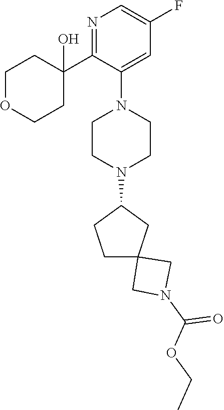 [SMILES: CCOC(=O)N1CC2(CC[C@H](N3CCN(c4cc(F)cnc4C4(O)CCOCC4)CC3)C2)C1] |
| A19 | 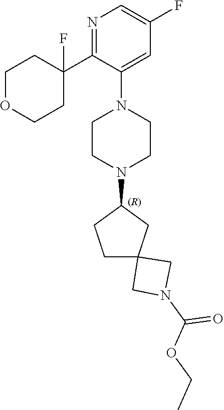 |
| A20 | 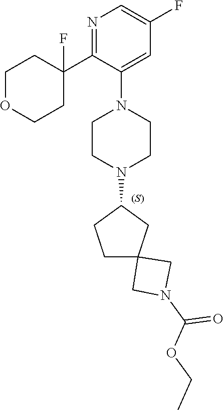 |
| A21 | 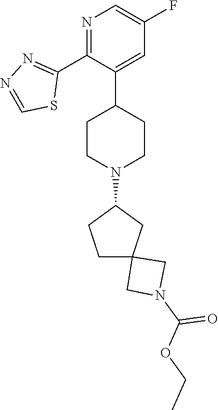 [SMILES: CCOC(=O)N1CC2(CC[C@H](N3CCC(c4cc(F)cnc4-c4nncs4)CC3)C2)C1] |
| A22 | 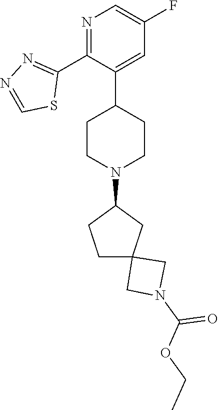 [SMILES: CCOC(=O)N1CC2(CC[C@@H](N3CCC(c4cc(F)cnc4-c4nncs4)CC3)C2)C1] |
| A23 | 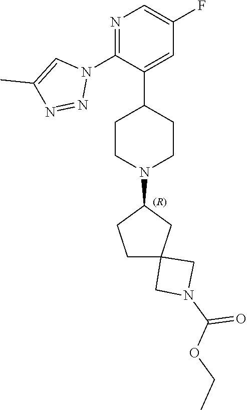 |
| A24 | 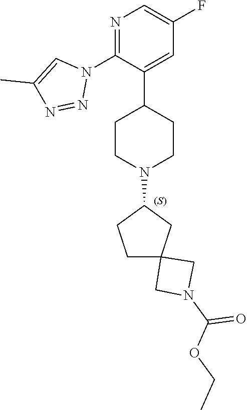 |
| A25 | 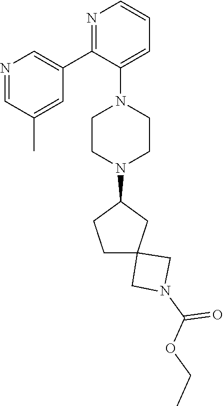 [SMILES: CCOC(=O)N1CC2(CC[C@@H](N3CCN(c4cccnc4-c4cncc(C)c4)CC3)C2)C1] |
| A26 | 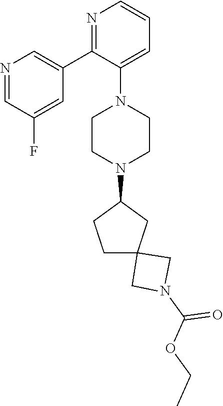 [SMILES: CCOC(=O)N1CC2(CC[C@@H](N3CCN(c4cccnc4-c4cncc(F)c4)CC3)C2)C1] |
| A27 | 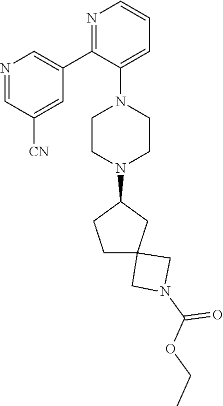 [SMILES: CCOC(=O)N1CC2(CC[C@@H](N3CCN(c4cccnc4-c4cncc(C#N)c4)CC3)C2)C1] |
| A28 | 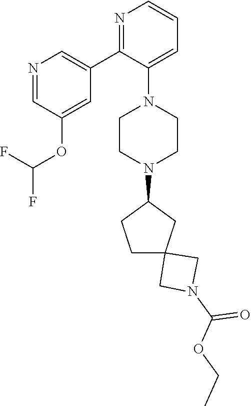 [SMILES: CCOC(=O)N1CC2(CC[C@@H](N3CCN(c4cccnc4-c4cncc(OC(F)F)c4)CC3)C2)C1] |
| A29 | 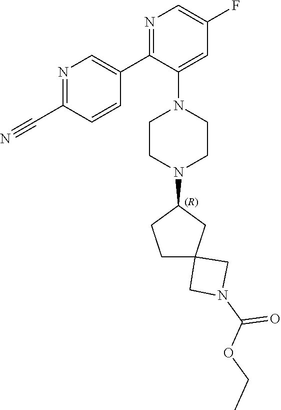 |
| A30 | 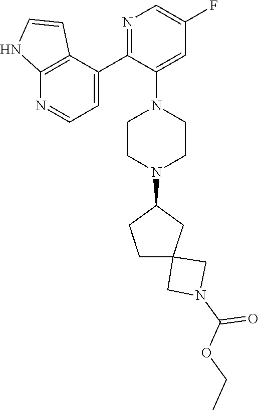 [SMILES: CCOC(=O)N1CC2(CC[C@@H](N3CCN(c4cc(F)cnc4-c4ccnc5[nH]ccc45)CC3)C2)C1] |
| A31 | 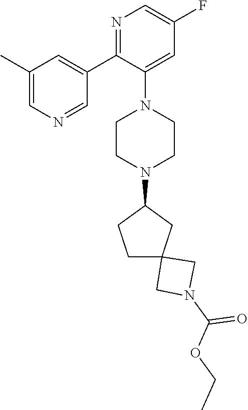 [SMILES: CCOC(=O)N1CC2(CC[C@@H](N3CCN(c4cc(F)cnc4-c4cncc(C)c4)CC3)C2)C1] |
| A32 | 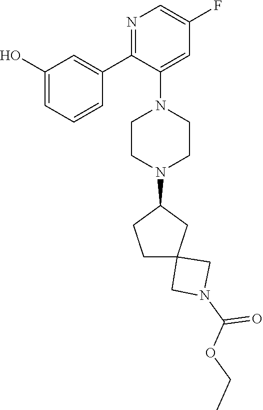 [SMILES: CCOC(=O)N1CC2(CC[C@@H](N3CCN(c4cc(F)cnc4-c4cccc(O)c4)CC3)C2)C1] |
| A33 | 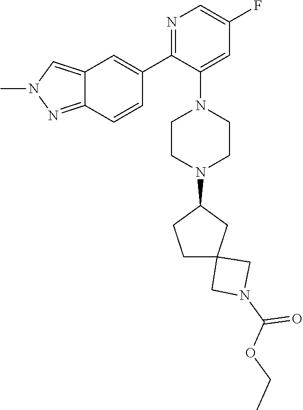 [SMILES: CCOC(=O)N1CC2(CC[C@@H](N3CCN(c4cc(F)cnc4-c4ccc5nn(C)cc5c4)CC3)C2)C1] |
| A34 | 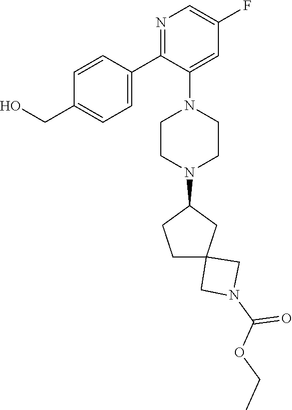 [SMILES: CCOC(=O)N1CC2(CC[C@@H](N3CCN(c4cc(F)cnc4-c4ccc(CO)cc4)CC3)C2)C1] |
| A35 | 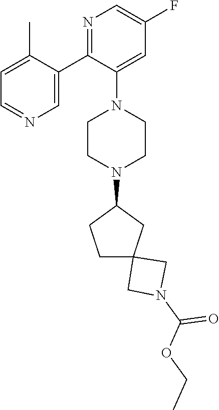 [SMILES: CCOC(=O)N1CC2(CC[C@@H](N3CCN(c4cc(F)cnc4-c4cnccc4C)CC3)C2)C1] |
| A36 | 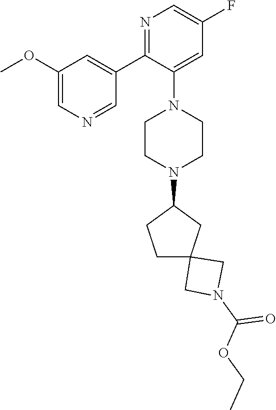 [SMILES: CCOC(=O)N1CC2(CC[C@@H](N3CCN(c4cc(F)cnc4-c4cncc(OC)c4)CC3)C2)C1] |
| A37 | 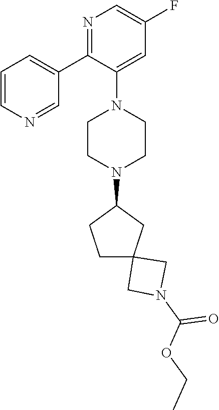 [SMILES: CCOC(=O)N1CC2(CC[C@@H](N3CCN(c4cc(F)cnc4-c4cccnc4)CC3)C2)C1] |
| A38 | 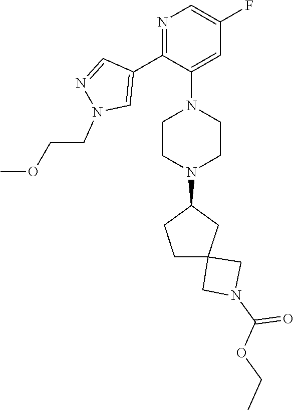 [SMILES: CCOC(=O)N1CC2(CC[C@@H](N3CCN(c4cc(F)cnc4-c4cnn(CCOC)c4)CC3)C2)C1] |
| A39 | 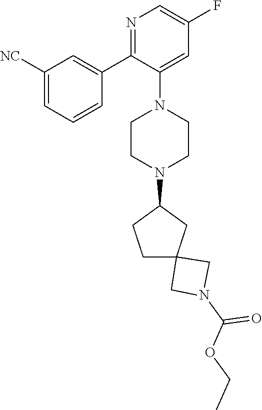 [SMILES: CCOC(=O)N1CC2(CC[C@@H](N3CCN(c4cc(F)cnc4-c4cccc(C#N)c4)CC3)C2)C1] |
| A40 | 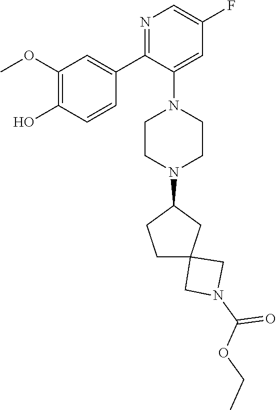 [SMILES: CCOC(=O)N1CC2(CC[C@@H](N3CCN(c4cc(F)cnc4-c4ccc(O)c(OC)c4)CC3)C2)C1] |
| A41 | 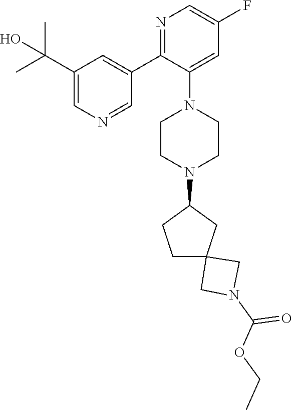 [SMILES: CCOC(=O)N1CC2(CC[C@@H](N3CCN(c4cc(F)cnc4-c4cncc(C(C)(C)O)c4)CC3)C2)C1] |
| A42 | 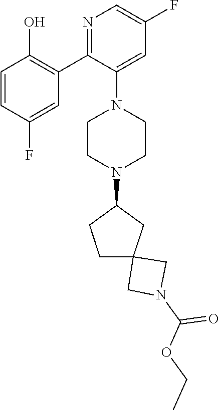 [SMILES: CCOC(=O)N1CC2(CC[C@@H](N3CCN(c4cc(F)cnc4-c4cc(F)ccc4O)CC3)C2)C1] |
| A43 | 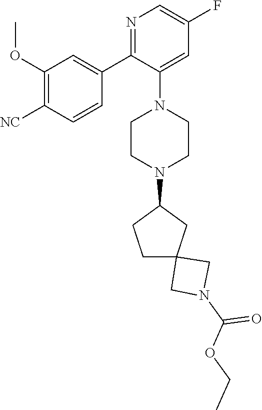 [SMILES: CCOC(=O)N1CC2(CC[C@@H](N3CCN(c4cc(F)cnc4-c4ccc(C#N)c(OC)c4)CC3)C2)C1] |
| A44 | 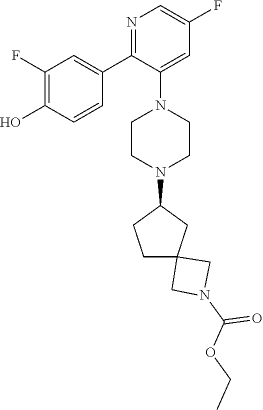 [SMILES: CCOC(=O)N1CC2(CC[C@@H](N3CCN(c4cc(F)cnc4-c4ccc(O)c(F)c4)CC3)C2)C1] |
| A45 | 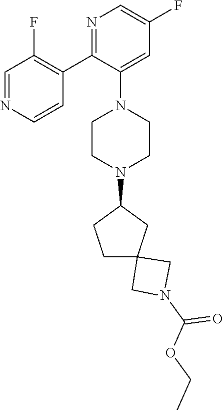 [SMILES: CCOC(=O)N1CC2(CC[C@@H](N3CCN(c4cc(F)cnc4-c4ccncc4F)CC3)C2)C1] |
| A46 | 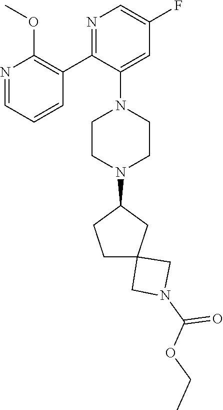 [SMILES: CCOC(=O)N1CC2(CC[C@@H](N3CCN(c4cc(F)cnc4-c4cccnc4OC)CC3)C2)C1] |
| A47 | 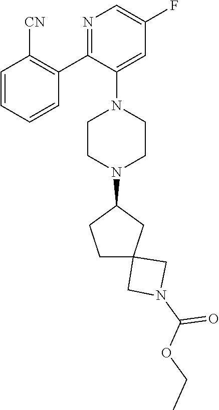 [SMILES: CCOC(=O)N1CC2(CC[C@@H](N3CCN(c4cc(F)cnc4-c4ccccc4C#N)CC3)C2)C1] |
| A48 | 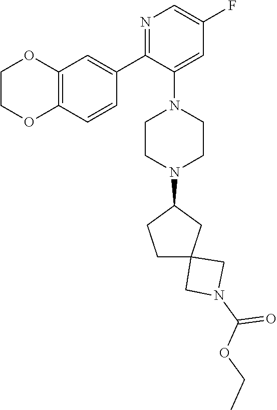 [SMILES: CCOC(=O)N1CC2(CC[C@@H](N3CCN(c4cc(F)cnc4-c4ccc5c(c4)OCCO5)CC3)C2)C1] |
| A49 | 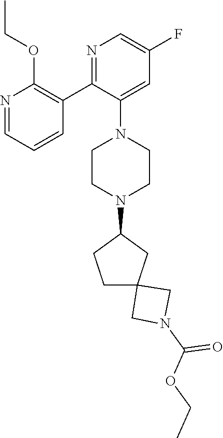 [SMILES: CCOC(=O)N1CC2(CC[C@@H](N3CCN(c4cc(F)cnc4-c4cccnc4OCC)CC3)C2)C1] |
| A50 | 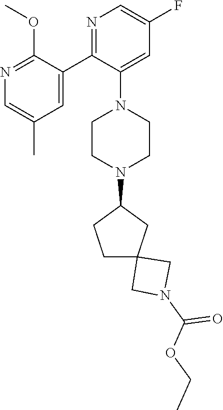 [SMILES: CCOC(=O)N1CC2(CC[C@@H](N3CCN(c4cc(F)cnc4-c4cc(C)cnc4OC)CC3)C2)C1] |
| A51 | 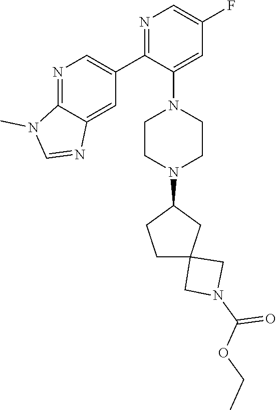 [SMILES: CCOC(=O)N1CC2(CC[C@@H](N3CCN(c4cc(F)cnc4-c4cnc5c(c4)ncn5C)CC3)C2)C1] |
| A52 | 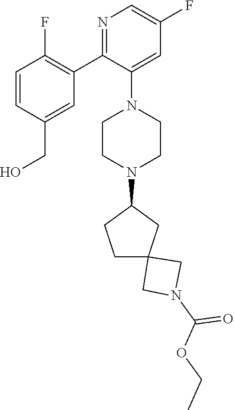 [SMILES: CCOC(=O)N1CC2(CC[C@@H](N3CCN(c4cc(F)cnc4-c4cc(CO)ccc4F)CC3)C2)C1] |
| A53 | 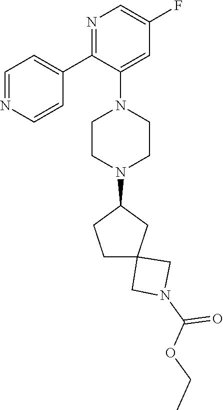 [SMILES: CCOC(=O)N1CC2(CC[C@@H](N3CCN(c4cc(F)cnc4-c4ccncc4)CC3)C2)C1] |
| A54 | 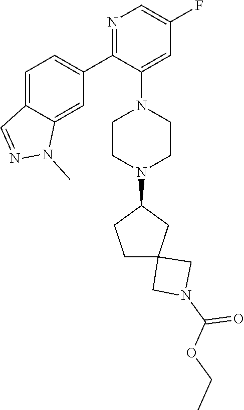 [SMILES: CCOC(=O)N1CC2(CC[C@@H](N3CCN(c4cc(F)cnc4-c4ccc5cnn(C)c5c4)CC3)C2)C1] |
| A55 | 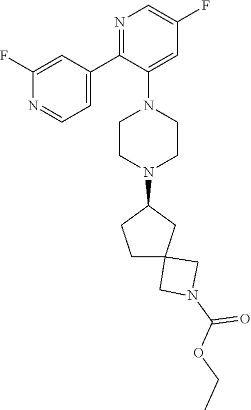 [SMILES: CCOC(=O)N1CC2(CC[C@@H](N3CCN(c4cc(F)cnc4-c4ccnc(F)c4)CC3)C2)C1] |
| A56 | 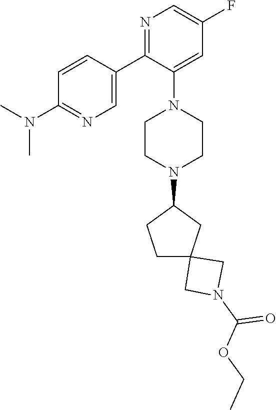 [SMILES: CCOC(=O)N1CC2(CC[C@@H](N3CCN(c4cc(F)cnc4-c4ccc(N(C)C)nc4)CC3)C2)C1] |
| A57 | 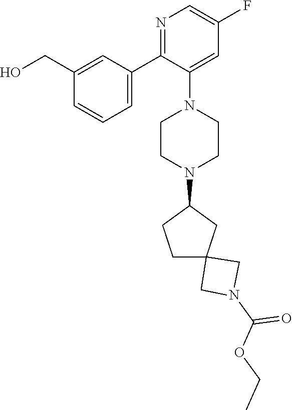 [SMILES: CCOC(=O)N1CC2(CC[C@@H](N3CCN(c4cc(F)cnc4-c4cccc(CO)c4)CC3)C2)C1] |
| A58 | 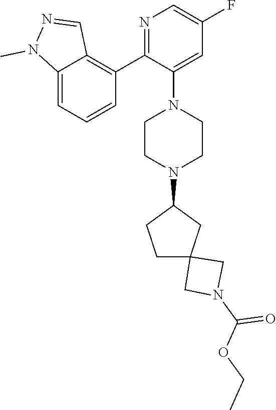 [SMILES: CCOC(=O)N1CC2(CC[C@@H](N3CCN(c4cc(F)cnc4-c4cccc5c4cnn5C)CC3)C2)C1] |
| A59 | 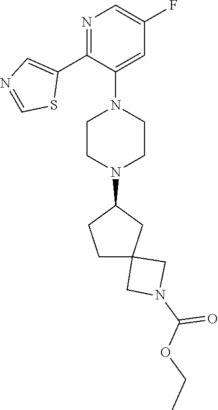 [SMILES: CCOC(=O)N1CC2(CC[C@@H](N3CCN(c4cc(F)cnc4-c4cncs4)CC3)C2)C1] |
| A60 | 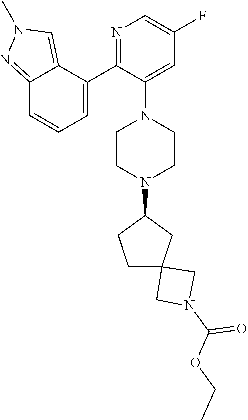 [SMILES: CCOC(=O)N1CC2(CC[C@@H](N3CCN(c4cc(F)cnc4-c4cccc5nn(C)cc45)CC3)C2)C1] |
| A61 | 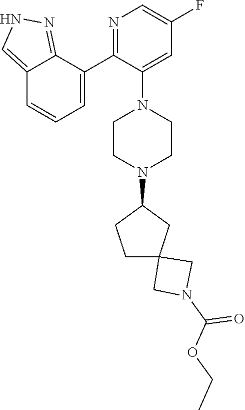 [SMILES: CCOC(=O)N1CC2(CC[C@@H](N3CCN(c4cc(F)cnc4-c4cccc5c[nH]nc45)CC3)C2)C1] |
| A62 | 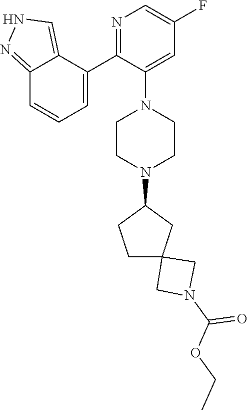 [SMILES: CCOC(=O)N1CC2(CC[C@@H](N3CCN(c4cc(F)cnc4-c4cccc5n[nH]cc45)CC3)C2)C1] |
| A63 | 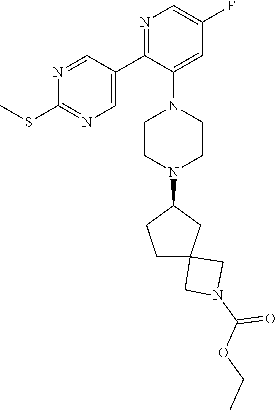 [SMILES: CCOC(=O)N1CC2(CC[C@@H](N3CCN(c4cc(F)cnc4-c4cnc(SC)nc4)CC3)C2)C1] |
| A64 | 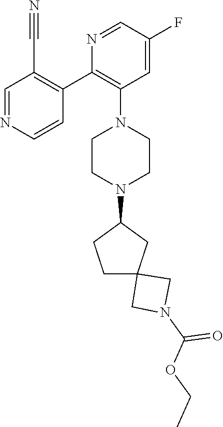 [SMILES: CCOC(=O)N1CC2(CC[C@@H](N3CCN(c4cc(F)cnc4-c4ccncc4C#N)CC3)C2)C1] |
| A65 | 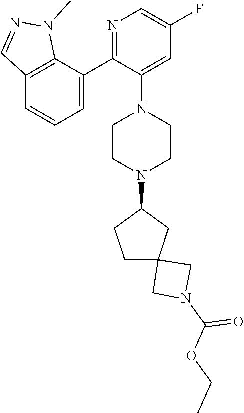 [SMILES: CCOC(=O)N1CC2(CC[C@@H](N3CCN(c4cc(F)cnc4-c4cccc5cnn(C)c45)CC3)C2)C1] |
| A66 | 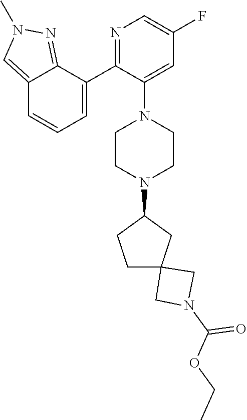 [SMILES: CCOC(=O)N1CC2(CC[C@@H](N3CCN(c4cc(F)cnc4-c4cccc5cn(C)nc45)CC3)C2)C1] |
| A67 | 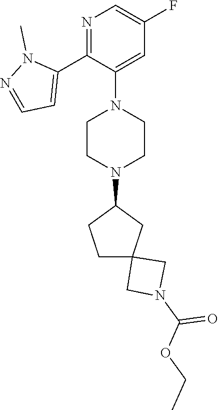 [SMILES: CCOC(=O)N1CC2(CC[C@@H](N3CCN(c4cc(F)cnc4-c4ccnn4C)CC3)C2)C1] |
| A68 | 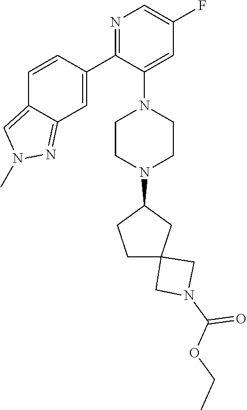 [SMILES: CCOC(=O)N1CC2(CC[C@@H](N3CCN(c4cc(F)cnc4-c4ccc5cn(C)nc5c4)CC3)C2)C1] |
| A69 | 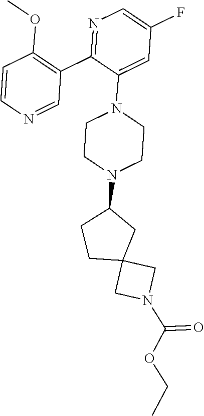 [SMILES: CCOC(=O)N1CC2(CC[C@@H](N3CCN(c4cc(F)cnc4-c4cnccc4OC)CC3)C2)C1] |
| A70 | 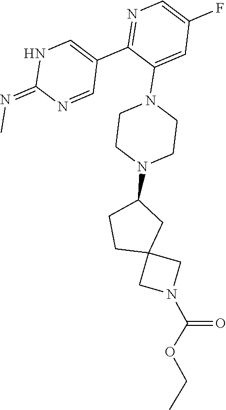 [SMILES: CCOC(=O)N1CC2(CC[C@@H](N3CCN(c4cc(F)cnc4-c4cn/c(=N\C)[nH]c4)CC3)C2)C1] |
| A71 | 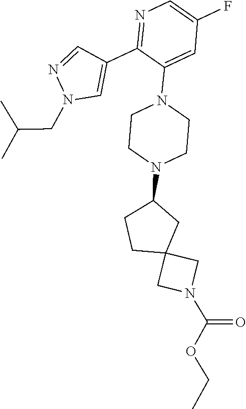 [SMILES: CCOC(=O)N1CC2(CC[C@@H](N3CCN(c4cc(F)cnc4-c4cnn(CC(C)C)c4)CC3)C2)C1] |
| A72 | 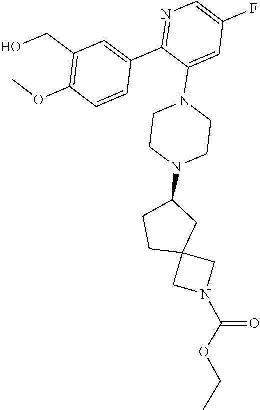 [SMILES: CCOC(=O)N1CC2(CC[C@@H](N3CCN(c4cc(F)cnc4-c4ccc(OC)c(CO)c4)CC3)C2)C1] |
| A73 | 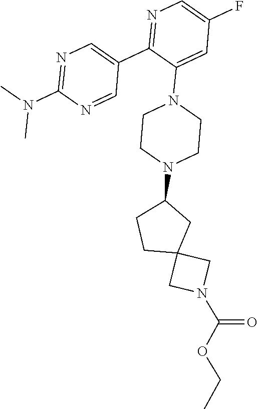 [SMILES: CCOC(=O)N1CC2(CC[C@@H](N3CCN(c4cc(F)cnc4-c4cnc(N(C)C)nc4)CC3)C2)C1] |
| A74 | 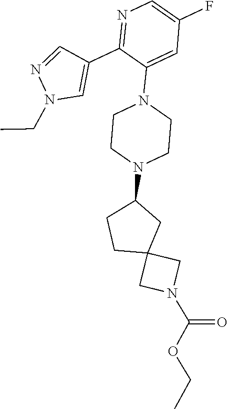 [SMILES: CCOC(=O)N1CC2(CC[C@@H](N3CCN(c4cc(F)cnc4-c4cnn(CC)c4)CC3)C2)C1] |
| A75 | 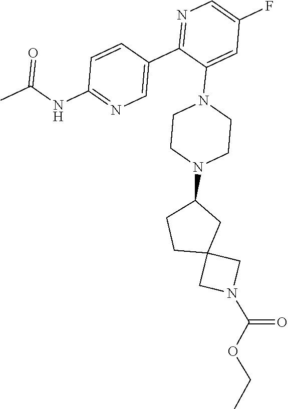 [SMILES: CCOC(=O)N1CC2(CC[C@@H](N3CCN(c4cc(F)cnc4-c4ccc(NC(C)=O)nc4)CC3)C2)C1] |
| A76 | 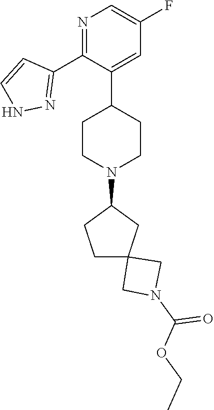 [SMILES: CCOC(=O)N1CC2(CC[C@@H](N3CCC(c4cc(F)cnc4-c4cc[nH]n4)CC3)C2)C1] |
| A77 | 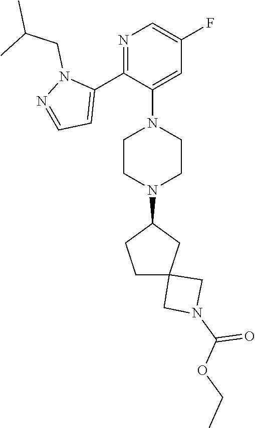 [SMILES: CCOC(=O)N1CC2(CC[C@@H](N3CCN(c4cc(F)cnc4-c4ccnn4CC(C)C)CC3)C2)C1] |
| A78 | 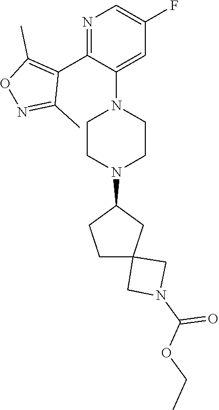 [SMILES: CCOC(=O)N1CC2(CC[C@@H](N3CCN(c4cc(F)cnc4-c4c(C)noc4C)CC3)C2)C1] |
| A79 | 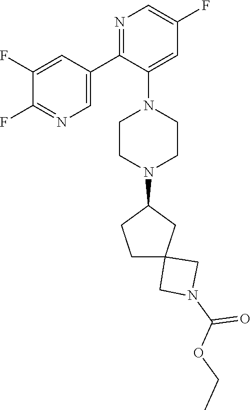 [SMILES: CCOC(=O)N1CC2(CC[C@@H](N3CCN(c4cc(F)cnc4-c4cnc(F)c(F)c4)CC3)C2)C1] |
| A80 | 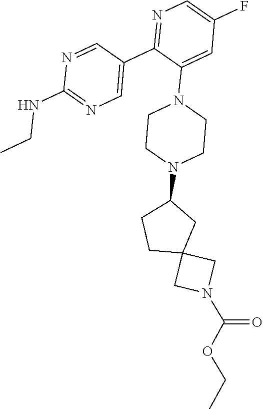 [SMILES: CCNc1ncc(-c2ncc(F)cc2N2CCN([C@@H]3CCC4(C3)CN(C(=O)OCC)C4)CC2)cn1] |
| A81 | 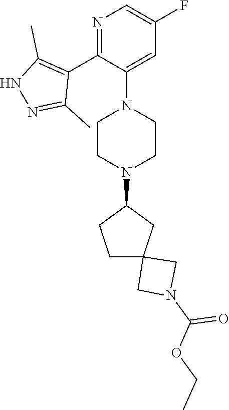 [SMILES: CCOC(=O)N1CC2(CC[C@@H](N3CCN(c4cc(F)cnc4-c4c(C)n[nH]c4C)CC3)C2)C1] |
| A82 | 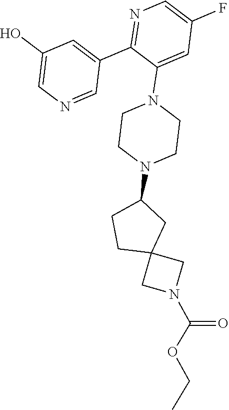 [SMILES: CCOC(=O)N1CC2(CC[C@@H](N3CCN(c4cc(F)cnc4-c4cncc(O)c4)CC3)C2)C1] |
| A83 | 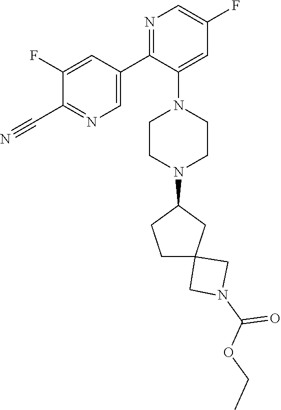 [SMILES: CCOC(=O)N1CC2(CC[C@@H](N3CCN(c4cc(F)cnc4-c4cnc(C#N)c(F)c4)CC3)C2)C1] |
| A84 | 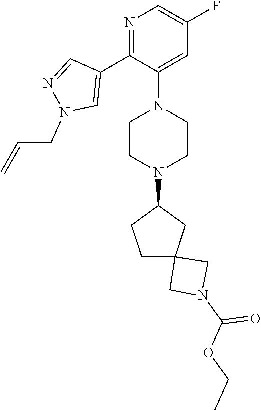 [SMILES: C=CCn1cc(-c2ncc(F)cc2N2CCN([C@@H]3CCC4(C3)CN(C(=O)OCC)C4)CC2)cn1] |
| A85 | 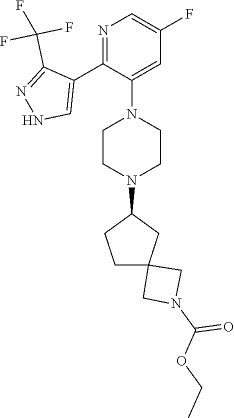 [SMILES: CCOC(=O)N1CC2(CC[C@@H](N3CCN(c4cc(F)cnc4-c4c[nH]nc4C(F)(F)F)CC3)C2)C1] |
| A86 | 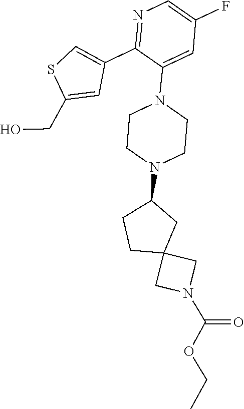 [SMILES: CCOC(=O)N1CC2(CC[C@@H](N3CCN(c4cc(F)cnc4-c4csc(CO)c4)CC3)C2)C1] |
| A87 | 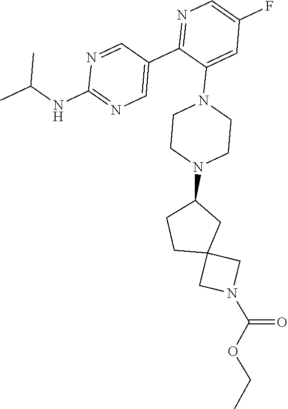 [SMILES: CCOC(=O)N1CC2(CC[C@@H](N3CCN(c4cc(F)cnc4-c4cnc(NC(C)C)nc4)CC3)C2)C1] |
| A88 | 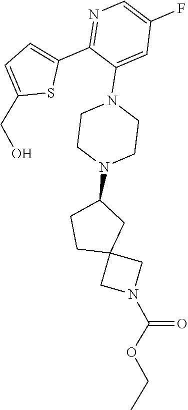 [SMILES: CCOC(=O)N1CC2(CC[C@@H](N3CCN(c4cc(F)cnc4-c4ccc(CO)s4)CC3)C2)C1] |
| A89 | 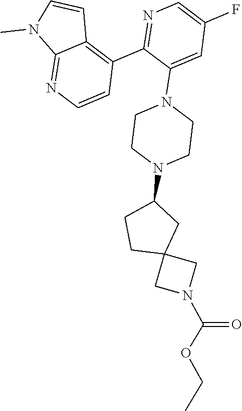 [SMILES: CCOC(=O)N1CC2(CC[C@@H](N3CCN(c4cc(F)cnc4-c4ccnc5c4ccn5C)CC3)C2)C1] |
| A90 | 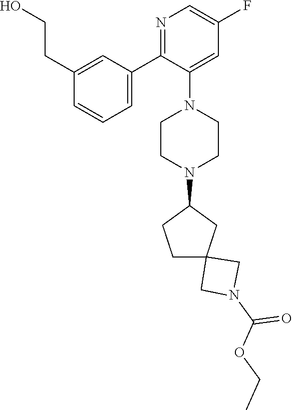 [SMILES: CCOC(=O)N1CC2(CC[C@@H](N3CCN(c4cc(F)cnc4-c4cccc(CCO)c4)CC3)C2)C1] |
| A91 | 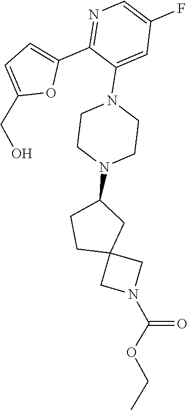 [SMILES: CCOC(=O)N1CC2(CC[C@@H](N3CCN(c4cc(F)cnc4-c4ccc(CO)o4)CC3)C2)C1] |
| A92 | 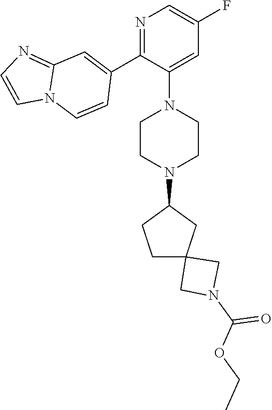 [SMILES: CCOC(=O)N1CC2(CC[C@@H](N3CCN(c4cc(F)cnc4-c4ccn5ccnc5c4)CC3)C2)C1] |
| A93 | 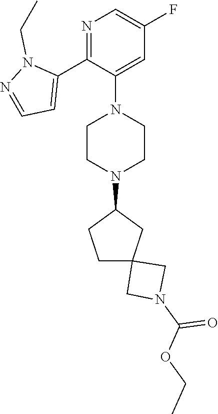 [SMILES: CCOC(=O)N1CC2(CC[C@@H](N3CCN(c4cc(F)cnc4-c4ccnn4CC)CC3)C2)C1] |
| A94 | 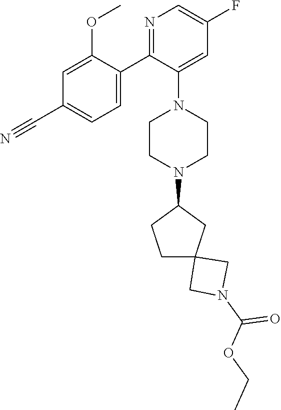 [SMILES: CCOC(=O)N1CC2(CC[C@@H](N3CCN(c4cc(F)cnc4-c4ccc(C#N)cc4OC)CC3)C2)C1] |
| A95 | 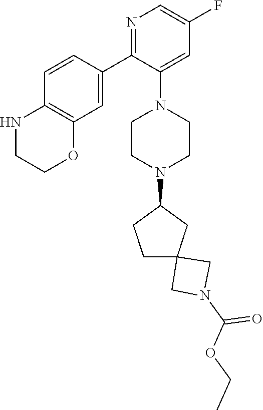 [SMILES: CCOC(=O)N1CC2(CC[C@@H](N3CCN(c4cc(F)cnc4-c4ccc5c(c4)OCCN5)CC3)C2)C1] |
| A96 | 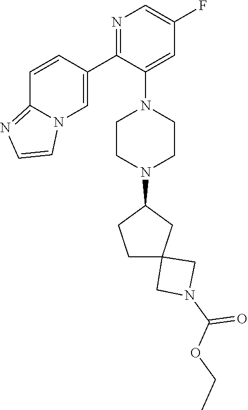 [SMILES: CCOC(=O)N1CC2(CC[C@@H](N3CCN(c4cc(F)cnc4-c4ccc5nccn5c4)CC3)C2)C1] |
| A97 | 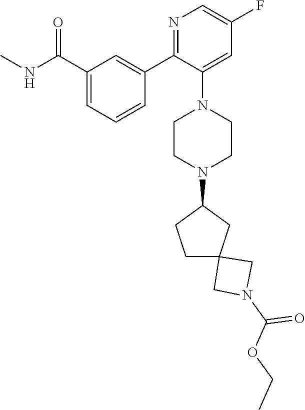 [SMILES: CCOC(=O)N1CC2(CC[C@@H](N3CCN(c4cc(F)cnc4-c4cccc(C(=O)NC)c4)CC3)C2)C1] |
| A98 | 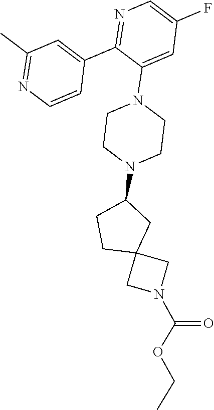 [SMILES: CCOC(=O)N1CC2(CC[C@@H](N3CCN(c4cc(F)cnc4-c4ccnc(C)c4)CC3)C2)C1] |
| A99 | 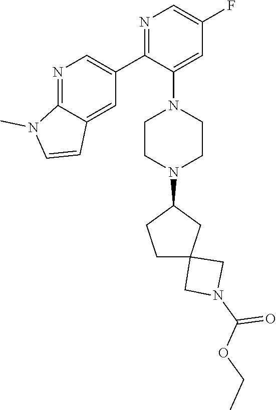 [SMILES: CCOC(=O)N1CC2(CC[C@@H](N3CCN(c4cc(F)cnc4-c4cnc5c(ccn5C)c4)CC3)C2)C1] |
| A100 | 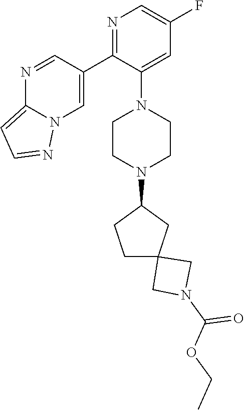 [SMILES: CCOC(=O)N1CC2(CC[C@@H](N3CCN(c4cc(F)cnc4-c4cnc5ccnn5c4)CC3)C2)C1] |
| A101 | 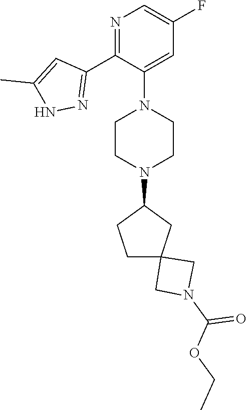 [SMILES: CCOC(=O)N1CC2(CC[C@@H](N3CCN(c4cc(F)cnc4-c4cc(C)[nH]n4)CC3)C2)C1] |
| A102 | 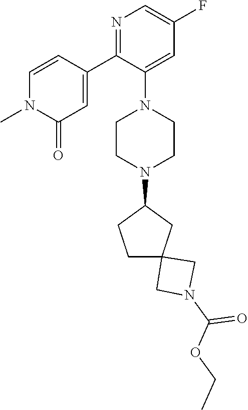 [SMILES: CCOC(=O)N1CC2(CC[C@@H](N3CCN(c4cc(F)cnc4-c4ccn(C)c(=O)c4)CC3)C2)C1] |
| A103 | 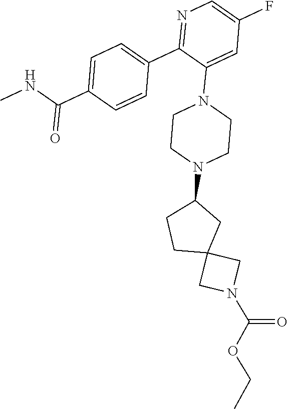 [SMILES: CCOC(=O)N1CC2(CC[C@@H](N3CCN(c4cc(F)cnc4-c4ccc(C(=O)NC)cc4)CC3)C2)C1] |
| A104 | 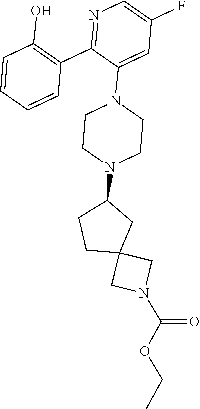 [SMILES: CCOC(=O)N1CC2(CC[C@@H](N3CCN(c4cc(F)cnc4-c4ccccc4O)CC3)C2)C1] |
| A105 | 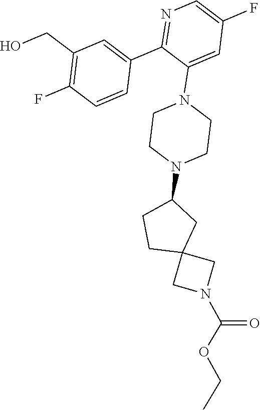 [SMILES: CCOC(=O)N1CC2(CC[C@@H](N3CCN(c4cc(F)cnc4-c4ccc(F)c(CO)c4)CC3)C2)C1] |
| A106 | 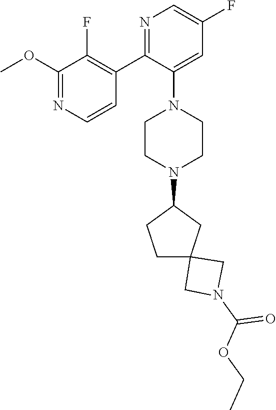 [SMILES: CCOC(=O)N1CC2(CC[C@@H](N3CCN(c4cc(F)cnc4-c4ccnc(OC)c4F)CC3)C2)C1] |
| A107 | 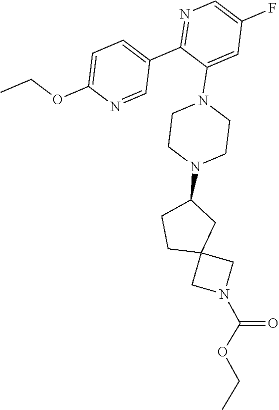 [SMILES: CCOC(=O)N1CC2(CC[C@@H](N3CCN(c4cc(F)cnc4-c4ccc(OCC)nc4)CC3)C2)C1] |
| A108 | 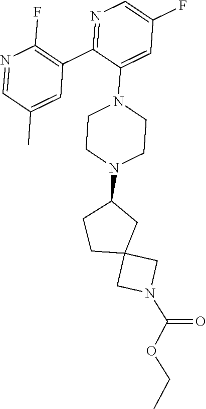 [SMILES: CCOC(=O)N1CC2(CC[C@@H](N3CCN(c4cc(F)cnc4-c4cc(C)cnc4F)CC3)C2)C1] |
| A109 | 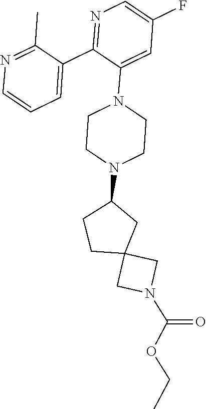 [SMILES: CCOC(=O)N1CC2(CC[C@@H](N3CCN(c4cc(F)cnc4-c4cccnc4C)CC3)C2)C1] |
| A110 | 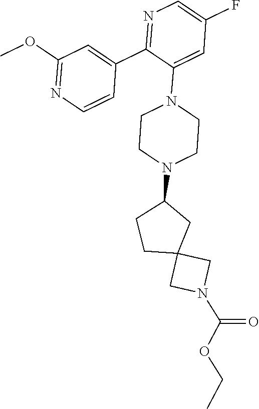 [SMILES: CCOC(=O)N1CC2(CC[C@@H](N3CCN(c4cc(F)cnc4-c4ccnc(OC)c4)CC3)C2)C1] |
| A111 | 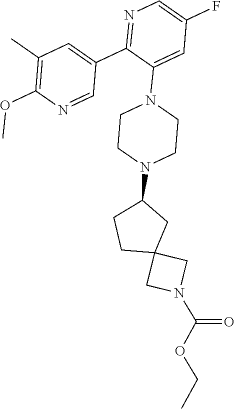 [SMILES: CCOC(=O)N1CC2(CC[C@@H](N3CCN(c4cc(F)cnc4-c4cnc(OC)c(C)c4)CC3)C2)C1] |
| A112 | 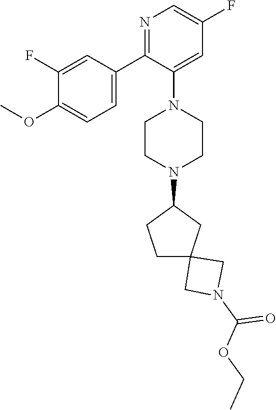 [SMILES: CCOC(=O)N1CC2(CC[C@@H](N3CCN(c4cc(F)cnc4-c4ccc(OC)c(F)c4)CC3)C2)C1] |
| A113 | 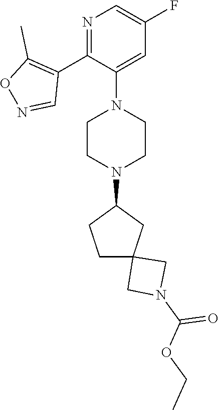 [SMILES: CCOC(=O)N1CC2(CC[C@@H](N3CCN(c4cc(F)cnc4-c4cnoc4C)CC3)C2)C1] |
| A114 | 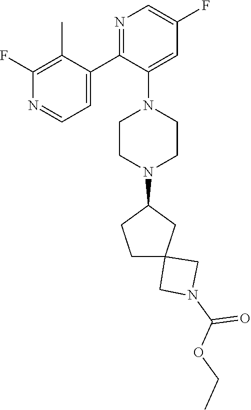 [SMILES: CCOC(=O)N1CC2(CC[C@@H](N3CCN(c4cc(F)cnc4-c4ccnc(F)c4C)CC3)C2)C1] |
| A115 | 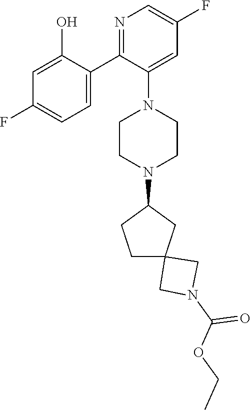 [SMILES: CCOC(=O)N1CC2(CC[C@@H](N3CCN(c4cc(F)cnc4-c4ccc(F)cc4O)CC3)C2)C1] |
| A116 | 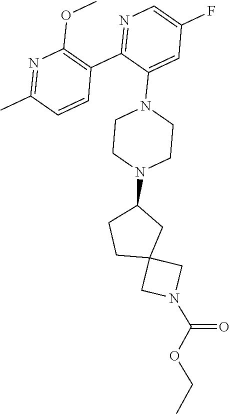 [SMILES: CCOC(=O)N1CC2(CC[C@@H](N3CCN(c4cc(F)cnc4-c4ccc(C)nc4OC)CC3)C2)C1] |
| A117 | 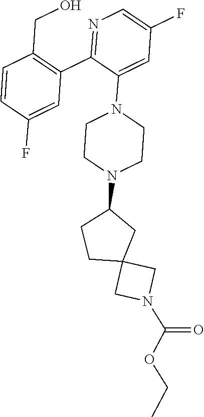 [SMILES: CCOC(=O)N1CC2(CC[C@@H](N3CCN(c4cc(F)cnc4-c4cc(F)ccc4CO)CC3)C2)C1] |
| A118 | 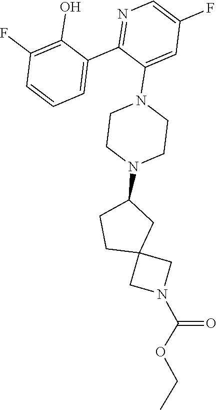 [SMILES: CCOC(=O)N1CC2(CC[C@@H](N3CCN(c4cc(F)cnc4-c4cccc(F)c4O)CC3)C2)C1] |
| A119 | 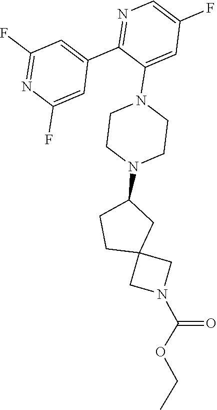 [SMILES: CCOC(=O)N1CC2(CC[C@@H](N3CCN(c4cc(F)cnc4-c4cc(F)nc(F)c4)CC3)C2)C1] |
| A120 | 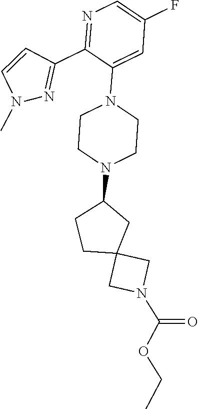 [SMILES: CCOC(=O)N1CC2(CC[C@@H](N3CCN(c4cc(F)cnc4-c4ccn(C)n4)CC3)C2)C1] |
| A121 | 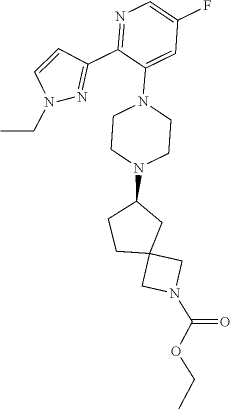 [SMILES: CCOC(=O)N1CC2(CC[C@@H](N3CCN(c4cc(F)cnc4-c4ccn(CC)n4)CC3)C2)C1] |
| A122 | 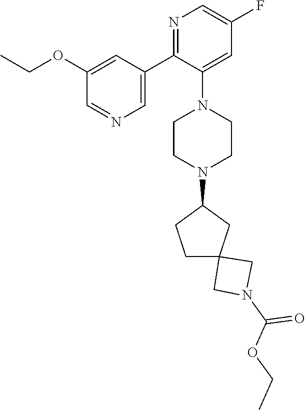 [SMILES: CCOC(=O)N1CC2(CC[C@@H](N3CCN(c4cc(F)cnc4-c4cncc(OCC)c4)CC3)C2)C1] |
| A123 | 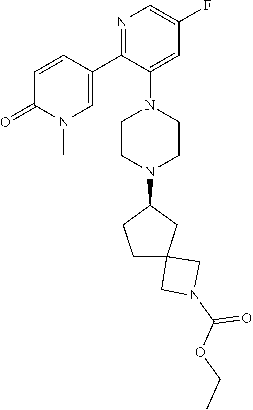 [SMILES: CCOC(=O)N1CC2(CC[C@@H](N3CCN(c4cc(F)cnc4-c4ccc(=O)n(C)c4)CC3)C2)C1] |
| A124 | 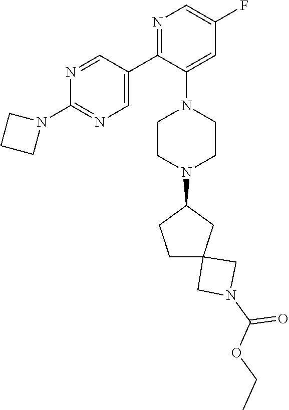 [SMILES: CCOC(=O)N1CC2(CC[C@@H](N3CCN(c4cc(F)cnc4-c4cnc(N5CCC5)nc4)CC3)C2)C1] |
| A125 | 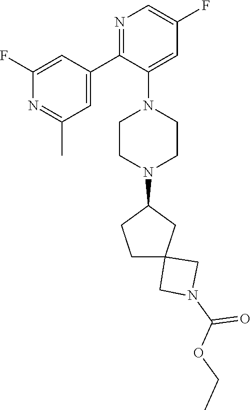 [SMILES: CCOC(=O)N1CC2(CC[C@@H](N3CCN(c4cc(F)cnc4-c4cc(C)nc(F)c4)CC3)C2)C1] |
| A126 | 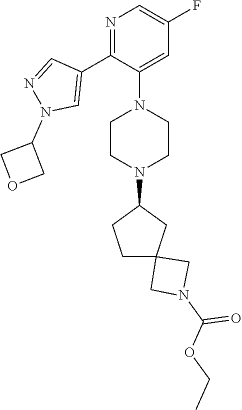 [SMILES: CCOC(=O)N1CC2(CC[C@@H](N3CCN(c4cc(F)cnc4-c4cnn(C5COC5)c4)CC3)C2)C1] |
| A127 | 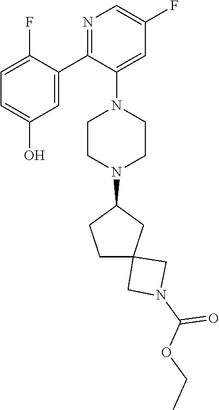 [SMILES: CCOC(=O)N1CC2(CC[C@@H](N3CCN(c4cc(F)cnc4-c4cc(O)ccc4F)CC3)C2)C1] |
| A128 | 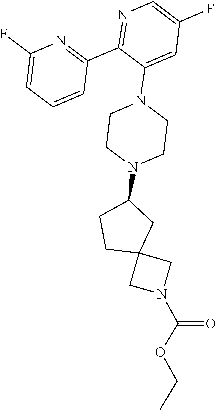 [SMILES: CCOC(=O)N1CC2(CC[C@@H](N3CCN(c4cc(F)cnc4-c4cccc(F)n4)CC3)C2)C1] |
| A129 | 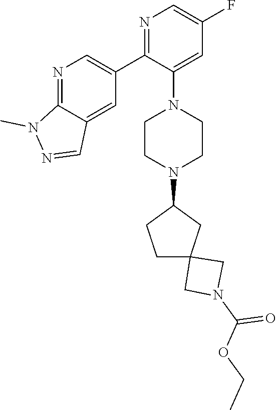 [SMILES: CCOC(=O)N1CC2(CC[C@@H](N3CCN(c4cc(F)cnc4-c4cnc5c(cnn5C)c4)CC3)C2)C1] |
| A130 | 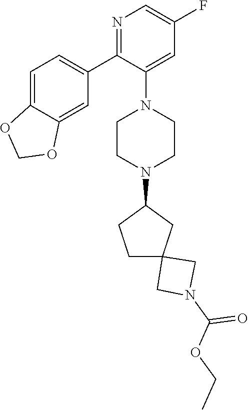 [SMILES: CCOC(=O)N1CC2(CC[C@@H](N3CCN(c4cc(F)cnc4-c4ccc5c(c4)OCO5)CC3)C2)C1] |
| A131 | 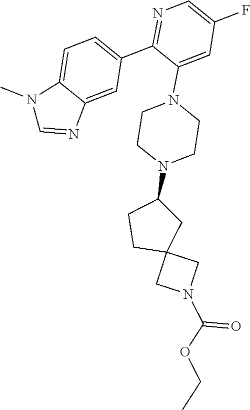 [SMILES: CCOC(=O)N1CC2(CC[C@@H](N3CCN(c4cc(F)cnc4-c4ccc5c(c4)ncn5C)CC3)C2)C1] |
| A132 | 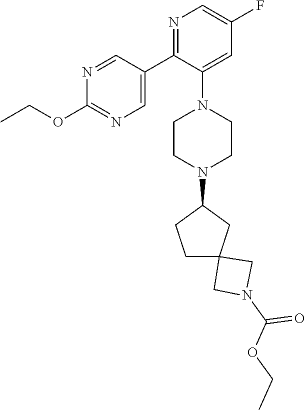 [SMILES: CCOC(=O)N1CC2(CC[C@@H](N3CCN(c4cc(F)cnc4-c4cnc(OCC)nc4)CC3)C2)C1] |
| A133 | 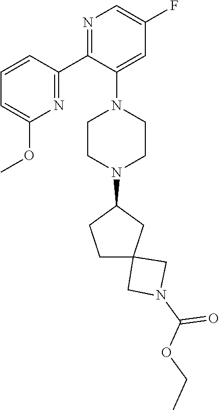 [SMILES: CCOC(=O)N1CC2(CC[C@@H](N3CCN(c4cc(F)cnc4-c4cccc(OC)n4)CC3)C2)C1] |
| A134 | 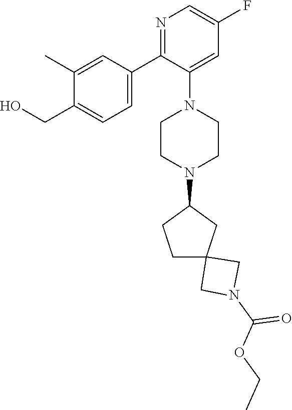 [SMILES: CCOC(=O)N1CC2(CC[C@@H](N3CCN(c4cc(F)cnc4-c4ccc(CO)c(C)c4)CC3)C2)C1] |
| A135 | 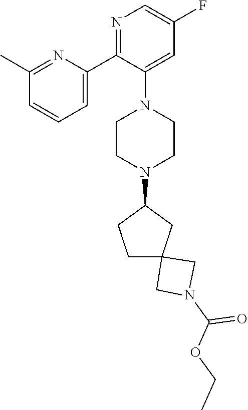 [SMILES: CCOC(=O)N1CC2(CC[C@@H](N3CCN(c4cc(F)cnc4-c4cccc(C)n4)CC3)C2)C1] |
| A136 | 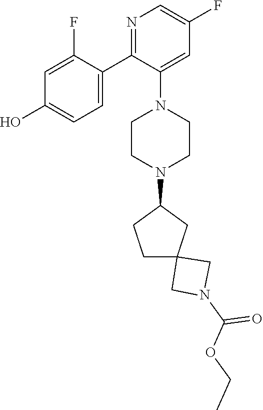 [SMILES: CCOC(=O)N1CC2(CC[C@@H](N3CCN(c4cc(F)cnc4-c4ccc(O)cc4F)CC3)C2)C1] |
| A137 | 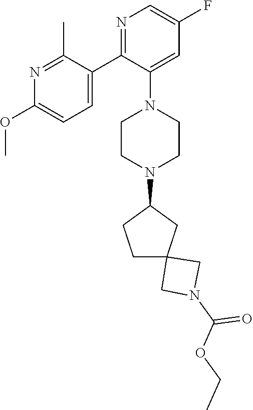 [SMILES: CCOC(=O)N1CC2(CC[C@@H](N3CCN(c4cc(F)cnc4-c4ccc(OC)nc4C)CC3)C2)C1] |
| A138 | 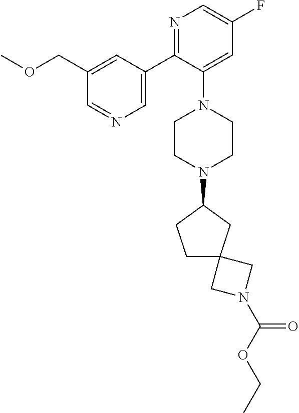 [SMILES: CCOC(=O)N1CC2(CC[C@@H](N3CCN(c4cc(F)cnc4-c4cncc(COC)c4)CC3)C2)C1] |
| A139 | 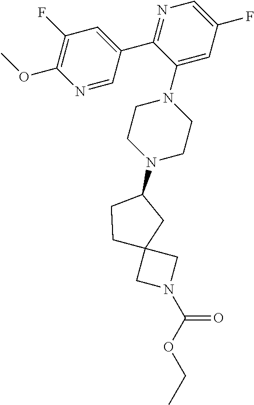 [SMILES: CCOC(=O)N1CC2(CC[C@@H](N3CCN(c4cc(F)cnc4-c4cnc(OC)c(F)c4)CC3)C2)C1] |
| A140 | 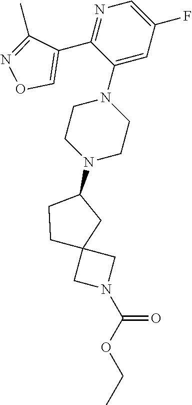 [SMILES: CCOC(=O)N1CC2(CC[C@@H](N3CCN(c4cc(F)cnc4-c4conc4C)CC3)C2)C1] |
| A141 | 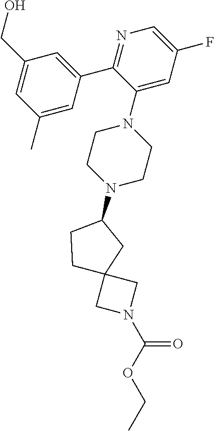 [SMILES: CCOC(=O)N1CC2(CC[C@@H](N3CCN(c4cc(F)cnc4-c4cc(C)cc(CO)c4)CC3)C2)C1] |
| A142 | 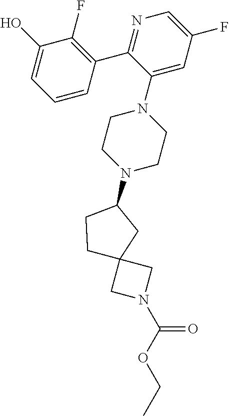 [SMILES: CCOC(=O)N1CC2(CC[C@@H](N3CCN(c4cc(F)cnc4-c4cccc(O)c4F)CC3)C2)C1] |
| A143 | 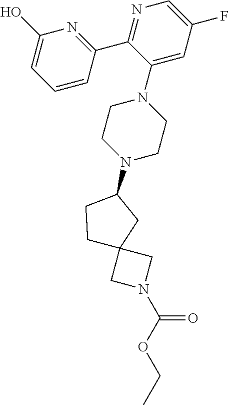 [SMILES: CCOC(=O)N1CC2(CC[C@@H](N3CCN(c4cc(F)cnc4-c4cccc(O)n4)CC3)C2)C1] |
| A144 | 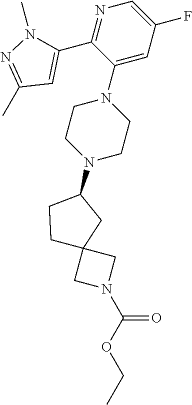 [SMILES: CCOC(=O)N1CC2(CC[C@@H](N3CCN(c4cc(F)cnc4-c4cc(C)nn4C)CC3)C2)C1] |
| A145 | 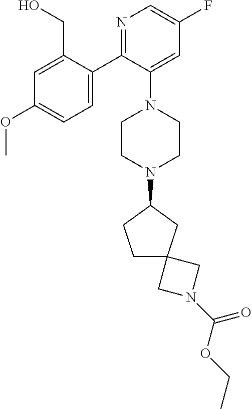 [SMILES: CCOC(=O)N1CC2(CC[C@@H](N3CCN(c4cc(F)cnc4-c4ccc(OC)cc4CO)CC3)C2)C1] |
| A146 | 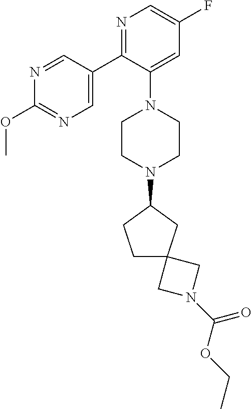 [SMILES: CCOC(=O)N1CC2(CC[C@@H](N3CCN(c4cc(F)cnc4-c4cnc(OC)nc4)CC3)C2)C1] |
| A147 | 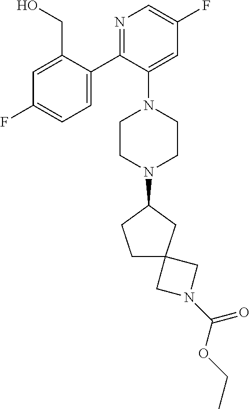 [SMILES: CCOC(=O)N1CC2(CC[C@@H](N3CCN(c4cc(F)cnc4-c4ccc(F)cc4CO)CC3)C2)C1] |
| A148 | 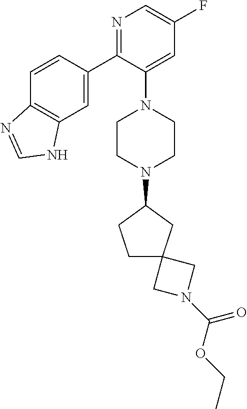 [SMILES: CCOC(=O)N1CC2(CC[C@@H](N3CCN(c4cc(F)cnc4-c4ccc5nc[nH]c5c4)CC3)C2)C1] |
| A149 | 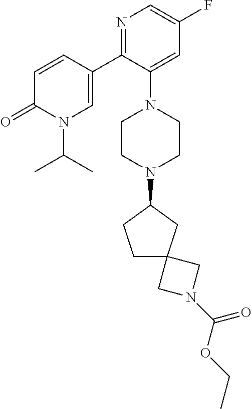 [SMILES: CCOC(=O)N1CC2(CC[C@@H](N3CCN(c4cc(F)cnc4-c4ccc(=O)n(C(C)C)c4)CC3)C2)C1] |
| A150 | 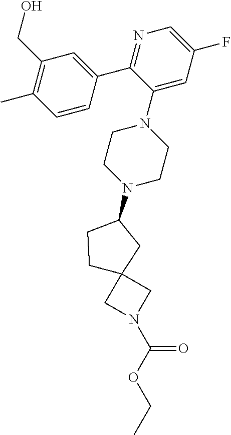 [SMILES: CCOC(=O)N1CC2(CC[C@@H](N3CCN(c4cc(F)cnc4-c4ccc(C)c(CO)c4)CC3)C2)C1] |
| A151 | 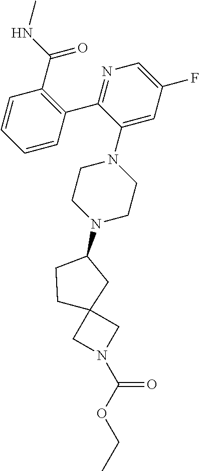 [SMILES: CCOC(=O)N1CC2(CC[C@@H](N3CCN(c4cc(F)cnc4-c4ccccc4C(=O)NC)CC3)C2)C1] |
| A152 | 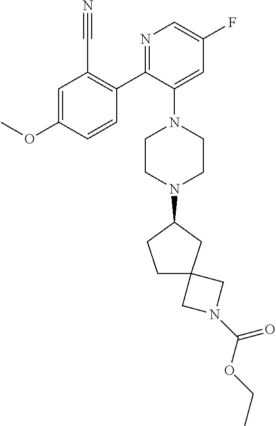 [SMILES: CCOC(=O)N1CC2(CC[C@@H](N3CCN(c4cc(F)cnc4-c4ccc(OC)cc4C#N)CC3)C2)C1] |
| A153 | 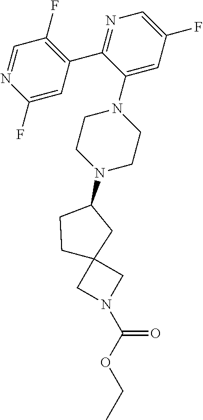 [SMILES: CCOC(=O)N1CC2(CC[C@@H](N3CCN(c4cc(F)cnc4-c4cc(F)ncc4F)CC3)C2)C1] |
| A154 | 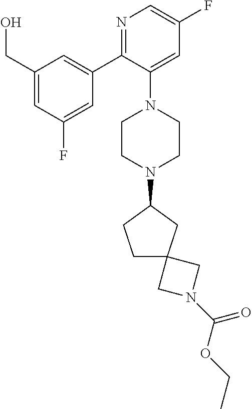 [SMILES: CCOC(=O)N1CC2(CC[C@@H](N3CCN(c4cc(F)cnc4-c4cc(F)cc(CO)c4)CC3)C2)C1] |
| A155 | 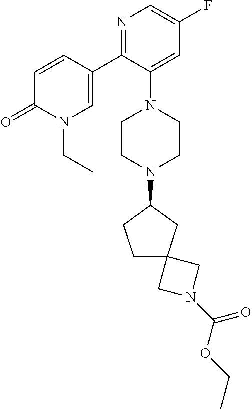 [SMILES: CCOC(=O)N1CC2(CC[C@@H](N3CCN(c4cc(F)cnc4-c4ccc(=O)n(CC)c4)CC3)C2)C1] |
| A156 | 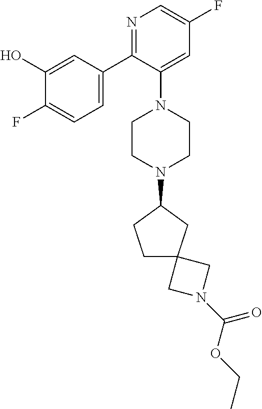 [SMILES: CCOC(=O)N1CC2(CC[C@@H](N3CCN(c4cc(F)cnc4-c4ccc(F)c(O)c4)CC3)C2)C1] |
| A157 | 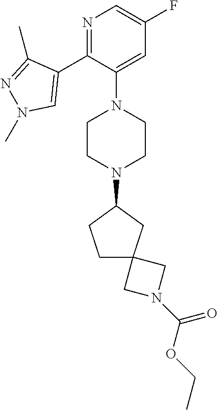 [SMILES: CCOC(=O)N1CC2(CC[C@@H](N3CCN(c4cc(F)cnc4-c4cn(C)nc4C)CC3)C2)C1] |
| A158 | 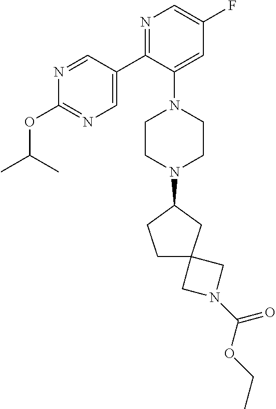 [SMILES: CCOC(=O)N1CC2(CC[C@@H](N3CCN(c4cc(F)cnc4-c4cnc(OC(C)C)nc4)CC3)C2)C1] |
| A159 | 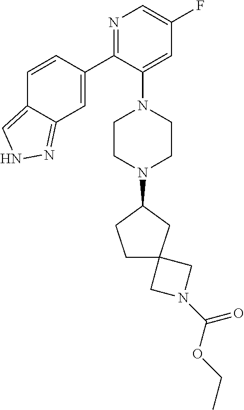 [SMILES: CCOC(=O)N1CC2(CC[C@@H](N3CCN(c4cc(F)cnc4-c4ccc5c[nH]nc5c4)CC3)C2)C1] |
| A160 | 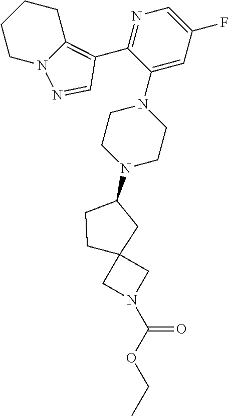 [SMILES: CCOC(=O)N1CC2(CC[C@@H](N3CCN(c4cc(F)cnc4-c4cnn5c4CCCC5)CC3)C2)C1] |
| A161 | 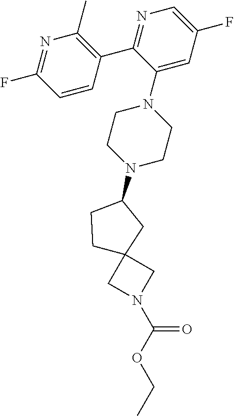 [SMILES: CCOC(=O)N1CC2(CC[C@@H](N3CCN(c4cc(F)cnc4-c4ccc(F)nc4C)CC3)C2)C1] |
| A162 | 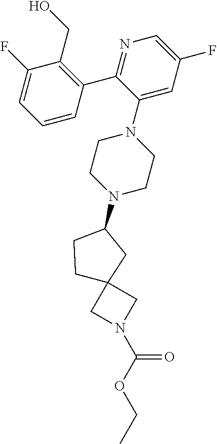 [SMILES: CCOC(=O)N1CC2(CC[C@@H](N3CCN(c4cc(F)cnc4-c4cccc(F)c4CO)CC3)C2)C1] |
| A163 | 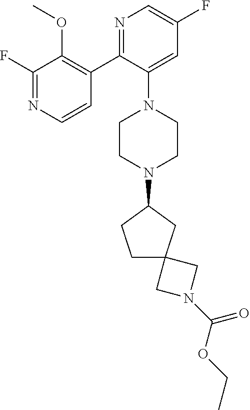 [SMILES: CCOC(=O)N1CC2(CC[C@@H](N3CCN(c4cc(F)cnc4-c4ccnc(F)c4OC)CC3)C2)C1] |
| A164 | 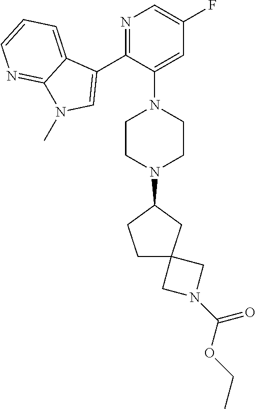 [SMILES: CCOC(=O)N1CC2(CC[C@@H](N3CCN(c4cc(F)cnc4-c4cn(C)c5ncccc45)CC3)C2)C1] |
| A165 | 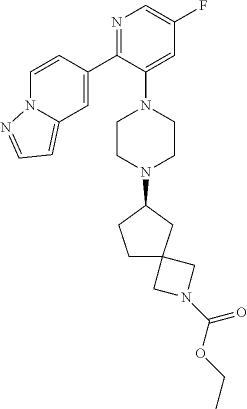 [SMILES: CCOC(=O)N1CC2(CC[C@@H](N3CCN(c4cc(F)cnc4-c4ccn5nccc5c4)CC3)C2)C1] |
| A166 | 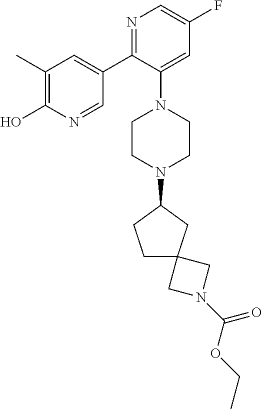 [SMILES: CCOC(=O)N1CC2(CC[C@@H](N3CCN(c4cc(F)cnc4-c4cnc(O)c(C)c4)CC3)C2)C1] |
| A167 | 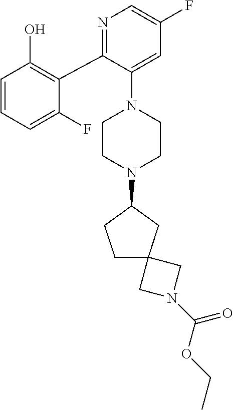 [SMILES: CCOC(=O)N1CC2(CC[C@@H](N3CCN(c4cc(F)cnc4-c4c(O)cccc4F)CC3)C2)C1] |
| A168 | 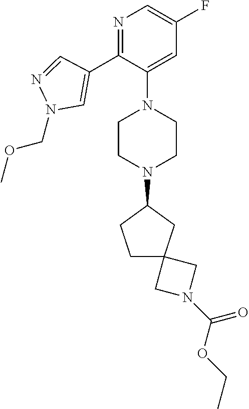 [SMILES: CCOC(=O)N1CC2(CC[C@@H](N3CCN(c4cc(F)cnc4-c4cnn(COC)c4)CC3)C2)C1] |
| A169 | 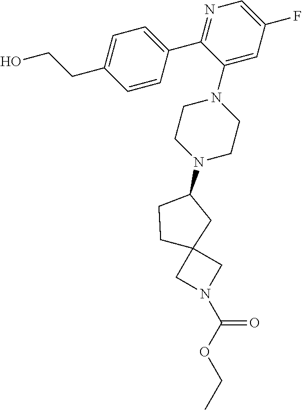 [SMILES: CCOC(=O)N1CC2(CC[C@@H](N3CCN(c4cc(F)cnc4-c4ccc(CCO)cc4)CC3)C2)C1] |
| A170 | 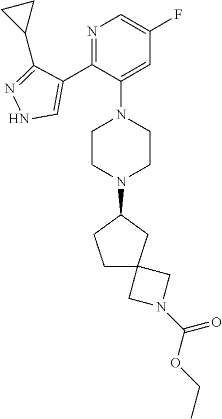 [SMILES: CCOC(=O)N1CC2(CC[C@@H](N3CCN(c4cc(F)cnc4-c4c[nH]nc4C4CC4)CC3)C2)C1] |
| A171 | 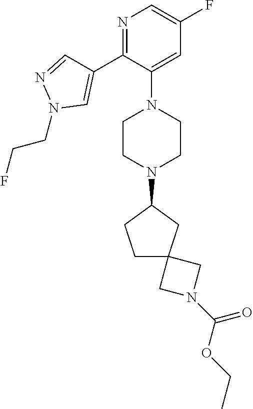 [SMILES: CCOC(=O)N1CC2(CC[C@@H](N3CCN(c4cc(F)cnc4-c4cnn(CCF)c4)CC3)C2)C1] |
| A172 | 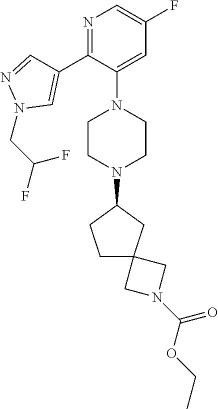 [SMILES: CCOC(=O)N1CC2(CC[C@@H](N3CCN(c4cc(F)cnc4-c4cnn(CC(F)F)c4)CC3)C2)C1] |
| A173 | 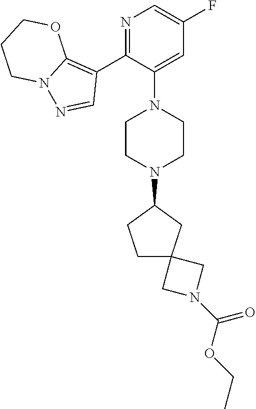 [SMILES: CCOC(=O)N1CC2(CC[C@@H](N3CCN(c4cc(F)cnc4-c4cnn5c4OCCC5)CC3)C2)C1] |
| A174 | 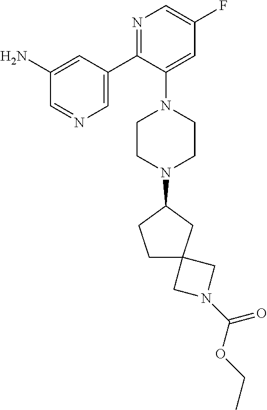 [SMILES: CCOC(=O)N1CC2(CC[C@@H](N3CCN(c4cc(F)cnc4-c4cncc(N)c4)CC3)C2)C1] |
| A175 | 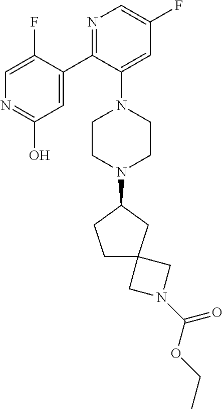 [SMILES: CCOC(=O)N1CC2(CC[C@@H](N3CCN(c4cc(F)cnc4-c4cc(O)ncc4F)CC3)C2)C1] |
| A176 | 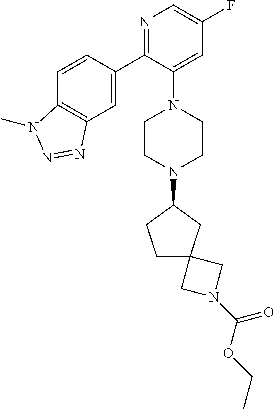 [SMILES: CCOC(=O)N1CC2(CC[C@@H](N3CCN(c4cc(F)cnc4-c4ccc5c(c4)nnn5C)CC3)C2)C1] |
| A177 | 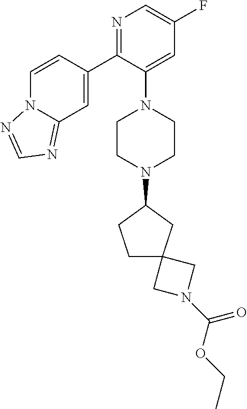 [SMILES: CCOC(=O)N1CC2(CC[C@@H](N3CCN(c4cc(F)cnc4-c4ccn5ncnc5c4)CC3)C2)C1] |
| A178 | 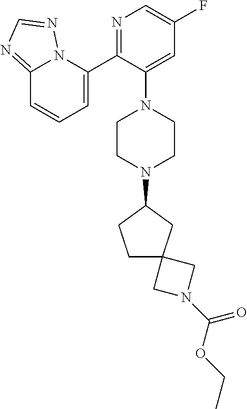 [SMILES: CCOC(=O)N1CC2(CC[C@@H](N3CCN(c4cc(F)cnc4-c4cccc5ncnn45)CC3)C2)C1] |
| A179 | 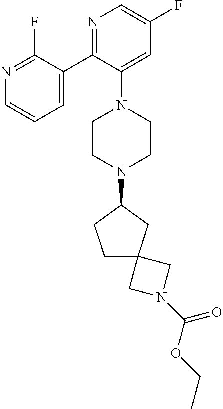 [SMILES: CCOC(=O)N1CC2(CC[C@@H](N3CCN(c4cc(F)cnc4-c4cccnc4F)CC3)C2)C1] |
| A180 | 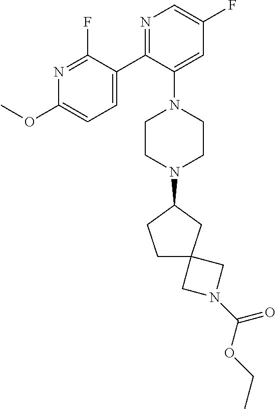 [SMILES: CCOC(=O)N1CC2(CC[C@@H](N3CCN(c4cc(F)cnc4-c4ccc(OC)nc4F)CC3)C2)C1] |
| A181 | 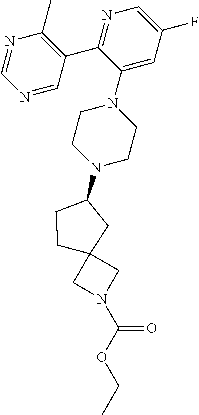 [SMILES: CCOC(=O)N1CC2(CC[C@@H](N3CCN(c4cc(F)cnc4-c4cncnc4C)CC3)C2)C1] |
| A182 | 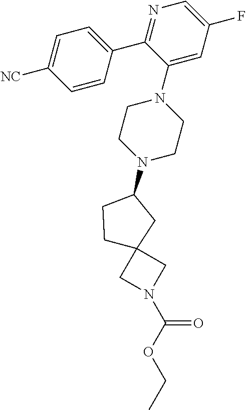 [SMILES: CCOC(=O)N1CC2(CC[C@@H](N3CCN(c4cc(F)cnc4-c4ccc(C#N)cc4)CC3)C2)C1] |
| A183 | 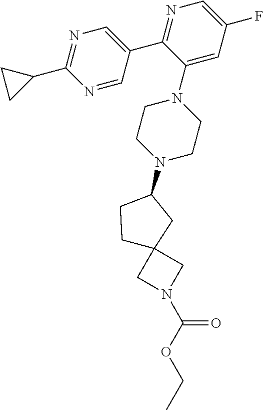 [SMILES: CCOC(=O)N1CC2(CC[C@@H](N3CCN(c4cc(F)cnc4-c4cnc(C5CC5)nc4)CC3)C2)C1] |
| A184 | 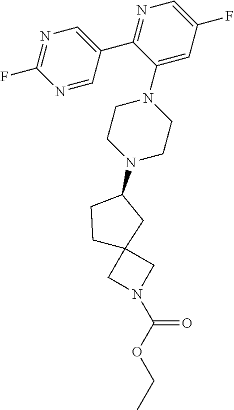 [SMILES: CCOC(=O)N1CC2(CC[C@@H](N3CCN(c4cc(F)cnc4-c4cnc(F)nc4)CC3)C2)C1] |
| A185 | 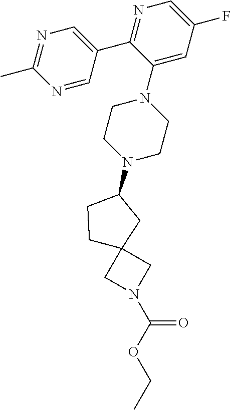 [SMILES: CCOC(=O)N1CC2(CC[C@@H](N3CCN(c4cc(F)cnc4-c4cnc(C)nc4)CC3)C2)C1] |
| A186 | 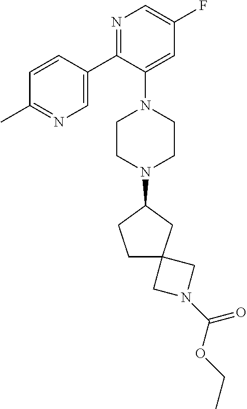 [SMILES: CCOC(=O)N1CC2(CC[C@@H](N3CCN(c4cc(F)cnc4-c4ccc(C)nc4)CC3)C2)C1] |
| A187 | 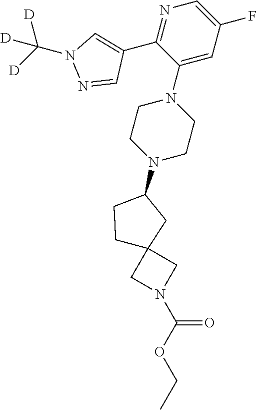 [SMILES: [2H]C([2H])([2H])n1cc(-c2ncc(F)cc2N2CCN([C@@H]3CCC4(C3)CN(C(=O)OCC)C4)CC2)cn1] |
| A188 | 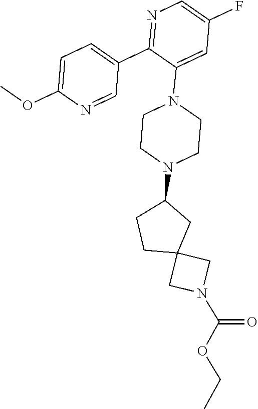 [SMILES: CCOC(=O)N1CC2(CC[C@@H](N3CCN(c4cc(F)cnc4-c4ccc(OC)nc4)CC3)C2)C1] |
| A189 | 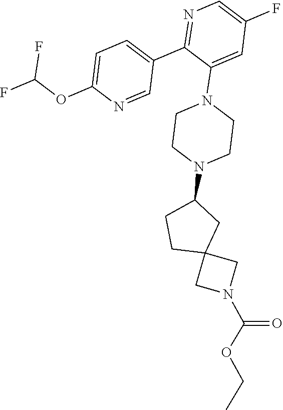 [SMILES: CCOC(=O)N1CC2(CC[C@@H](N3CCN(c4cc(F)cnc4-c4ccc(OC(F)F)nc4)CC3)C2)C1] |
| A190 | 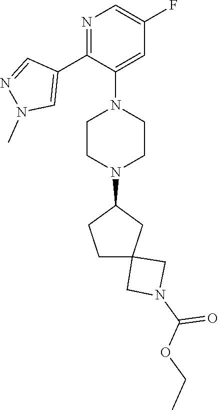 [SMILES: CCOC(=O)N1CC2(CC[C@@H](N3CCN(c4cc(F)cnc4-c4cnn(C)c4)CC3)C2)C1] |
| A191 | 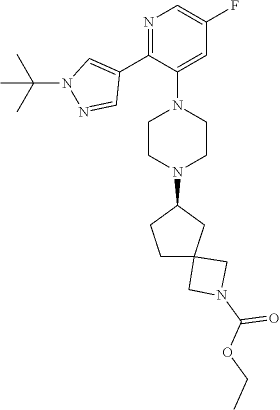 [SMILES: CCOC(=O)N1CC2(CC[C@@H](N3CCN(c4cc(F)cnc4-c4cnn(C(C)(C)C)c4)CC3)C2)C1] |
| A192 | 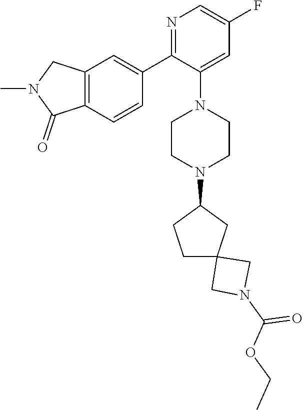 [SMILES: CCOC(=O)N1CC2(CC[C@@H](N3CCN(c4cc(F)cnc4-c4ccc5c(c4)CN(C)C5=O)CC3)C2)C1] |
| A193 | 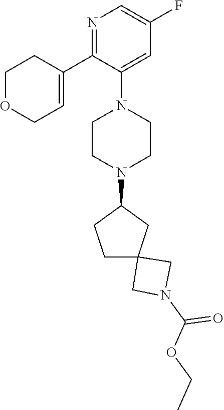 [SMILES: CCOC(=O)N1CC2(CC[C@@H](N3CCN(c4cc(F)cnc4C4=CCOCC4)CC3)C2)C1] |
| A194 | 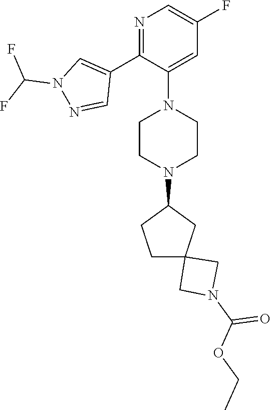 [SMILES: CCOC(=O)N1CC2(CC[C@@H](N3CCN(c4cc(F)cnc4-c4cnn(C(F)F)c4)CC3)C2)C1] |
| A195 | 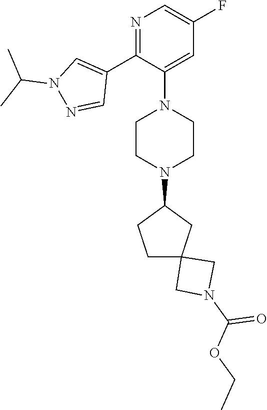 [SMILES: CCOC(=O)N1CC2(CC[C@@H](N3CCN(c4cc(F)cnc4-c4cnn(C(C)C)c4)CC3)C2)C1] |
| A196 | 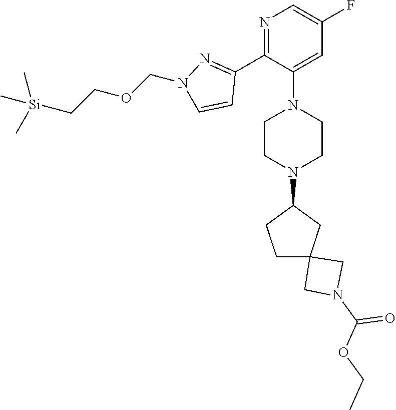 [SMILES: CCOC(=O)N1CC2(CC[C@@H](N3CCN(c4cc(F)cnc4-c4ccn(COCC[Si](C)(C)C)n4)CC3)C2)C1] |
| A197 | 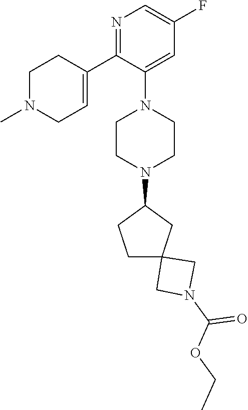 [SMILES: CCOC(=O)N1CC2(CC[C@@H](N3CCN(c4cc(F)cnc4C4=CCN(C)CC4)CC3)C2)C1] |
| A198 | 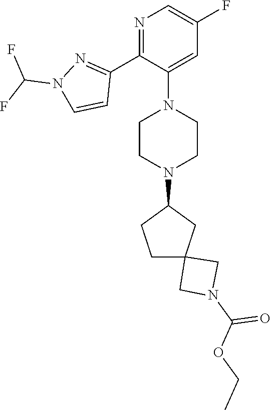 [SMILES: CCOC(=O)N1CC2(CC[C@@H](N3CCN(c4cc(F)cnc4-c4ccn(C(F)F)n4)CC3)C2)C1] |
| A199 | 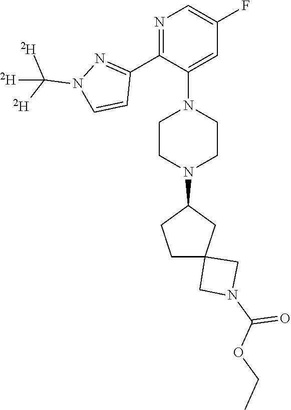 [SMILES: [2H]C([2H])([2H])n1ccc(-c2ncc(F)cc2N2CCN([C@@H]3CCC4(C3)CN(C(=O)OCC)C4)CC2)n1] |
| A200 | 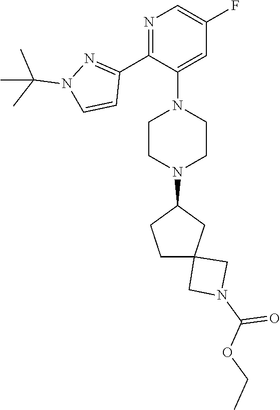 [SMILES: CCOC(=O)N1CC2(CC[C@@H](N3CCN(c4cc(F)cnc4-c4ccn(C(C)(C)C)n4)CC3)C2)C1] |
| A201 | 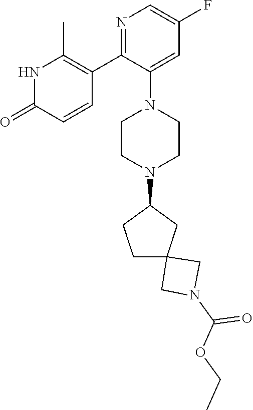 [SMILES: CCOC(=O)N1CC2(CC[C@@H](N3CCN(c4cc(F)cnc4-c4ccc(=O)[nH]c4C)CC3)C2)C1] |
| A202 | 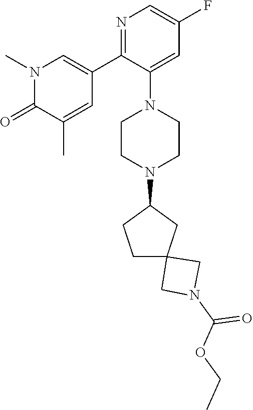 [SMILES: CCOC(=O)N1CC2(CC[C@@H](N3CCN(c4cc(F)cnc4-c4cc(C)c(=O)n(C)c4)CC3)C2)C1] |
| A203 | 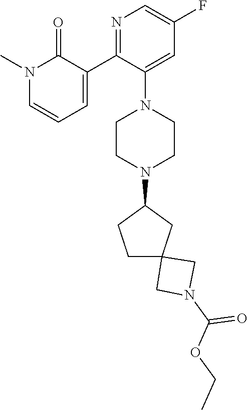 [SMILES: CCOC(=O)N1CC2(CC[C@@H](N3CCN(c4cc(F)cnc4-c4cccn(C)c4=O)CC3)C2)C1] |
| A204 | 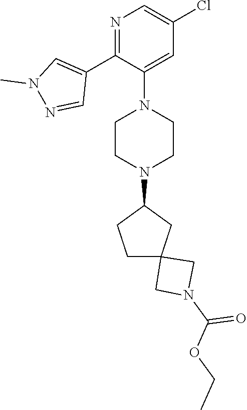 [SMILES: CCOC(=O)N1CC2(CC[C@@H](N3CCN(c4cc(Cl)cnc4-c4cnn(C)c4)CC3)C2)C1] |
| A205 | 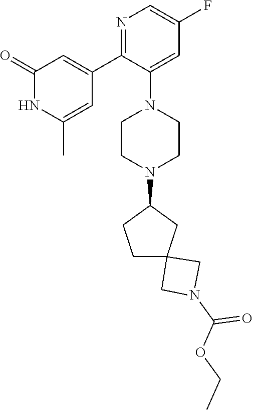 [SMILES: CCOC(=O)N1CC2(CC[C@@H](N3CCN(c4cc(F)cnc4-c4cc(C)[nH]c(=O)c4)CC3)C2)C1] |
| A206 | 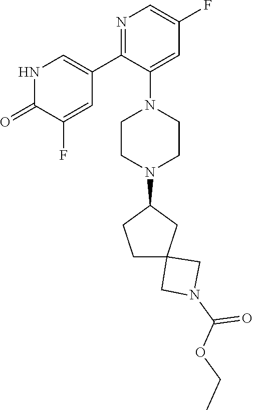 [SMILES: CCOC(=O)N1CC2(CC[C@@H](N3CCN(c4cc(F)cnc4-c4c[nH]c(=O)c(F)c4)CC3)C2)C1] |
| A207 | 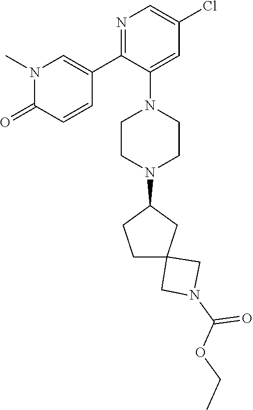 [SMILES: CCOC(=O)N1CC2(CC[C@@H](N3CCN(c4cc(Cl)cnc4-c4ccc(=O)n(C)c4)CC3)C2)C1] |
| A208 | 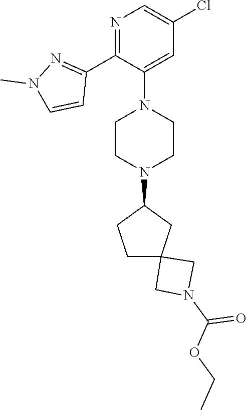 [SMILES: CCOC(=O)N1CC2(CC[C@@H](N3CCN(c4cc(Cl)cnc4-c4ccn(C)n4)CC3)C2)C1] |
| A209 | 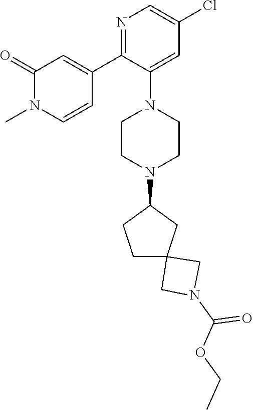 [SMILES: CCOC(=O)N1CC2(CC[C@@H](N3CCN(c4cc(Cl)cnc4-c4ccn(C)c(=O)c4)CC3)C2)C1] |
| A210 | 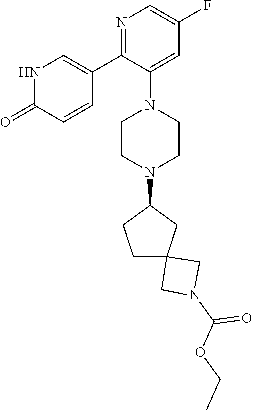 [SMILES: CCOC(=O)N1CC2(CC[C@@H](N3CCN(c4cc(F)cnc4-c4ccc(=O)[nH]c4)CC3)C2)C1] |
| A211 | 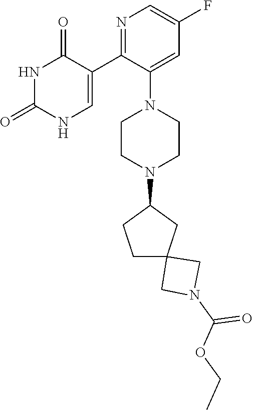 [SMILES: CCOC(=O)N1CC2(CC[C@@H](N3CCN(c4cc(F)cnc4-c4c[nH]c(=O)[nH]c4=O)CC3)C2)C1] |
| A212 | 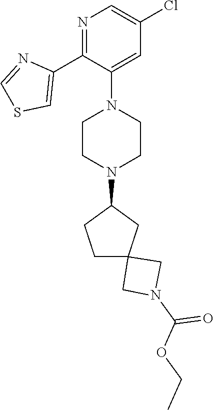 [SMILES: CCOC(=O)N1CC2(CC[C@@H](N3CCN(c4cc(Cl)cnc4-c4cscn4)CC3)C2)C1] |
| A213 | 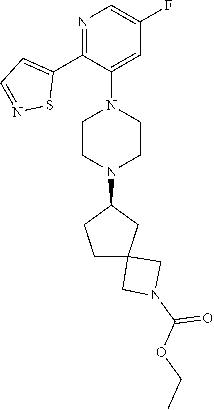 [SMILES: CCOC(=O)N1CC2(CC[C@@H](N3CCN(c4cc(F)cnc4-c4ccns4)CC3)C2)C1] |
| A214 | 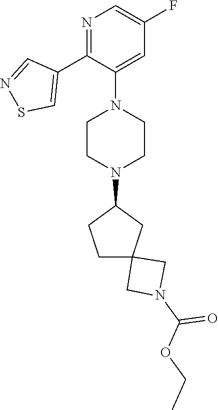 [SMILES: CCOC(=O)N1CC2(CC[C@@H](N3CCN(c4cc(F)cnc4-c4cnsc4)CC3)C2)C1] |
| A215 | 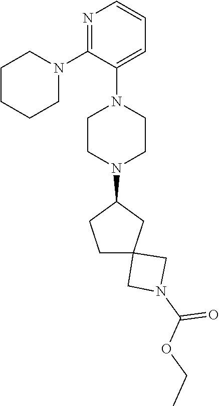 [SMILES: CCOC(=O)N1CC2(CC[C@@H](N3CCN(c4cccnc4N4CCCCC4)CC3)C2)C1] |
| A216 | 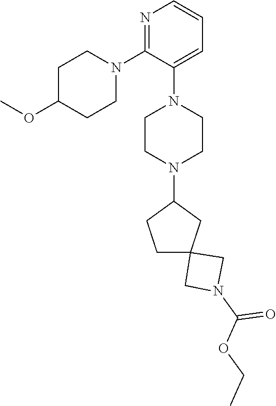 [SMILES: CCOC(=O)N1CC2(CCC(N3CCN(c4cccnc4N4CCC(OC)CC4)CC3)C2)C1] |
| A217 | 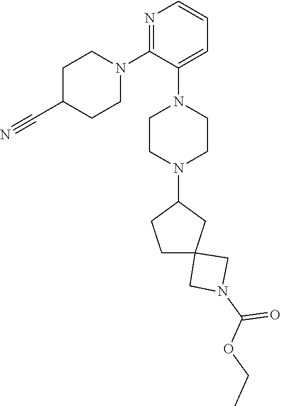 [SMILES: CCOC(=O)N1CC2(CCC(N3CCN(c4cccnc4N4CCC(C#N)CC4)CC3)C2)C1] |
| A218 | 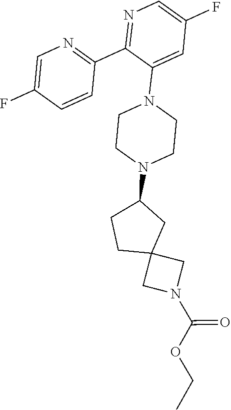 [SMILES: CCOC(=O)N1CC2(CC[C@@H](N3CCN(c4cc(F)cnc4-c4ccc(F)cn4)CC3)C2)C1] |
| A219 | 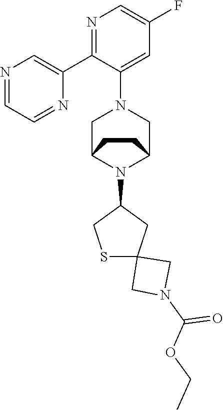 |
| A220 | 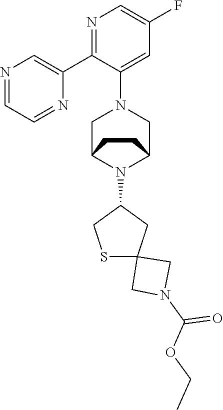 |
| A221 | 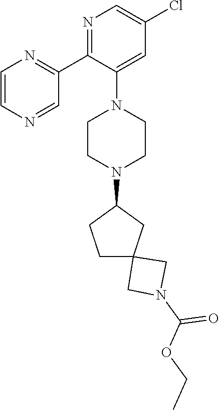 [SMILES: CCOC(=O)N1CC2(CC[C@@H](N3CCN(c4cc(Cl)cnc4-c4cnccn4)CC3)C2)C1] |
| A222 | 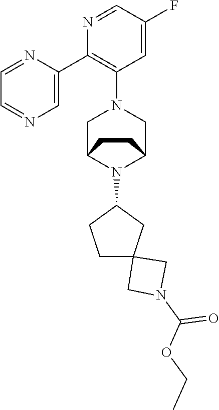 |
| A223 | 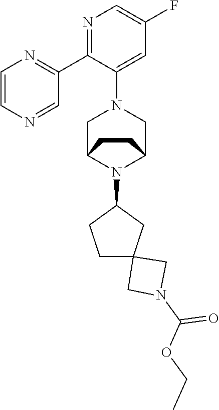 |
| A224 | 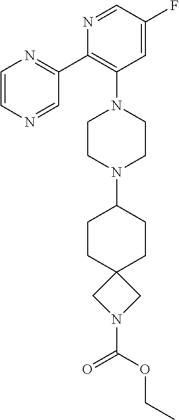 [SMILES: CCOC(=O)N1CC2(CCC(N3CCN(c4cc(F)cnc4-c4cnccn4)CC3)CC2)C1] |
| A225 | 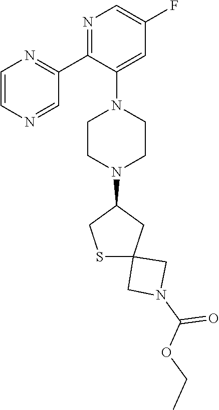 [SMILES: CCOC(=O)N1CC2(C[C@H](N3CCN(c4cc(F)cnc4-c4cnccn4)CC3)CS2)C1] |
| A226 | 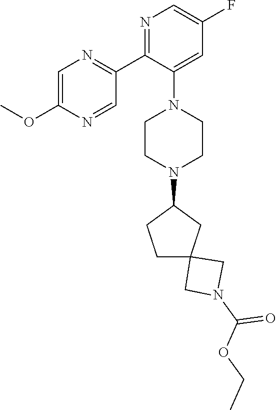 [SMILES: CCOC(=O)N1CC2(CC[C@@H](N3CCN(c4cc(F)cnc4-c4cnc(OC)cn4)CC3)C2)C1] |
| A227 | 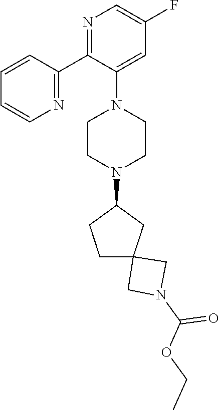 [SMILES: CCOC(=O)N1CC2(CC[C@@H](N3CCN(c4cc(F)cnc4-c4ccccn4)CC3)C2)C1] |
| A228 | 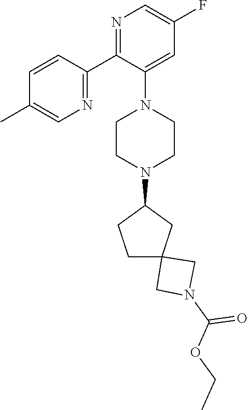 [SMILES: CCOC(=O)N1CC2(CC[C@@H](N3CCN(c4cc(F)cnc4-c4ccc(C)cn4)CC3)C2)C1] |
| A229 | 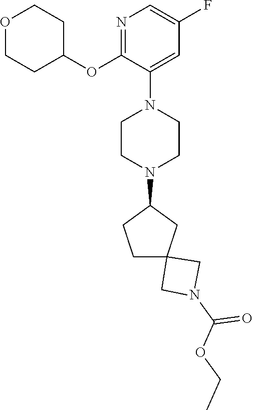 [SMILES: CCOC(=O)N1CC2(CC[C@@H](N3CCN(c4cc(F)cnc4OC4CCOCC4)CC3)C2)C1] |
| A230 | 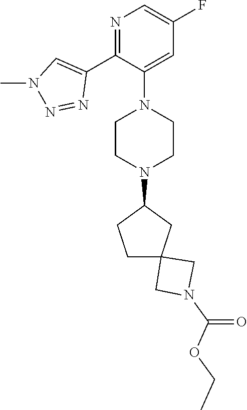 [SMILES: CCOC(=O)N1CC2(CC[C@@H](N3CCN(c4cc(F)cnc4-c4cn(C)nn4)CC3)C2)C1] |
| A231 | 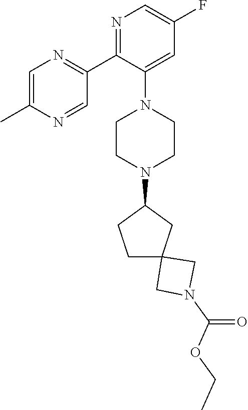 [SMILES: CCOC(=O)N1CC2(CC[C@@H](N3CCN(c4cc(F)cnc4-c4cnc(C)cn4)CC3)C2)C1] |
| A232 | 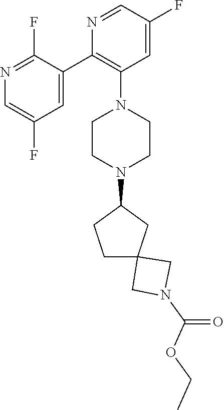 [SMILES: CCOC(=O)N1CC2(CC[C@@H](N3CCN(c4cc(F)cnc4-c4cc(F)cnc4F)CC3)C2)C1] |
| A233 | 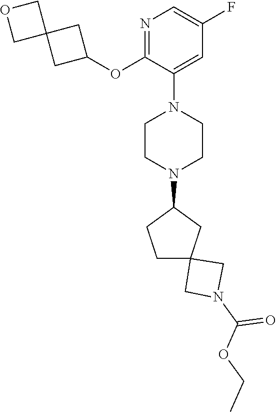 [SMILES: CCOC(=O)N1CC2(CC[C@@H](N3CCN(c4cc(F)cnc4OC4CC5(COC5)C4)CC3)C2)C1] |
| A234 | 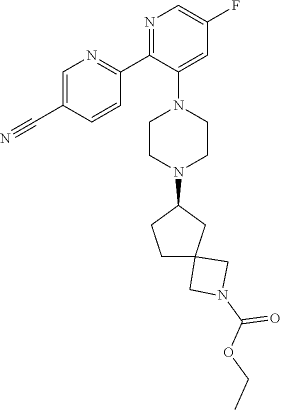 [SMILES: CCOC(=O)N1CC2(CC[C@@H](N3CCN(c4cc(F)cnc4-c4ccc(C#N)cn4)CC3)C2)C1] |
| A235 | 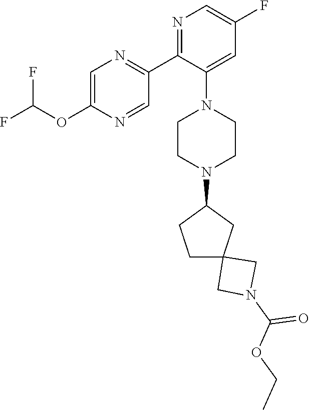 [SMILES: CCOC(=O)N1CC2(CC[C@@H](N3CCN(c4cc(F)cnc4-c4cnc(OC(F)F)cn4)CC3)C2)C1] |
| A236 | 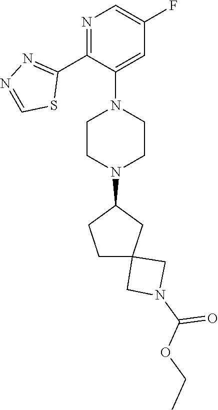 [SMILES: CCOC(=O)N1CC2(CC[C@@H](N3CCN(c4cc(F)cnc4-c4nncs4)CC3)C2)C1] |
| A237 | 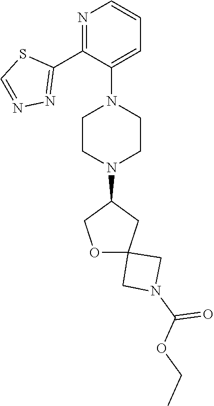 [SMILES: CCOC(=O)N1CC2(C[C@H](N3CCN(c4cccnc4-c4nncs4)CC3)CO2)C1] |
| A238 | 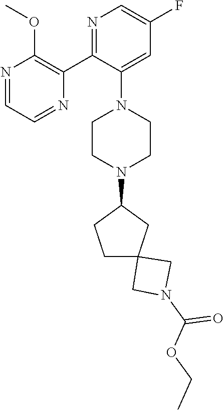 [SMILES: CCOC(=O)N1CC2(CC[C@@H](N3CCN(c4cc(F)cnc4-c4nccnc4OC)CC3)C2)C1] |
| A239 | 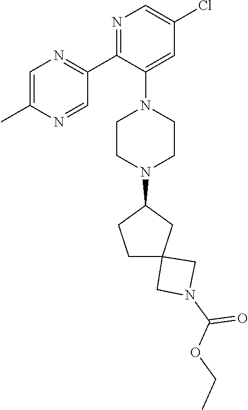 [SMILES: CCOC(=O)N1CC2(CC[C@@H](N3CCN(c4cc(Cl)cnc4-c4cnc(C)cn4)CC3)C2)C1] |
| A240 | 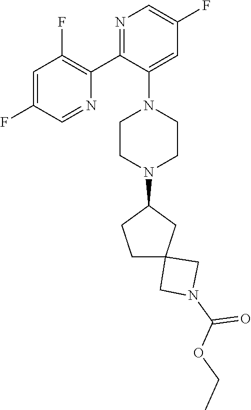 [SMILES: CCOC(=O)N1CC2(CC[C@@H](N3CCN(c4cc(F)cnc4-c4ncc(F)cc4F)CC3)C2)C1] |
| A241 | 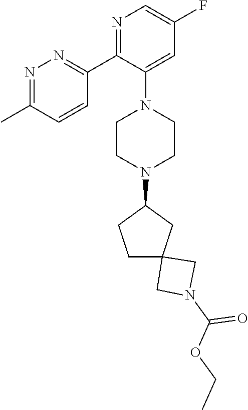 [SMILES: CCOC(=O)N1CC2(CC[C@@H](N3CCN(c4cc(F)cnc4-c4ccc(C)nn4)CC3)C2)C1] |
| A242 | 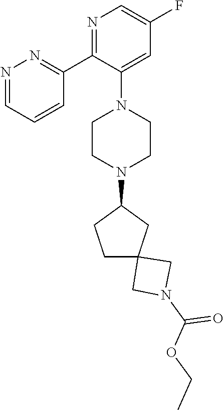 [SMILES: CCOC(=O)N1CC2(CC[C@@H](N3CCN(c4cc(F)cnc4-c4cccnn4)CC3)C2)C1] |
| A243 | 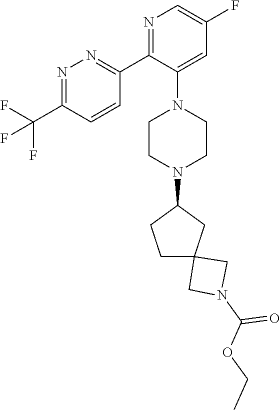 [SMILES: CCOC(=O)N1CC2(CC[C@@H](N3CCN(c4cc(F)cnc4-c4ccc(C(F)(F)F)nn4)CC3)C2)C1] |
| A244 | 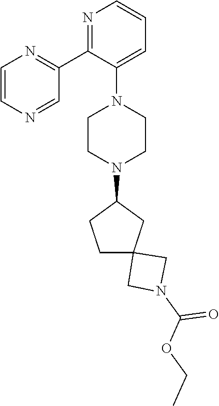 [SMILES: CCOC(=O)N1CC2(CC[C@@H](N3CCN(c4cccnc4-c4cnccn4)CC3)C2)C1] |
| A245 | 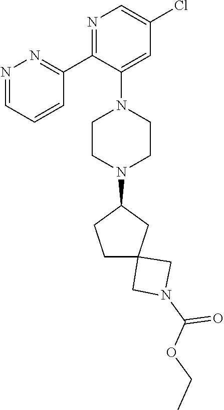 [SMILES: CCOC(=O)N1CC2(CC[C@@H](N3CCN(c4cc(Cl)cnc4-c4cccnn4)CC3)C2)C1] |
| A246 | 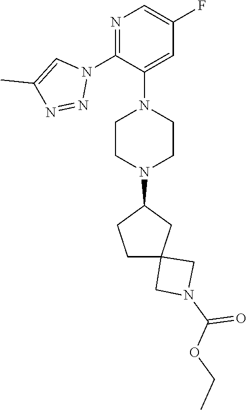 [SMILES: CCOC(=O)N1CC2(CC[C@@H](N3CCN(c4cc(F)cnc4-n4cc(C)nn4)CC3)C2)C1] |
| A247 | 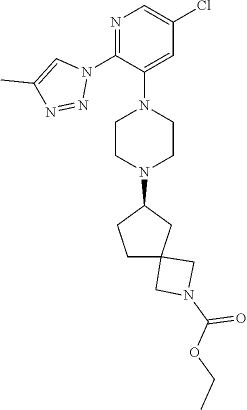 [SMILES: CCOC(=O)N1CC2(CC[C@@H](N3CCN(c4cc(Cl)cnc4-n4cc(C)nn4)CC3)C2)C1] |
| A248 | 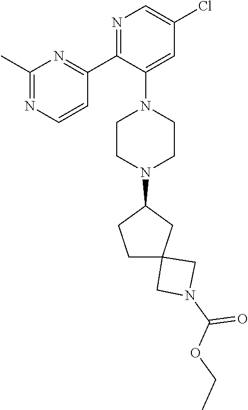 [SMILES: CCOC(=O)N1CC2(CC[C@@H](N3CCN(c4cc(Cl)cnc4-c4ccnc(C)n4)CC3)C2)C1] |
| A249 | 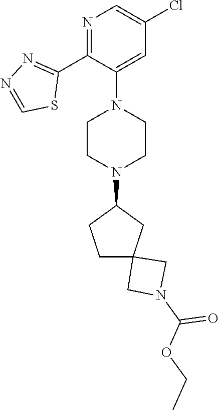 [SMILES: CCOC(=O)N1CC2(CC[C@@H](N3CCN(c4cc(Cl)cnc4-c4nncs4)CC3)C2)C1] |
| A250 | 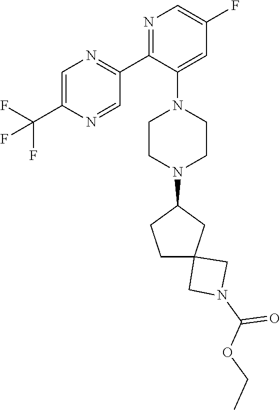 [SMILES: CCOC(=O)N1CC2(CC[C@@H](N3CCN(c4cc(F)cnc4-c4cnc(C(F)(F)F)cn4)CC3)C2)C1] |
| A251 | 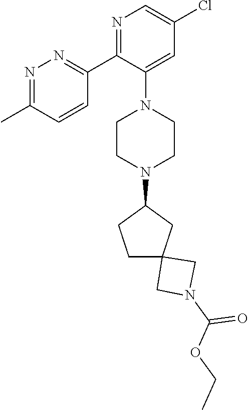 [SMILES: CCOC(=O)N1CC2(CC[C@@H](N3CCN(c4cc(Cl)cnc4-c4ccc(C)nn4)CC3)C2)C1] |
| A252 | 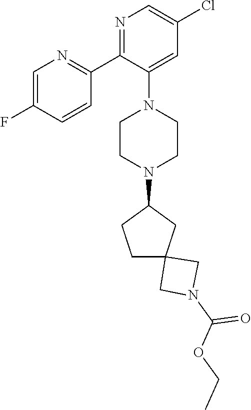 [SMILES: CCOC(=O)N1CC2(CC[C@@H](N3CCN(c4cc(Cl)cnc4-c4ccc(F)cn4)CC3)C2)C1] |
| A253 | 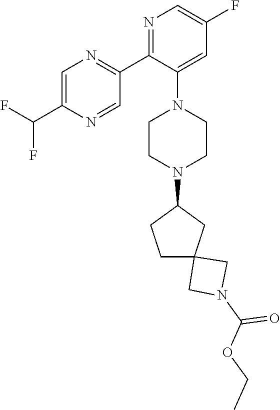 [SMILES: CCOC(=O)N1CC2(CC[C@@H](N3CCN(c4cc(F)cnc4-c4cnc(C(F)F)cn4)CC3)C2)C1] |
| A254 | 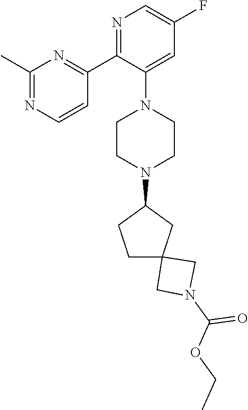 [SMILES: CCOC(=O)N1CC2(CC[C@@H](N3CCN(c4cc(F)cnc4-c4ccnc(C)n4)CC3)C2)C1] |
| A255 | 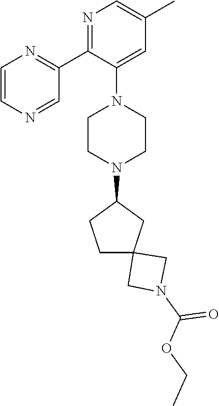 [SMILES: CCOC(=O)N1CC2(CC[C@@H](N3CCN(c4cc(C)cnc4-c4cnccn4)CC3)C2)C1] |
| A256 | 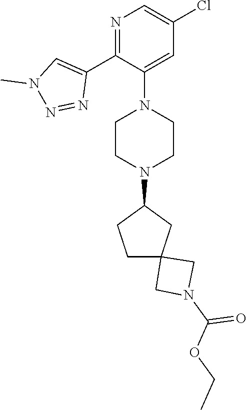 [SMILES: CCOC(=O)N1CC2(CC[C@@H](N3CCN(c4cc(Cl)cnc4-c4cn(C)nn4)CC3)C2)C1] |
| A257 | 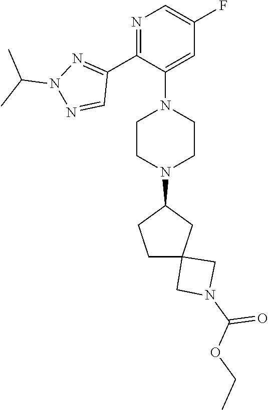 [SMILES: CCOC(=O)N1CC2(CC[C@@H](N3CCN(c4cc(F)cnc4-c4cnn(C(C)C)n4)CC3)C2)C1] |
| A258 | 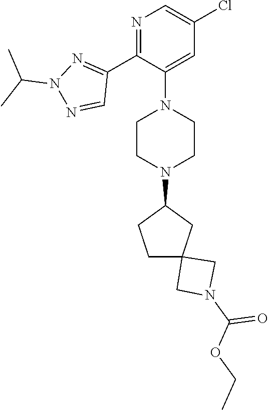 [SMILES: CCOC(=O)N1CC2(CC[C@@H](N3CCN(c4cc(Cl)cnc4-c4cnn(C(C)C)n4)CC3)C2)C1] |
| A259 | 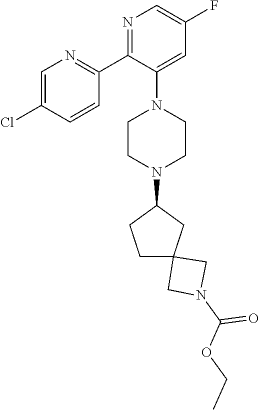 [SMILES: CCOC(=O)N1CC2(CC[C@@H](N3CCN(c4cc(F)cnc4-c4ccc(Cl)cn4)CC3)C2)C1] |
| A260 | 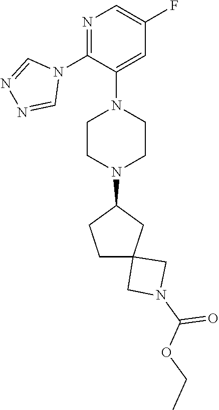 [SMILES: CCOC(=O)N1CC2(CC[C@@H](N3CCN(c4cc(F)cnc4-n4cnnc4)CC3)C2)C1] |
| A261 | 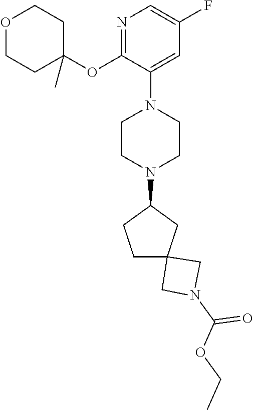 [SMILES: CCOC(=O)N1CC2(CC[C@@H](N3CCN(c4cc(F)cnc4OC4(C)CCOCC4)CC3)C2)C1] |
| A262 | 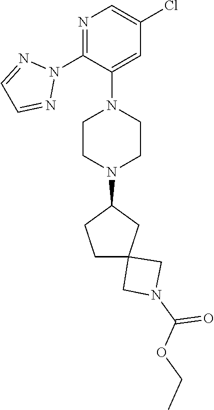 [SMILES: CCOC(=O)N1CC2(CC[C@@H](N3CCN(c4cc(Cl)cnc4-n4nccn4)CC3)C2)C1] |
| A263 | 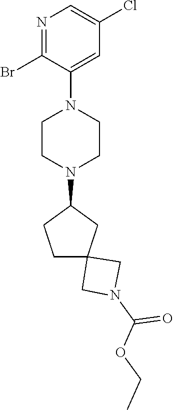 [SMILES: CCOC(=O)N1CC2(CC[C@@H](N3CCN(c4cc(Cl)cnc4Br)CC3)C2)C1] |
| A264 | 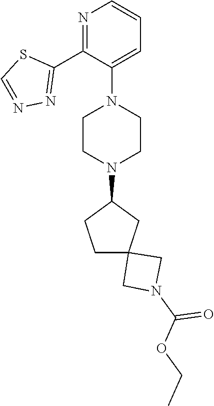 [SMILES: CCOC(=O)N1CC2(CC[C@@H](N3CCN(c4cccnc4-c4nncs4)CC3)C2)C1] |
| A265 | 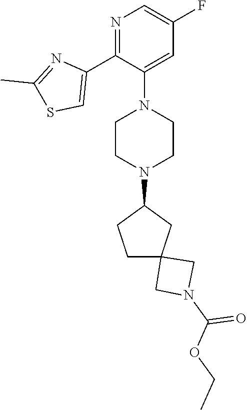 [SMILES: CCOC(=O)N1CC2(CC[C@@H](N3CCN(c4cc(F)cnc4-c4csc(C)n4)CC3)C2)C1] |
| A266 | 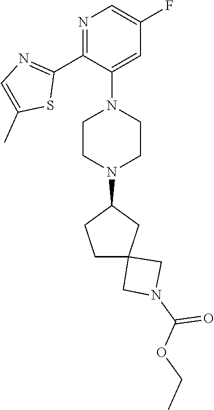 [SMILES: CCOC(=O)N1CC2(CC[C@@H](N3CCN(c4cc(F)cnc4-c4ncc(C)s4)CC3)C2)C1] |
| A267 | 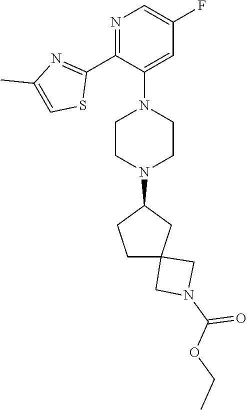 [SMILES: CCOC(=O)N1CC2(CC[C@@H](N3CCN(c4cc(F)cnc4-c4nc(C)cs4)CC3)C2)C1] |
| A268 | 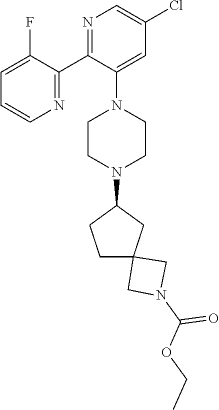 [SMILES: CCOC(=O)N1CC2(CC[C@@H](N3CCN(c4cc(Cl)cnc4-c4ncccc4F)CC3)C2)C1] |
| A269 | 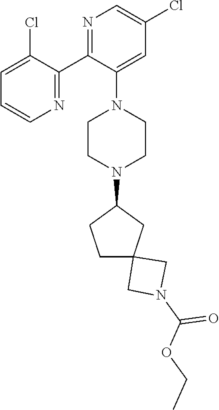 [SMILES: CCOC(=O)N1CC2(CC[C@@H](N3CCN(c4cc(Cl)cnc4-c4ncccc4Cl)CC3)C2)C1] |
| A270 | 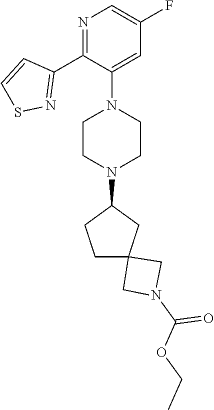 [SMILES: CCOC(=O)N1CC2(CC[C@@H](N3CCN(c4cc(F)cnc4-c4ccsn4)CC3)C2)C1] |
| A271 | 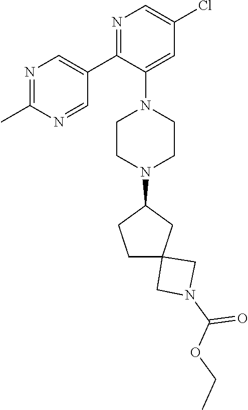 [SMILES: CCOC(=O)N1CC2(CC[C@@H](N3CCN(c4cc(Cl)cnc4-c4cnc(C)nc4)CC3)C2)C1] |
| A272 | 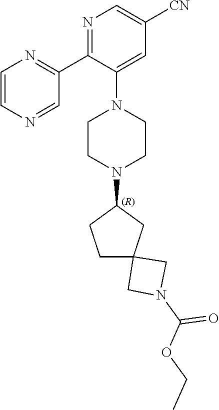 |
| A273 | 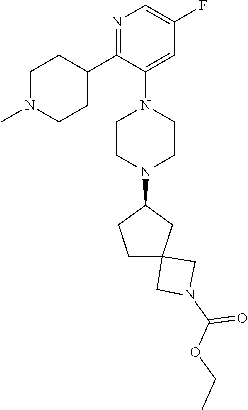 [SMILES: CCOC(=O)N1CC2(CC[C@@H](N3CCN(c4cc(F)cnc4C4CCN(C)CC4)CC3)C2)C1] |
| A274 | 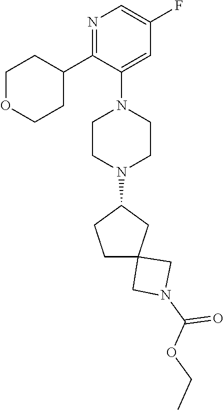 [SMILES: CCOC(=O)N1CC2(CC[C@H](N3CCN(c4cc(F)cnc4C4CCOCC4)CC3)C2)C1] |
| A275 | 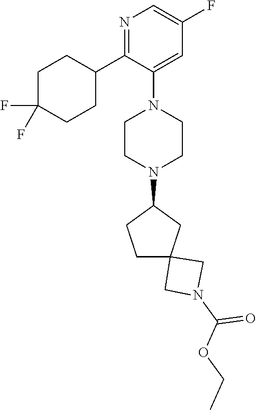 [SMILES: CCOC(=O)N1CC2(CC[C@@H](N3CCN(c4cc(F)cnc4C4CCC(F)(F)CC4)CC3)C2)C1] |
| A276 | 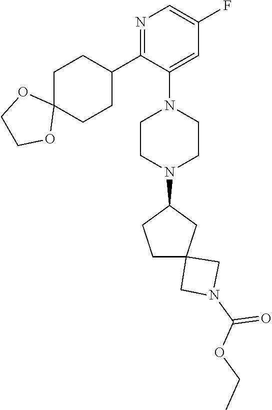 [SMILES: CCOC(=O)N1CC2(CC[C@@H](N3CCN(c4cc(F)cnc4C4CCC5(CC4)OCCO5)CC3)C2)C1] |
| A277 | 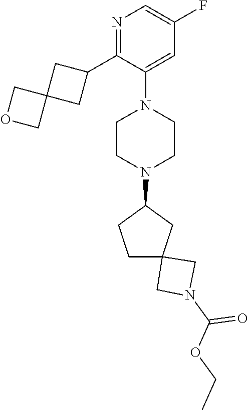 [SMILES: CCOC(=O)N1CC2(CC[C@@H](N3CCN(c4cc(F)cnc4C4CC5(COC5)C4)CC3)C2)C1] |
| A278 | 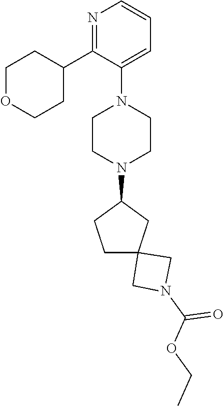 [SMILES: CCOC(=O)N1CC2(CC[C@@H](N3CCN(c4cccnc4C4CCOCC4)CC3)C2)C1] |
| A279 | 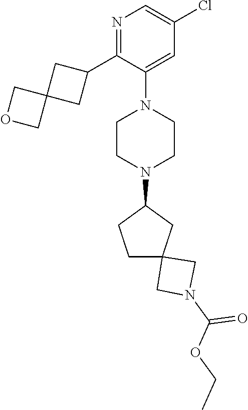 [SMILES: CCOC(=O)N1CC2(CC[C@@H](N3CCN(c4cc(Cl)cnc4C4CC5(COC5)C4)CC3)C2)C1] |
| A280 | 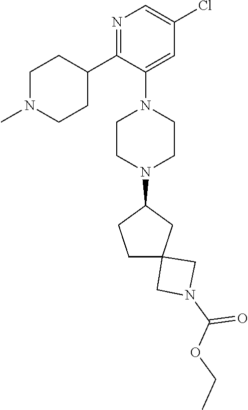 [SMILES: CCOC(=O)N1CC2(CC[C@@H](N3CCN(c4cc(Cl)cnc4C4CCN(C)CC4)CC3)C2)C1] |
| A281 | 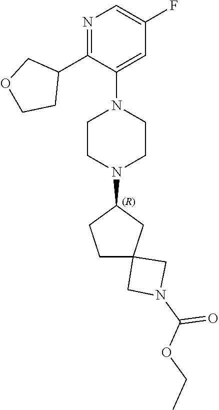 |
| A282 | 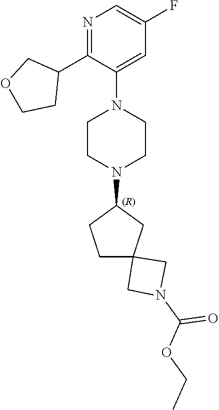 |
| A283 | 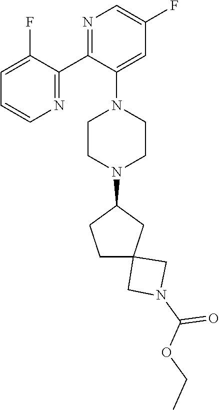 [SMILES: CCOC(=O)N1CC2(CC[C@@H](N3CCN(c4cc(F)cnc4-c4ncccc4F)CC3)C2)C1] |
| A284 | 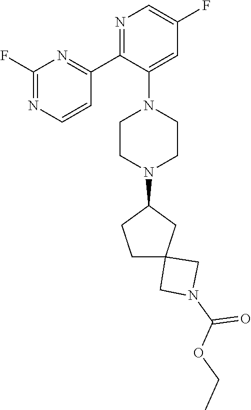 [SMILES: CCOC(=O)N1CC2(CC[C@@H](N3CCN(c4cc(F)cnc4-c4ccnc(F)n4)CC3)C2)C1] |
| A285 | 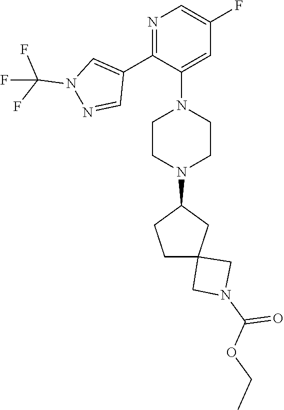 [SMILES: CCOC(=O)N1CC2(CC[C@@H](N3CCN(c4cc(F)cnc4-c4cnn(C(F)(F)F)c4)CC3)C2)C1] |
| A286 | 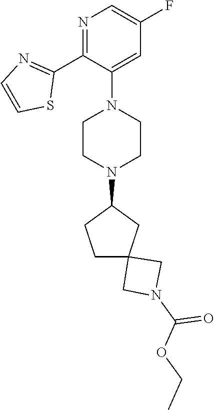 [SMILES: CCOC(=O)N1CC2(CC[C@@H](N3CCN(c4cc(F)cnc4-c4nccs4)CC3)C2)C1] |
| A287 | 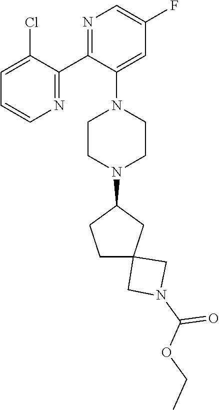 [SMILES: CCOC(=O)N1CC2(CC[C@@H](N3CCN(c4cc(F)cnc4-c4ncccc4Cl)CC3)C2)C1] |
| A288 | 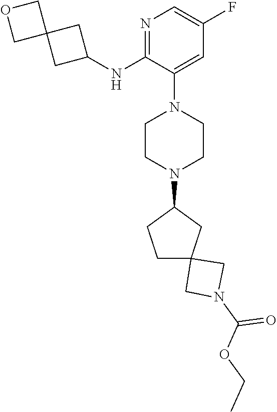 [SMILES: CCOC(=O)N1CC2(CC[C@@H](N3CCN(c4cc(F)cnc4NC4CC5(COC5)C4)CC3)C2)C1] |
| A289 | 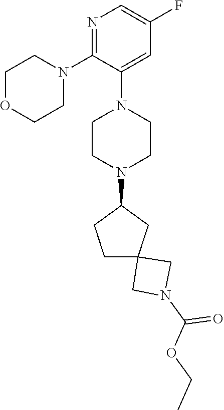 [SMILES: CCOC(=O)N1CC2(CC[C@@H](N3CCN(c4cc(F)cnc4N4CCOCC4)CC3)C2)C1] |
| A290 | 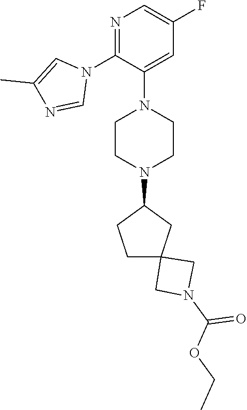 [SMILES: CCOC(=O)N1CC2(CC[C@@H](N3CCN(c4cc(F)cnc4-n4cnc(C)c4)CC3)C2)C1] |
| A291 | 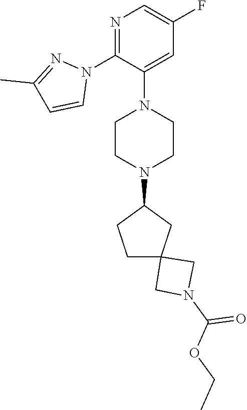 [SMILES: CCOC(=O)N1CC2(CC[C@@H](N3CCN(c4cc(F)cnc4-n4ccc(C)n4)CC3)C2)C1] |
| A292 | 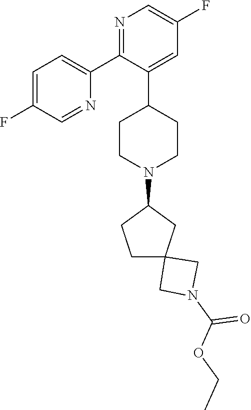 [SMILES: CCOC(=O)N1CC2(CC[C@@H](N3CCC(c4cc(F)cnc4-c4ccc(F)cn4)CC3)C2)C1] |
| A293 | 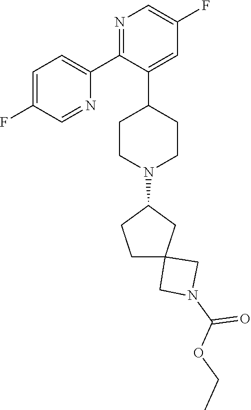 [SMILES: CCOC(=O)N1CC2(CC[C@H](N3CCC(c4cc(F)cnc4-c4ccc(F)cn4)CC3)C2)C1] |

The present disclosure also includes all pharmaceutically acceptable isotopically labeled compounds, which are identical to those recited herein, wherein one or more atoms are replaced by an atom having the same atomic number, but an atomic mass or mass number different from the atomic mass or mass number which predominates in nature. Examples of isotopes suitable for inclusion in the compounds of the present disclosure include, but are not limited to, isotopes of hydrogen, such as ²H, ³H; carbon, such as ¹¹C, ¹³C, and ¹⁴C; chlorine, such as ³¹Cl; fluorine, such as ¹⁸F; iodine, such as ¹²³I and ¹²⁵I: nitrogen, such as ¹³N and ¹⁵N; oxygen, such as ¹⁵O, ¹⁷O, and ¹⁸O; phosphorus, such as ³²P; and sulfur, such as ³⁵S. Isotopically-labeled compounds may include combinations of two or more of the above, same or different, isotopes. Certain isotopically-labeled compounds of the present disclosure, for example, those incorporating a radioactive isotope, are useful in drug and/or substrate tissue distribution studies (e.g., assays). The radioactive isotopes tritium, i.e., ³H or “T”, and carbon-14, i.e., ¹⁴C, are particularly useful for this purpose in view of their ease of incorporation and ready means of detection. Substitution with heavier isotopes such as deuterium, i.e., ²H, or “D”, may afford certain therapeutic advantages resulting from greater metabolic stability, for example, increased in vivo half-life or reduced dosage requirements and, hence, may be preferred in some circumstances. Substitution with positron-emitting isotopes, such as ¹¹C, ¹⁸F, ¹⁵O and ¹³N, can be useful in positron emission tomography (PET) studies for examining substrate receptor occupancy. Isotopically labeled compounds of the present disclosure can generally be prepared by conventional techniques known to those skilled in the art or by processes analogous to those described in the accompanying Schemes and/or in the Examples and Preparations using an appropriate isotopically labeled reagent in place of the non-labeled reagent previously employed. Pharmaceutically acceptable solvates in accordance with the disclosure include those wherein the solvent of crystallization may be isotopically substituted, e.g., D₂O, acetone-d₆, or DMSO-d₆.

The chemical structures having one or more stereocenters depicted with dashed and bold wedged bonds (i.e., [symbol] and [symbol] ) are meant to indicate absolute stereochemistry of the stereocenter(s) present in the chemical structure. Bonds symbolized by a simple line do not indicate a stereo-preference. Bonds symbolized by dashed or bold straight bonds (i.e. [symbol] and [symbol] ) are meant to indicate a relative stereochemistry of the stereocenter(s) present in the chemical structure. Unless otherwise indicated to the contrary, chemical structures that include one or more stereocenters which are illustrated herein without indicating absolute or relative stereochemistry, encompass all possible stereoisomeric forms of the compound (e.g., diastereomers, enantiomers) and mixtures thereof. Structures with a single bold or dashed wedged line, and at least one additional simple line, encompass a single enantiomeric series of all possible diastereomers. Similarly, the chemical structures having alkenyl groups are meant to encompass both cis and trans orientations, or when substituted, E- and Z-isomers of the chemical structure.

### Pharmaceutically Acceptable Salts and Co-Crystals

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

As used herein, “Formula (I)”, “Formula (Ia)”, “Formula (Ib)”, “Formula (Ic)”, “Formula (Id)”, and “Formula (Ie)”, are also defined to include all forms of the compounds of the disclosure including, but not limited to, hydrates, solvates, isomers (including for example rotational stereoisomers), crystalline and non-crystalline forms, isomorphs, polymorphs, metabolites, prodrugs thereof. For example, the compounds disclosed herein, or pharmaceutically acceptable salts thereof, may exist in unsolvated and solvated forms with pharmaceutically acceptable solvents such as water, ethanol and the like. When the solvent or water is tightly bound, the complex will have a well-defined stoichiometryindependent of humidity. When, however, the solvent or water is weakly bound, as in channel solvates and hygroscopic compounds, the water/solvent content will be dependent on humidity and drying conditions. In such cases, non-stoichiometry will be the norm. In general, the solvated forms are considered equivalent to the unsolvated forms for the purposes of the present disclosure.

The compounds of the disclosure may exist as clathrates or other complexes (e.g., co-crystals). Included within the scope of the disclosure are complexes such as clathrates, drug-host inclusion complexes wherein the drug and host are present in stoichiometric or non-stoichiometric amounts. Also included are complexes of the compounds of the disclosure containing two or more organic and/or inorganic components, which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975). Co-crystals are typically defined as crystalline complexes of neutral molecular constituents that are bound together through non-covalent interactions, but could also be a complex of a neutral molecule with a salt. Co-crystals may be prepared by melt crystallization, by recrystallization from solvents, or by physically grinding the components together; see O. Almarsson and M. J. Zaworotko, Chem. Commun. 2004, 17, 1889-1896. For a general review of multi-component complexes, see J. K. Haleblian, J. Pharm. Sci. 1975, 64, 1269-1288.

In some cases, the compounds of the present disclosure may exist in and/or be isolated as atropisomers (e.g., one or more atropenantiomers). Those skilled in the art would recognize that atropisomerism may exist in a compound that has two or more aromatic rings (for example, two aromatic rings linked through a single bond). See e.g., Freedman, T. B. et al., Absolute Configuration Determination of Chiral Molecules in the Solution State Using Vibrational Circular Dichroism. Chirality 2003, 15, 743-758; and Bringmann, G. et al., Atroposelective Synthesis of Axially Chiral Biaryl Compounds. Angew. Chem., Int. Ed. 2005, 44, 5384-5427.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of the present disclosure may also exist as an N-oxide thereof, or a pharmaceutically acceptable salt of the compound or N-oxide.

As it is known to the person skilled in the art, amine compounds (i.e., those comprising one or more nitrogen atoms), for example tertiary amines, can form N-oxides (also known as amine oxides or amine N-oxides). Generally, An N-oxide has the formula of R₃N⁺—O— wherein the parent amine R₃N can be for example, a tertiary amine (for example, each R is independently alkyl, arylalkyl, aryl, heteroaryl, or the like), a heterocyclic or heteroaromatic amine (for example, R₃N together forms 1-alkylpiperidine, 1-alkylpyrrolidine, 1-benzylpyrrolidine, or pyridine). For instance, an imine nitrogen, especially heterocyclic or heteroaromatic imine nitrogen, or pyridine-type nitrogen (═N—) atom, such as a nitrogen atom in pyridine, pyridazine, or pyrazine, can be N-oxidized to form the N-oxide comprising the ≡N⁺—O— group. Thus, a compound according to the present disclosure comprising one or more nitrogen atoms (e.g., an imine nitrogen atom) may be capable of forming an N-oxide thereof (e.g., mono-N-oxides, bis-N-oxides or multi-N-oxides, or mixtures thereof depending on the number of nitrogen atoms suitable to form stable N-oxides).

As used herein, the term “N-oxide(s)” refer to all possible, and in particular all stable, N-oxide forms of the amine compounds (e.g., compounds comprising one or more imine nitrogen atoms) described herein, such as mono-N-oxides (including different isomers when more than one nitrogen atom of an amine compound can form a mono-N-oxide) or multi-N-oxides (e.g., bis-N-oxides), or mixtures thereof in any ratio.

As noted above, the compounds of the disclosure (or N-oxides thereof) may exist in the form of pharmaceutically acceptable salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of the present disclosure with an acid whose anion, or a base whose cation, is generally considered suitable for human consumption. Pharmaceutically acceptable salts are particularly useful as products of the methods of the present disclosure because of their greater aqueous solubility relative to the parent compound.

Suitable pharmaceutically acceptable acid addition salts of the compounds of the present disclosure when possible include those derived from inorganic acids, such as, but not limited to, hydrochloric, hydrobromic, hydrofluoric, boric, fluoroboric, phosphoric, meta-phosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include but are not limited to aliphatic, cycloaliphatic, aromatic, araliphatic, heterocyclic, carboxylic, and sulfonic classes of organic acids.

Specific examples of suitable organic acids include but are not limited to acetate, trifluoroacetate, formate, propionate, succinate, glycolate, gluconate, digluconate, lactate, malate, tartrate, citrate, ascorbate, glucuronate, maleate, fumarate, pyruvate, aspartate, glutamate, benzoate, anthranilate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylamino-sulfonate, algenic acid, R-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalene-sulfonate, oxalate, palmoate, pectinate, 3-phenylpropionate, picrate, pivalate, thiocyanate, and undecanoate.

Furthermore, where the compounds of the disclosure carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N-dibenzylethylenediamine, chloroprocaine, choline, diethanol-amine, ethylenediamine, meglumine (N-methylglucamine), and procaine. Basic nitrogen-containing groups may be quaternized with agents such as lower alkyl (C1-Cs) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

In some cases, hemisalts of acids and bases may also be formed, for example, hemisulphate and hemicalcium salts, or sequifumarate.

For a review on suitable salts, see “Handbook of Pharmaceutical Salts: Properties, Selection, and Use” by Stahl and Wermuth (Wiley-VCH, 2002). Methods for making pharmaceutically acceptable salts of compounds of the disclosure are known to one of skill in the art.

Compounds of the disclosure may exist in a continuum of solid states ranging from fully amorphous to fully crystalline. The term ‘amorphous’ refers to a state in which the material lacks long-range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from apparent solid to a material with liquid properties occurs, which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO′Na*, —COOK*, or —SO3s′Na*) or non-ionic (such as —N″N*(CHs)3) polar head group. For more information, see Crystals and the Polarizing Microscope by N. H. Hartshorne and A. Stuart, 4” Edition (Edward Arnold, 1970).

The disclosure also relates to prodrugs of the compounds of the present disclosure. Thus certain derivatives of compounds of the disclosure which may have little or no pharmacological activity themselves can, when administered into or onto the body, be converted into compounds of Formula I having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the disclosure can, for example, be produced by replacing appropriate functionalities present in the compounds of the disclosure with certain moieties known to those skilled in the art as ‘pro-moieties’ as described, for example, in Design of Prodrugs by H. Bundgaard (Elsevier, 1985), or in Prodrugs: Challenges and Reward, 2007 edition, edited by Valentino Stella, Ronald Borchardt, Michael Hageman, Reza Oliyai, Hans Maag, Jefferson Tilley, pages 134-175 (Springer, 2007).

Moreover, certain compounds of the disclosure may themselves act as prodrugs of other compounds of the disclosure. This disclosure also encompasses compounds of the disclosure containing protective groups. One skilled in the art will also appreciate that compounds of the disclosure can also be prepared with certain protecting groups that are useful for purification or storage and can be removed before administration to a patient. The protection and deprotection of functional groups is described in “Protective Groups in Organic Chemistry”, edited by J. W. F. McOmie, Plenum Press (1973) and “Protective Groups in Organic Synthesis”, 3rd edition, T. W. Greene and P. G. M. Wuts, Wiley-Interscience (1999).

Also included within the scope of the disclosure are metabolites of compounds of the disclosure, that is, compounds formed in vivo upon administration of the drug.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g., orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g., eneteral or parenteral). All methods that can be used by those skilled in the art to administer a pharmaceutically active agent are contemplated. In some cases, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Depot injection formulations are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

### Methods of Treatment

The compounds disclosed herein (e.g., the compounds of Formula (I), Formula (Ia), Formula (Ib), Formula (Ic), and Formula (Id)), and pharmaceutically acceptable salts thereof, can act on the M4 receptor. The muscarinic acetylcholine receptor M4 (also known as muscarinic 4 or CHRM4) is a protein in humans that is encoded for the CHRM4 gene. M4 receptors are predominantly expressed in the brain. Key regions of the brain where M4 receptor expression occurs are the striatum, cortex, and hippocampus with the highest expression occurring in the striatum (approx. 46%) where M4 is the major muscarinic subtype. M4 is sporadically expressed in the periphery (e.g., testis, skin and colon).

M4 receptors are coupled to G_{q/i} proteins and function as inhibitory autoreceptors in the striatum and midbrain (Zhang et. al; “Multiple Muscarinic Acetylcholine Receptor Subtypes Modulate Striatal Dopamine Release, as Studied with M1-M5 Muscarinic Receptor Knock-Out Mice”; Journal of Neuroscience 1 Aug. 2002, 22 (15) 6347-6352; Tzavara. et. al.; “M₄ muscarinic receptors regulate the dynamics of cholinergic and dopaminergic neurotransmission: relevance to the pathophysiology and treatment of related central nervous system pathologies”; The FASEB Journal, 2004; 18: 1410-1412), and as postsynaptic modulatory receptors in the striatum, neocortex and hippocampus (Levey et. al; Identification and localization of muscarinic acetylcholine receptor proteins in brain with subtype-specific antibodies; Journal of Neuroscience 1 Oct. 1991, 11 (10) 3218-3226; Gil, et al; “Muscarinic receptor subtypes in human iris-ciliary body measured by immunoprecipitation.” Investigative ophthalmology & visual science 38.7 (1997): 1434-1442). M4 receptors are also found presynaptically on glutamatergic synapses from cortex to striatum (Pancani, T., et al., “Allosteric activation of M4 improve behavioral and physiological alterations in early symptomatic YAC128 mice”, Proceedings of the National Academy of the Sciences of the United States of America, 2015 Nov. 10; 112(45):14078-83), and on hippocampal glutamate neurons where presynaptic M4 modulates glutamate release. The highest expression of M4 receptors is found in the striatum, M4 receptors also possess a regulatory effect on dopaminergic neurotransmission, and are coexpressed with D1 dopamine receptors in a subset of striatal medium spiny neurons which contain GABA as a major neurotransmitter (Bernard, et al; “Phenotypical characterization of the rat striatal neurons expressing muscarinic receptor genes”; Journal of Neuroscience 1 Sep. 1992, 12 (9) 3591-3600; Di Chiara, et. al; “Modulatory functions of neurotransmitters in the striatum: ACh/dopamine/NMDA interactions; Trends in Neurosciences; Volume 17, Issue 6, 1994, Pages 228-233; Ince, et. al; “Differential expression of D1 and D2 dopamine and m4 muscarinic acetylchoine receptor proteins in identified striatonigral neurons”: Synapse, (1997) 27: 357-366).

It has been hypothesized that administration of a selective M4 agonist would provide antipsychotic activity for the treatment of schizophrenia (Felder et al. “Elucidating the Role of Muscarinic Receptors in Psychosis”, Life Sci. 68:2605-2613, 2001). This belief was further supported by studies that demonstrated M4 receptors modulate the dynamics of dopaminergic and cholinergic neurotransmission and that a state of dopamine hyperfunctions results with a loss of M4 function (Tzavara et al., 2004).

The compounds of the present disclosure may also be useful for treating/alleviating the neuropsychiatric symptoms (i.e., behavioral symptoms) associated with Alzheimer's Disease and Schizophrenia (Foster et. al., “Activation of M1 and M4 muscarinic receptors as potential treatments for Alzheimer's disease and schizophrenia”, Neuropsychiatric Disease and Treatment; Volume 2014.10, pp. 183-191). These behavioral symptoms include, but are not limited to, agitation, vocal outburst, compulsiveness, anxiety, irritability, combativeness, disorientation, illusion, delusion, hallucination, suspiciousness, apathy, depression, disinhibition, aberrant motor and obsessive-compulsive behaviors, as well as sleep disorders (Dillon, Carol, et. al. “Behavioral symptoms related to cognitive impairment”, Neuropsychiatric Disease and Treatment; 2013:9 1443-1455). By treating/alleviating the above-mentioned behavioral symptoms, it is believed that the compounds of the present disclosure will also enhance cognition.

In view of the above, the compounds of the present disclosure may be useful for the treatment of schizophrenia and Alzheimer's Disease. The compounds of the present disclosure may also be useful for the treatment of Parkinson's Disease, Huntington's Disease, addiction, substance abuse disorder, depression and epilepsy. The compounds of the present disclosure may also be useful for Alzheimer's disease psychosis.

It is believed the M4 selective activators of the present disclosure may also have a wide range of other therapeutic applications for the treatment of conditions or diseases of the central nervous system which include neurologic, neurodegenerative and/or psychiatric disorders. Neurologic, neurodegenerative and/or psychiatric disorders include but are not limited to, (1) mood [affective]disorders; (2) neurotic, stress-related and somatoform disorders including anxiety disorders; (3) disorders comprising the symptom of cognitive deficiency in a mammal, including a human; (4) disorders comprising attention deficits, executive function deficits (working memory deficits), dysfunction of impulse control, extrapyramidal symptoms, disorders that are based on a malfunction of basal ganglia, hippocampus and prefrontal cortex; (5) behavioral and emotional disorders with onset usually occurring in childhood and adolescence; (6) disorders of psychological development; (7) systemic atrophies primarily affecting the central nervous system; (8) extrapyramidal and movement disorders; (9) behavioral syndromes associated with physiological disturbances and physical factors; (10) disorders of adult personality and behavior; (11) schizophrenia and other psychotic disorders; (12) mental and behavioral disorders due to psychoactive substance use; (13) sexual dysfunction comprising excessive sexual drive; (14) mental retardation; (15) factitious disorders, e.g., acute hallucinatory mania; (16) episodic and paroxysmal disorders, epilepsy; (17) narcolepsy; (18) dementia, and (19) amyotrophic lateral sclerosis.

Examples of mood (affective) disorders that can be treated according to the present disclosure include, but are not limited to, bipolar disorder I, hypomania (manic and mixed form), bipolar disorder II; depressive disorders such as single depressive episode or recurrent major depressive disorder, chronic depression, psychotic depression, minor depressive disorder, depressive disorder with postpartum onset, depressive disorders with psychotic symptoms; persistent mood [affective]disorders such as cyclothymia, dysthymia, euthymia; premenstrual syndrome (PMS) and premenstrual dysphoric disorder.

Examples of neurotic, stress-related and somatoform disorders that can be treated according to the present disclosure include, but are not limited to, anxiety disorders, social anxiety disorder, general anxiety disorder, panic disorder with or without agoraphobia, specific phobia, social phobia, chronic anxiety disorders; obsessive compulsive disorder; reaction to severe stress and adjustment disorders, such as post-traumatic stress disorder (PTSD), acute stress disorder, other neurotic disorders such as depersonalization-derealization syndrome.

The phrase “cognitive deficiency” as used herein and “disorders comprising the symptom of cognitive deficiency” refers to a subnormal functioning or a suboptimal functioning in one or more cognitive aspects such as memory, intellect, learning and logic ability, or attention and executive function (working memory) in a particular individual comparative to other individuals within the same general age population.

Examples of “disorders comprising the symptom of cognitive deficiency” that can be treated according to the present disclosure include, but are not limited to, cognitive deficits primarily but not exclusively related to amnesia, psychosis (schizophrenia), Parkinson's disease, Alzheimer's Disease, multi-infarct dementia, senile dementia, Lewis body dementia, stroke, frontotemporal dementia, progressive supranuclear palsy, Huntington's disease, HIV disease (HIV-associated dementia), cerebral trauma and drug abuse; mild cognitive disorder ADHD, Asperger's syndrome, and age-associated memory impairment; cognitive decline or delerium post-operative or in association with intensive care therapy.

Examples of disorders usually first diagnosed in infancy, childhood and adolescence that can be treated according to the present disclosure include, but are not limited to, hyperkinetic disorders including disturbance of activity and attention, attention deficit/hyperactivity disorder (ADHD), hyperkinetic conduct disorder; attention deficit disorder (ADD); conduct disorders, including but not limited to depressive conduct disorder; tic disorders including transient tic disorder, chronic motor or vocal tic disorder, combined vocal and multiple motor tic disorder (Gilles de la Tourette's syndrome), substance-induced tic disorders; autistic disorders; Batten disease, excessive masturbation, nail-biting, nose-picking and thumb-sucking.

Examples of disorders of psychological development that can be treated according to the present disclosure include, but are not limited to pervasive developmental disorders, including but not limited to Asperger's syndrome and Rett syndrome, autistic disorders, childhood autism and overactive disorder associated with mental retardation and stereotyped movements, specific developmental disorder of motor function, specific developmental disorders of scholastic skills.

Examples of systemic atrophies primarily affecting the central nervous system that can be treated according to the present disclosure include, but are not limited to, multiple sclerosis systemic atrophies primarily affecting the basal ganglia including Huntington's disease, and amyotrophic lateral sclerosis.

Examples of extrapyramidal and movement disorders with malfunction and/or degeneration of basal ganglia that can be treated according to the present disclosure include, but are not limited to, Huntington's disease; Parkinson's disease; second Parkinsonism such as postencephalitic Parkinsonism; Parkinsonism comprised in other disorders; Niemann-Pick disease, Lewy body disease; degenerative diseases of the basal ganglia; other extrapyramidal and movement disorders including tremor, essential tremor and drug-induced tremor, myoclonus, chorea and drug-induced chorea, drug-induced tics and tics of organic origin, drug-induced acute dystonia, drug-induced tardive dyskinesia, muscular spasms and disorders associated with muscular spasticity or weakness including tremors; mental deficiency (including spasticity, Down syndrome and fragile X syndrome), L-dopa-induced dyskinesia; restless leg syndrome and Stiff-man syndrome.

Further examples of movement disorders with malfunction and/or degeneration of basal ganglia that can be treated according to the present disclosure include, but are not limited to, dystonia including but not limited to focal dystonia, multiple-focal or segmental dystonia, torsion dystonia, hemispheric, generalized and tardive dystonia (induced by psychopharmacological drugs). Focal dystonia include cervical dystonia (torticolli), blepharospasm (cramp of the eyelid), appendicular dystonia (cramp in the extremities, like the writer's cramp), or mandibular dystonia and spasmodic dysphonia (cramp of the vocal cord); neuroleptic-induced movement disorders including but not limited to neuroleptic malignant syndrome (NMS), neuroleptic-induced Parkinsonism, neuroleptic-induced early onset or acute dyskinesia, neuroleptic-induced acute dystonia, neuroleptic-induced acute akathisia, neuroleptic-induced tardive dyskinesia, and neuroleptic-induced tremor.

Examples of behavioral syndromes associated with physiological disturbances and physical factors according to the present disclosure include, but are not limited to, nonorganic sleep disorders, including but not limited to nonorganic hypersomnia, nonorganic disorder of the sleep-wake schedule (circadian rhythm sleep disorder), insomnia, parasomnia and sleep deprivation; mental and behavioral disorders associated with the puerperium including postnatal and postpartum depression; eating disorders, including but not limited to anorexia nervosa, bulimia nervosa, binge eating disorder, hyperphagia, obesity, compulsive eating disorders and pagophagia.

Examples of disorders of adult personality and behavior that can be treated according to the present disclosure include, but are not limited to, personality disorders, including but not limited to emotionally unstable, borderline, obsessive-compulsive, anankastic, dependent and passive-aggressive personality disorder; habit and impulse disorders (impulse-control disorder) including intermittent explosive disorder, pathological gambling, pathological fire-setting (pyromania), pathological stealing (kleptomania), trichotillomania; Munchausen syndrome.

Examples of schizophrenia and other psychotic disorders that can be treated according to the present disclosure include, but are not limited to, continuous or episodic schizophrenia of different types (for instance paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; acute, transient and persistent psychotic disorders; induced delusional disorders; schizoaffective disorders of different type (for instance manic depressive or mixed type); puerperal psychosis and other and unspecified nonorganic psychosis such as social withdrawal in schizophrenia.

Examples of mental and behavioral disorders due to psychoactive substance use that can be treated according to the present disclosure include, but are not limited to, mental and behavioral disorders due to use of alcohol, opioids, cannabinoids, sedatives or hypnotics, cocaine; mental and behavioral disorders due to the use of other stimulants including caffeine, mental and behavioral disorders due to drug dependence and abuse (e.g., narcotic dependence, alcoholism, amphetamine and methamphetamine dependence, opioid dependence, cocaine addiction, nicotine dependence, and drug withdrawal syndrome, and relapse prevention), use of hallucinogens, tobacco (nicotine), volatile solvents and mental and behavioral disorders due to multiple drug use and use of other psychoactive substances including the following subtype symptoms: harmful use, dependence syndrome, withdrawal state, and withdrawal state with delirium.

Examples of dementia that can be treated according to the present disclosure include, but are not limited to, vascular dementia, dementia due to Creutzfeld-Jacob disease, HIV, head trauma, Parkinson's, Huntington's, Pick's disease, dementia of the Alzheimer's type.

Schizophrenia or psychosis for which the compounds, N-oxide thereof, and pharmaceutically acceptable salts of the foregoing of the disclosure may be useful includes one or more of the following conditions: schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthesia, amphetamine and other psychostimulants and cocaine) psychosis/psychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's Disease and post-traumatic stress syndrome), including both the positive and the negative symptoms of schizophrenia and other psychoses; cognitive disorders including dementia (associated with Alzheimer's Disease, ischemia, multi-infarct dementia, trauma, vascular problems or stroke, HIV disease, Parkinson's disease, Huntington's disease, Pick's disease, Creutzfeld-Jacob disease, perinatal hypoxia, other general medical conditions or substance abuse); delirium, amnestic disorders, or age related cognitive decline.

In addition to the central nervous system disorders mentioned above, the compounds of the present disclosure may be used to treat other M4-mediated (or M4-associated) disorders such as, but not limited to, addiction (e.g., substance addiction such as addiction to opioids, cocaine, or alcohol), pain (e.g., acute pain, inflammatory pain, and neuropathic pain), and a sleep disorder (such as those related to REM sleep regulation, for example, those related to REM sleep onset). Additional M4-mediated (or M4-associated) disorders or conditions that may be treated by the compounds of the disclosure include, dry mouth, a cognitive disorder (e.g., mild cognitive impairment), dyskinesia, pulmonary hypertension, chronic obstructive pulmonary disease (COPD), asthma, urinary incontinence, glaucoma, Trisomy 21 (Down Syndrome), cerebral amyloid angiopathy, dementia (e.g. degenerative dementia), Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes, autism, and atherosclerosis. See e.g. U.S. Pat. No. 8,664,234.

Potential sleep disorders for which the compounds, N-oxide thereof, and pharmaceutically acceptable salts of the foregoing of the disclosure may be useful include: enhancing sleep quality; improving sleep quality; augmenting sleep maintenance; increasing the value which is calculated from the time that a subject sleeps divided by the time that a subject is attempting to sleep; decreasing sleep latency or onset (the time it takes to fall asleep); decreasing difficulties in falling asleep; increasing sleep continuity; decreasing the number of awakenings during sleep; decreasing nocturnal arousals; decreasing the time spent awake following the initial onset of sleep; increasing the total amount of sleep; reducing the fragmentation of sleep; altering the timing, frequency or duration of REM sleep bouts; altering the timing, frequency or duration of slow wave (i.e. stages 3 or 4) sleep bouts; increasing the amount and percentage of stage 2 sleep; promoting slow wave sleep; enhancing EEG-delta activity during sleep; increasing daytime alertness; reducing daytime drowsiness; treating or reducing excessive daytime sleepiness; insomnia; hypersomnia; narcolepsy; interrupted sleep; sleep apnea; wakefulness; nocturnal myoclonus; REM sleep interruptions; jet-lag; shift workers' sleep disturbances; dyssomnias; night terror; insomnias associated with depression, emotional/mood disorders, as well as sleep walking and enuresis, and sleep disorders which accompany aging; Alzheimer's sundowning; conditions associated with circadian rhythmicity as well as mental and physical disorders associated with travel across time zones and with rotating shift-work schedules; conditions due to drugs which cause reductions in REM sleep as a side effect; syndromes which are manifested by non-restorative sleep and muscle pain or sleep apnea which is associated with respiratory disturbances during sleep; and conditions which result from a diminished quality of sleep.

Pain disorders for which the compounds, N-oxide thereof, and pharmaceutically acceptable salts of the foregoing of the disclosure may be useful include neuropathic pain (such as postherpetic neuralgia, nerve injury, the “dynias”, e.g., vulvodynia, phantom limb pain, root avulsions, painful diabetic neuropathy, painful traumatic mononeuropathy, painful polyneuropathy); central pain syndromes (potentially caused by virtually any lesion at any level of the nervous system); postsurgical pain syndromes (e.g., postmastectomy syndrome, postthoracotomy syndrome, stump pain); bone and joint pain (osteoarthritis), repetitive motion pain, dental pain, cancer pain, myofascial pain (muscular injury, fibromyalgia); perioperative pain (general surgery, gynecological), chronic pain, dysmennorhea, as well as pain associated with angina, and inflammatory pain of varied origins (e.g., osteoarthritis, rheumatoid arthritis, rheumatic disease, teno-synovitis and gout), headache, migraine and cluster headache, headache, primary hyperalgesia, secondary hyperalgesia, primary allodynia, secondary allodynia, or other pain caused by central sensitization.

The compounds, N-oxides thereof, and pharmaceutically acceptable salts of the foregoing of the disclosure may be used to decrease tolerance and/or dependence to opioid treatment of pain, and for treatment of withdrawal syndrome of e.g., alcohol, opioids, and cocaine.

In various cases, the M4-mediated (or M4-associated) disease or disorder can be selected from the group consisting of Alzheimer's disease, schizophrenia or psychosis, pain, addiction, a sleep disorder, a cognitive disorder (e.g. mild cognitive impairment), Parkinson's disease, Parkinson's disease-levodopa-induced dyskinesia, Huntington's disease, dyskinesia, tardive dyskinesia, dry mouth, pulmonary hypertension, chronic obstructive pulmonary disease (COPD), asthma, urinary incontinence, glaucoma, Trisomy 21 (Down syndrome), cerebral amyloid angiopathy, dementia, hereditary cerebral hemorrhage with amyloidosis of the Dutch type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes, autism, and atherosclerosis. In some cases, the M4-mediated (or M4-associated) disease or disorder is selected from the group consisting of Alzheimer's disease, schizophrenia, pain, addiction, Parkinson's disease, Parkinson's disease-levodopa-induced dyskinesia, and a sleep disorder.

### SYNTHESIS OF THE COMPOUNDS OF THE DISCLOSURE

The compounds of the disclosure can be synthesized by any method known in the art. For example, the compounds of the disclosure (compounds of Formula (I)) can be synthesized according to Schemes 1, 2, 3, 4, 5, and 6.

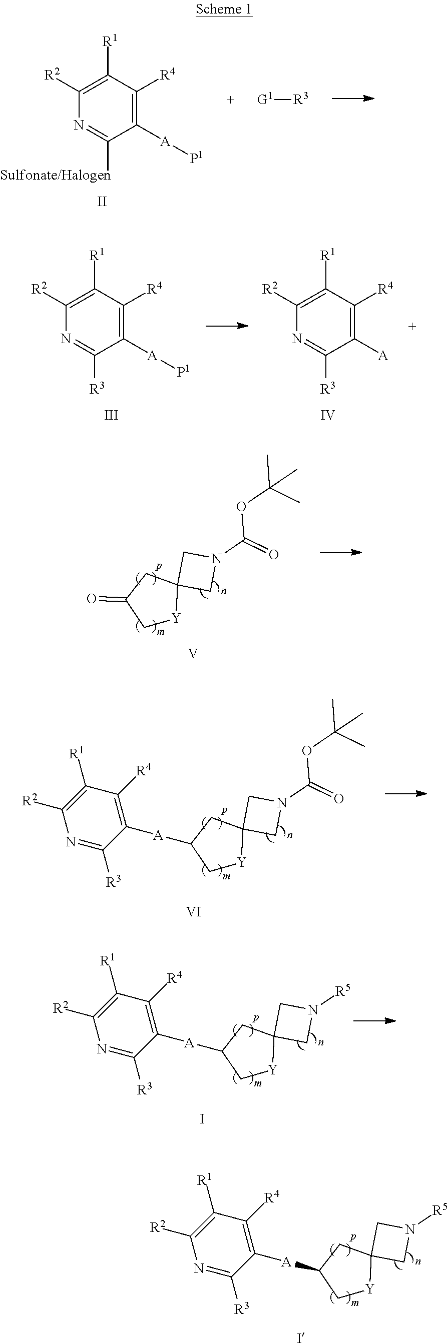

Scheme 1 refers to one synthetic sequence for the preparation of compounds of Formula I and I′. According to Scheme 1, compound II can be coupled to a heteroaryl, aryl, alkenyl, heterocycloalkyl, cycloalkyl, spiroheteroalkyl, spiroalkyl, alkyl and heteroalkyl boronic acids, boronate esters or potassium trifluoroborates via Suzuki-Miyaura coupling reaction. The scope of the reaction types is not restricted to Suzuki-Miyaura, but includes Stille, Negishi, Hiyama, and decarboxylative coupling; Sonogashira coupling with alkyne/silyl alkynes; and nucleophilic aromatic substitutions (S_{N}Ar) of amines and alcohols, wherein the R¹, R², R³, R⁴ and A substituents should be represented by the same moieties as desired in the final product or protected variation thereof, to produce compound III via a coupling reaction or the S_{N}Ar reaction with G¹ whereas G¹ is a boronic acid/ester/trifluoroborate, stannane, magnesium, zinc, carboxylic acid, carboxylate, or an alcohol/amine; or a terminal alkyne/silyl alkyne, using a standard selection of metal source, ligand, and base, in a standard solvent/cosolvent, but not limited to DMF, acetonitrile, 1,4-dioxane, THF, pyridine, toluene, ethanol, n-butanol, t-butanol. Examples of the Pd/ligand/base combination in coupling reaction include but are not limited to tetrakis(triphenylphosphine)palladium(0) plus sodium carbonate and tris(dibenzylideneacetone)dipalladium(0) plus dicyclohexylphosphino-2′,4′,6′-triisopropylbiphenyl plus potassium carbonate. In an S_{N}Ar reaction, examples of base include, but are not limited to triethylamine, N,N-diisopropylethylamine, K₂CO₃, Cs₂CO₃, K₃PO₄, K₃PO₄·H₂O, tBuOK, and NaH. Removal of protecting group P¹ results in compound IV. Protecting group P¹ in this case refers to groups well known to those skilled in the art for an amine protection. For example, P¹ may be a tert-butoxycarbonyl (Boc), which can be cleaved via acidic conditions in an appropriate solvent, including but not limited to treatment with a solution of trifluoroacetic acid (TFA) in dichloromethane (DCM). Alternatively P1 may be one of many other protecting groups suitable for amines, including carboxybenzyl (Cbz) or benzoyl (Bz) groups and can be cleaved under standard conditions known to one skilled in the art. Compounds IV can be coupled with compound V, wherein m, n and p are independently represented by an integer selected from 1 or 2, to produce racemic compounds VI using a standard reductive amination procedure, for example but not limited to, a combination of sodium cyanoborohydride and titanium(IV) ethoxide or sodium triacetoxyborohydride in a suitable solvent. Protecting group Boc can be cleaved via acidic conditions in an appropriate solvent, including but not limited to treatment with a solution of trifluoroacetic acid (TFA) in dichloromethane (DCM), then followed by a treatment with R⁵ to install the carbamates or carbamate bioisosteres, wherein the R⁵ should be represented by the same moieties as desired in the final product or protected variation thereof in dichloromethane or other appropriate solvents, to produce compounds of Formula I as a racemic mixture. Chiral separation of the racemic mixture, for example, a chiral chromatographic method such as a chiral HPLC or a chiral supercritical fluid chromatography (SFC), can produce enantiomerically enriched or enantiomerically pure (e.g., % ee of at least 98%) compounds of Formula I′. Enantiomerically enriched compounds are ones in which the enantiomeric excess (% ee) is at least 80% (i.e., an enantiomeric ratio of 9 to 1 of one enantiomer for the other). The % ee of a compound that is enantiomerically enriched can be at least 85%, at least 90%, at least 95%, at least 96%, at least 97%, at least 98%, or at least 99%. Measurement of % ee can be performed using known laboratory techniques.

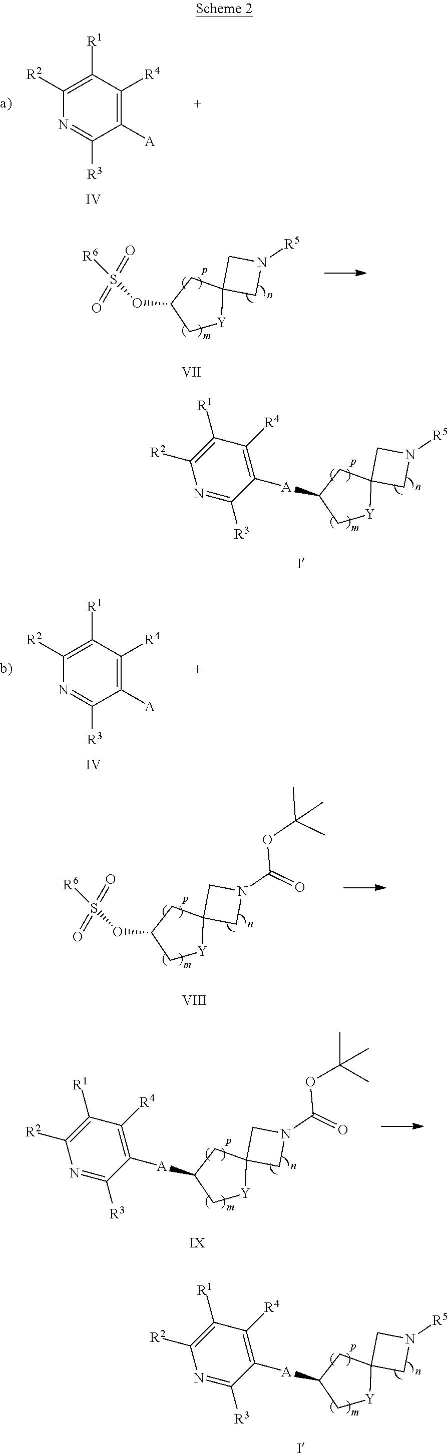

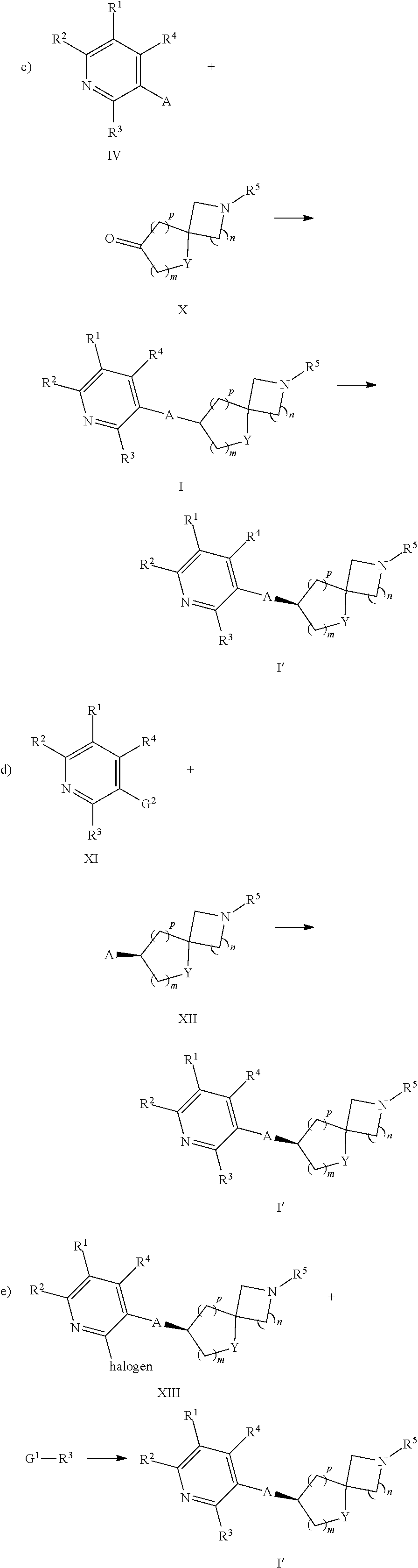

Scheme 2 refers to alternative synthetic routes for the preparation of compounds of Formula I and I′. Referring to Scheme 2a, compound IV, wherein the R¹, R², R³, R⁴ and A substituents of Formula IV should be represented by the same moieties as desired in the final product or protected variation thereof, can displace the sulfonate of enantiomerically pure compound VII, wherein the R⁵ of Formula VII should be represented by the same moieties as desired in the final product or protected variation thereof, where R⁶ is an aryl, alkyl, or fluoroalkyl substituent, for example 4-methylphenyl, methyl, nonafluorobutyl; and m, n and p are independently represented by an integer selected from 1 or 2, and Y should be represented by the same moieties as desired in the final product in the presence of a base such as potassium carbonate or potassium phosphate tribasic in an appropriate solvent, including but not limited to DMSO, DMF, MeCN, or THF. Referring to Scheme 2b, compound IV, wherein the R¹, R², R³, R⁴ and A should be represented by the same moieties as desired in the final product or protected variation thereof, can similarly displace the aryl alkyl/fluoroalkyl sulfonate on chiral compound VIII, where R⁶ is an aryl, alkyl, or fluoroalkyl substituent, for example 4-methylphenyl, methyl, nonafluorobutyl; and m, n and p are independently represented by an integer selected from 1 or 2, and Y should be represented by the same moieties as desired in the final product to produce compound IX. Removal of the Boc group, which can be cleaved via acidic conditions in an appropriate solvent, including but not limited to trifluoroacetic acid in dichloromethane (DCM), followed by treatment with R⁵ to install the carbamate or carbamate bioisosteres, wherein the R⁵ should be represented by the same moieties as desired in the final product or protected variation thereof, to produce compounds of Formula I′ as a single enantiomer. Alternately, as shown in scheme 2c, compound IV, wherein the R¹, R², R³, R⁴ and A substituents of Formula IV should be represented by the same moieties as desired in the final product or protected variation thereof, can be coupled to compound X, wherein the R⁵ of Formula X should be represented by the same moieties as desired in the final product or protected variation thereof, m, n and p are independently represented by an integer selected from 1 or 2, and Y should be represented by the same moieties as desired in the final product to produce compounds of the general Formula I a racemic mixture using a standard reductive amination procedure, for example but not limited to, a combination of sodium cyanoborohydride and titanium(IV) ethoxide or sodium triacetoxyborohydride in a suitable solvent. Chiral separation of the racemate of Formula I can be accomplished by a chiral SFC to provide compounds of Formula I′ as a single enantiomer. In Scheme 2d, compound XI, wherein the R¹, R², R³ and R⁴ should be represented by the same moieties as desired in the final product or protected variation thereof, where G² is a sulfonate, or halogen, can be coupled to enantiomerically pure compound XII, wherein the A and R⁵ of Formula XII should be represented by the same moieties as desired in the final product or protected variation thereof, m, n and p are independently represented by an integer selected from 1 or 2, and Y should be represented by the same moieties as desired in the final product to produce compounds of the Formula I′ using a standard coupling procedure, for example but not limited to Suzuki-Miyaura coupling or Buchwald-Hartwig coupling reactions as well as S_{N}Ar reaction of amines. In addition, referring to Scheme 2e, enantiomerically pure compound XIII, can be coupled to a heteroaryl, aryl, alkenyl, heterocyclcoalkyl, cycloalkyl, spiroheteroalkyl, spiroalkyl, alkyl and heteroalkyl boronic acids, boronate esters or potassium trifluoroborates via Suzuki-Miyaura coupling reaction. The scope of the reaction types is not restricted to Suzuki-Miyaura, but includes Stille, Negishi, Hiyama, and decarboxylative coupling; Sonogashira coupling with alkyne/silyl alkynes; and nucleophilic aromatic substitutions (S_{N}Ar) of amines and alcohols, to generate the Formula I′ as a single enantiomer, wherein the R¹, R², R³, R⁴, R⁵ and A substituents should be represented by the same moieties as desired in the final product or protected variation thereof, m, n and p are independently represented by an integer selected from 1 or 2, and Y should be represented by the same moieties as desired in the final product.

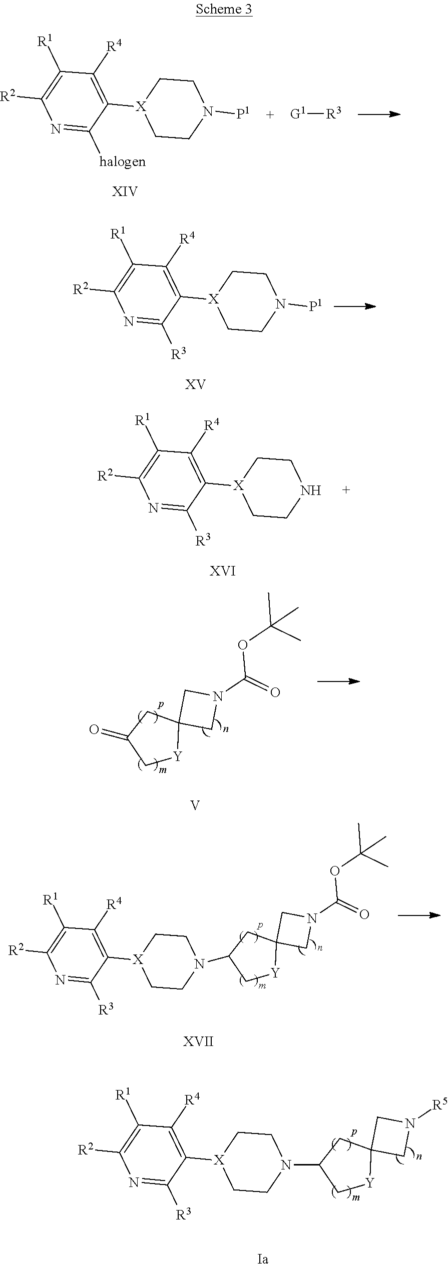

Scheme 3 refers to one synthetic sequence for the preparation of compounds of Formula Ia. According to scheme 3, compound XIV can be coupled to a heteroaryl, aryl, alkenyl, heterocyclcoalkyl, cycloalkyl, spiroheteroalkyl, spiroalkyl, alkyl and heteroalkyl boronic acids, boronate esters or potassium trifluoroborates via the Suzuki-Miyaura coupling reaction. The scope of the reaction types is not restricted to the Suzuki-Miyaura, but includes the Stille, Negishi, Hiyama, and decarboxylative coupling; the Sonogashira coupling with alkyne/silyl alkynes; and nucleophilic aromatic substitutions (S_{N}Ar) of amines and alcohols, and to produce compound XV, wherein the R¹, R², R³ and R⁴ substituents, and Y should be represented by the same moieties as desired in the final product or protected variation thereof, via a coupling reaction or S_{N}Ar reaction, using a standard selection of metal source, ligand, and base, in a standard solvent, for example but not limited to DMF, acetonitrile, 1,4-dioxane, THF, pyridine, toluene, ethanol, n-butanol, t-butanol. Examples of the Pd/ligand/base combination in coupling reaction include but are not limited to tetrakis(triphenylphosphine)palladium(0) plus potassium carbonate and tris(dibenzylideneacetone)dipalladium(0) plus dicyclohexylphosphino-2′,4′,6′-triisopropylbiphenyl plus potassium carbonate. In a S_{N}Ar reaction, examples of base include but are not limited to triethylamine, N,N-diisopropylethylamine, K₂CO₃, Cs₂CO₃, K₃PO₄, t-BuOK, and NaH. Removal of protecting group P1 results in compound XVI. Protecting group P¹ in this case refers to groups well known to those skilled in the art for an amine protection. For example, P¹ may be a tert-butoxycarbonyl (Boc), which can be cleaved via acidic conditions in an appropriate solvent, including but not limited to treatment with a solution of trifluoroacetic acid (TFA) in dichloromethane (DCM). Alternatively P¹ may be one of many other protecting groups suitable for amines, including carboxybenzyl (Cbz) or benzoyl (Bz) groups and can be cleaved under standard conditions known to one skilled in the art. Compounds XVI, can be coupled to compound V, wherein m, n and p are independently represented by an integer selected from 1 or 2, and Y should be represented by the same moieties as desired in the final product to produce racemic compounds XVII using a standard reductive amination procedure, for example but not limited to, a combination of sodium cyanoborohydride and titanium(IV) ethoxide or sodium triacetoxyborohydride in a suitable solvent, followed by deprotection of Boc-protecting group and treatment with R⁵ to install the carbamates or carbamate bioisosteres, wherein the R⁵ should be represented by the same moieties as desired in the final product or protected variation thereof, to produce compounds of Formula Ia.

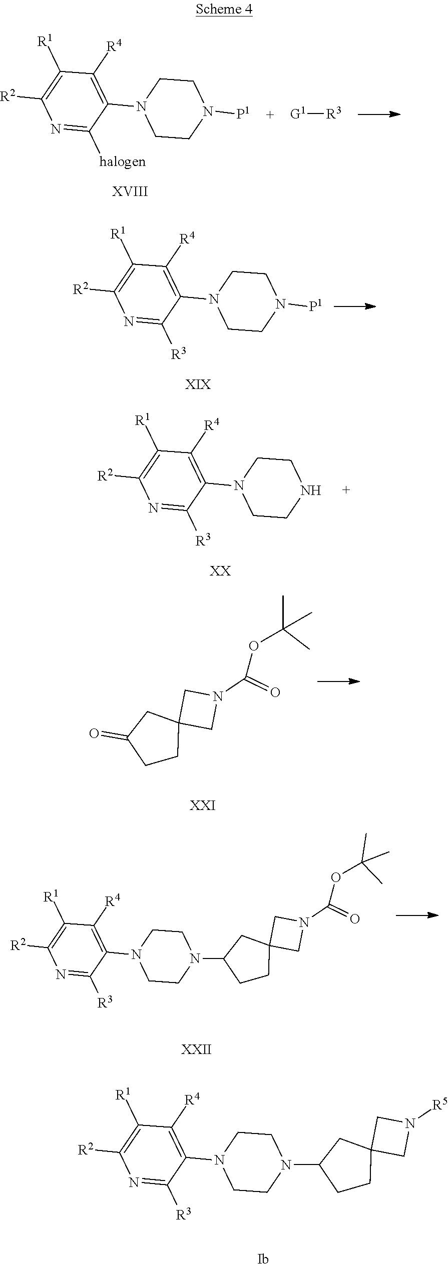

Scheme 4 refers to one synthetic sequence for the preparation of compounds of Formula Ib. Referring to scheme 4, compound XVIII can be coupled to a heteroaryl, aryl, alkenyl, heterocyclcoalkyl, cycloalkyl, spiroheteroalkyl, spiroalkyl, alkyl and heteroalkyl boronic acids, boronate esters or potassium trifluoroborates via Suzuki-Miyaura coupling reaction. The scope of the reaction types is not restricted to Suzuki-Miyaura, but includes Stille, Negishi, Hiyama, and decarboxylative coupling; Sonogashira coupling with alkyne/silyl alkynes; and nucleophilic aromatic substitutions (S_{N}Ar) of amines and alcohols, and to produce compound XIX, wherein the R¹, R², R³, and R⁴ substituents should be represented by the same moieties as desired in the final product or protected variation thereof, via a coupling reaction or S_{N}Ar reaction, using a standard selection of metal source, ligand, and base, in a standard solvent, for example but not limited to DMF, acetonitrile, 1,4-dioxane, THF, pyridine, toluene, ethanol, n-butanol, t-butanol. Examples of the Pd/ligand/base combination in coupling reaction include but are not limited to tetrakis(triphenylphosphine)palladium(0) plus sodium carbonate and tris(dibenzylideneacetone)dipalladium(0) plus dicyclohexylphosphino-2′,4′,6′-triisopropylbiphenyl plus potassium carbonate. In a S_{N}Ar reaction, examples of base include but are not limited to triethylamine, N, N-diisopropylethylamine, K₂CO₃, Cs₂CO₃, K₃PO₄, tBuOK, and NaH. Removal of protecting group P¹ results in compound XX. Protecting group P¹ in this case refers to groups well known to those skilled in the art for amine protection. For example, P¹ may be a tert-butoxycarbonyl (Boc), which can be cleaved via an acidic condition in an appropriate solvent, including but not limited to treatment with a solution of trifluoroacetic acid (TFA) in dichloromethane (DCM). Alternatively P¹ may be one of many other protecting groups suitable for amines, including carboxybenzyl (Cbz) or benzoyl (Bz) groups and can be cleaved under standard conditions known to one skilled in the art. Compounds XX, can be coupled to compound XXI to produce racemic compounds XXII using a standard reductive amination procedure, for example but not limited to, a combination of sodium cyanoborohydride and titanium(IV) ethoxide or sodium triacetoxyborohydride in a suitable solvent, followed by deprotection of the Boc group and treatment with R⁵ to install the carbamate or carbamate bioisosteres, wherein the R⁵ should be represented by the same moieties as desired in the final product or protected variation thereof in dichloromethane or other appropriate solvents, to produce compounds of Formula Ib.

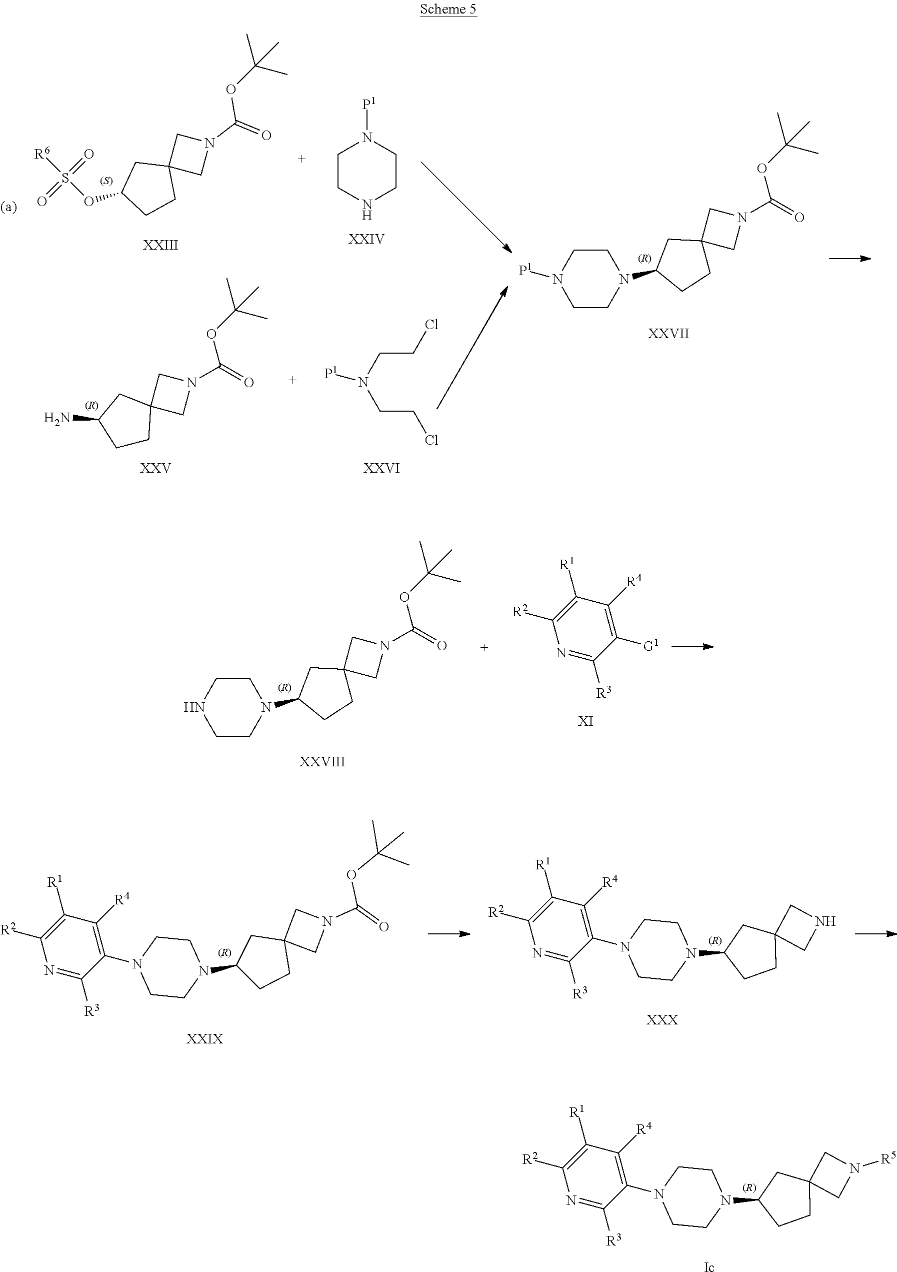

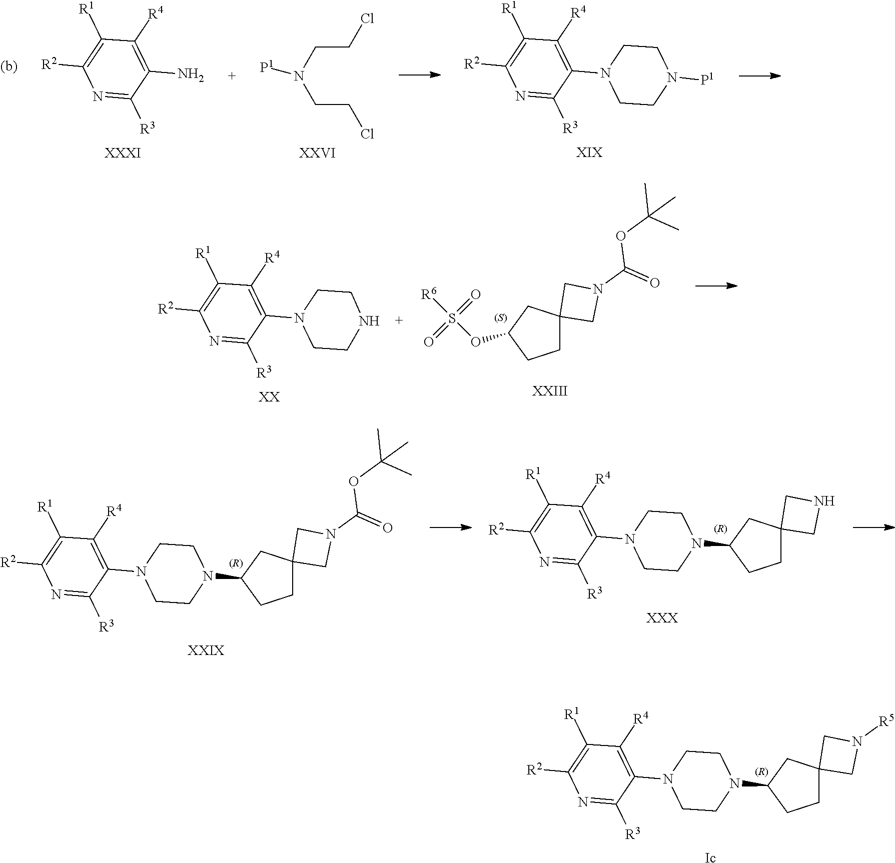

Scheme 5 refers to alternative synthetic routes for the preparation of compounds of Formula Ic. Referring to Scheme 5a, compound XXVII, can be prepared via two synthetic methods, one is that enantiomerical compound XXIII, wherein the R⁶ of Formula XXIII should be represented by an aryl, alkyl or fluoroalkyl substituent, for example 4-methylphenyl, methyl or nonafluorobutyl can be coupled with XXIV in the presence of a base such as potassium carbonate or potassium phosphate tribasic in an appropriate solvent, including but not limited to MeCN, DMSO, DMF, or THF. In addition, the alternative route is accomplished by the coupling of compounds XXV and XXVI. Removal of protecting group P¹ results in compound XXVIII. Protecting group P¹ in this case refers to groups well known to those skilled in the art for amine protection. For example, P¹ may be a carboxybenzyl (Cbz), which can be cleaved via H₂ gas conditions in an appropriate solvent, including but not limited to treatment with a solution of wet 10% Pd/C in methanol (MeOH). Alternatively P¹ may be one of many other protecting groups suitable for amines, including benzyl (Bn) or benzoyl (Bz) groups and can be cleaved under standard conditions known to one skilled in the art. Compound XXVIII can be coupled to compound XI, wherein the R¹, R², R³ and R⁴ should be represented by the same moieties as desired in the final product or protected variation thereof, where G¹ is a sulfonate, or a halogen, to produce compounds of compound XXIX using a standard C—N coupling procedure, for example but not limited to Buchwald-Hartwig coupling reactions and S_{N}Ar reaction of amines where appropriate. Subsequently, the tert-butoxycarbonyl (Boc) of compound XXIX can be cleaved via acidic conditions in an appropriate solvent, including but not limited to treatment with a solution of trifluoroacetic acid (TFA) in dichloromethane (DCM), to provide enantiomerical compound XXX, further followed by treatment with R⁵ to install the carbamate or carbamate bioisosteres, wherein the R⁵ should be represented by the same moieties as desired in the final product or protected variation thereof in dichloromethane or other appropriate solvents, to produce compounds of Formula Ic. Alternately, as shown in scheme 5b, compound XXXI, wherein the R¹, R², R³ and R⁴ should be represented by the same moieties as desired in the final product or protected variation thereof, can be coupled to XXVI through the double S_{N}2 cyclization, to produce compound XIX. Removal of protecting group P¹ results in compound XX. Protecting group P¹ in this case refers to groups well known to those skilled in the art for amine protection. For example, P¹ may be a tert-butoxycarbonyl (Boc), which can be cleaved via acidic conditions in an appropriate solvent, including but not limited to treatment with a solution of trifluoroacetic acid (TFA) in dichloromethane (DCM). Alternatively P¹ may be one of many other protecting groups suitable for amines, including carboxybenzyl (Cbz) or benzoyl (Bz) groups and can be cleaved under standard conditions known to one skilled in the art. Compound XX can displace the sulfonate of enantiomerically pure compound XXIII, where R⁶ is an aryl, alkyl, or fluoroalkyl substituent, for example 4-methylphenyl, methyl, nonafluorobutyl; for example 4-methylphenyl or methyl in the presence of a base such as potassium carbonate in an appropriate solvent, including but not limited to DMSO, DMF, or THF, to generate compound XXIX. Subsequently, the tert-butoxycarbonyl (Boc) of compound XXIX can be cleaved via acidic conditions in an appropriate solvent, including but not limited to treatment with a solution of trifluoroacetic acid (TFA) in dichloromethane (DCM), to provide enantiomerical compound XXX, further followed by treatment with R⁵ to install the carbamates or carbamate bioisosteres, wherein the R⁵ should be represented by the same moieties as desired in the final product or protected variation thereof in dichloromethane or other appropriate solvents, to produce compounds of Formula Ic.

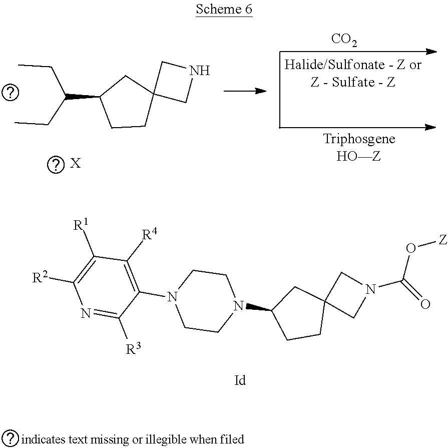

Scheme 6 refers to the synthetic sequences for the preparation of compounds of Formula Id. Referring to scheme 6, compound XXX, wherein the R¹, R², R³ and R⁴ substituents of Formula XXX should be represented by the same moieties as desired in the final product or protected variation thereof, can be followed by treatment with CO₂ gas, a base such as DBU, and Z-sulfate-Z, or Halide/Sulfonate-Z, wherein the Z should be represented by the same moieties as desired in the final product or protected variation thereof, in DMF or other appropriate solvents, to produce compounds of Formula Id. The alternative synthetic routes for the preparation of Formula Id, is to use triphosgene and ZOH, in an appropriate solvent such as DCM and a base such as pyridine wherein the Z should be represented by the same moieties as desired in the final product or protected variation thereof, to react with compound XXX for synthesizing the carbamates of Formula Id.

### EMBODIMENTS OF THE DISCLOSURE

1. A compound having a structure of Formula (I):

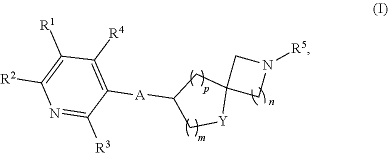

or an N-oxide thereof, or a pharmaceutically acceptable salt of the compound or the N-oxide thereof, wherein:
A is a 6-8 membered heterocycle comprising 1 or 2 ring nitrogen atoms and optionally substituted with 1 to 3 substituents independently selected from halogen, OH, and C₁₋₃alkyl;
Y is a bond, O, S, CH₂, CHF, CF₂, or C(OH)H;
m is 1 or 2;
n is 1 or 2;
p is 1 or 2;
R¹ is H, halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle or —[O]₀₋₁-5-10 membered heteroaryl, wherein the heterocycle and heteroaryl each comprises 1, 2, or 3 ring heteroatoms selected from N, O, and S, and when R¹ is C₁₋₆alkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, —[O]₀₋₁-5-10 membered heteroaryl, —NH—C₃₋₆cycloalkyl, —NH—C₆₋₁₀aryl, —NH-4-8 membered heterocycle, —NH-5-10 membered heteroaryl, —N(C₁₋₆alkyl)-C₃₋₆cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, R¹ is optionally substituted with 1, 2, or 3 substituents independently selected from halogen, CN, OH, ═O, SO₂, and C₁₋₃alkyl,
R² is H, halogen, CN, OH, —N(R⁶)(R⁷), C₁₋₆alkyl, C₁₋₆haloalkyl, C₁₋₆alkoxy, C₁₋₆haloalkoxy, C₂₋₆heteroalkyl, C₃₋₆cycloalkyl, or 4-8 membered heterocycle comprising 1, 2, or 3 ring heteroatoms selected from N, O, and S;
R³ is halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, —NH—C₃₋₆ cycloalkyl, —NH—C₆₋₁₀aryl, —NH-4-8 membered heterocycle, —NH-5-10 membered heteroaryl, —N(C₁₋₆alkyl)-C₃₋₆ cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, wherein the heterocycle and heteroaryl each comprises 1, 2, or 3 ring heteroatoms selected from N, O, and S, and the C₃₋₆cycloalkyl, C₆₋₁₀aryl, 4-8 membered heterocycle, or 5-10 membered heteroaryl is substituted with 0, 1, 2, or 3 R^{3a} substituents;
each R^{3a} is independently selected from halogen, CN, OH, ═O, ═N(C₁₋₃alkyl), SO₂, C₁₋₆alkyl, C₂₋₁₀alkene, C₁₋₆hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-NH₂, C₀₋₆alkylene-NH(C₁₋₆alkyl), C₀₋₆alkylene-N(C₁₋₆alkyl)₂, —S—C₁₋₆alkyl, C₀₋₆alkylene-SO₂C₁₋₆alkyl, C₀₋₆alkylene-C(O)NH₂, C₀₋₆alkylene-C(O)NH(C₁₋₆alkyl), C₀₋₆alkylene-C(O)N(C₁₋₆alkyl)₂, C₀₋₆alkylene-NHC(O)C₁₋₆alkyl, C₀₋₆alkylene-COOH, C₀₋₆alkylene-C₃₋₆cycloalkyl, C₁₋₆alkylene-O—C₁₋₆alklyeneSi(C₁₋₃alkyl)₃, and C₀₋₆alkylene-3-6 membered heterocycle comprising 1, 2, or 3 heteroatoms selected from N, O, and S;
R⁴ is H, halogen, CN, or OH;
R⁵ is —CO₂—Z, or a bioisostere thereof;
each R⁶ and R⁷ is independently H, C₁₋₆alkyl, C(O)—C₁₋₆alkyl, spiro or bicyclic C₈₋₁₄cycloalkyl, 8-14 membered heterocycle comprising 1, 2, or 3 ring heteroatoms selected from N, O, and S, and when R⁶ or R⁷ is other than H, it can be optionally substituted with 1, 2, or 3 substituents independently selected from the group consisting of halogen, CN, OH, ═O, SO₂, C₀₋₆alkylene-NH₂, C₀₋₆alkylene-NH(C₁₋₆alkyl), C₀₋₆alkylene-N(C₁₋₆alkyl)₂, C₀₋₆alkylene-SO₂C₁₋₆alkyl, C₁₋₆alkyl and C₁₋₆alkoxy, or
   R⁶ and R⁷, together with the nitrogen to which they are attached, form a 4-10 membered heterocycle comprising 0-2 additional ring heteroatoms independently selected from N, O, and S; and
Z is C₁₋₇alkyl, C₁₋₇haloalkyl, C₃₋₆cycloalkyl, or C₂₋₆alkyne, and Z is optionally substituted with C₁₋₆alkoxy or C₃₋₆cycloalkyl;
with the proviso that when R¹, R², and R⁴ are each H, Y is CH₂, m, n, and p are each 1, A is

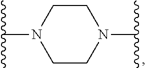 [SMILES: *N1CCN(*)CC1]

and R³ is trifluoroethoxy, trifluoromethoxy, difluoromethoxy, methoxy or

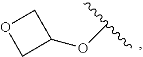 [SMILES: *OC1COC1]

then R⁵ is not CO₂CH₂CH₃.
2. The compound or salt of embodiment 1, wherein A is

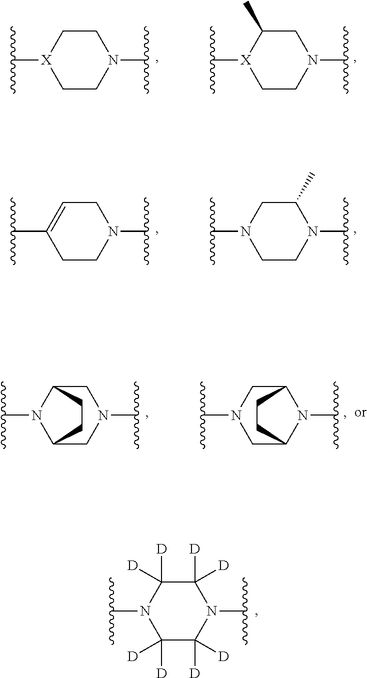

### and X is N, CF, C(OH), or CH.

3. The compound or salt of embodiment 2, having a structure of Formula (Ia), or a pharmaceutically acceptable salt thereof:

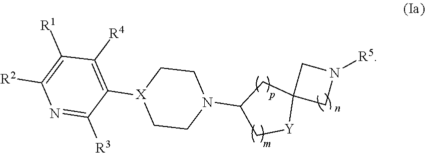

4. The compound or salt of embodiment 3, having a structure of Formula (Ib):

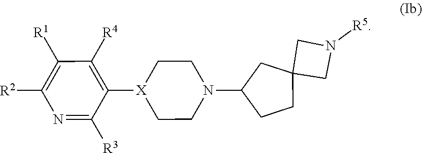

5. The compound or salt of any one of embodiments 1 to 3, wherein Y is CH₂, CHF, CF₂, or C(OH)H.
6. The compound or salt of embodiment 5, wherein Y is CH₂.
7. The compound or salt of any one of embodiments 1 to 3, 5 and 6 wherein m is 1.
8. The compound or salt of any one of embodiments 1 to 3, and 5 to 7 wherein n is 1.
9. The compound or salt of any one of embodiments 1 to 3, and 5 to 8 wherein p is 1.
10. The compound or salt of embodiment 4, having a structure of Formula (Ic):

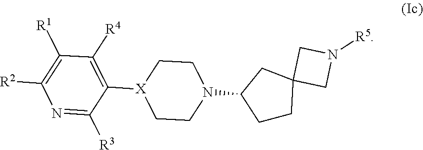

11. The compound or salt of embodiment 4, having a structure of Formula (Id):

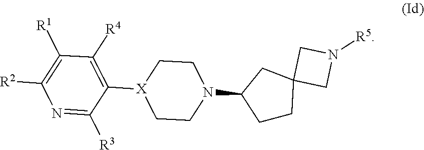

12. The compound or salt of any one of embodiments 1 to 11, wherein R⁵ is a CO₂Z bioisostere and is selected from the group consisting of

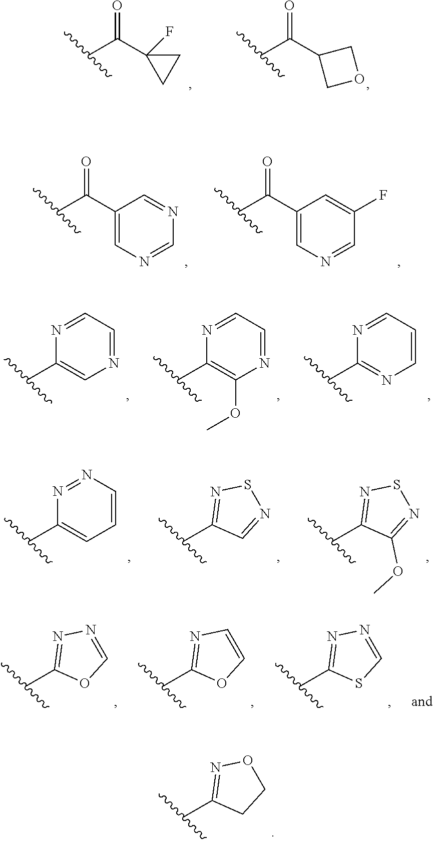

13. The compound or salt of embodiment 1, having a structure of Formula (Ie), or a pharmaceutically acceptable salt thereof:

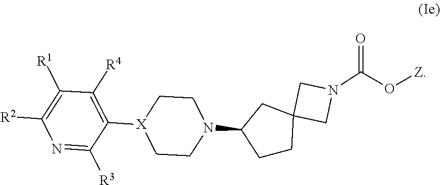

14. The compound or salt of any one of embodiments 1 to 11 and 13, wherein R⁵ is selected from the group consisting of CO₂C₁₋₇alkyl,

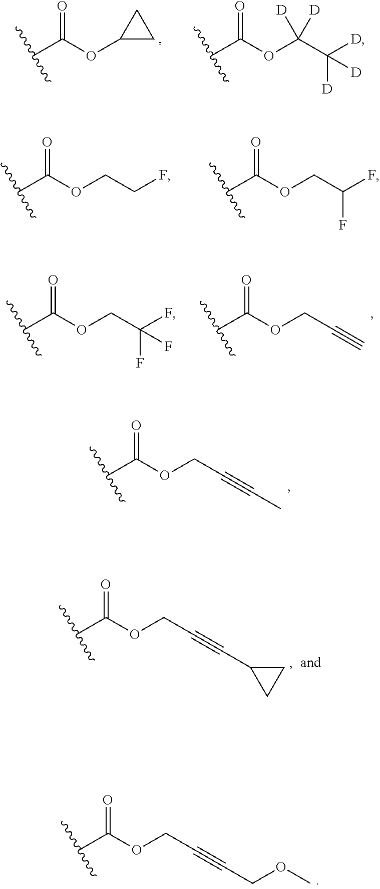

15. The compound or salt of embodiment 14, wherein R⁵ is CO₂CH₂CH₃.
16. The compound or salt of any one of embodiments 1 to 15, wherein R⁴ is H or halogen.
17. The compound or salt of any one of embodiments 1 to 16, wherein R¹ is H, halogen, CN, OH, —N(R⁶)(R⁷), C₁₋₆alkyl, or C₁₋₆alkoxy.
18. The compound or salt of embodiment 17, wherein R¹ is H or halogen.
19. The compound or salt of any one of embodiments 1 to 18, wherein R² is H, halogen, CN, OH, —N(R⁶)(R⁷), C₁₋₆alkyl, C₁₋₆haloalkyl, C₁₋₆alkoxy, or C₁₋₆haloalkoxy.
20. The compound or salt of embodiment 19, wherein R² is H, halogen, C₁₋₆alkyl, C₁₋₆alkoxy, C₁₋₆ haloalkyl, or C₁₋₆haloalkoxy.
21. The compound or salt of embodiment 20, wherein R² is H or halogen.
22. The compound or salt of any one of embodiments 1 to 21, wherein each R⁶ and R⁷ are independently H, C₁₋₆alkyl, or C(O)—C₁₋₆alkyl.
23. The compound or salt of embodiment 22, wherein each R⁶ and R⁷ are independently H or C₁₋₆ alkyl.
24. The compound or salt of any one of embodiments 1 to 21, wherein at least one R⁶ and R⁷, together with the nitrogen to which they are attached, form a 4-10 membered heterocycle comprising 0-2 additional ring heteroatoms independently selected from N and O.
25. The compound or salt of any one of embodiments 1 to 24, wherein at least one of R¹, R², and R⁴ is halogen.
26. The compound or salt of embodiment 25, wherein at least one of R¹, R², and R⁴ is F or Cl.
27. The compound or salt of any one of embodiments 1 to 26, wherein R³ is —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, and is optionally substituted with 1, 2, or 3 R^{3a}.
28. The compound or salt of any one of embodiments 1 to 27, wherein R³ is C₃₋₆ cycloalkyl, 5-10 membered heteroaryl, or 4-8 membered heterocycle, optionally substituted with 1, 2, or 3 R^{3a}.
29. The compound or salt of any one of embodiments 25 to 28, wherein R³ is

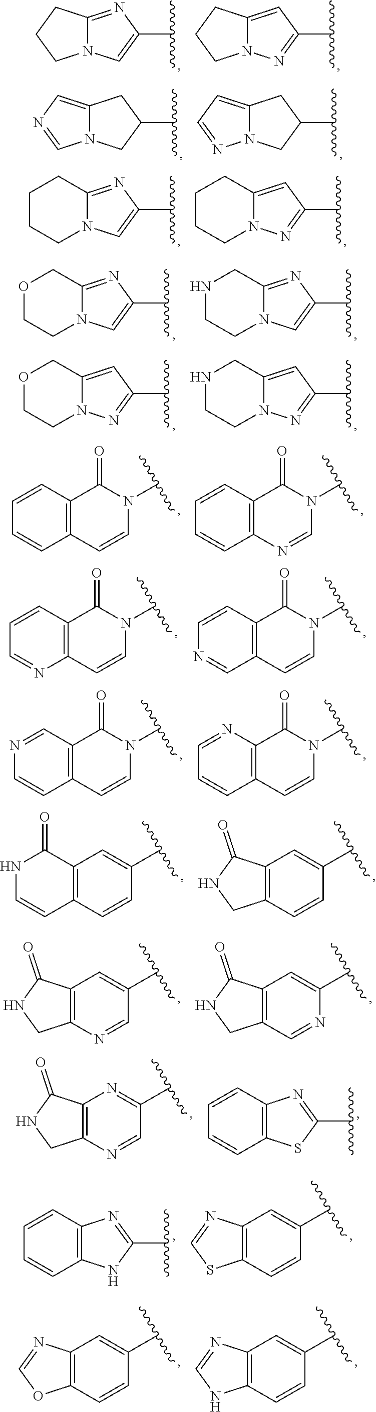

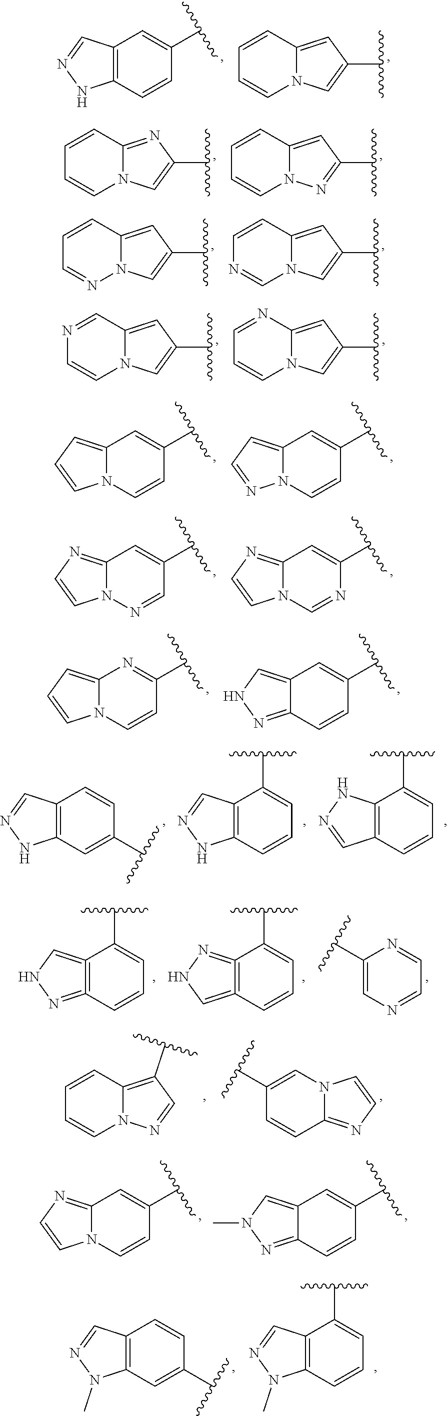

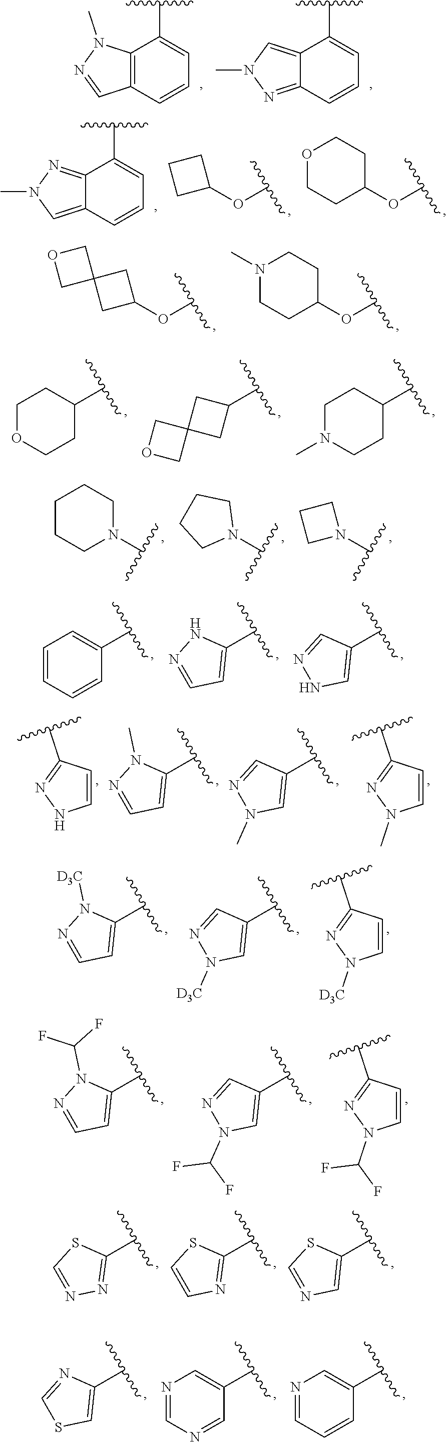

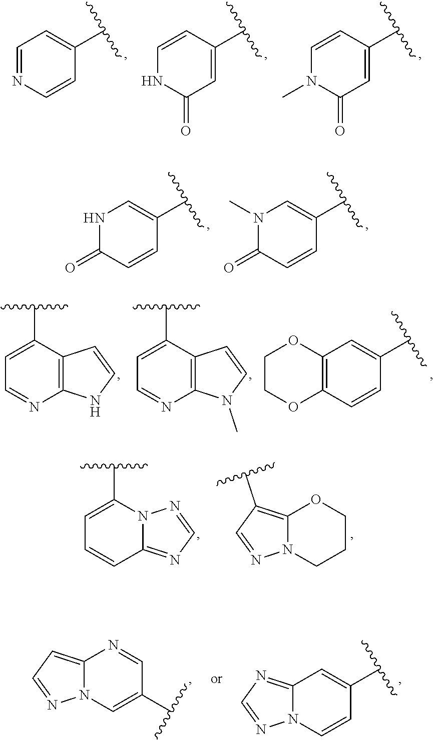

and is optionally substituted with 1, 2, or 3 R^{3a}.
30. The compound or salt of embodiment 29, wherein R³ is

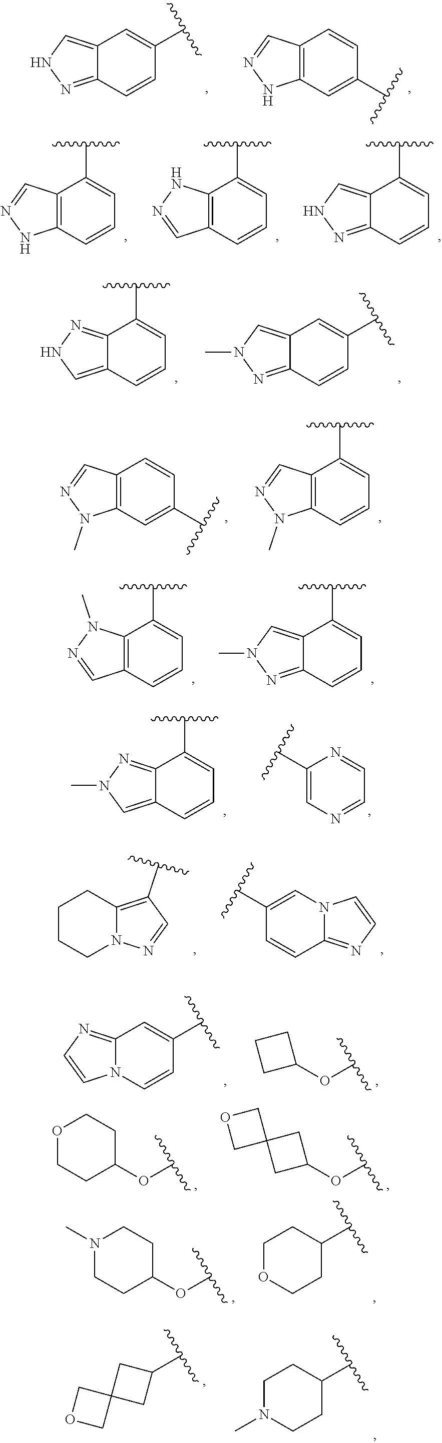

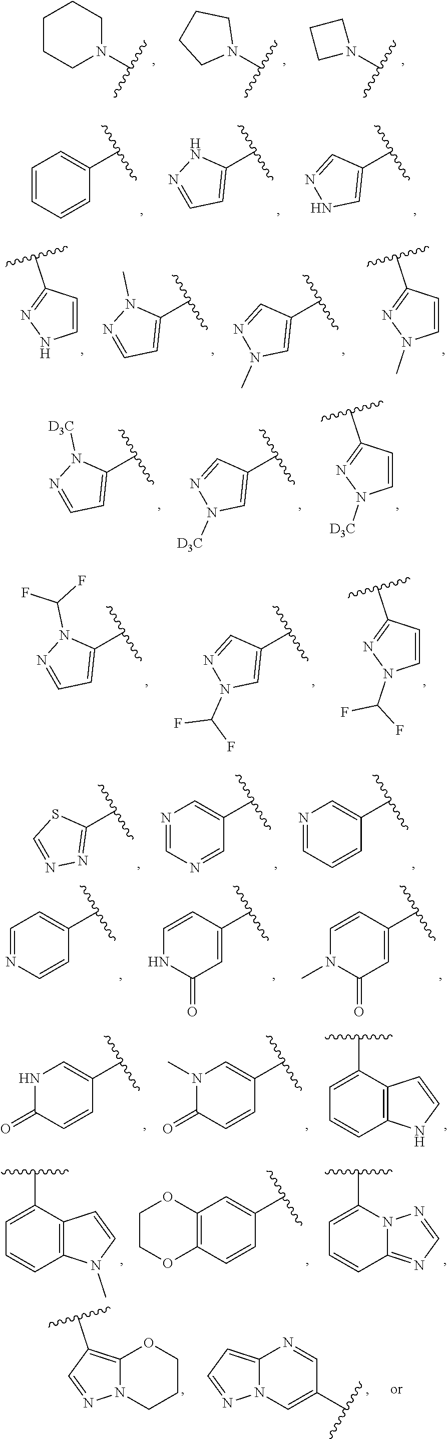

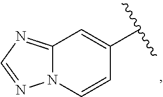 [SMILES: *c1ccn2ncnc2c1]

and is optionally substituted with 1, 2, or 3 R^{3a}.
31. The compound or salt of any one of embodiments 1 to 30, wherein R³ is unsubstituted.
32. The compound or salt of any one of embodiments 1 to 30, wherein R³ is substituted with 1 or 2 R^{3a}.
33. The compound or salt of any one of embodiments 1 to 30, and 32 wherein R³ is substituted with 1 R^{3a}.
34. The compound or salt of any one of embodiments 1 to 30, 32 and 33, wherein at least one R^{3a} is halogen, CN, OH, ═O, SO₂, C₁₋₆alkyl, C₂₋₁₀alkene, C₁₋₆hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-N(C₁₋₆alkyl)₂, —S—C₁₋₆alkyl, C₀₋₆alkylene-NHC(O)C₁₋₆alkyl, or C₀₋₆alkylene-3-6 membered heterocycle comprising 1, 2, or 3 heteroatoms selected from N, O, and S.
35. The compound or salt of embodiment 34, wherein at least one R^{3a} is CH₃, CH₂CH₃, CH(CH₃)₂, CH₂CH(CH₃)₂, CF₃, CHF₂, CH₂CH₂F, CH₂CHF₂, CH₂OH, C(CH₃)₂OH, CH₂OCH₃, CH₂CH₂OCH₃, CH₂OCH₂CH₃, F, CN, ═O, SO₂, OH, OCH₃, OCH₂CH₃, OCH(CH₃)₂, OCHF₂, CH₂OCH₃, CH₂OCF₃, SCH₃, NH₂, N(CH₃)₂, NHCOCH₃, CD₃,

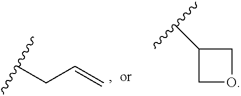

36. The compound or salt of embodiment 35, wherein at least one R^{3a} is CH₃, CH₂CH₃, F, CN, OH, OCH₃, CF₃, CH₂OH, or OCHF₂.
37. The compound or salt of embodiment 1, having a structure of Formula (If), or a pharmaceutically acceptable salt thereof:

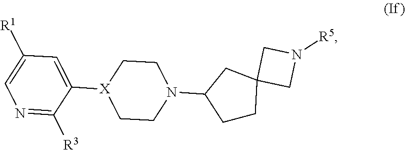

wherein
   R¹ is halogen;
   R³ is —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, wherein the heterocycle and heteroaryl each comprises 1, 2, or 3 ring heteroatoms selected from N, O, and S, and R³ is substituted with 0, 1, 2, or 3 R^{3a} substituents;
   R⁵ is CO₂Z. or a bioisostere thereof; and
   Z is C₁₋₇alkyl, C₁₋₇haloalkyl, C₃₋₆cycloalkyl, or C₂₋₆alkyne, and is optionally substituted with C₁₋₆ alkoxy or C₃₋₆cycloalkyl.
38. The compound or salt of embodiment 37, wherein R¹ is Cl or F.
39. The compound or salt of embodiment 37 or 28, wherein R³ is 5-6-membered heterocycle comprising 1 ring heteroatom selected from S and O, or 5-6-membered heteroaryl comprising 2 or 3 ring heteroatoms independently selected from N and S, and R³ is substituted with 0, 1 or 2 R^{3a} substituents independently selected from halogen, CN, OH, and C₁₋₆alkyl.
40. The compound or salt of embodiment 37, 38, or 39, wherein R⁵ is CO₂C₁₋₇alkyl.
41. The compound or salt of embodiment 40, wherein R⁵ is CO₂Et.
42. A compound, or pharmaceutically acceptable salt thereof, as recited in Table A.
43. A pharmaceutical formulation comprising a therapeutically effective amount of the compound or salt of any one of embodiments 1 to 42, and a pharmaceutically acceptable excipient.
44. A method for treating an M4-mediated (or M4-associated) disease or disorder in a patient comprising administering to the patient a therapeutically effective amount of the compound or salt of any one of embodiments 1 to 42.
45. The method of embodiment 44, wherein the M4-mediated (or M4-associated) disease or disorder is selected from the group consisting of Alzheimer's disease, schizophrenia or psychosis, pain, addiction, a sleep disorder, a cognitive disorder (e.g., mild cognitive impairment), Parkinson's disease, Parkinson's disease-levodopa-induced dyskinesia, Huntington's disease, dyskinesia, dry mouth, pulmonary hypertension, chronic obstructive pulmonary disease (COPD), asthma, urinary incontinence, glaucoma, Trisomy 21 (Down syndrome), cerebral amyloid angiopathy, Alzheimer's disease psychosis, dementia-related psychosis, bipolar I disorder, bipolar II disorder, bipolar depression, missed and/or manic-episodes associated with bipolar disorder, hereditary cerebral hemorrhage with amyloidosis of the Dutch type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes, autism, and atherosclerosis.
46. The method of embodiment 45, wherein the M4-mediated (or M4-associated) disease or disorder is selected from the group consisting of Alzheimer's disease, schizophrenia, pain, addiction, Parkinson's disease, Parkinson's disease-levodopa-induced dyskinesia, and sleep disorder.

### EXAMPLES

The following examples are provided for illustration and are not intended to limit the scope of the disclosure.

As used throughout these examples, common organic abbreviations are defined as follows:

| Abbreviation | Chemical |
|---|---|
| Ac | Acetyl |
| AcOH | Acetic Acid |
| OAc | Acetate |
| ACN | Acetonitrile |
| Bn | Benzyl |
| Bz | Benzoyl |
| BOC or Boc | tert-Butoxycarbonyl |
| br | broad |
| t-Bu | tert-Butyl |
| Cbz | carboxybenzyl |
| CDI | carbonyldiimidazole |
| Cmpd | Compound |
| DAST | (diethylamino)sulfur trifluoride |
| DBU | 1,8-diazabicyclo(5.4.0)undec-7-ene |
| DCE | dichloroethane |
| DCM | dichloromethane |
| DIBAL-H | Diisobutylaluminum hydride |
| DIPEA | N,N-Diisopropylethylamine |
| DMA | dimethylacetamide |
| DMF | Dimethylformamide |
| DMSO | Dimethylsulfoxide |
| EtOAc/EA | Ethyl acetate |
| ESI | electrospray ionization |
| EtOH | Ethanol |
| FLIPR | fluorescence imaging plate reader |
| GPCR | G-protein coupled receptor |
| h | hours |
| HATU | 1-[Bis(dimethylamino)methylene]- |
| | 1H-1,2,3-triazolo[4,5-b]pyridinium 3- |
| | oxide hexafluorophosphate |
| HBSS | Hanks' balanced salt solution |
| HPLC | high performance liquid |
| | chromatography |
| Hz | hertz |
| IPA | propan-2-ol |
| LCMS | liquid chromatography mass |
| | spectrometry |
| LiHMDS | lithium bis(trimethylsilyl)amide |
| m | multiplet |
| mAChR | muscarinic acetylcholine receptor |
| mCBPA | meta-Chloroperoxybenzoic acid |
| MeOH | Methanol |
| min | minute(s) |
| MTBE | methyl tert-butyl ether |
| NCS | N-chlorosuccinimide |
| nm | nanometer(s) |
| NMR | nuclear magnetic resonance |
| Pd/C | Palladium on carbon |
| Pd₂(dba)₃ | Tris(dibenzylideneacetone)dipalladium(0) |
| Pd(dppf)Cl₂• | [1,1′- |
| | bis(diphenylphosphino)ferrocene]dichloro- |
| | palladium(II), complex |
| PE | Petroleum ether |
| py | Pyridine |
| QC | Quality control |
| rt | Room temperature |
| Rt | Retention Time |
| s | singlet |
| SFC | Supercritical Fluid Chromatography |
| t | triplet |
| TEA or NEt₃ | Triethylamine |
| TFA | Trifluoroacetic acid |
| THF | Tetrahydrofuran |
| TR-FRET | time-resolved fluorescence |
| | resonance energy transfer |
| XantPhos | (9,9-dimethyl-9H-xanthene-4,5- |
| | diyl)bis(diphenylphosphane) |
| XPhos | dicyclohexyl[2′,4′,6′-tris(propan-2- |
| | yl)[1,1′-biphenyl]-2-yl]phosphane |

Inert atmosphere (nitrogen or argon) was generally required, particularly in cases where oxygen- or moisture-sensitive reagents such as dry Pd/C, intermediates, and/or inert conditions were employed. Commercial solvents and reagents were generally used without further purification. Anhydrous solvents were employed where appropriate, generally from WuXi-EHS, which the following QC specifications for water were attained: a) <50 ppm for N′N-dimethylformamide; b)<100 ppm for dichloromethane, toluene, and tetrahydrofuran; c)<200 ppm for methanol, ethanol, 1,4-dioxane and diisopropylamine. Products were generally dried under vacuum before being carried on to further reactions or submitted for biological testing. Mass spectrometry data is reported from either liquid chromatography-mass spectrometry (LCMS) and high-performance liquid chromatography (HPLC). ¹H NMR spectra were recorded at 400 MHz on a Bruker instrument. Chemical shifts (δ) for nuclear magnetic resonance (NMR) data are expressed in parts per million (ppm) referenced to residual peaks from the deuterated solvents employed. In addition, chiral separations were carried out to separate enantiomers of certain compounds of the disclosure by supercritical fluid chromatography (SFC). In some examples, the separated enantiomers are designated as Peak 1 and Peak 2, according to their order of elution. Based upon their potency, the chirality may be implemented as (R)- or (S)-isomer. Reactions proceeding through detectable intermediates were generally followed by LCMS and allowed to proceed to full conversion prior to addition of subsequent reagents.

For syntheses referencing procedures in other Examples or Methods, reaction conditions (reaction time and temperature) may vary. Microwave-mediate reactions were performed in Biotage Initiator microwave reactors for some compounds. In general, reactions were monitored by thin-layer chromatography or mass spectrometry, and subjected to work-up when appropriate. Purifications may vary between experiments: in general, solvents and the solvent ratios used for eluents/gradients were chosen to provide appropriate Rf or retention times. All starting materials in these Preparations and Examples are either commercially available or can be prepared by methods known in the art or as described herein.

LCMC Method 1: Instrument: SHIMADZU LC20-MS2010; Mobile Phase: 1.5 mL/4LTFA in water (solvent A) and 0.75 mL/4LTFA in acetonitrile (solvent B), using the elution gradient 5%-95% (solvent B) over 0.7 minutes and holding at 95% for 0.4 minutes at a flow rate of 1.5 mL/min; Column: MERCK, RP-18e 25-2 mm; Wavelength: UV 220 nm & 254 nm; Column temperature: 50° C.; MS ionization: ESI.

LCMC Method 2: Instrument: SHIMADZU LC20-MS2020; Mobile Phase: 0.8 mL/4 L NH₃·H₂O in water (solvent A) and acetonitrile (solvent B), using the elution gradient 10%-80% (solvent B) over 6 minutes and holding at 80% for 0.5 minutes at a flow rate of 0.8 mL/min; Column: Titank C18, 5 μm, 2.1×50 mm; Wavelength: UV 220 nm & 254 nm; Column temperature: 50° C.; MS ionization: ESI.

LCMC Method 3: Instrument: SHIMADZU LCMS-2020; Mobile phase: Ramp from 5% ACN (0.01875% TFA) in water (0.0375% TFA) to 95% ACN in water in 0.60 min, flow rate is set at 2.0 mL/min; then hold at 95% ACN for 0.18 minutes, flow rate is set at 2.0 mL/min; return back to 5% ACN in water and hold for 0.02 min, Flow rate is set at 2.0 mL/min; Column: Kinetex® EVO C18 2.1×30 mm, 5 um. Column temperature: 50° C.

HPLC Method 1:Instrument: SHIMADZU LC20-MS2020; Mobile Phase: 0.2 ML/1 L NH₃·H₂O in water (solvent A) and acetonitrile (solvent B), using the elution gradient 10%-80% (solvent B) over 6 minutes and holding at 80% for 2 minutes at a flow rate of 0.8 mL/min; Column: Titank C18, 5 μm, 2.1×50 mm; Wavelength: UV 220 nm, 215 nm & 254 nm; Column temperature: 50° C.

HPLC Method 2: Instrument: SHIMADZU LC-20AD; Mobile phase: Ramp from 10% ACN (0.018% TFA) in water (0.037% TFA) to 80% ACN in water in 3.00 min, flow rate is set at 1.5 mL/min; then hold at 80% ACN for 0.70 minutes, flow rate is set at 1.5 mL/min; return back to 10% ACN in water and hold for 0.30 min, flow rate is set at 2.0 mL/min; Column: Kinetex C18 LC Column 4.6×50 mm, Sum. Wavelength: UV 220 nm & 254 nm. Column temperature: 50° C.

SFC Method 1: Instrument: CAS-SH-ANA-SFC-G (Agilent 1260 with DAD detector); Column: ChiralPak AD-3 150×4.6 mm I.D., 3 um; Mobile phase: A: CO₂ B: Methanol (0.05% DEA) Isocratic: 40% B; Flow rate: 2.5 mL/min Column temp.: 40° C.; Back pressure: 100 bar.

SFC Method 2: Instrument: CAS-SH-ANA-SFC-L (Waters UPCC with PDA Detector); Column: Chiralcel OD-3 150ᵢ Á4.6 mm I.D., 3 um; Mobile phase: A: CO₂ B: methanol (0.05% DEA) Gradient: from 5% to 40% of B in 4 min and from 40% to 5% of B in 0.2 min, then hold 5% of B for 1.8 min; Flow rate: 2.5 mL/min; Column temp.: 35° C.; Back pressure: 1500 psi.

### Example #1: Synthesis of ethyl (6R)-6-piperazin-1-yl-2-azaspiro[3.4]octane-2-carboxylate (Intermediate P1)

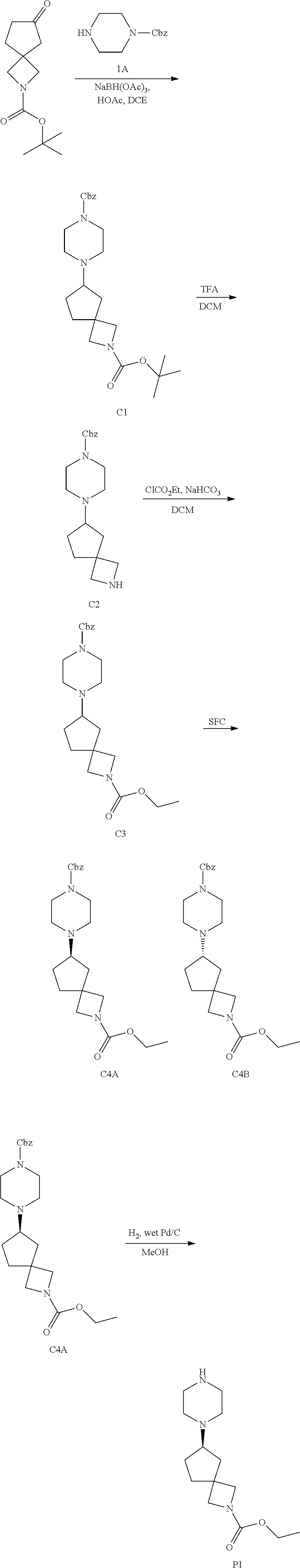

Step 1. Synthesis of tert-butyl 6-(4-benzyloxycarbonylpiperazin-1-yl)-2-azaspiro[3.4]octane-2-carboxylate (C1) To a mixture of tert-butyl 6-oxo-2-azaspiro[3.4]octane-2-carboxylate (commercially available, 100 g, 444 mmol), benzyl piperazine-1-carboxylate (commercially available, 117 g, 532 mmol) and 4 Å molecular sieve (67.5 g) in DCE (2.30 L) was added CH₃COOH (13.2 g, 221 mmol) at 25° C. The mixture was stirred at 25° C. for 60 min, then NaBH(OAc)₃ (235 g, 1.10 mol) was added in portions and stirred at 25° C. for 15 hours. The mixture was poured into saturated aqueous NaHCO₃ (1.70 L), stirred for 10 min and separated. The aqueous phase was extracted with DCM (3×800 mL). The combined organic phase was washed with brine (1.20 L), dried over anhydrous Na₂SO₄, filtered and concentrated in vacuum at 50° C. The crude product was triturated with petroleum ether (1.80 L) at 25° C. for 60 min. The suspension was filtered off and filter cake was dried in vacuum at 50° C. to afford Intermediate C1.

Step 2. Synthesis of benzyl 4-(2-azaspiro[3.4]octan-6-yl)piperazine-1-carboxylate (C2) To a solution of tert-butyl 6-(4-benzyloxycarbonylpiperazin-1-yl)-2-azaspiro[3.4]octane-2-carboxylate (170 g, 381 mmol) in DCM (800 mL) was added TFA (286 g, 2.51 mol). The mixture was stirred at 25° C. for 12 hours. The reaction mixture was concentrated under reduced pressure at 50° C. to give Intermediate C2(TFA salt), which was used directly in the next step without further purification. LCMS [M+H]+ 330.

Step 3. Synthesis of ethyl 6-(4-benzyloxycarbonylpiperazin-1-yl)-2-azaspiro[3.4]octane-2-carboxylate (C3) To a mixture of benzyl 4-(2-azaspiro[3.4]octan-6-yl)piperazine-1-carboxylate (220 g, 496 mmol, TFA salt) in DCM (1.50 L) and H₂O (750 mL) was added NaHCO₃ (541 g, 6.45 mol) at 25° C. The reaction mixture was stirred at 25° C. for 10 min and ethyl carbonochloridate (178 g, 1.64 mol) was added dropwise at 25° C. The mixture was stirred at 25° C. for 1 hour. The reaction mixture was poured into water (500 mL), stirred for 30 min at 25° C., and separated. The aqueous phase was extracted with DCM (2×600 mL). The combined organic phase was washed with brine (500 mL), dried over anhydrous Na₂SO₄, filtered and concentrated in vacuum to afford Intermediate C3.

Step 4. Ethyl (6R)-6-(4-benzyloxycarbonylpiperazin-1-yl)-2-azaspiro[3.4]octane-2-carboxylate and ethyl (6S)-6-(4-benzyloxycarbonylpiperazin-1-yl)-2-azaspiro[3.4]octane-2-carboxylate (C4A and C4B)

A racemic mixture of ethyl 6-(4-benzyloxycarbonylpiperazin-1-yl)-2-azaspiro[3.4]octane-2-carboxylate (430 g, 1.07 mol) was separated by SFC (column: DAICEL CHIRALCEL OJ (250 mm*50 mm, 10 um); mobile phase: [0.1% NH₃H₂O MeOH]; B %: 20%, min) to give Intermediate C4A and Intermediate C4B. LCMS [M+H]⁺ 402.

Step 5. Synthesis of ethyl (6R)-6-piperazin-1-yl-2-azaspiro[3.4]octane-2-carboxylate (P1)

To a solution of ethyl (6R)-6-(4-benzyloxycarbonylpiperazin-1-yl)-2-azaspiro[3.4]octane-2-carboxylate (120 g, 299 mmol) in MeOH (1200 mL) was added wet 10% Pd/C (15.0 g) at 20° C. The suspension was degassed under vacuum and purged with H₂ gas several times. The mixture was stirred under H₂ gas (15 psi) at 25° C. for 16 hours. The reaction mixture was filtered and washed with MeOH (2×1,000 mL). The filter was concentrated to give Intermediate P1.

### Example #2: Synthesis of ethyl (6R)-6-[4-(2-bromo-5-fluoro-3-pyridyl)piperazin-1-yl]-2-azaspiro[3.4]octane-2-carboxylate (Intermediate P2)

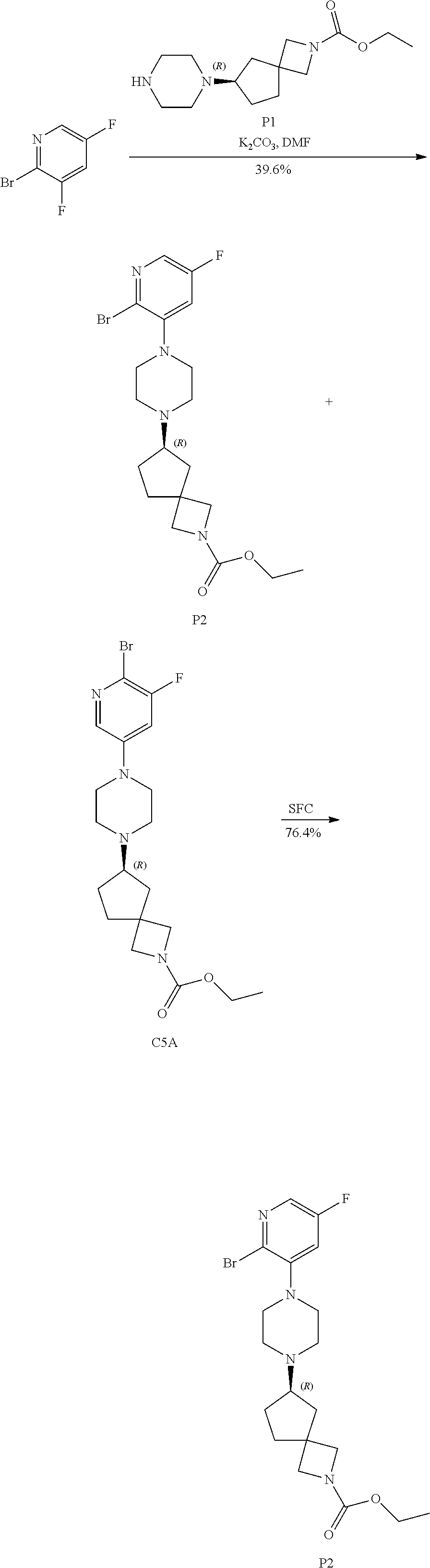

Step 1. Synthesis of ethyl (6R)-6-[4-(2-bromo-5-fluoro-3-pyridyl)piperazin-1-yl]-2-azaspiro[3.4]octane-2-carboxylate (P2) and ethyl (6R)-6-[4-(6-bromo-5-fluoro-3-pyridyl)piperazin-1-yl]-2-azaspiro[3.4]octane-2-carboxylate (C5A) A mixture of 2-bromo-3,5-difluoro-pyridine (5.00 g, 25.7 mmol), K₂CO₃ (7.13 g, 51.5 mmol) in DMF (100 mL) was added ethyl (6R)-6-piperazin-1-yl-2-azaspiro[3.4]octane-2-carboxylate (7.24 g, 27.0 mmol). Then the mixture was heated to 110° C. and stirred at 110° C. for 22 hours under N₂ atmosphere. The mixture was cooled to 25° C. and filtered. The filtrate was concentrated under reduced pressure to give a residue. The residue was purified by flash silica gel chromatography (Eluent of 0˜3% MeOH/DCM) to give a mixture of P2 and C5A.

Step 2. Synthesis of ethyl (6R)-6-[4-(2-bromo-5-fluoro-3-pyridyl)piperazin-1-yl]-2-azaspiro[3.4]octane-2-carboxylate (P2)

The mixture of P2 and C5A was further separated by SFC (column: DAICEL CHIRALCEL OD (250 mm×50 mm, 10 um); mobile phase: [0.1% NH₃H₂O ETOH]; B %: 35%-35%, B3.5; 60 min) to afford Intermediate P2. ¹H NMR (CDCl₃ 400 MHz) δ_{H}=7.97 (s, 1H), 7.05 (d, J=8.0 Hz, 1H), 4.11 (q, J=7.2 Hz, 2H), 3.98-3.84 (m, 2H), 3.82-3.77 (m, 2H), 3.16 (brs, 4H), 2.72 (brs, 5H), 2.20-2.15 (m, 1H), 2.05-1.75 (m, 5H), 1.24 (t, J=6.8 Hz, 3H). ¹⁹F NMR (CDCl₃ 400 MHz) δ_{F}=−128.178.

### Example #3: Synthesis of ethyl (6R)-6-[4-[2-(5-methoxy-3-pyridyl)-3-pyridyl]piperazin-1-yl]-2-azaspiro[3.4]octane-2-carboxylate (A1)

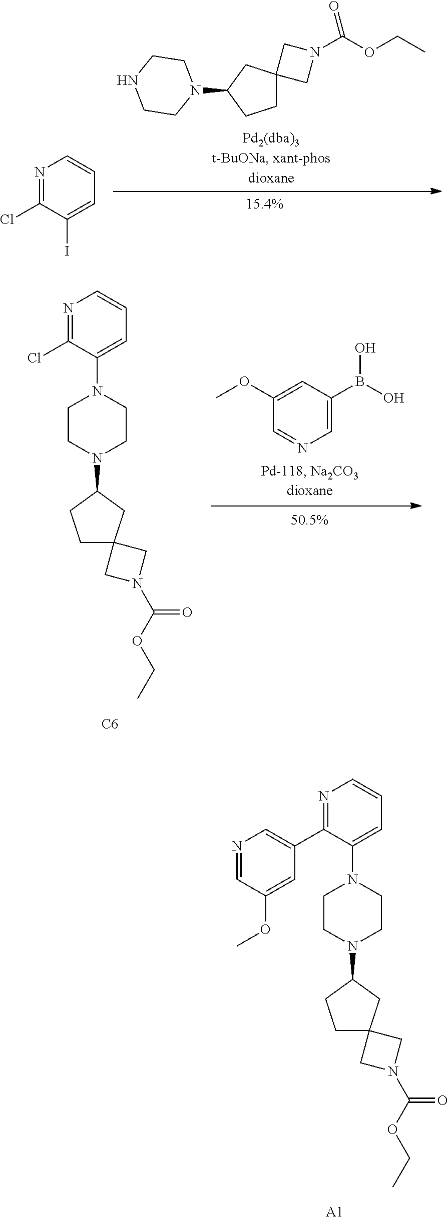

Step 1. Synthesis of ethyl (6R)-6-[4-(2-chloro-3-pyridyl)piperazin-1-yl]-2-azaspiro[3.4]-octane-2-carboxylate (C6) A mixture of 2-chloro-3-iodo-pyridine (373 mg, 1.56 mmol), Intermediate P1 (500 mg, 1.87 mmol), Pd₂(dba)₃ (143 mg, 156 umol), XantPhos (180 mg, 312 umol) and t-BuONa (225 mg, 2.34 mmol in 1,4-dioxane (10.0 mL) was degassed under vacuum and purged with a N₂ gas several times. The reaction mixture was heated to 110° C. and stirred at 110° C. for 16 hours under a N₂ atmosphere. The mixture was cooled to 20° C. and dichloromethane (50 mL) was added to the mixture, filtered and concentrated in vacuum. The residue was purified by flash silica gel chromatography (Eluent of 0˜10% methanol in dichloromethane) to afford Intermediate C6. LCMS m/z [M+H]+ 379.

Step 2. Synthesis of ethyl (6R)-6-[4-[2-(5-methoxy-3-pyridyl)-3-pyridyl]piperazin-1-yl]-2-azaspiro[3.4]octane-2-carboxylate (A1) A mixture of Intermediate C6 (50.0 mg, 132 umol), (5-methoxy-3-pyridyl) boronic acid (40.4 mg, 264 umol), Na₂CO₃ (42.0 mg, 396 umol) and PdCl2(dtpbf) (8.60 mg, 13.2 umol) in 1,4-dioxane (4.00 mL) and H₂O (1.00 mL) was degassed under vacuum and purged with N₂ several times. The reaction mixture was heated to 85° C. and stirred at 85° C. for 16 hours under a N₂ atmosphere. The mixture was filtered and concentrated in vacuum to afford the crude product. The crude product was purified by pre-HPLC (column: Phenomenex Gemini-NX 80×40 mm×3 um; mobile phase: [water (0.05% NH₃H₂O+10 mM NH₄HCO₃)-ACN]; B %: 26%-50%, 8 min) to afford A1. ¹H NMR (CDCl₃ 400 MHz) _{δ}H=8.90 (s, 1H), 8.41 (s, 1H), 8.30 (d, J=2.8 Hz, 1H), 7.73 (s, 1H), 7.50-7.34 (m, 1H), 7.26-7.22 (m, 1H), 4.09 (q, J=7.2 Hz, 2H), 3.98-3.82 (m, 5H), 3.80-3.74 (m, 2H), 3.28-2.86 (m, 4H), 2.83-2.25 (m, 5H), 2.21-1.80 (m, 5H), 1.57-1.47 (m, 1H), 1.23 (t, J=7.2 Hz, 3H). LCMS m/z [M+H]⁺ 452.

### Example #4: Synthesis of ethyl (6R)-6-[4-[5-fluoro-2-(6-fluoro-3-pyridyl)-3-pyridyl]piperazin-1-yl]-2-azaspiro[3.4]octane-2-carboxylate (A2)

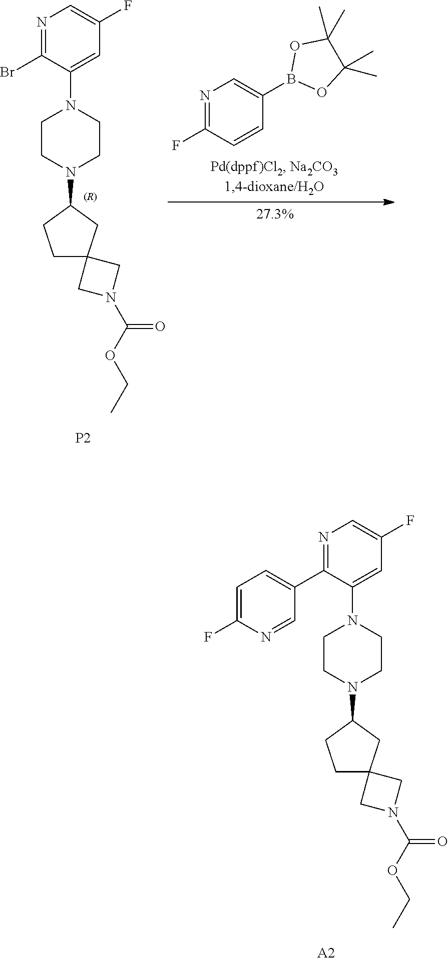

Step 1: Synthesis of ethyl (6R)-6-[4-[5-fluoro-2-(6-fluoro-3-pyridyl)-3-pyridyl]piperazin-1-yl]-2-azaspiro[3.4]octane-2-carboxylate (A2) A mixture of Intermediate P2 (40.0 mg, 90.6 umol), 2-fluoro-5-(4,4,5,5-tetramethyl-1,3,2-dioxaborolan-2-yl)pyridine (30.3 mg, 136 umol), Pd(dppf)Cl₂ (6.63 mg, 9.06 umol), and Na₂CO₃ (28.8 mg, 272 umol) in 1,4-dioxane (2.00 mL) and H₂O (0.500 mL) was degassed and purged with N₂ gas for 3 times, and then the mixture was stirred at 90° C. for 16 hours under a N₂ atmosphere. The reaction mixture was concentrated under reduced pressure. The residue was purified by prep-HPLC (column: Welch Ultimate C18 150×25 mm×5 um; mobile phase: [water (FA)-ACN]; B %: 7%-37%, 10 min) to give A2. ¹H NMR (CDCl₃ 400 MHz) _{δ}H=8.86 (d, J=2.4 Hz, 1H), 8.32 (dt, J=2.4 Hz, 8.0 Hz, 1H), 8.24 (d, J=2.0 Hz, 1H), 7.17-7.10 (m, 1H), 7.01 (dd, J=2.8 Hz, 8.4 Hz, 1H), 4.09 (q, J=7.2 Hz, 2H), 3.92-3.74 (m, 4H), 3.04-2.78 (m, 4H), 2.76-2.34 (m, 5H), 2.17-1.66 (m, 6H), 1.23 (t, J=7.2 Hz, 3H). ¹⁹F NMR (CDCl₃ 400 MHz) _{δ}F=−68.347, −126.520. LCMS m/z [M+H]⁺ 458.

### Example #5: Synthesis of ethyl (6R)-6-[4-(2-pyrrolidin-1-yl-3-pyridyl)piperazin-1-yl]-2-azaspiro[3.4]octane-2-carboxylate (A3)

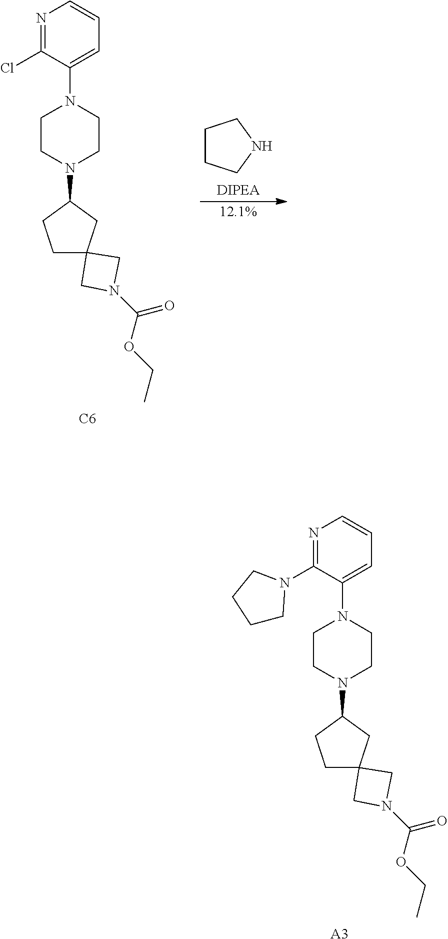 [SMILES: CCOC(=O)N1CC2(CC[C@@H](N3CCN(c4cccnc4Cl)CC3)C2)C1 ; C1CCNC1 ; "C6" ; CCOC(=O)N1CC2(CC[C@@H](N3CCN(c4cccnc4N4CCCC4)CC3)C2)C1 ; "A3"]

Step 1: Synthesis of ethyl (6R)-6-[4-(2-pyrrolidin-1-yl-3-pyridyl)piperazin-1-yl]-2-azaspiro-[3.4]octane-2-carboxylate (A3) A mixture of Intermediate C6 (29.0 mg, 76.5 umol) and pyrrolidine (10.9 mg, 153 umol, 12.8 uL) in DIPEA (0.500 mL) was heated to 100° C. and stirred for 16 hours. Then the mixture was stirred for another 32 hours at 120° C.-130° C. Then the mixture was cooled to 20° C. and concentrated in vacuum. The residue was purified by prep-HPLC (column: Phenomenex Gemini-NX 80×40 mm×3 um; mobile phase: [water (0.05% NH₃H₂O+10 mM NH₄HCO₃)-ACN]; B %: 40%-70%, 8 min) to afford A3. ¹H NMR (CDCl₃ 400 MHz) δ_{H}=7.93-7.88 (m, 1H), 719-7.14 (m, 1H), 6.64 (dd, J=4.8 Hz, 7.6 Hz, 1H), 4.10 (q, J=7.2 Hz, 2H), 3.94-3.83 (m, 2H), 3.82-3.76 (m, 2H), 3.56-3.50 (m, 4H), 3.11-2.79 (m, 4H), 2.78-2.36 (m, 5H), 2.21-2.10 (m, 1H), 2.02-1.64 (m, 9H), 1.24 (t, J=7.2 Hz, 3H). LCMS m/z [M+H]⁺ 414.

### Example #6: Synthesis of ethyl 6-(4-(2-(azetidin-1-yl)pyridin-3-yl)piperazin-1-yl)-2-azaspiro[3.4]octane-2-carboxylate (A4)

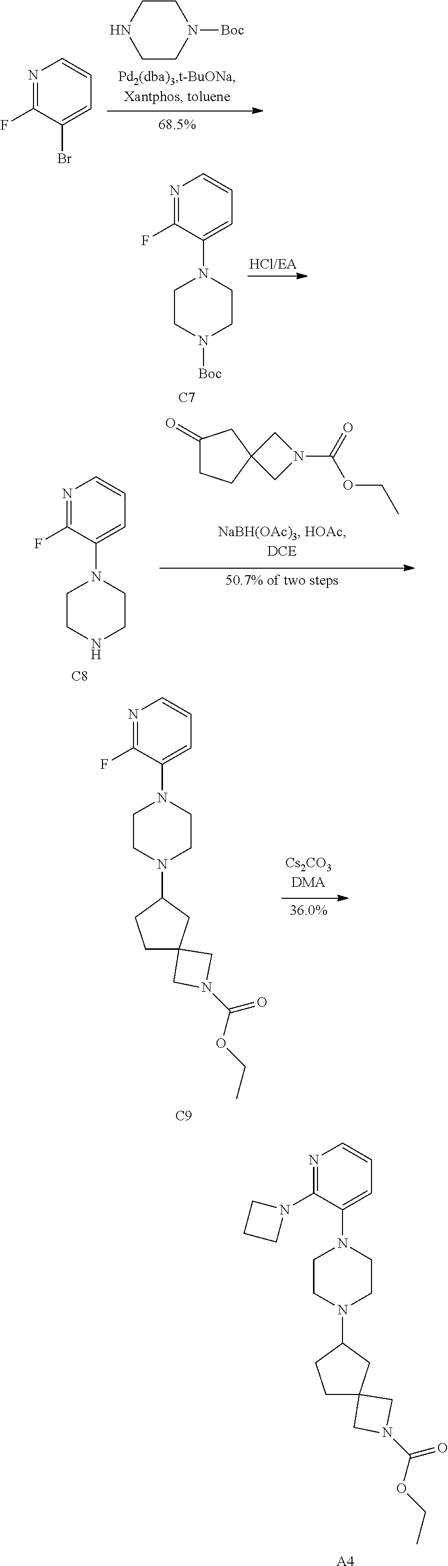

Step 1. Synthesis of tert-butyl 4-(2-fluoro-3-pyridyl)piperazine-1-carboxylate (C7). To a mixture of 3-bromo-2-fluoro-pyridine (2.00 g, 11.4 mmol), tert-butyl piperazine-1-carboxylate (2.54 g, 13.6 mmol), tert-butyl piperazine-1-carboxylate (2.54 g, 13.6 mmol) and tBuONa (1.64 g, 17.1 mmol) in toluene (30.0 mL) were added Pd₂(dba)₃ (520 mg, 568 umol) and XantPhos (658 mg, 1.14 mmol). The mixture was degassed under vacuum and purged with N₂ gas several times. Then the reaction mixture was heated to 100° C. and stirred at 100° C. for 16 hours under a N₂ atmosphere. The mixture was cooled to 25° C., then H₂O (50 mL) was added to the mixture. The aqueous phase was extracted with ethyl acetate (4×50 mL). The combined organic phase was washed with brine (30 mL), dried with anhydrous Na₂SO₄, filtered and concentrated in vacuum. The residue was purified by flash silica gel chromatography (Eluent of 0˜20% Ethyl acetate/Petroleum ether) to afford Intermediate C7. LCMS m/z 282 [M+H]⁺. ¹H NMR (CDCl₃ 400 MHz) δ_{H}=7.82-7.74 (m, 1H), 7.25-7.20 (m, 1H), 7.14-7.10 (m, 1H), 3.62-3.58 (m, 4H), 3.10-3.00 (m, 4H), 1.48 (s, 9H). LCMS m/z [M+H]⁺ 282.

Step 2. Synthesis of 1-(2-fluoro-3-pyridyl)piperazine (C8) To a mixture of Intermediate C7 (2.19 g, 7.78 mmol) in EA (25.0 mL) was added a solution of HCl in EA (4 M, 15.0 mL). The mixture was stirred at 25° C. for 3 hours. The mixture was concentrated in vacuum to get a crude C8 HCl salt, which was used in the next step without further purification.

Step 3. Synthesis of ethyl 6-[4-(2-fluoro-3-pyridyl)piperazin-1-yl]-2-azaspiro[3.4]octane-2-carboxylate (C9) A mixture of Intermediate C8 (1.69 g, 7.76 mmol, HCl salt) and Et₃N (3.93 g, 38.8 mmol) in DCE (20.0 mL) was stirred at 25° C. for 10 min. Then ethyl 6-oxo-2-azaspiro[3.4]octane-2-carboxylate (3.06 g, 15.5 mmol) and AcOH (233 mg, 3.88 mmol) were added to the mixture. The mixture was stirred at 25° C. for 20 min. Then NaBH(OAc)₃ (4.94 g, 23.3 mmol) was added into the mixture at 25° C. and stirred at 25° C. for 16 hours. Saturated aqueous NaHCO₃ (80 mL) was added to the mixture and then the mixture was extracted with dichloromethane (3×50 mL). The combined organic phase was dried over sodium sulfate and concentrated in vacuum. The residue was purified by flash silica gel chromatography (Eluent of 0˜5% methanol in dichloromethane) to afford Intermediate C9. LCMS m/z [M+H]⁺ 363.

Step 4: Synthesis of ethyl 6-[4-[2-(azetidin-1-yl)-3-pyridyl]piperazin-1-yl]-2-azaspiro[3.4]-octane-2-carboxylate (A4) A mixture of Intermediate C9 (100 mg, 276 umol), Cs₂CO₃ (360 mg, 1.10 mmol) and azetidine (77.4 mg, 828 umol, HCl salt) in DMA (5.00 mL) was heated to 130-140° C. and stirred for 26 hours at 130-140° C. The mixture was cooled to 25° C. Then H₂O (20 mL) was added to the mixture. The aqueous phase was extracted with dichloromethane (3×20 mL). The combined organic phase was washed with H₂O (3×50 mL) and concentrated in vacuum. The residue was purified by prep-HPLC (column: Phenomenex Gemini-NX 80×40 mm×3 um; mobile phase: [water (0.05% NH₃H₂O)-ACN]; B %: 33%-59%, 8 min) to afford A4. ¹H NMR (CDCl₃ 400 MHz) δ_{H}=7.88 (dd, J=1.6 Hz, 5.2 Hz, 1H), 7.09 (dd, J=1.2 Hz, 7.6 Hz, 1H), 6.62 (dd, J=5.2 Hz, 7.6 Hz, 1H), 4.14-4.02 (m, 6H), 3.90-3.81 (m, 2H), 3.80-3.72 (m, 2H), 3.09-2.78 (m, 4H), 2.75-2.37 (m, 5H), 2.27-2.18 (m, 2H), 2.16-2.08 (m, 1H), 1.94-1.78 (m, 3H), 1.76-1.68 (m, 1H), 1.62-1.49 (m, 1H), 1.22 (t, J=6.8 Hz, 3H). LCMS m/z [M+H]⁺ 400.

### Example #7: Synthesis of (R)-ethyl 6-(4-(2-cyclobutoxypyridin-3-yl)piperazin-1-yl)-2-azaspiro[3.4]octane-2-carboxylate (A5)

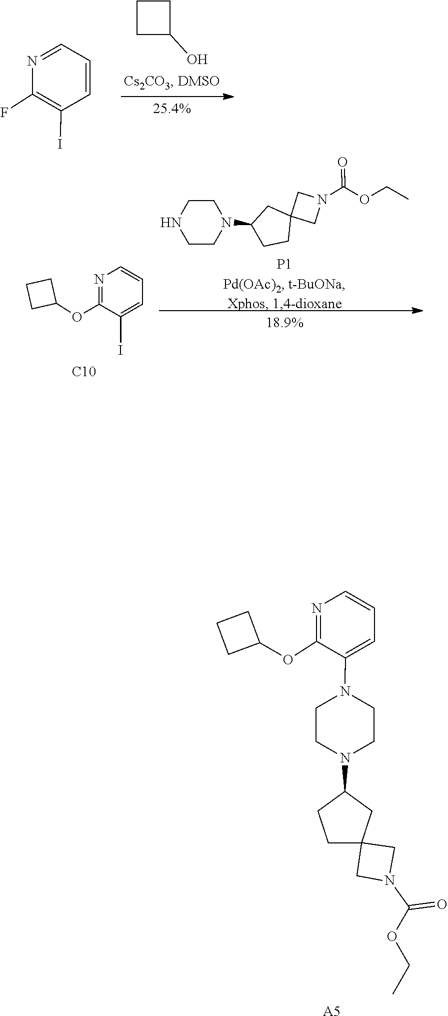

Step 1. Synthesis of 2-(cyclobutoxy)-3-iodo-pyridine (C10) A mixture of 2-fluoro-3-iodo-pyridine (100 mg, 448 umol), cyclobutanol (32.3 mg, 448 umol) and Cs₂CO₃ (292 mg, 897 umol) in DMSO (1.00 mL) was heated to 90° C. and stirred at ˜90° C.-100° C. for 17 hours. The reaction mixture was cooled to 20° C. Then H₂O (10 mL) was added to the mixture. The aqueous phase was extracted with ethyl acetate (3×20 mL). The combined organic phase was washed with brine (10 mL), dried with anhydrous Na₂SO₄, filtered and concentrated in vacuum. The residue was purified by flash silica gel chromatography (Eluent of Petroleum ether) to give Intermediate C10. LCMS m/z [M+H]⁺ 276.

Step 2. Synthesis of ethyl (6R)-6-[4-[2-(cyclobutoxy)-3-pyridyl]piperazin-1-yl]-2-azaspiro-[3.4]octane-2-carboxylate (A5) To a mixture of Intermediate P1 (38.5 mg, 144 umol), Intermediate C10 (33.0 mg, 120 umol) and t-BuONa (23.1 mg, 240 umol) in 1,4-dioxane (0.500 mL), were added XPhos (11.4 mg, 24.0 umol) and Pd(OAc)₂ (5.39 mg, 24.0 umol). Then the mixture was degassed under vacuum and purged with N₂ for several times. The mixture was heated to 90° C. and stirred for 16 hours. The mixture was cooled to 20° C., filtered and concentrated in vacuum and purified by prep-HPLC (column: Phenomenex Gemini-NX 80×40 mm×3 um; mobile phase: [water (0.05% NH₃H₂O+10 mM NH₄HCO₃)-ACN]; B %: 40%-70%, 8 min) to afford A5. ¹H NMR (CDCl₃ 400 MHz) δ_{H}=7.75 (d, J=4.0 Hz, 1H), 7.07 (dd, J=1.6 Hz, 7.6 Hz, 1H), 6.80 (dd, J=4.8 Hz, 7.6 Hz, 1H), 5.29-5.19 (m, 1H), 4.10 (q, J=7.2 Hz, 2H), 3.95-3.84 (m, 2H), 3.83-3.77 (m, 2H), 3.33-3.01 (m, 4H), 2.95-2.55 (m, 5H), 2.54-2.43 (m, 2H), 2.25-2.05 (m, 3H), 2.03-1.77 (m, 5H), 1.75-1.65 (m, 2H), 1.24 (t, J=7.2 Hz, 3H). LCMS m/z [M+H]⁺ 415.

### Example #8: Synthesis of (R)-ethyl 6-(4-(5-fluoro-2-(pyrazin-2-yl)pyridin-3-yl)piperazin-1-yl)-2-azaspiro[3.4]octane-2-carboxylate (A6)

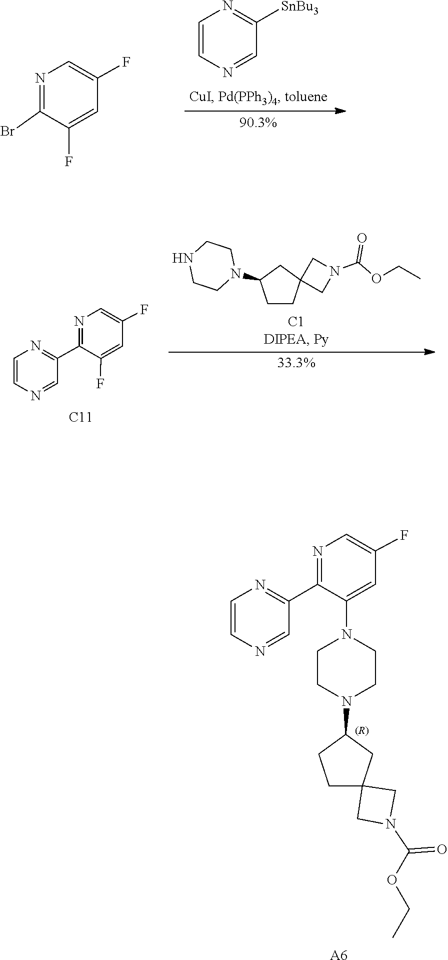

Step 1. Synthesis of 2-(3,5-difluoropyridin-2-yl) pyrazine (C11) A mixture of 2-bromo-3,5-difluoro-pyridine (50.0 mg, 258 umol), tributyl(pyrazin-2-yl)-stannane (142 mg, 387 umol), CuI (4.91 mg, 25.8 umol) and Pd(PPh₃)₄ (29.8 mg, 25.8 umol) in toluene (2.00 mL) was degassed and purged with N₂ for several times at 20° C. Then the mixture was heated to 110° C. and stirred at 110° C. for 12 hours. The mixture was cooled to 20° C. and filtered. The filtrate was concentrated under reduce pressure and purified by flash silica gel chromatography (Eluent of 0˜3% methanol in dichloromethane) to give Intermediate C11. ¹H NMR (CDCl₃ 400 MHz) δ_{H}=9.24 (s, 1H), 8.74 (s, 1H), 8.64 (d, J=2.4 Hz, 1H), 8.54 (d, J=2.0 Hz, 1H), 7.45-7.35 (m, 1H). LCMS m/z [M+H]⁺ 194.

Step 2: Synthesis of (R)-ethyl 6-(4-(5-fluoro-2-(pyrazin-2-yl)pyridin-3-yl)piperazin-1-yl)-2-azaspiro[3.4]octane-2-carboxylate (A6) A mixture of Intermediate P1 (69.2 mg, 259 umol), Intermediate C11 (50.0 mg, 259 umol), DIPEA (0.500 mL, 2.87 mmol) in pyridine (0.500 mL) was stirred at 130° C. for 24 hours. The mixture was concentrated under vacuum and purified by flash silica gel chromatography and prep-HPLC (column: Welch Xtimate C18 150×25 mm×5 um; mobile phase: [water (NH₃H₂O+NH₄HCO₃)-ACN]; B %: 40%-70%, 7 min) to give A6. ¹H NMR (CDCl₃ 400 MHz) δ_{H}=9.23 (s, 1H), 8.73 (s, 1H), 8.55 (d, J=2.4 Hz, 1H), 8.31 (s, 1H), 7.16 (br d, J=10.0 Hz, 1H), 4.09 (q, J=7.2 Hz, 2H), 3.88-3.74 (m, 4H), 2.91 (br s, 4H), 2.63-2.38 (m, 5H), 2.12-2.03 (m, 1H), 1.95-1.75 (m, 3H), 1.70-1.60 (m, 1H), 1.52-1.42 (br s, 1H), 1.23 (t, J=7.2 Hz, 3H). ¹⁹F NMR (CDCl₃ 400 MHz) δ_{F}=−124.49. LCMS m/z [M+H]⁺ 441.

### Example #9: Synthesis of ethyl (6R)-6-[4-(5-fluoro-2-tetrahydropyran-4-yl-3-pyridyl)piperazin-1-yl]-2-azaspiro[3.4]octane-2-carboxylate (A7)

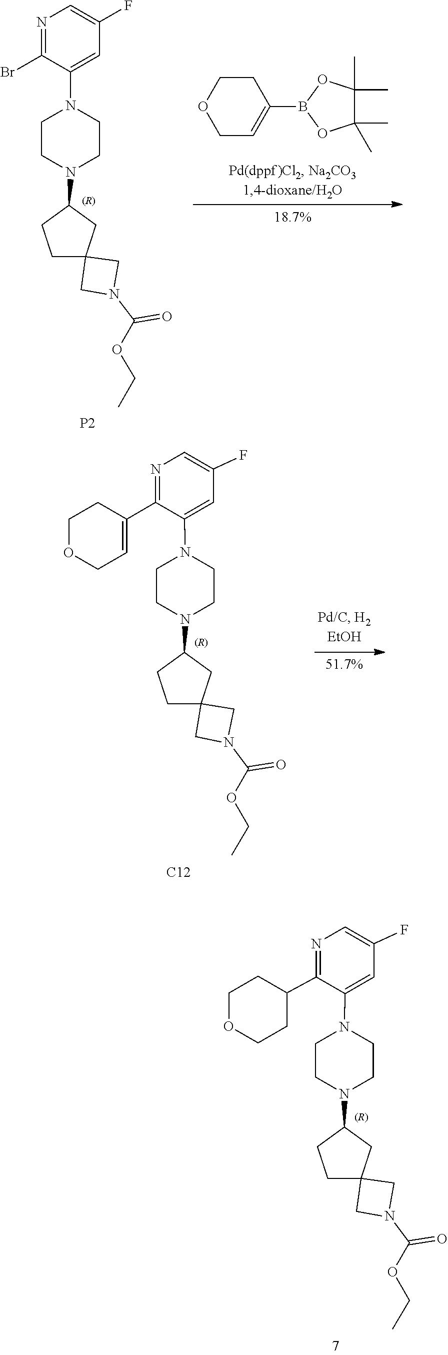

Step 1: Synthesis of ethyl (6R)-6-[4-[2-(3,6-dihydro-2H-pyran-4-yl)-5-fluoro-3-pyridyl]piperazin-1-yl]-2-azaspiro[3.4]octane-2-carboxylate (C12) A mixture of ethyl (6R)-6-[4-(2-bromo-5-fluoro-3-pyridyl)piperazin-1-yl]-2-azaspiro[3.4]octane-2-carboxylate (100 mg, 227 umol), 2-(3,6-dihydro-2H-pyran-4-yl)-4,4,5,5-tetramethyl-1,3,2-dioxaborolane (71.4 mg, 340 umol), Pd(dppf)Cl₂ (16.6 mg, 22.7 umol) and Na₂CO₃ (72.0 mg, 680 umol) in 1,4-dioxane (2.00 mL) and H₂O (0.250 mL) was degassed and purged with N₂ for several times at 20° C. Then the mixture was heated to 90° C. and stirred at 90° C. for 12 hours. The mixture was cooled to 20° C. and concentrated under vacuum and purified by flash silica gel chromatography (Eluent of 0˜5% MeOH/DCM) to give a crude. The crude was purified by prep-HPLC (column: Welch Xtimate C18 150×30 mm×5 um; mobile phase: [water (NH₃H₂O+NH₄HCO₃)-ACN]; B %: 40%-70%, 7 min) to give C12 (19.8 mg, 42.3 umol, 18.7% yield, 94.9% purity). ¹H NMR (CDCl₃ 400 MHz) δ_{H}=8.08 (d, J=2.0 Hz, 1H), 6.99 (dd, J=2.4 Hz, 10.4 Hz, 1H), 6.36 (br s, 1H), 4.33 (d, J=2.8 Hz, 2H), 4.10 (q, J=7.2 Hz, 2H), 3.95-3.72 (m, 6H), 3.03 (br s, 4H), 2.70-2.49 (m, 7H), 2.14 (dd, J=7.2 Hz, 12.8 Hz, 1H), 2.02-1.79 (m, 3H), 1.77-1.71 (m, 1H), 1.62-1.49 (m, 1H), 1.23 (t, J=7.2 Hz, 3H). LCMS m/z [M+H]⁺ 445.

Step 2: Synthesis of ethyl (6R)-6-[4-(5-fluoro-2-tetrahydropyran-4-yl-3-pyridyl)piperazin-1-yl]-2-azaspiro[3.4]octane-2-carboxylate (A7) To a solution of ethyl (6R)-6-[4-[2-(3,6-dihydro-2H-pyran-4-yl)-5-fluoro-3-pyridyl]piperazin-1-yl]-2-azaspiro[3.4]octane-2-carboxylate (50.0 mg, 112 umol) in EtOH (5.00 mL) was added wet Pd/C (50.0 mg, 10.0% purity) at 20° C. under N₂ and the mixture was degassed and purged with H₂ for three times. Then the mixture was stirred at 40° C. for 16 hours under H₂ (15 psi) atmosphere. The mixture was filtered and the filtrate was concentrated under vacuum and purified by prep-HPLC (column: Welch Xtimate C18 150×30 mm×5 um; mobile phase: [water (NH₃H₂O+NH₄HCO₃)-ACN]; B %: 40%-70%, 7 min) to give compound 7 (27.0 mg, 58.1 umol, 51.7% yield, 96.1% purity). ¹H NMR (CDCl₃ 400 MHz) δ_{H}=8.20 (br s, 1H), 7.11 (br d, J=9.6 Hz, 1H), 4.10 (q, J=7.2 Hz, 4H), 3.96-3.77 (m, 4H), 3.53 (br t, J=11.6 Hz, 2H), 3.32 (br t, J=12.4 Hz, 1H), 2.90 (br s, 4H), 2.66 (br s, 5H), 2.18-1.69 (m, 6H), 1.58 (s, 4H), 1.24 (t, J=7.2 Hz, 3H). LCMS m/z [M+H]⁺ 447.

### Example #10: Synthesis of ethyl (6R)-6-[4-(5-fluoro-2-thiazol-4-yl-3-pyridyl)piperazin-1-yl]-2-azaspiro[3.4]octane-2-carboxylate (A8)

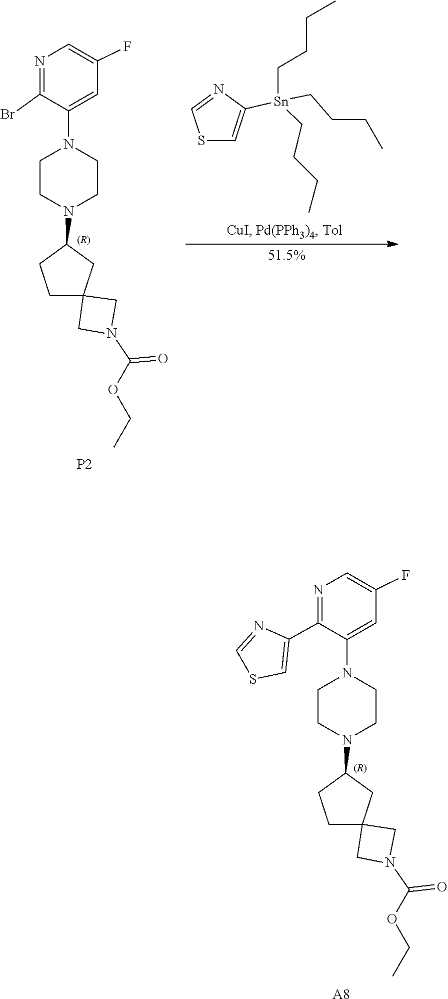

To a mixture of ethyl (6R)-6-[4-(2-bromo-5-fluoro-3-pyridyl)piperazin-1-yl]-2-azaspiro[3.4]octane-2-carboxylate (100 mg, 227 μmol), tributyl(thiazol-4-yl)stannane (127 mg, 340 μmol) in toluene (1.00 mL) was added CuI (4.32 mg, 22.7 μmol) and Pd(PPh₃)₄ (26.2 mg, 22.7 μmol). The mixture was degassed under a vacuum and purged with N₂ gas for several times. The reaction mixture was stirred at 110° C. for 12 h under a N₂ gas atmosphere. The mixture was concentrated under a reduced pressure to give a residue. The residue was purified by flash silica gel chromatography (Eluent of 0˜5% MeOH/DCM) and further purified by prep-HPLC (column: Welch Xtimate C18, 150×30 mm×5 um; mobile phase: [water (FA)-ACN]; gradient: 0%-40% B over 9 min) to give ethyl (6R)-6-[4-(5-fluoro-2-thiazol-4-yl-3-pyridyl)piperazin-1-yl]-2-azaspiro[3.4]octane-2-carboxylate (52.0 mg, 117 μmol, 51.5% yield, 100% purity) as a yellow oil. ¹H NMR (CDCl₃ 400 MHz) δ_{H}=8.92 (s, 1H), 8.26 (s, 1H), 8.10 (s, 1H), 7.19-7.09 (m, 1H), 4.10 (q, J=7.2 Hz, 2H), 3.95-3.73 (m, 4H), 3.04 (br s, 4H), 2.90-2.70 (m, 5H), 2.23-2.11 (m, 1H), 2.03-1.90 (m, 3H), 1.88-1.78 (m, 1H), 1.77-1.68 (m, 1H), 1.23 (t, J=7.2 Hz, 3H). LCMS m/z [M+H]⁺ 446.

### Example #11: Synthesis of ethyl (6R)-6-[4-[5-fluoro-2-(tetrahydropyran-4-ylamino)-3-pyridyl]piperazin-1-yl]-2-azaspiro[3.4]octane-2-carboxylate (A9)

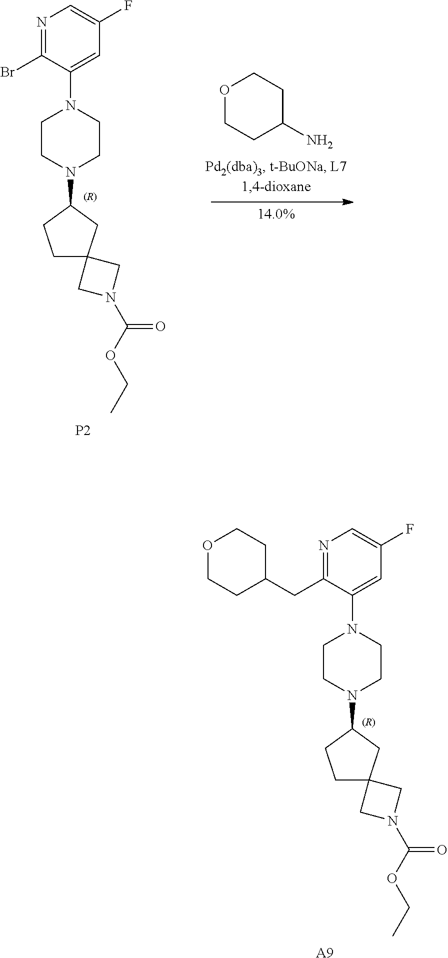

A mixture of tetrahydropyran-4-amine (68.8 mg, 680 μmol), ethyl (6R)-6-[4-(2-bromo-5-fluoro-3-pyridyl)piperazin-1-yl]-2-azaspiro[3.4]octane-2-carboxylate (150 mg, 340 μmol), Pd₂(dba)₃ (31.1 mg, 34.0 μmol), t-BuONa (98.0 mg, 1.02 mmol), 2-[Bis(3,5-trifluoromethylphenylphosphino)-3,6-dimethoxy]-2,6-dimethylamino-1,1-biphenyl) (L7, 25.7 mg, 34.0 μmol) in 1,4-dioxane (2.00 mL) was degassed under a vacuum and purged with N₂ gas for 3 times, and then the mixture was stirred at 100° C. for 12 h under a N₂ gas atmosphere. The mixture was concentrated under a reduced pressure to give a residue. The residue was purified by flash silica gel chromatography (Eluent of 0˜3% MeOH/DCM) and further purified by prep-HPLC (column: Boston Prime C18, 150×30 mm×5 um; mobile phase: [water (NH₃H₂O+NH₄HCO₃)-ACN]; B %: 52%-82%, 7 min) to give ethyl (6R)-6-[4-[5-fluoro-2-(tetrahydropyran-4-ylamino)-3-pyridyl]piperazin-1-yl]-2-azaspiro[3.4]octane-2-carboxylate (28.7 mg, 61.7 umol, 14.0% yield, 99.3% purity) as an off-white solid. ¹H NMR (CDCl₃ 400 MHz) δH=7.73 (s, 1H), 6.94 (dd, J=2.4 Hz, 9.2 Hz, 1H), 4.83 (d, J=7.2 Hz, 1H), 4.18-4.03 (m, 3H), 4.01-3.77 (m, 6H), 3.58 (t, J=10.8 Hz, 2H), 2.89 (br s, 4H), 2.70-2.55 (m, 5H), 2.20-2.10 (m, 1H), 2.05 (d, J=11.2 Hz, 2H), 1.98-1.80 (m, 3H), 1.78-1.66 (m, 1H), 1.56-1.46 (m, 3H), 1.24 (t, J=7.2 Hz, 3H). LCMS m/z [M+H]⁺ 462.

### Example #12: Synthesis of ethyl (6R)-6-[4-[5-fluoro-2-(4-methylpyrazol-1-yl)-3-pyridyl]piperazin-1-yl]-2-azaspiro[3.4]octane-2-carboxylate (A10)

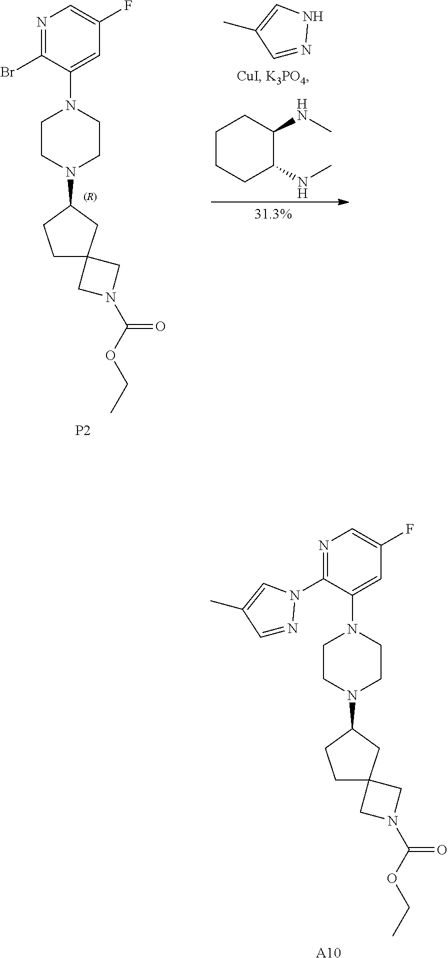

To a mixture of ethyl (6R)-6-[4-(2-bromo-5-fluoro-3-pyridyl)piperazin-1-yl]-2-azaspiro[3.4]octane-2-carboxylate (30.0 mg, 68.0 μmol), 4-methyl-1H-pyrazole (8.37 mg, 102 μmol) and (1R,2R)—N1,N2-dimethylcyclohexane-1,2-diamine (9.67 mg, 68.0 μmol) in DMF (1.00 mL) was added CuI (13.0 mg, 68.0 μmol) and K₃PO₄ (43.3 mg, 204 μmol) in one portion. The mixture was degassed under a vacuum and purged with N₂ gas for several times. The reaction mixture was stirred at 130° C. for 12 h under a N₂ gas atmosphere. The mixture was concentrated under a reduced pressure to give a residue. The residue was purified by prep-HPLC (column: Phenomenex C18, 80×40 mm×3 um; mobile phase: [water (NH₃H₂O+NH₄HCO₃)-ACN]; gradient: 39%-69% B over 8 min) to give ethyl (6R)-6-[4-[5-fluoro-2-(4-methylpyrazol-1-yl)-3-pyridyl]piperazin-1-yl]-2-azaspiro[3.4]octane-2-carboxylate (9.40 mg, 21.2 μmol, 31.3% yield, 100% purity) as an off-white solid. ¹H NMR (CD₃OD 400 MHz) δ_{H}=8.09-7.83 (m, 2H), 7.60 (s, 1H), 7.45 (d, J=8.0 Hz, 1H), 4.07 (q, J=7.2 Hz, 2H), 3.93-3.70 (m, 4H), 2.75 (d, J=4.4 Hz, 4H), 2.69-2.45 (m, 5H), 2.20 (s, 3H)), 2.18-2.11 (m, 1H), 1.99-1.81 (m, 3H), 1.70 (dd, J=9.6 Hz, 12.8 Hz, 1H), 1.57-1.46 (m, 1H), 1.22 (t, J=7.2 Hz, 3H). LCMS m/z [M+H]⁺ 443.

### Example #13: Synthesis of ethyl (6R)-6-[4-(5-fluoro-2-pyrazin-2-yl-3-pyridyl)-1-piperidyl]-2-azaspiro[3.4]octane-2-carboxylate (A11) and ethyl (6S)-6-[4-(5-fluoro-2-pyrazin-2-yl-3-pyridyl)-1-piperidyl]-2-azaspiro[3.4]octane-2-carboxylate (A12)

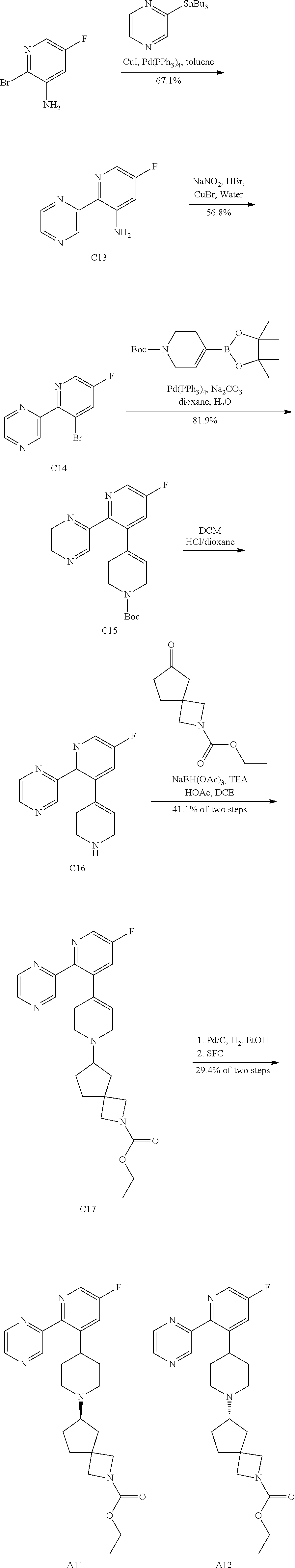

### Step 1: Synthesis of 5-fluoro-2-(pyrazin-2-yl)pyridin-3-amine (C13)

A mixture of 2-bromo-5-fluoro-pyridin-3-amine (1.00 g, 5.24 mmol), tributyl(pyrazin-2-yl)stannane (1.93 g, 5.24 mmol), CuI (99.7 mg, 524 umol) and Pd(PPh₃)₄ (605 mg, 524 umol) in toluene (20.0 mL) was degassed under a reduced pressure and purged with N₂ gas for 3 times, then the mixture was stirred at 100° C. for 12 h under a N₂ gas atmosphere. The mixture was filtered and concentrated under a reduced pressure to remove toluene. The residue was diluted with water (80 mL) and extracted with ethyl acetate (60 mL×3). The combined organic phase was washed with brine (50 mL), dried over anhydrous Na₂SO₄, filtered and concentrated under a reduced pressure. The residue was purified by flash silica gel chromatography (Eluent of 0˜17% Ethyl acetate/Petroleum ether) to give 5-fluoro-2-pyrazin-2-yl-pyridin-3-amine (700 mg, 3.52 mmol, 67.1% yield, 95.5% purity) as an off-white solid. LCMS m/z [M+H]⁺ 191.

### Step 2: Synthesis of 2-(3-bromo-5-fluoropyridin-2-yl)pyrazine (C14)

A solution of 5-fluoro-2-pyrazin-2-yl-pyridin-3-amine (700 mg, 3.68 mmol) in water (4.00 mL) was added a solution of HBr in H₂O (4.00 mL, 48% purity) in one portion at 0° C., the mixture was stirred at 0° C. for 0.5 h. Then a solution of NaNO₂ (279 mg, 4.05 mmol) in water (2.00 mL) was added in one portion at 0° C., the mixture was stirred at 0° C. for another 0.5 h. The mixture was added to the solution of CuBr (581 mg, 4.05 mmol) in water (2.00 mL) at 60° C. The mixture was stirred at 60° C. for 2 h. The mixture was poured into saturated aqueous NaHCO₃ solution (100 mL) and stirred for 5 min. The mixture was extracted with ethyl acetate (60 mL×3). The combined organic phase was washed with brine (60 mL), dried over anhydrous Na₂SO₄, filtered and concentrated under a reduced pressure. The residue was purified by flash silica gel chromatography (Eluent of 0˜11% Ethyl acetate/Petroleum ether) to give 2-(3-bromo-5-fluoro-2-pyridyl)pyrazine (580 mg, 2.09 mmol, 56.8% yield, 91.5% purity) as an off-white solid. LCMS m/z [M+H]⁺ 254, 256.

### Step 3: Synthesis of tert-butyl 5-fluoro-2-(pyrazin-2-yl)-5′,6′-dihydro-[3,4′-bipyridine]-1′(2′H)-carboxylate (C15)

A mixture of 2-(3-bromo-5-fluoro-2-pyridyl)pyrazine (580 mg, 2.28 mmol), tert-butyl 4-(4,4,5,5-tetramethyl-1,3,2-dioxaborolan-2-yl)-3,6-dihydro-2H-pyridine-1-carboxylate (706 mg, 2.28 mmol), Pd(PPh₃)₄ (264 mg, 228 umol) and Na₂CO₃ (726 mg, 6.85 mmol) in water (1.00 mL) and 1,4-dioxane (8.00 mL) was degassed under a reduced pressure and purged with N₂ gas for 3 times, then the mixture was stirred at 100° C. for 12 h under a N₂ gas atmosphere. The mixture was filtered and the organic phase was concentrated under a reduced pressure to give a residue. The residue was diluted with water (80 mL) and extracted with ethyl acetate (3×60 mL). The combined organic phase was washed with brine (50 mL), dried over anhydrous Na₂SO₄, filtered and concentrated under a reduced pressure. The residue was purified by flash silica gel chromatography (Eluent of 0˜14% Ethyl acetate/Petroleum ether) to give tert-butyl 4-(5-fluoro-2-pyrazin-2-yl-3-pyridyl)-3,6-dihydro-2H-pyridine-1-carboxylate (840 mg, 1.87 mmol, 81.9% yield, 79.3% purity) as a yellow oil. ¹H NMR (CDCl₃ 400 MHz) δ_{H}=9.10 (s, 1H), 8.67-8.42 (m, 3H), 7.37 (dd, J=2.8 Hz, 8.8 Hz, 1H), 5.60 (s, 1H), 3.97 (s, 2H), 3.51 (t, J=5.2 Hz, 2H), 2.15 (s, 2H), 1.48 (s, 9H). LCMS m/z [M+H]⁺ 357.

### Step 4: Synthesis of 5-fluoro-2-(pyrazin-2-yl)-1′,2′,3′,6′-tetrahydro-3,4′-bipyridine (C16)

To a solution of tert-butyl 4-(5-fluoro-2-pyrazin-2-yl-3-pyridyl)-3,6-dihydro-2H-pyridine-1-carboxylate (800 mg, 2.24 mmol) in DCM (12.0 mL) was added HCl/1,4-dioxane (4 M, 4.00 mL). The mixture was stirred at room temperature for 12 h. The mixture was adjusted to pH=8 by saturated aqueous NaHCO₃ solution (50 mL) and extracted with DCM (3×40 mL). The combined organic phase was washed with brine (40 mL), dried over anhydrous Na₂SO₄, filtered and concentrate under a reduced pressure to give 2-[5-fluoro-3-(1,2,3,6-tetrahydropyridin-4-yl)-2-pyridyl]-pyrazine (510 mg, crude) as a yellow oil. The crude product was used in the next step without further purification. LCMS m/z [M+H]⁺ 257.

### Step 5: Synthesis of ethyl 6-(5-fluoro-2-(pyrazin-2-yl)-5′,6′-dihydro-[3,4′-bipyridin]-1′(2′H)-yl)-2-azaspiro[3.4]octane-2-carboxylate (C17)

A mixture of 2-[5-fluoro-3-(1,2,3,6-tetrahydropyridin-4-yl)-2-pyridyl]pyrazine (510 mg, 1.99 mmol) and ethyl 6-oxo-2-azaspiro[3.4]octane-2-carboxylate (432 mg, 2.19 mmol) in DCE (16.0 mL) was stirred at room temperature for 1 h. Then NaBH(OAc)₃ (1.27 g, 5.97 mmol) and AcOH (12.0 mg, 199 umol) was added. The mixture was stirred at room temperature for 2 h. The mixture was poured into saturated aqueous NaHCO₃ solution (80 mL) and stirred for 5 min. The mixture was extracted with DCM (3×60 mL). The combined organic phase was washed with brine (60 mL), dried over anhydrous Na₂SO₄, filtered and concentrated under a reduced pressure to give a residue. The residue was purified by flash silica gel chromatography (Eluent of 0˜4% MeOH/DCM) to give ethyl 6-[4-(5-fluoro-2-pyrazin-2-yl-3-pyridyl)-3,6-dihydro-2H-pyridin-1-yl]-2-azaspiro[3.4]octane-2-carboxylate (600 mg, 1.35 mmol, 67.6% yield, 98.1% purity) as a yellow oil. The yellow oil (100 mg) was purified by prep-HPLC (column: Boston Prime C18, 150×30 mm×5 um; mobile phase: [water (NH₃H₂O+NH₄HCO₃)-ACN]; B %: 45%-75%, 7 min) to give ethyl 6-[4-(5-fluoro-2-pyrazin-2-yl-3-pyridyl)-3,6-dihydro-2H-pyridin-1-yl]-2-azaspiro[3.4]octane-2-carboxylate (41.1 mg, 93.9 umol, 41.1% yield, 100% purity) as a yellow oil. ¹H NMR (CDCl₃ 400 MHz) δ_{H}=9.03 (s, 1H), 8.81-8.30 (m, 3H), 7.47-7.33 (m, 1H), 5.58 (s, 1H), 4.10 (q, J=7.2 Hz, 2H), 3.96-3.83 (m, 2H), 3.84-3.74 (s, 2H), 3.09 (br s, 2H), 2.80-2.40 (m 3H), 2.30-2.05 (m, 3H), 2.02-1.80 (m, 3H), 1.89-1.60 (m, 2H), 1.24 (t, J=7.2 Hz, 3H). LCMS m/z [M+H]⁺ 438.

### Step 6: Synthesis of ethyl (R)-6-(4-(5-fluoro-2-(pyrazin-2-yl)pyridin-3-yl)piperidin-1-yl)-2-azaspiro[3.4]octane-2-carboxylate (A11) and ethyl (S)-6-(4-(5-fluoro-2-(pyrazin-2-yl)pyridin-3-yl)piperidin-1-yl)-2-azaspiro[3.4]octane-2-carboxylate (A12)

A mixture of ethyl 6-[4-(5-fluoro-2-pyrazin-2-yl-3-pyridyl)-3,6-dihydro-2H-pyridin-1-yl]-2-azaspiro[3.4]octane-2-carboxylate (200 mg, 457 umol), Pd/C (50.0 mg, 10% purity) in EtOH (6.00 mL) was degassed under a reduced pressure and purged with H₂ gas for 3 times, then the mixture was stirred at 40° C. for 48 h under a H₂ gas atmosphere (40 psi). The mixture was filtered and the organic phase was concentrated under a reduced pressure to give a residue. The residue was purified by flash silica gel chromatography (Eluent of 0˜5% MeOH/DCM) and further separated by SFC (column: DAICEL CHIRALPAK IG (250 mm×30 mm, 10 um); mobile phase: [0.1% NH₃H₂O MeOH]; B %: 50%-50%) to give Peak 1 ethyl (6R)-6-[4-(5-fluoro-2-pyrazin-2-yl-3-pyridyl)-1-piperidyl]-2-azaspiro[3.4]octane-2-carboxylate (14.7 mg, 33.2 umol, 14.6% yield, 99.4% purity) as a yellow oil. ¹H NMR (CDCl₃ 400 MHz) δ_{H}=9.07 (s, 1H), 8.70-8.52 (m, 2H), 8.43 (d, J=2.4 Hz, 1H), 7.68-7.38 (m, 1H), 4.09 (q, J=7.2 Hz, 2H), 3.98-3.80 (m, 2H), 3.77 (s, 2H), 3.45-2.88 (m, 3H), 2.81-2.29 (m, 2H), 2.27-2.07 (m, 1H), 2.05-1.62 (m, 10H), 1.23 (t, J=7.2 Hz, 3H). LCMS m/z [M+H]⁺ 440. Peak 2 ethyl (6S)-6-[4-(5-fluoro-2-pyrazin-2-yl-3-pyridyl)-1-piperidyl]-2-azaspiro[3.4]octane-2-carboxylate (15.7 mg, 33.6 umol, 14.8% yield, 93.9% purity) as a yellow oil. ¹H NMR (CDCl₃ 400 MHz) δ_{H}=9.08 (s, 1H), 8.68-8.54 (m, 2H), 8.44 (d, J=2.8 Hz, 1H), 7.71-7.39 (m, 1H), 4.09 (q, J=7.2 Hz, 2H), 4.02-3.81 (m, 2H), 3.77 (s, 2H), 3.51-2.84 (m, 3H), 2.83-2.30 (m, 2H), 2.23-2.14 (m, 1H), 2.11-1.62 (m, 10H), 1.23 (t, J=7.2 Hz, 3H). LCMS m/z [M+H]⁺ 440.

### Example #14: Synthesis of ethyl (6S)-6-[4-(5-fluoro-2-pyrazin-2-yl-3-pyridyl)-4-hydroxy-1-piperidyl]-2-azaspiro[3.4]octane-2-carboxylate (A13) and ethyl (6R)-6-[4-(5-fluoro-2-pyrazin-2-yl-3-pyridyl)-4-hydroxy-1-piperidyl]-2-azaspiro[3.4]octane-2-carboxylate (A14)

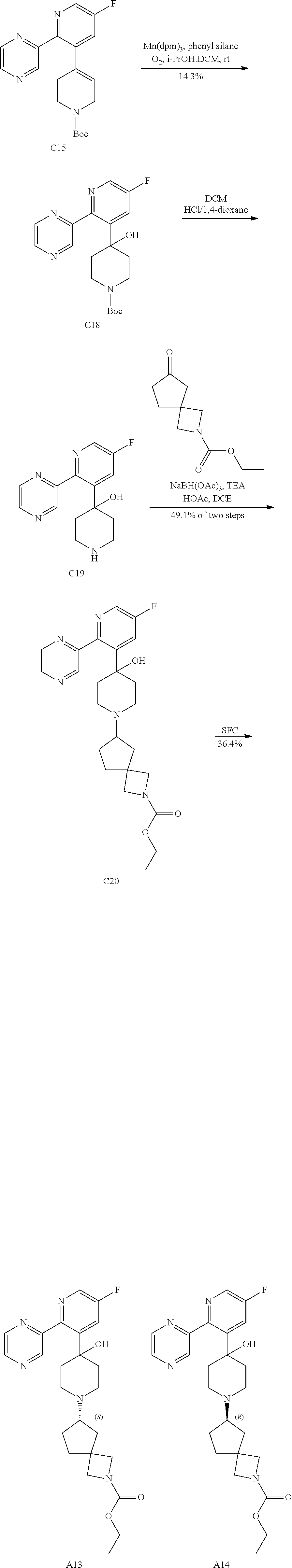

### Step 1: Synthesis of tert-butyl 4-(5-fluoro-2-(pyrazin-2-yl)pyridin-3-yl)-4-hydroxypiperidine-1-carboxylate(C18)

To a solution of tert-butyl 4-(5-fluoro-2-pyrazin-2-yl-3-pyridyl)-3,6-dihydro-2H-pyridine-1-carboxylate (400 mg, 1.12 mmol) in i-PrOH (20.0 mL) and DCM (4.00 mL) was added phenylsilane (486 mg, 4.49 mmol) and tris[(Z)-1-tert-butyl-4,4-dimethyl-3-oxo-pent-1-enoxy]manganese (204 mg, 337 μmol). The reaction mixture was stirred at room temperature for 12 h under an O₂ gas atmosphere (15 psi). The reaction mixture was concentrated under a reduced pressure to give a residue. The residue was purified by flash silica gel chromatography (Eluent of 0˜30% Ethyl acetate/Petroleum ether) to give tert-butyl 4-(5-fluoro-2-pyrazin-2-yl-3-pyridyl)-4-hydroxy-piperidine-1-carboxylate (60.0 mg, 160 μmol, 14.3% yield) as a yellow oil. ¹H NMR (CD₃OD 400 MHz) δ_{H}=9.05-8.85 (m, 1H), 8.66 (d, J=2.4 Hz, 1H), 8.63-8.60 (m, 1H), 8.52 (d, J=2.4 Hz, 1H), 7.95 (dd, J=2.4 Hz, 10.4 Hz, 1H), 3.89 (d, J=12.8 Hz, 2H), 3.24-3.07 (m, 2H), 1.97-1.74 (m, 4H), 1.44 (s, 9H). LCMS m/z [M+H-56]⁺319.

### Step 2: Synthesis of 4-(5-fluoro-2-(pyrazin-2-yl)pyridin-3-yl)piperidin-4-ol (C19)

To a solution of tert-butyl 4-(5-fluoro-2-pyrazin-2-yl-3-pyridyl)-4-hydroxy-piperidine-1-carboxylate (60.0 mg, 160 μmol) in DCM (8.00 mL) was added HCl/1,4-dioxane (4 M, 4.00 mL). The reaction mixture was stirred at room temperature for 2 h. The reaction mixture was concentrated under a reduced pressure to give 4-(5-fluoro-2-pyrazin-2-yl-3-pyridyl)piperidin-4-ol (50.0 mg, crude, HCl salt) as a yellow solid. The crude product was used into the next step without further purification. LCMS m/z [M+H]⁺ 275.

### Step 3: Synthesis of ethyl 6-(4-(5-fluoro-2-(pyrazin-2-yl)pyridin-3-yl)-4-hydroxypiperidin-1-yl)-2-azaspiro[3.4]octane-2-carboxylate (C20)

A mixture of 4-(5-fluoro-2-pyrazin-2-yl-3-pyridyl)piperidin-4-ol (50.0 mg, 161 μmol, HCl salt) and ethyl 6-oxo-2-azaspiro[3.4]octane-2-carboxylate (31.7 mg, 161 μmol) and Et₃N (48.8 mg, 483 μmol) in DCE (10.0 mL) was stirred at room temperature for 1 h. Then AcOH (9.66 mg, 16.1 μmol) and NaBH(OAc)₃ (102 mg, 483 μmol) were added in one portion. The reaction mixture was stirred at room temperature for 11 h. The mixture was poured into a saturated aqueous NaHCO₃ solution (30 mL) and extracted with DCM (3×30 mL). The combined organic phase was washed with brine (50 mL), dried over anhydrous Na₂SO₄, filtered and concentrated under a reduced pressure. The residue was purified by flash silica gel chromatography (Eluent of 0˜6% MeOH/DCM) to give ethyl 6-[4-(5-fluoro-2-pyrazin-2-yl-3-pyridyl)-4-hydroxy-1-piperidyl]-2-azaspiro[3.4]octane-2-carboxylate (36.0 mg, 79.0 μmol, 49.1% yield) as a yellow oil. ¹H NMR (CD₃OD 400 MHz) δ_{H}=9.10-8.90 (m, 1H), 8.81-8.51 (m, 3H), 7.91 (dd, J=2.4 Hz, 10.4 Hz, 1H), 4.11-4.03 (m, 2H), 3.94-3.75 (m, 4H), 3.07-2.85 (m, 3H), 2.80-2.60 (m, 2H), 2.35-2.15 (m, 1H), 2.12-2.02 (m, 2H), 2.00-1.73 (m, 6H), 1.66-1.47 (m, 1H), 1.25-1.20 (m, 3H). LCMS m/z [M+H]⁺ 456.

### Step 4: Synthesis of ethyl (6R)-6-[4-(5-fluoro-2-pyrazin-2-yl-3-pyridyl)-4-hydroxy-1-piperidyl]-2-azaspiro[3.4]octane-2-carboxylate (A13) and ethyl (6S)-6-[4-(5-fluoro-2-pyrazin-2-yl-3-pyridyl)-4-hydroxy-1-piperidyl]-2-azaspiro[3.4]octane-2-carboxylate (A14)

The yellow oil was purified by SFC (column: DAICEL CHIRALPAK IC, (250 mm×30 mm, 10 um); mobile phase: [CO₂-EtOH (0.1% NH₃H₂O)]; B %: 55%%, isocratic elution mode) to afford Peak 1 of ethyl (6S)-6-[4-(5-fluoro-2-pyrazin-2-yl-3-pyridyl)-4-hydroxy-1-piperidyl]-2-azaspiro[3.4]octane-2-carboxylate (6.50 mg, 13.7 μmol, 95.9% purity) as an off-white solid. ¹H NMR (CDCl₃400 MHz) δ_{H}=9.37 (s, 1H), 8.70 (d, J=2.8 Hz, 1H), 8.56-8.45 (m, 2H), 7.65 (dd, J=2.4 Hz, 10.2 Hz, 1H), 7.44 (br s, 1H), 4.09 (q, J=7.2 Hz, 2H), 3.91-3.72 (m, 4H), 2.89-2.38 (m, 5H), 2.17-2.05 (m, 1H), 1.98-1.74 (m, 7H), 1.73-1.66 (m, 1H), 1.57-1.46 (m, 1H), 1.23 (t, J=7.2 Hz, 3H). LCMS m/z [M+H]+ 456. Peak 2 of ethyl (6R)-6-[4-(5-fluoro-2-pyrazin-2-yl-3-pyridyl)-4-hydroxy-1-piperidyl]-2-azaspiro[3.4]octane-2-carboxylate (6.60 mg, 13.2 μmol, 91.4% purity) as an off-white solid. ¹H NMR (CDCl₃ 400 MHz) δ_{H}=9.37 (s, 1H), 8.70 (d, J=2.4 Hz, 1H), 8.55-8.46 (m, 2H), 7.65 (dd, J=2.4 Hz, 10.4 Hz, 1H), 7.44 (br s, 1H), 4.09 (q, J=6.8 Hz, 2H), 3.91-3.72 (m, 4H), 2.89-2.34 (m, 5H), 2.17-2.06 (m, 1H), 1.97-1.79 (m, 6H), 1.76-1.63 (m, 2H), 1.57-1.41 (m, 1H), 1.23 (t, J=6.8 Hz, 3H). LCMS m/z [M+H]⁺ 456.

### Example #15: Synthesis of ethyl (6R)-6-[4-(5-chloro-2-tetrahydropyran-4-yl-3-pyridyl)piperazin-1-yl]-2-azaspiro[3.4]octane-2-carboxylate (A15) and ethyl (6S)-6-[4-(5-chloro-2-tetrahydropyran-4-yl-3-pyridyl)piperazin-1-yl]-2-azaspiro[3.4]octane-2-carboxylate (A16)

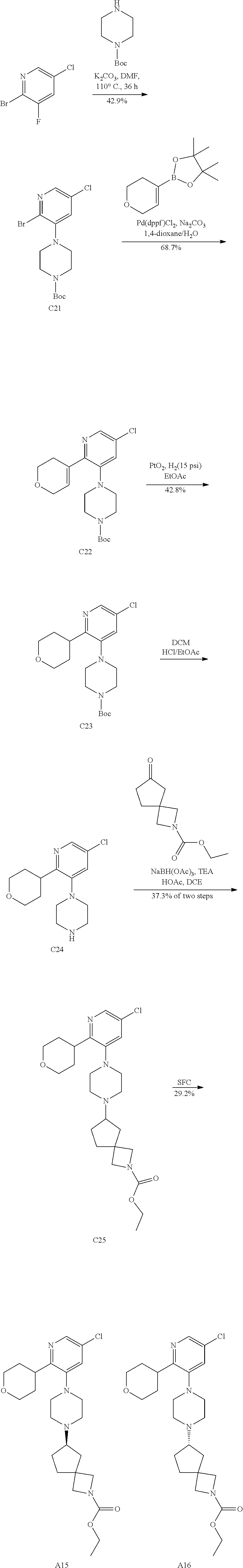

### Step 1: Synthesis of tert-butyl 4-(5-chloro-3-fluoro-2-pyridyl)piperazine-1-carboxylate (C21)

A mixture of tert-butyl piperazine-1-carboxylate (4.43 g, 23.8 mmol), 2-bromo-5-chloro-3-fluoro-pyridine (5.00 g, 23.8 mmol) and K₂CO₃ (6.57 g, 47.5 mmol) in DMSO (50.0 mL) was stirred at 110° C. for 36 h. The reaction mixture was poured into water (50 mL) and extracted with ethyl acetate (3×50 mL). The combined organic layer was washed with brine (100 mL), dried over anhydrous Na₂SO₄, filtered and concentrated under a reduced pressure to give a residue. The residue was purified by flash silica gel chromatography (Eluent of 0˜3% Ethyl acetate/Petroleum ether) to give tert-butyl 4-(5-chloro-3-fluoro-2-pyridyl)piperazine-1-carboxylate (3.89 g, 10.2 mmol, 42.9% yield, 82.7% purity) as a yellow oil. ¹H NMR (CDCl₃ 400 MHz) δ_{H}=8.04 (d, J=2.4 Hz, 1H), 7.22 (d, J=2.4 Hz, 1H), 3.74-3.50 (m, 4H), 3.11-2.90 (m, 4H), 1.48 (s, 9H). LCMS m/z [M+H]⁺ 376, 378.

### Step 2: Synthesis of tert-butyl 4-[5-chloro-2-(3,6-dihydro-2H-pyran-4-yl)-3-pyridyl]piperazine-1-carboxylate (C22)

The solution of tert-butyl 4-(2-bromo-5-chloro-3-pyridyl)piperazine-1-carboxylate (1.00 g, 2.65 mmol), 2-(3,6-dihydro-2H-pyran-4-yl)-4,4,5,5-tetramethyl-1,3,2-dioxaborolane (558 mg, 2.65 mmol), Pd(dppf)Cl₂ (194 mg, 265 μmol) and Na₂CO₃ (563 mg, 5.31 mmol) in 1,4-dioxane (10.0 mL) and water (2.50 mL) was degassed and purged with N₂ gas for 3 times. The reaction mixture was stirred at 90° C. for 12 h under a N₂ gas atmosphere. The mixture was concentrated in a vacuum to give a residue. The residue was purified by flash silica gel chromatography (Eluent of 0˜30% Ethyl acetate/Petroleum ether) to give tert-butyl 4-[5-chloro-2-(3,6-dihydro-2H-pyran-4-yl)-3-pyridyl]piperazine-1-carboxylate (775 mg, 1.82 mmol, 68.7% yield, 89.4% purity) as a colorless oil. LCMS m/z [M+H]⁺ 380.

### Step 3: Synthesis of tert-butyl 4-(5-chloro-2-tetrahydropyran-4-yl-3-pyridyl)piperazine-1-carboxylate (C23)

To a solution of tert-butyl 4-[5-chloro-2-(3,6-dihydro-2H-pyran-4-yl)-3-pyridyl]piperazine-1-carboxylate (775 mg, 2.04 mmol) in EtOAc (8.00 mL) was added PtO₂ (100 mg, 440 μmol). The reaction mixture was degassed under a vacuum and purged with H₂ gas for several times and stirred at room temperature for 12 h under a H₂ gas atmosphere (15 psi). The mixture was filtered and the filter cake was washed with MeOH (10 mL). The filtrate was concentrated under a vacuum to give a residue. The residue was purified by flash silica gel chromatography (Eluent of 0˜30% Ethyl acetate/Petroleum ether) to give tert-butyl 4-(5-chloro-2-tetrahydropyran-4-yl-3-pyridyl)piperazine-1-carboxylate (357 mg, 874 μmol, 42.8% yield, 93.5% purity) as a colorless oil. ¹H NMR (CDCl₃ 400 MHz) δ_{H}=8.31 (d, J=2.0 Hz, 1H), 7.33 (d, J=2.0 Hz, 1H), 4.17-4.06 (m, 4H), 3.60 (br s, 4H), 3.39-3.33 (m, 1H), 2.82 (t, J=4.4 Hz, 4H), 2.13-2.06 (m, 2H), 1.59 (d, J=12.8 Hz, 2H), 1.49 (s, 9H). LCMS m/z [M+H]⁺ 383.

### Step 4: Synthesis of 1-(5-chloro-2-tetrahydropyran-4-yl-3-pyridyl)piperazine (C24)

The tert-butyl 4-(5-chloro-2-tetrahydropyran-4-yl-3-pyridyl)piperazine-1-carboxylate (357 mg, 935 μmol) in DCM (3.00 mL) was added HCl/EtOAc (4 M, 3.00 mL), and then the mixture was stirred at 20° C. for 12 h. The mixture was concentrated under a reduced pressure to give 1-(5-chloro-2-tetrahydropyran-4-yl-3-pyridyl)piperazine (298 mg, 935 μmol, 100% yield, HCl salt) as a yellow oil, which was used into next step as theory amount without purification.

### Step 5: Synthesis of ethyl 6-[4-(5-chloro-2-tetrahydropyran-4-yl-3-pyridyl)piperazin-1-yl]-2-azaspiro[3.4]octane-2-carboxylate (C25)

A mixture of 1-(5-chloro-2-tetrahydropyran-4-yl-3-pyridyl)piperazine (298 mg, 935 μmol, HCl salt), ethyl 6-oxo-2-azaspiro[3.4]octane-2-carboxylate (184 mg, 935 μmol) and TEA (473 mg, 4.67 mmol) in DCE (3.00 mL) was stirred at room temperature for 1 h. Then AcOH (56.1 mg, 935 μmol) and NaBH(OAc)₃ (594 mg, 2.80 mmol) were added. The reaction mixture was stirred at room temperature for 12 h. The reaction mixture was quenched with a saturated aqueous NaHCO₃ solution (10 mL) and extracted with DCM (3×10 mL). The combined organic layer was washed with brine (20 mL), dried over anhydrous Na₂SO₄, filtered and concentrated under a reduced pressure to give a residue. The residue was purified by flash silica gel chromatography (Eluent of 0˜3% Methanol/Dichloromethane) to give ethyl 6-[4-(5-chloro-2-tetrahydropyran-4-yl-3-pyridyl)piperazin-1-yl]-2-azaspiro[3.4]octane-2-carboxylate (176 mg, 349 μmol, 37.3% yield, 91.8% purity) as an off-white solid. LCMS m/z [M+H]⁺ 463.

### Step 6: Synthesis of give ethyl (6R)-6-[4-(5-chloro-2-tetrahydropyran-4-yl-3-pyridyl)piperazin-1-yl]-2-azaspiro[3.4]octane-2-carboxylate (A15) and ethyl (6S)-6-[4-(5-chloro-2-tetrahydropyran-4-yl-3-pyridyl)piperazin-1-yl]-2-azaspiro[3.4]octane-2-carboxylate (A16)

The ethyl 6-[4-(5-chloro-2-tetrahydropyran-4-yl-3-pyridyl)piperazin-1-yl]-2-azaspiro[3.4]octane-2-carboxylate (176 mg, 380 μmol) was purified by SFC(column: DAICEL CHIRALPAK IG (250 mm×30 mm, 10 um); mobile phase: [CO₂-MeOH (0.1% NH₃H₂O)]; B %: 60%, isocratic elution mode) to give Peak 1 ethyl (6R)-6-[4-(5-chloro-2-tetrahydropyran-4-yl-3-pyridyl)piperazin-1-yl]-2-azaspiro[3.4]octane-2-carboxylate (25.7 mg, 54.3 μmol, 14.3% yield, 97.8% purity) as an off-white solid. ¹H NMR (CDCl₃ 400 MHz) δ_{H}=8.29 (s, 1H), 7.34 (s, 1H), 4.10 (q, J=7.2 Hz, 4H), 3.99-3.76 (m, 4H), 3.52 (t, J=11.6 Hz, 2H), 3.39-3.24 (m, 1H), 3.22-2.24 (m, 9H), 2.23-2.10 (m, 1H), 2.11-1.98 (m, 3H), 1.97-1.62 (m, 4H), 1.61-1.59 (m, 1H), 1.52-1.50 (m, 1H), 1.24 (t, J=7.2 Hz, 3H). LCMS m/z [M+H]+ 463. Peak 2 ethyl (6S)-6-[4-(5-chloro-2-tetrahydropyran-4-yl-3-pyridyl)piperazin-1-yl]-2-azaspiro[3.4]octane-2-carboxylate (27.3 mg, 56.8 μmol, 14.9% yield, 96.3% purity) as an off-white solid. ¹H NMR (CDCl₃ 400 MHz) δ_{H}=8.29 (s, 1H), 7.34 (s, 1H), 4.10 (q, J=7.2 Hz, 4H), 3.99-3.75 (m, 4H), 3.52 (t, J=11.6 Hz, 2H), 3.37-3.25 (m, 1H), 3.21-2.41 (m, 9H), 2.20-2.10 (m, 1H), 2.12-1.98 (m, 3H), 1.98-1.62 (m, 4H), 1.62-1.59 (m, 1H), 1.52-1.50 (m, 1H), 1.24 (t, J=7.2 Hz, 3H). LCMS m/z [M+H]⁺ 463.

### Example #16: Synthesis of ethyl (6R)-6-[4-[5-fluoro-2-(4-hydroxytetrahydropyran-4-yl)-3-pyridyl]piperazin-1-yl]-2-azaspiro[3.4]octane-2-carboxylate (A17) and ethyl (68)-6-[4-[5-fluoro-2-(4-hydroxytetrahydropyran-4-yl)-3-pyridyl]piperazin-1-yl]-2-azaspiro[3.4]octane-2-carboxylate (A18)

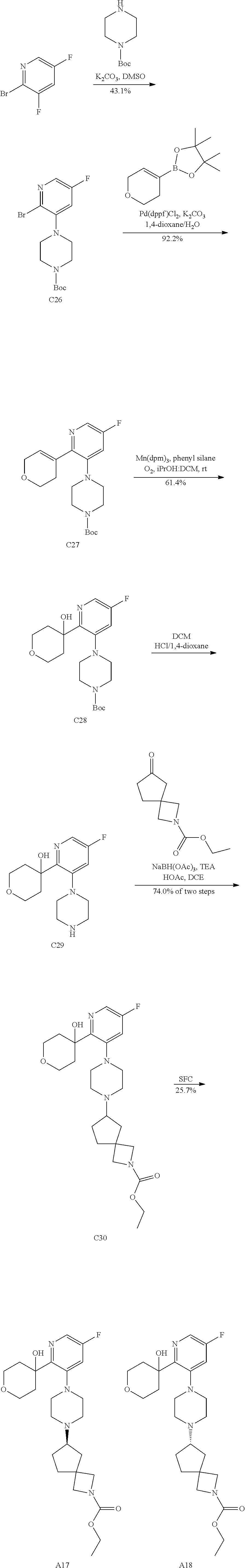

### Step 1: Synthesis of tert-butyl 4-(2-bromo-5-fluoropyridin-3-yl)piperazine-1-carboxylate (C26)

To a solution of 2-bromo-3,5-difluoro-pyridine (10.0 g, 51.6 mmol) in DMSO (100 mL) was added tert-butyl piperazine-1-carboxylate (11.5 g, 61.9 mmol) and K₂CO₃ (14.3 g, 103 mmol) in one portion. The mixture was stirred at 110° C. for 12 h. The reaction mixture was cooled to room temperature. H₂O (300 mL) was added into the mixture. The mixture was extracted with EtOAc (3×100 mL). The combined organic phase was washed with a 3% aqueous LiCl solution (250 mL), dried over anhydrous Na₂SO₄, filtered and concentrated under a reduced pressure. The residue was purified by flash silica gel chromatography (Eluent of 0˜18% Ethyl acetate/Petroleum ether) to give tert-butyl 4-(2-bromo-5-fluoro-3-pyridyl)piperazine-1-carboxylate (8.00 g, 22.2 mmol, 43.1% yield) as an off-white solid. ¹H NMR (CDCl₃ 400 MHz) δ_{H}=7.97 (d, J=2.0 Hz, 1H), 7.02 (dd, J=2.8 Hz, 9.2 Hz, 1H), 3.67-3.57 (m, 4H), 3.09-2.91 (m, 4H), 1.46 (s, 9H). LCMS m/z [M+H]+ 360, 362.

### Step 2: Synthesis of tert-butyl 4-(2-(3,6-dihydro-2H-pyran-4-yl)-5-fluoropyridin-3-yl)piperazine-1-carboxylate(C27)

A mixture of tert-butyl 4-(2-bromo-5-fluoro-3-pyridyl)piperazine-1-carboxylate (1.00 g, 2.78 mmol), 2-(3,6-dihydro-2H-pyran-4-yl)-4,4,5,5-tetramethyl-1,3,2-dioxaborolane (700 mg, 3.33 mmol), K₂CO₃ (767 mg, 5.55 mmol) and Pd(dppf)Cl₂ (203 mg, 278 μmol) in 1,4-dioxane (10.0 mL) and water (1.00 mL) was degassed under a vacuum and purged with N₂ gas for several times. The reaction mixture was stirred at 100° C. for 12 h under a N₂ gas atmosphere. The reaction mixture was concentrated under a reduced pressure to give a residue. The residue was purified by flash silica gel chromatography (Eluent of 0˜14% Ethyl acetate/Petroleum ether) to give tert-butyl 4-[2-(3,6-dihydro-2H-pyran-4-yl)-5-fluoro-3-pyridyl]piperazine-1-carboxylate (930 mg, 2.56 mmol, 92.2% yield) as an off-white solid. ¹H NMR (CD₃OD 400 MHz) δ_{H}=8.05 (d, J=2.4 Hz, 1H), 7.31 (dd, J=2.4 Hz, 10.4 Hz, 1H), 6.35-6.25 (m, 1H), 4.32 (q, J=2.4 Hz, 2H), 3.92 (t, J=5.6 Hz, 2H), 3.66-3.46 (m, 4H), 3.07-2.91 (m, 4H), 2.67-2.55 (m, 2H), 1.47 (s, 9H). LCMS m/z [M+H]⁺ 364.

### Step 3: Synthesis of tert-butyl 4-(5-fluoro-2-(4-hydroxytetrahydro-2H-pyran-4-yl)pyridin-3-yl)piperazine-1-carboxylate (C28)

To a mixture of tert-butyl 4-[2-(3,6-dihydro-2H-pyran-4-yl)-5-fluoro-3-pyridyl]piperazine-1-carboxylate (1.20 g, 3.30 mmol) and tris[(Z)-1-tert-butyl-4,4-dimethyl-3-oxo-pent-1-enoxy]-manganese (39.9 mg, 66.0 μmol) in DCM (1.00 mL) and i-PrOH (8.00 mL) was added phenylsilane (715 mg, 6.60 mmol) at 0° C. The mixture was stirred at room temperature for 2 h under an O₂ gas atmosphere (15 psi). The reaction mixture was concentrated under a reduced pressure to give a residue. The residue was purified by flash silica gel chromatography (Eluent of 0˜26% Ethyl acetate/Petroleum ether) to give tert-butyl 4-[5-fluoro-2-(4-hydroxytetrahydropyran-4-yl)-3-pyridyl]piperazine-1-carboxylate (430 mg, 1.13 mmol, 61.4% yield) as an off-white solid. LCMS m/z [M+H]⁺ 382.

### Step 4: Synthesis of 4-(5-fluoro-3-(piperazin-1-yl)pyridin-2-yl)tetrahydro-2H-pyran-4-ol (C29)

To a solution of tert-butyl 4-[5-fluoro-2-(4-hydroxytetrahydropyran-4-yl)-3-pyridyl]piperazine-1-carboxylate (150 mg, 393 μmol) in DCM (6.00 mL) was added HCl/1,4-dioxane (4 M, 1.96 mL). The mixture was stirred at room temperature for 2 h. The reaction mixture was concentrated under a reduced pressure to give a 4-(5-fluoro-3-piperazin-1-yl-2-pyridyl)tetrahydropyran-4-ol (130 mg, crude, HCl salt) as an off-white solid. The crude product was used in the next step without further purification. LCMS m/z [M+H]+ 282.

### Step 5: Synthesis of ethyl 6-(4-(5-fluoro-2-(4-hydroxytetrahydro-2H-pyran-4-yl)pyridin-3-yl)piperazin-1-yl)-2-azaspiro[3.4]octane-2-carboxylate (C30)

A mixture of 4-(5-fluoro-3-piperazin-1-yl-2-pyridyl)tetrahydropyran-4-ol (130 mg, 409 μmol, HCl salt), ethyl 6-oxo-2-azaspiro[3.4]octane-2-carboxylate (80.7 mg, 409 μmol) and Et₃N (124 mg, 1.23 mmol) in DCE (10.0 mL) was stirred at room temperature for 1 h. Then AcOH (2.46 mg, 40.91 μmol) and NaBH(OAc)₃ (260 mg, 1.23 mmol) were added in one portion. The reaction mixture was stirred at room temperature for 11 h. The mixture was poured into a saturated aqueous NaHCO₃ solution (30 mL) and extracted with DCM (3×30 mL). The combined organic phase was washed with brine (50 mL), dried over anhydrous Na₂SO₄, filtered and concentrated under a reduced pressure to give a residue. The residue was purified by flash silica gel chromatography (Eluent of 0˜7% MeOH/DCM) to give ethyl 6-[4-[5-fluoro-2-(4-hydroxytetrahydropyran-4-yl)-3-pyridyl]piperazin-1-yl]-2-azaspiro[3.4]octane-2-carboxylate (140 mg, 303 μmol, 74.0% yield) as a colorless oil. LCMS m/z [M+H]⁺ 463.

### Step 6: Synthesis of ethyl (6R)-6-[4-[5-fluoro-2-(4-hydroxytetrahydropyran-4-yl)-3-pyridyl]piperazin-1-yl]-2-azaspiro[3.4]octane-2-carboxylate (A17) and ethyl (6S)-6-[4-[5-fluoro-2-(4-hydroxytetrahydropyran-4-yl)-3-pyridyl]piperazin-1-yl]-2-azaspiro[3.4]octane-2-carboxylate (A18)

The sample of ethyl 6-[4-[5-fluoro-2-(4-hydroxytetrahydropyran-4-yl)-3-pyridyl]piperazin-1-yl]-2-azaspiro[3.4]octane-2-carboxylate (140 mg, 303 μmol) was separated by SFC (column: DAICEL CHIRALPAK AD, 250 mm×30 mm, 10 um; mobile phase: [CO₂-MeOH (0.1% NH₃H₂O)]; B %: 25%, isocratic elution mode) to give Peak 1 give ethyl (6R)-6-[4-[5-fluoro-2-(4-hydroxytetrahydropyran-4-yl)-3-pyridyl]piperazin-1-yl]-2-azaspiro[3.4]octane-2-carboxylate as an off-white solid (24.1 mg, 51.3 μmol, 98.5% purity) as an off-white solid. ¹H NMR (CD₃OD 400 MHz) δ_{H}=8.37 (d, J=2.4 Hz, 1H), 7.82 (dd, J=2.4 Hz, 10.0 Hz, 1H), 4.08 (q, J=7.2 Hz, 2H), 4.00-3.89 (m, 3H), 3.88-3.77 (m, 5H), 3.24-2.59 (m, 8H), 2.57-2.39 (m, 3H), 2.25-2.16 (m, 1H), 2.04-1.86 (m, 3H), 1.80-1.71 (m, 1H), 1.64-1.52 (m, 3H), 1.23 (t, J=6.8 Hz, 3H). LCMS m/z [M+H]⁺ 463, found 463. Peak 2 ethyl (6S)-6-[4-[5-fluoro-2-(4-hydroxytetrahydropyran-4-yl)-3-pyridyl]piperazin-1-yl]-2-azaspiro[3.4]octane-2-carboxylate (25.0 mg, 54.1 μmol, 17.9% yield) as an off-white solid. ¹H NMR (CD₃OD 400 MHz) δ_{H}=8.37 (d, J=2.0 Hz, 1H), 7.82 (dd, J=2.8 Hz, 9.6 Hz, 1H), 4.08 (q, J=7.2 Hz, 2H), 4.01-3.78 (m, 8H), 3.20-2.85 (m, 6H), 2.83-2.64 (m, 2H), 2.61-2.37 (m, 3H), 2.22 (dd, J=7.2 Hz, 12.8 Hz, 1H), 2.09-1.84 (m, 3H), 1.77 (dd, J=9.2 Hz, 12.8 Hz, 1H), 1.69-1.48 (m, 3H), 1.24 (t, J=7.2 Hz, 3H). LCMS m/z [M+H]⁺ 463.

### Example #17: Synthesis of ethyl (6R)-6-[4-[5-fluoro-2-(4-fluorotetrahydropyran-4-yl)-3-pyridyl]piperazin-1-yl]-2-azaspiro[3.4]octane-2-carboxylate (A19) and ethyl (6S)-6-[4-[5-fluoro-2-(4-fluorotetrahydropyran-4-yl)-3-pyridyl]piperazin-1-yl]-2-azaspiro[3.4]octane-2-carboxylate (A20)

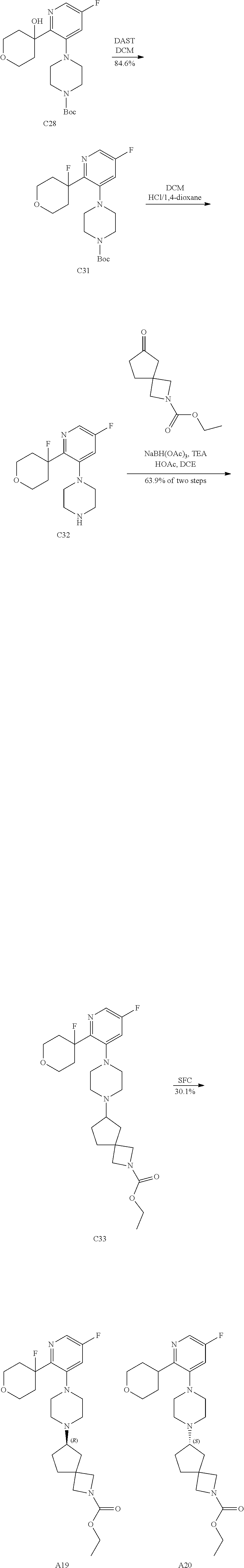

### Step 1: Synthesis of tert-butyl 4-(5-fluoro-2-(4-fluorotetrahydro-2H-pyran-4-yl)pyridin-3-yl)piperazine-1-carboxylate (C31)

To a solution of tert-butyl 4-[5-fluoro-2-(4-hydroxytetrahydropyran-4-yl)-3-pyridyl]piperazine-1-carboxylate (200 mg, 524 μmol) in DCM (8.00 mL) was added dropwise DAST (338 mg, 2.10 mmol) at 0° C. The reaction mixture was stirred at 0° C. for 1 h. The reaction mixture was poured into a saturated aqueous NaHCO₃ solution (30 mL) and extracted with DCM (20 mL×3). The combined organic layer was washed with brine (20 mL), dried over anhydrous Na₂SO₄, filtered and concentrated under a reduced pressure to give a residue. The residue was purified by flash silica gel chromatography (Eluent of 0˜14% Ethyl acetate/Petroleum ether) to give tert-butyl 4-[5-fluoro-2-(4-fluorotetrahydropyran-4-yl)-3-pyridyl]piperazine-1-carboxylate (170 mg, 443 μmol, 84.6% yield) as a colorless oil. ¹H NMR (CD₃OD 400 MHz) b_{H}=8.27 (d, J=2.0 Hz, 1H), 7.68 (dd, J=2.4 Hz, 10.0 Hz, 1H), 3.96-3.81 (m, 4H), 3.56 (br s, 4H), 2.90 (t, J=4.8 Hz, 4H), 2.51-2.25 (m, 4H), 1.48 (s, 9H). LCMS m/z [M+H]⁺ 384.

### Step 2: Synthesis of 1-(5-fluoro-2-(4-fluorotetrahydro-2H-pyran-4-yl)pyridin-3-yl)piperazine (C32)

To a solution of tert-butyl 4-[5-fluoro-2-(4-fluorotetrahydropyran-4-yl)-3-pyridyl]piperazine-1-carboxylate (170 mg, 443 μmol) in DCM (8.00 mL) was added HCl/1,4-dioxane (4 M, 3.00 mL). The reaction mixture was stirred at 25° C. for 2 h. The reaction mixture was concentrated under a reduced pressure to give 1-[5-fluoro-2-(4-fluorotetrahydropyran-4-yl)-3-pyridyl]-piperazine (140 mg, crude, HCl salt) as an off-white solid. The crude product was used in the next step without further purification. ¹H NMR (CD₃OD 400 MHz) δ_{H}=8.41 (d, J=2.4 Hz, 1H), 7.89 (br d, J=9.6 Hz, 1H), 3.99-3.79 (m, 4H), 3.44-3.34 (m, 4H), 3.26-3.15 (m, 4H), 2.57-2.18 (m, 4H). LCMS m/z [M+H]⁺ 284.

### Step 3: Synthesis of ethyl 6-(4-(5-fluoro-2-(4-fluorotetrahydro-2H-pyran-4-yl)pyridin-3-yl)piperazin-1-yl)-2-azaspiro[3.4]octane-2-carboxylate (C33)

A mixture of 1-[5-fluoro-2-(4-fluorotetrahydropyran-4-yl)-3-pyridyl]piperazine (140 mg, 438 μmol, HCl salt) and ethyl 6-oxo-2-azaspiro[3.4]octane-2-carboxylate (86.4 mg, 438 μmol) and Et₃N (133 mg, 1.31 mmol) in DCE (10.0 mL) was stirred at room temperature for 1 h. Then AcOH (2.63 mg, 43.8 μmol) and NaBH(OAc)₃ (278 mg, 1.31 mmol) were added in one portion. The mixture was stirred at room temperature for 11 h. The mixture was poured into a saturated aqueous NaHCO₃ solution (30 mL) and extracted with DCM (3×30 mL). The combined organic phase was washed with brine (50 mL), dried over anhydrous Na₂SO₄, filtered, and concentrated under a reduced pressure to give a residue. The residue was purified by flash silica gel chromatography (Eluent of 0˜3% MeOH/DCM) to give ethyl 6-[4-[5-fluoro-2-(4-fluorotetrahydropyran-4-yl)-3-pyridyl]piperazin-1-yl]-2-azaspiro[3.4]octane-2-carboxylate (130 mg, 280 μmol, 63.9% yield) as a colorless oil. ¹H NMR (CDCl₃ 400 MHz) δ_{H}=8.30-8.15 (m, 1H), 7.38 (dd, J=2.4 Hz, 9.6 Hz, 1H), 4.10 (q, J=7.2 Hz, 2H), 4.01-3.70 (m, 8H), 2.95 (br s, 4H), 2.78-2.22 (m, 9H), 2.21-2.07 (m, 1H), 2.02-1.71 (m, 4H), 1.70-1.62 (m, 1H), 1.24 (t, J=7.2 Hz, 3H). LCMS m/z [M+H]⁺ 465.

### Step 4: Synthesis of ethyl (6R)-6-[4-[5-fluoro-2-(4-fluorotetrahydropyran-4-yl)-3-pyridyl]piperazin-1-yl]-2-azaspiro[3.4]octane-2-carboxylate (19) and ethyl (6S)-6-[4-[5-fluoro-2-(4-fluorotetrahydropyran-4-yl)-3-pyridyl]piperazin-1-yl]-2-azaspiro[3.4]octane-2-carboxylate (20)

The sample of ethyl 6-[4-[5-fluoro-2-(4-fluorotetrahydropyran-4-yl)-3-pyridyl]piperazin-1-yl]-2-azaspiro[3.4]octane-2-carboxylate (130 mg, 280 μmol) was separated by SFC (column: DAICEL CHIRALPAK IG, 250 mm×30 mm, 10 um; mobile phase: [CO₂-MeOH (0.1% NH₃H₂O)]; B %: 60%, isocratic elution mode) to give Peak 1 ethyl (6R)-6-[4-[5-fluoro-2-(4-fluorotetrahydropyran-4-yl)-3-pyridyl]piperazin-1-yl]-2-azaspiro[3.4]octane-2-carboxylate (26.0 mg, 51.1 μmol, 29.7% yield, 91.2% purity) as an off-white solid. ¹H NMR (CDCl₃ 400 MHz) δ_{H}=8.29-8.18 (m, 1H), 7.38 (dd, J=2.4 Hz, 9.6 Hz, 1H), 4.10 (q, J=7.2 Hz, 2H), 3.99-3.72 (m, 8H), 3.06-2.87 (m, 4H), 2.85-2.45 (m, 5H), 2.45-2.24 (m, 4H), 2.14 (dd, J=6.8 Hz, 12.4 Hz, 1H), 2.03-1.79 (m, 3H), 1.78-1.66 (m, 1H), 1.59-1.51 (m, 1H), 1.24 (t, J=7.2 Hz, 3H). LCMS m/z [M+H]⁺ 465. Peak 2 ethyl (6S)-6-[4-[5-fluoro-2-(4-fluorotetrahydropyran-4-yl)-3-pyridyl]piperazin-1-yl]-2-azaspiro[3.4]octane-2-carboxylate (27.1 mg, 51.8 μmol, 30.1% yield, 88.7% purity) as an off-white solid. ¹H NMR (CDCl₃ 400 MHz) δ_{H}=8.29-8.18 (m, 1H), 7.38 (dd, J=2.4 Hz, 9.6 Hz, 1H), 4.10 (q, J=7.2 Hz, 2H), 3.98-3.73 (m, 8H), 3.03-2.84 (m, 4H), 2.82-2.45 (m, 5H), 2.45-2.22 (m, 4H), 2.14 (dd, J=7.2 Hz, 12.8 Hz, 1H), 2.03-1.79 (m, 3H), 1.77-1.67 (m, 1H), 1.57-1.47 (m, 1H), 1.24 (t, J=7.2 Hz, 3H). LCMS m/z [M+H]⁺ 465.

### Example #18: Synthesis of ethyl (6S)-6-[4-[5-fluoro-2-(1,3,4-thiadiazol-2-yl)-3-pyridyl]-1-piperidyl]-2-azaspiro[3.4]octane-2-carboxylate (A21) and ethyl (6R)-6-[4-[5-fluoro-2-(1,3,4-thiadiazol-2-yl)-3-pyridyl]-1-piperidyl]-2-azaspiro[3.4]octane-2-carboxylate (A22)

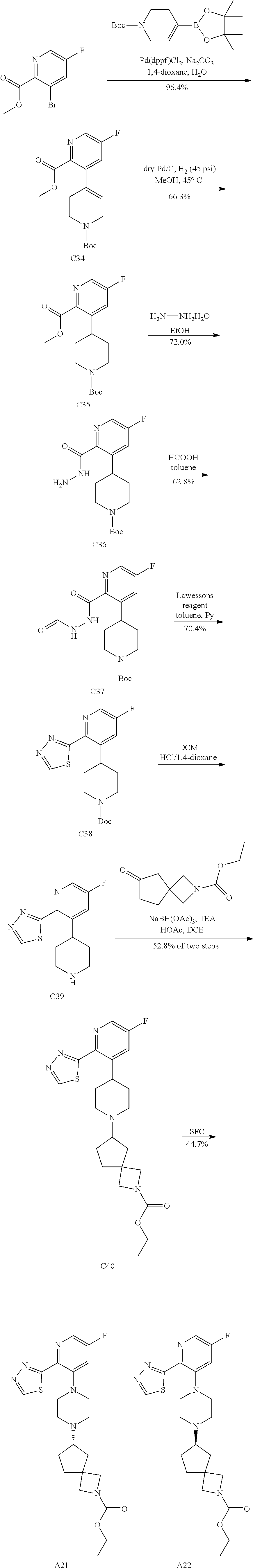

### Step 1: Synthesis of methyl 3-(1-tert-butoxycarbonyl-3,6-dihydro-2H-pyridin-4-yl)-5-fluoro-pyridine-2-carboxylate (C34)

To a solution of methyl 3-bromo-5-fluoro-pyridine-2-carboxylate (3.50 g, 15.0 mmol) and tert-butyl 4-(4,4,5,5-tetramethyl-1,3,2-dioxaborolan-2-yl)-3,6-dihydro-2H-pyridine-1-carboxylate (5.09 g, 16.4 mmol) in H₂O (4.00 mL) and 1,4-dioxane (32.0 mL) was added Na₂CO₃ (3.17 g, 29.9 mmol) and Pd(dppf)Cl₂ (1.09 g, 1.50 mmol) in one portion. The mixture was degassed under a vacuum and purged with N₂ gas for 3 times. The mixture was stirred at 80° C. for 16 h. The mixture was poured into water (30 mL) and stirred for 5 min. The mixture was extracted with EtOAc (3×60 mL). The combined organic layer was washed with brine (150 mL), dried over anhydrous Na₂SO₄, filtered and concentrated under a reduced pressure to give a residue. The residue was purified by flash silica gel chromatography (Eluent of 0˜16% Ethyl acetate/Petroleum ether) to give methyl 3-(1-tert-butoxycarbonyl-3,6-dihydro-2H-pyridin-4-yl)-5-fluoro-pyridine-2-carboxylate (6.50 g, 19.3 mmol, 96.4% yield) as a colorless oil. LCMS m/z [M+H]⁺ 337.

### Step 2: Synthesis of methyl 3-(1-tert-butoxycarbonyl-4-piperidyl)-5-fluoro-pyridine-2-carboxylate (C35)

To a solution of methyl 3-(1-tert-butoxycarbonyl-3,6-dihydro-2H-pyridin-4-yl)-5-fluoro-pyridine-2-carboxylate (3.00 g, 8.92 mmol) in MeOH (30.0 mL) was added dry Pd/C (300 mg, 10% w/w) under an Ar gas atmosphere. The suspension was degassed and purged with H₂gas for 3 times. The mixture was stirred at 45° C. for 72 h under a H₂ gas atmosphere (45 psi). The mixture was filtered and the filter cake was washed with MeOH (3×10 mL). The filtrate was concentrated under a reduced pressure to give a residue. The residue was purified by flash silica gel chromatography (Eluent of 0˜25% Ethyl acetate/Petroleum ether) to give methyl 3-(1-tert-butoxycarbonyl-4-piperidyl)-5-fluoro-pyridine-2-carboxylate (2.00 g, 5.91 mmol, 66.3% yield) as a colorless oil. LCMS m/z [M+H]⁺ 339.

### Step 3: Synthesis of tert-butyl 4-[5-fluoro-2-(hydrazinecarbonyl)-3-pyridyl]piperidine-1-carboxylate (C36)

To a solution of methyl 3-(1-tert-butoxycarbonyl-4-piperidyl)-5-fluoro-pyridine-2-carboxylate (2.00 g, 5.91 mmol) in EtOH (20.0 mL) was added NH₂NH₂—H₂O (6.00 g, 102 mmol, 85% purity) in one portion. Then the mixture was stirred at 60° C. for 6 h. The mixture was concentrated under a reduced pressure to give a residue. The residue was purified by flash silica gel chromatography (Eluent of 0˜3% Methanol/Dichloromethane) to give tert-butyl 4-[5-fluoro-2-(hydrazinecarbonyl)-3-pyridyl]piperidine-1-carboxylate (1.44 g, 4.26 mmol, 72.0% yield) as a colorless oil. LCMS m/z [M-Boc]⁺239.

### Step 4: Synthesis of tert-butyl 4-[5-fluoro-3-(1,3,4-thiadiazol-2-yl)-2-pyridyl]piperazine-1-carboxylate (C37)

To a solution of tert-butyl 4-[5-fluoro-2-(hydrazinecarbonyl)-3-pyridyl]piperidine-1-carboxylate (1.44 g, 4.26 mmol) in toluene (15.0 mL) was added HCO₂H (2.09 g, 42.6 mmol). The solution was degassed under a vacuum and purged with N₂ gas for 3 times, and then the mixture was stirred at 50° C. for 1 h. The mixture was adjusted to pH=8 with a saturated aqueous NaHCO₃ solution. The mixture was extracted with EtOAc (3×20 mL). The combined organic phase was washed with brine (50 mL), dried over anhydrous Na₂SO₄, filtered and concentrated under a reduced pressure to give a colorless oil. The oil was purified by flash silica gel chromatography (Eluent of 0˜78% Methanol/Dichloromethane) to give tert-butyl 4-[5-fluoro-2-(formamidocarbamoyl)-3-pyridyl]piperidine-1-carboxylate (980 mg, 2.67 mmol, 62.8% yield) as a colorless oil. LCMS m/z [M-Boc]⁺267.

### Step 5: Synthesis of tert-butyl 4-[5-fluoro-2-(1,3,4-thiadiazol-2-yl)-3-pyridyl]piperidine-1-carboxylate (C38)

To a solution of tert-butyl 4-[5-fluoro-2-(formamidocarbamoyl)-3-pyridyl]piperidine-1-carboxylate (200 mg, 546 μmol) in toluene (2.00 mL) and pyridine (0.500 mL) was added Lawesson's reagent (265 mg, 655 μmol) in one portion. The reaction mixture was stirred at 90° C. for 12 h. The mixture was poured into water (5 mL) and stirred for 5 min. The mixture was extracted with EtOAc (3×10 mL). The combined organic phase was washed with brine (30 mL), dried over anhydrous Na₂SO₄, filtered and concentrated under a reduced pressure to give a residue. The residue was purified by flash silica gel chromatography (Eluent of 0˜19% Ethyl acetate/Petroleum ether) to give tert-butyl 4-[5-fluoro-2-(1,3,4-thiadiazol-2-yl)-3-pyridyl]piperidine-1-carboxylate (140 mg, 384 μmol, 70.4% yield) as an off-white solid. ¹H NMR (CDCl₃ 400 MHz) δ_{H}=9.16 (s, 1H), 8.38 (d, J=2.4 Hz, 1H), 7.50 (dd, J=2.4 Hz, 9.6 Hz, 1H), 4.48-4.38 (m, 1H), 4.37-4.10 (m, 2H), 3.09-2.84 (m, 2H), 1.95 (d, J=12.8 Hz, 2H), 1.65-1.55 (m, 2H), 1.49 (s, 9H). LCMS m/z [M-Boc]⁺265.

### Step 6: Synthesis of 2-[5-fluoro-3-(4-piperidyl)-2-pyridyl]-1,3,4-thiadiazole (C39)

To a solution of tert-butyl 4-[5-fluoro-2-(1,3,4-thiadiazol-2-yl)-3-pyridyl]piperidine-1-carboxylate (140 mg, 384 μmol) in DCM (1.50 mL) was added HCl/1,4-dioxane (4 M, 1.50 mL) and the mixture was stirred at room temperature for 1 h. The mixture was concentrated under a reduced pressure to give 2-[5-fluoro-3-(4-piperidyl)-2-pyridyl]-1,3,4-thiadiazole (115 mg, crude, HCl salt) as an off-white solid, which was used into next step without purification. LCMS m/z [M+H]⁺ 265.

### Step 7: Synthesis of ethyl (6S)-6-[4-[5-fluoro-2-(1,3,4-thiadiazol-2-yl)-3-pyridyl]-1-piperidyl]-2-azaspiro[3.4]octane-2-carboxylate (A21) and ethyl (6R)-6-[4-[5-fluoro-2-(1,3,4-thiadiazol-2-yl)-3-pyridyl]-1-piperidyl]-2-azaspiro[3.4]octane-2-carboxylate (A22)

To a mixture of 2-[5-fluoro-3-(4-piperidyl)-2-pyridyl]-1,3,4-thiadiazole (115 mg, 382 μmol, HCl salt) in DCE (1.50 mL) was added TEA (193 mg, 1.91 mmol) and ethyl 6-oxo-2-azaspiro[3.4]octane-2-carboxylate (75.4 mg, 382 μmol) in one portion. Then, the mixture was stirred at room temperature for 2 h. NaBH(OAc)₃ (243 mg, 1.15 mmol) and AcOH (2.30 mg, 38.2 μmol) were added into the mixture in one portion. The mixture was stirred at room temperature for 32 h. The mixture was poured into a saturated aqueous NaHCO₃ solution (5 mL) and extracted with DCM (3×10 mL). The combined organic phase was washed with brine (50 mL), dried over anhydrous Na₂SO₄, filtered and concentrated under a reduced pressure to give a residue. The residue was purified by flash silica gel chromatography (Eluent of 0˜5% Methanol/Dichloromethane) and further separated by SFC (column: DAICEL CHIRALCEL OD-H, 250 mm×30 mm, 5 um; mobile phase: [CO₂-i-PrOH (0.1% NH₃H₂O)]; B %: 45%, isocratic elution mode) to give Peak 1 ethyl (6S)-6-[4-[5-fluoro-2-(1,3,4-thiadiazol-2-yl)-3-pyridyl]-1-piperidyl]-2-azaspiro[3.4]octane-2-carboxylate (21.7 mg, 48.7 μmol, 24.1% yield) as an off-white solid. ¹H NMR (CDCl₃ 400 MHz) δ_{H}=9.15 (s, 1H), 8.36 (d, J=2.4 Hz, 1H), 7.56 (dd, J=2.4 Hz, 9.6 Hz, 1H), 4.25 (t, J=11.4 Hz, 1H), 4.10 (q, J=7.2 Hz, 2H), 3.92-3.76 (m, 4H), 3.11 (t, J=10.4 Hz, 2H), 2.73-2.58 (m, 1H), 2.26-2.12 (m, 3H), 2.02-1.90 (m, 4H), 1.88-1.71 (m, 4H), 1.62-1.53 (m, 1H), 1.24 (t, J=7.2 Hz, 3H). LCMS m/z [M+H]+ 446. Peak 2 ethyl (6R)-6-[4-[5-fluoro-2-(1,3,4-thiadiazol-2-yl)-3-pyridyl]-1-piperidyl]-2-azaspiro[3.4]octane-2-carboxylate (18.5 mg, 41.5 μmol, 20.6% yield) as an off-white solid. ¹H NMR (CDCl₃ 400 MHz) δ_{H}=9.15 (s, 1H), 8.36 (d, J=2.4 Hz, 1H), 7.56 (dd, J=2.4 Hz, 10.0 Hz, 1H), 4.25 (t, J=11.2 Hz, 1H), 4.10 (q, J=7.2 Hz, 2H), 3.92-3.75 (m, 4H), 3.11 (t, J=10.4 Hz, 2H), 2.73-2.59 (m, 1H), 2.28-2.11 (m, 3H), 2.01-1.89 (m, 4H), 1.88-1.71 (m, 4H), 1.62-1.52 (m, 1H), 1.24 (t, J=7.2 Hz, 3H). LCMS m/z [M+H]⁺ 446. The chirality was later set based upon the potency.

### Example #19: Synthesis of ethyl (6R)-6-[4-[5-fluoro-2-(4-methyltriazol-1-yl)-3-pyridyl]-1-piperidyl]-2-azaspiro[3.4]octane-2-carboxylate (A23) and ethyl (6S)-6-[4-[5-fluoro-2-(4-methyltriazol-1-yl)-3-pyridyl]-1-piperidyl]-2-azaspiro[3.4]octane-2-carboxylate (A24)

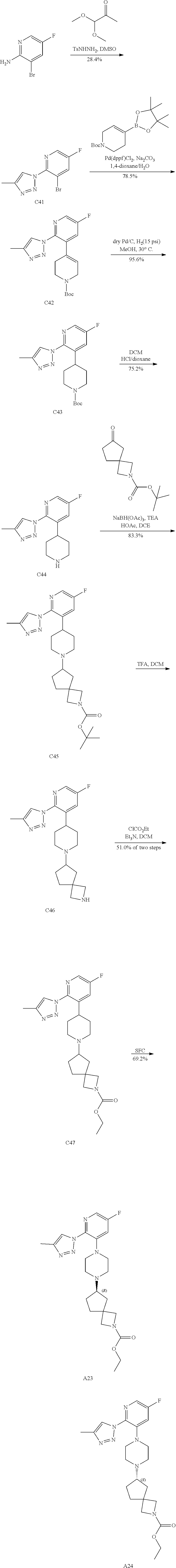

### Step 1: Synthesis of 3-bromo-5-fluoro-2-(4-methyltriazol-1-yl)pyridine (C41)

A mixture of 1, 1-dimethoxypropan-2-one (618 mg, 5.24 mmol) and 4-methylbenzenesulfonohydrazide (975 mg, 5.24 mmol) in DMSO (10.0 mL) was stirred at room temperature for 1 h. Then 3-bromo-5-fluoro-pyridin-2-amine (1.00 g, 5.24 mmol) was added to the mixture. The reaction mixture was stirred at 90° C. for 15 h. The reaction mixture was cooled to room temperature. Water (20 mL) was added and the mixture was stirred for 15 min. The mixture was extracted with EtOAc (3×15 mL). The combined organic phase was washed with brine (20 mL), dried over anhydrous Na₂SO₄, filtered and concentrated under a reduced pressure. The residue was purified by flash silica gel chromatography (Eluent of 0˜40% Ethyl acetate/Petroleum ether) to give 3-bromo-5-fluoro-2-(4-methyltriazol-1-yl)pyridine (428 mg, 1.49 mmol, 28.4% yield, 89.4% purity) as a yellow oil. LCMS m/z [M+H]⁺ 257, 259.

### Step 2: Synthesis of tert-butyl 4-[5-fluoro-2-(4-methyltriazol-1-yl)-3-pyridyl]-3,6-dihydro-2H-pyridine-1-carboxylate (C42)

A mixture of 3-bromo-5-fluoro-2-(4-methyltriazol-1-yl)pyridine (428 mg, 1.66 mmol), tert-butyl 4-(4,4,5,5-tetramethyl-1,3,2-dioxaborolan-2-yl)-3,6-dihydro-2H-pyridine-1-carboxylate (515 mg, 1.66 mmol), Pd(dppf)Cl₂ (122 mg, 167 μmol), Na₂CO₃ (353 mg, 3.33 mmol) in 1,4-dioxane (5.00 mL) and H₂O (1.00 mL) was degassed under a vacuum and purged with N₂ gas for 3 times. Then the reaction mixture was stirred at 90° C. for 12 h under a N₂ gas atmosphere. The reaction mixture was concentrated under a reduced pressure. The residue was purified by flash silica gel chromatography (Eluent of 0˜40% Ethyl acetate/Petroleum ether) to give tert-butyl 4-[5-fluoro-2-(4-methyltriazol-1-yl)-3-pyridyl]-3,6-dihydro-2H-pyridine-1-carboxylate (490 mg, 1.31 mmol, 78.5% yield, 95.9% purity) as a yellow oil. ¹H NMR (CDCl₃ 400 MHz) δ_{H}=8.31 (d, J=2.8 Hz, 1H), 7.88 (s, 1H), 7.48 (dd, J=2.8 Hz, 8.0 Hz, 1H), 5.73 (s, 1H), 4.02 (br s, 2H), 3.51 (t, J=5.6 Hz, 2H), 2.43 (s, 3H), 1.94 (br s, 3H), 1.47 (s, 9H). LCMS m/z [M+H]⁺ 360.

### Step 3: Synthesis of tert-butyl 4-[5-fluoro-2-(4-methyltriazol-1-yl)-3-pyridyl]piperidine-1-carboxylate (C43)

To a solution of tert-butyl 4-[5-fluoro-2-(4-methyltriazol-1-yl)-3-pyridyl]-3,6-dihydro-2H-pyridine-1-carboxylate (490 mg, 1.36 mmol) in MeOH (5.00 mL) was added dry Pd/C (300 mg, 10% w/w). The mixture was degassed and purged with H₂ gas for 3 times. The reaction was stirred at 30° C. for 16 h under a H₂ gas atmosphere (15 psi). The mixture was filtered and the filtrate was concentrated under a reduced pressure to give tert-butyl 4-[5-fluoro-2-(4-methyltriazol-1-yl)-3-pyridyl]piperidine-1-carboxylate (476 mg, 1.30 mmol, 95.6% yield, 99.0% purity) as a yellow oil. ¹H NMR (CDCl₃ 400 MHz) δ_{H}=8.26 (d, J=2.8 Hz, 1H), 7.88 (d, J=0.8 Hz, 1H), 7.54 (dd, J=2.4 Hz, 8.8 Hz, 1H), 4.20 (br s, 2H), 3.37-3.26 (m, 1H), 2.74 (br s, 2H), 2.46 (s, 3H), 1.87 (d, J=12.8 Hz, 2H), 1.63-1.52 (m, 2H), 1.47 (s, 9H). LCMS m/z [M+H]⁺ 362.

### Step 4: Synthesis of 5-fluoro-2-(4-methyltriazol-1-yl)-3-(4-piperidyl)pyridine (C44)

To a solution of tert-butyl 4-[5-fluoro-2-(4-methyltriazol-1-yl)-3-pyridyl]piperidine-1-carboxylate (476 mg, 1.32 mmol) in DCM (5.00 mL) was added dropwise HCl/1,4-dioxane (4 M, 2.00 mL). The mixture was stirred at room temperature for 1 h. The reaction mixture was concentrated under a reduced pressure to give 5-fluoro-2-(4-methyltriazol-1-yl)-3-(4-piperidyl)pyridine (300 mg, 990 μmol, 75.2% yield, 98.2% purity, HCl salt) as a yellow oil. ¹H NMR (MeOD 400 MHz) δ_{H}=8.43 (d, J=2.8 Hz, 1H), 8.28 (s, 1H), 7.97 (dd, J=2.8 Hz, 8.8 Hz, 1H), 3.50 (br d, J=12.8 Hz, 2H), 3.25-3.16 (m, 1H), 3.10-3.01 (m, 2H), 2.46 (s, 3H), 2.19-2.11 (m, 2H), 2.06-1.95 (m, 2H). LCMS m/z [M+H]⁺ 262.

### Step 5: Synthesis of tert-butyl 6-[4-[5-fluoro-2-(4-methyltriazol-1-yl)-3-pyridyl]-1-piperidyl]-2-azaspiro[3.4]octane-2-carboxylate (C45)

To a mixture of 5-fluoro-2-(4-methyltriazol-1-yl)-3-(4-piperidyl)pyridine (300 mg, 1.01 mmol, HCl salt) in DCE (5.00 mL) was added TEA (420 μL, 3.02 mmol) and then added tert-butyl 6-oxo-2-azaspiro[3.4]octane-2-carboxylate (227 mg, 1.01 mmol). The mixture was stirred at room temperature for 0.5 h. HOAc (28.8 μL, 504 μmol) and NaBH(OAc)₃ (641 mg, 3.02 mmol) were added. The mixture was stirred at room temperature for 11.5 h. The reaction mixture was poured into a 10% aqueous NaHCO₃ solution (15 mL), and then extracted with DCM (3×5 mL). The combined organic layer was washed with brine (10 mL), dried over Na₂SO₄, filtered and concentrated under a reduced pressure to give tert-butyl 6-[4-[5-fluoro-2-(4-methyltriazol-1-yl)-3-pyridyl]-1-piperidyl]-2-azaspiro[3.4]octane-2-carboxylate (450 mg, 956 μmol, 83.3% yield) as a yellow oil. LCMS m/z [M+H]⁺ 471.

### Step 6: Synthesis of ethyl 6-[4-[5-fluoro-2-(4-methyltriazol-1-yl)-3-pyridyl]-1-piperidyl]-2-azaspiro[3.4]octane-2-carboxylate (C46)

To a solution of tert-butyl 6-[4-[5-fluoro-2-(4-methyltriazol-1-yl)-3-pyridyl]-1-piperidyl]-2-azaspiro[3.4]octane-2-carboxylate (450 mg, 956 μmol) in DCM (5.00 mL) was added dropwise TFA (1.20 mL, 16.2 mmol). The reaction mixture was stirred at room temperature for 3 h. The reaction mixture was concentrated under a reduced pressure to give 6-[4-[5-fluoro-2-(4-methyltriazol-1-yl)-3-pyridyl]-1-piperidyl]-2-azaspiro[3.4]octane-2-carboxylate (463 mg, crude, TFA salt) as a yellow oil, which was used in next step as theoretical amount.

### Step 7: Synthesis of 6-[4-[5-fluoro-2-(4-methyltriazol-1-yl)-3-pyridyl]-1-piperidyl]-2-azaspiro[3.4]octane-2-carboxylate (C47)

A mixture of 6-[4-[5-fluoro-2-(4-methyltriazol-1-yl)-3-pyridyl]-1-piperidyl]-2-azaspiro [3.4]octane (463 mg, 956 μmol, TFA salt) in DCM (4.00 mL) was added dropwise TEA (666 μL, 4.78 mmol) at 0° C. Then ethyl carbonochloridate (690 mg, 6.36 mmol) was added dropwise into the mixture at 0° C. The mixture was stirred at room temperature for 12 h. The reaction mixture was quenched with a saturated aqueous NaHCO₃ solution (20 mL) slowly at 0° C. and then extracted with DCM (3×5 mL). The combined organic phase was washed with brine (10 mL), dried over anhydrous Na₂SO₄, filtered and concentrated under a reduced pressure. The residue was purified by flash silica gel chromatography (Eluent of 0˜5% MeOH/DCM) to give ethyl 6-[4-[5-fluoro-2-(4-methyltriazol-1-yl)-3-pyridyl]-1-piperidyl]-2-azaspiro[3.4]octane-2-carboxylate (250 mg, 551 μmol, 51.0% yield, 97.5% purity) as a yellow oil. LCMS m/z [M+H]+ 443.

### Step 8: Synthesis of ethyl (6R)-6-[4-[5-fluoro-2-(4-methyltriazol-1-yl)-3-pyridyl]-1-piperidyl]-2-azaspiro[3.4]octane-2-carboxylate (A23) and ethyl (6S)-6-[4-[5-fluoro-2-(4-methyltriazol-1-yl)-3-pyridyl]-1-piperidyl]-2-azaspiro[3.4]octane-2-carboxylate (A24)

The ethyl 6-[4-[5-fluoro-2-(4-methyltriazol-1-yl)-3-pyridyl]-1-piperidyl]-2-azaspiro[3.4]-octane-2-carboxylate (120 mg, 271 μmol) was purified by SFC (column: DAICEL CHIRALPAK IG (250 mm×30 mm, 10 um); mobile phase: [CO₂-EtOH (0.1% NH₃H₂O)]; B %: 55%, isocratic elution mode) to give Peak 1 ethyl (6R)-6-[4-[5-fluoro-2-(4-methyltriazol-1-yl)-3-pyridyl]-1-piperidyl]-2-azaspiro[3.4]octane-2-carboxylate (37.1 mg, 81.6 μmol, 30.1% yield, 97.4% purity) as an off-white solid. ¹H NMR (CDCl₃ 400 MHz) δ_{H}=8.23 (d, J=2.8 Hz, 1H), 7.85 (s, 1H), 7.60 (dd, J=2.8 Hz, 9.2 Hz, 1H), 4.09 (q, J=7.2 Hz, 2H), 3.89-3.80 (m, 2H), 3.80-3.74 (m, 2H), 3.18-2.99 (m, 31H), 2.62-2.52 (m, 1H), 2.45 (s, 3), 2.16-2.07 (m, 1H), 2.05-1.75 (m, 8H), 1.75-1.78 (m, 2H), 1.58-1.47 (i, 1H), 1.23 (t, J=7.2 Hz, 3H). LCMS m/z [M+H]⁺ 443. Peak 2 ethyl (6S)-6-[4-[5-fluoro-2-(4-methyltriazol-1-yl)-3-pyridyl]-1-piperidyl]-2-azaspiro[3.4]octane-2-carboxylate (46.9 mg, 106 μmol, 39.1% yield, 100% purity) as an off-white solid. ¹H NMR (CDCl₃ 400 MHz) δ_{H}=8.23 (d, J=2.8 Hz, 1H), 7.86 (s, 1H), 7.61 (dd, J=2.4 Hz, 8.8 Hz, 1H), 4.09 (q, J=7.2 Hz, 2H), 3.90-3.81 (m, 2H), 3.80-3.74 (mi, 2H), 3.17-2.99 (m, 3H), 2.64-2.52 (mi, 1H), 2.46 (s, 3H), 2.16-2.07 (m, 1H), 2.03-1.75 (m, 8H), 1.74-1.68 (m, 21), 1.56-1.47 (m, 1H), 1.23 (t, J=7.2 Hz, 31H). LCMS m/z [M+H]⁺ 443.

Using the methodology described above for Examples A1-A24 and analogous starting material as noted in the table, Compounds A25-A293 were synthesized, as noted in the below Table.

| Compound | Synthesized Like | ¹H NMR. Mass spectrum |
|---|---|---|
| A25 | A1 | ¹H NMR (CDCl₃ 400 MHz) δ_{H} = 9.11 (s, 1H), 8.46-8.26 (m, 2H), 8.02 (s, |
| | | 1H), 7.37 (d, J = 8.0 Hz, 1H), 7.24-7.19 (m, 1H), 4.09 (q, J = 6.8 Hz, |
| | | 2H), 3.91-3.79 (m, 2H), 3.78-3.72 (m, 2H), 3.00-2.75 (m, 4H), 2.66-2.31 |
| | | (m, 8H), 2.14-2.03 (m, 1H), 1.94-1.64 (m, 4H), 1.53-1.44 (m, 1H), 1.23 |
| | | (t, J = 6.8 Hz, 3H). LCMS m/z [M + H]⁺ 436. |
| A26 | A1 | ¹H NMR (CDCl₃ 400 MHz) δ_{H} = 9.13 (s, 1H), 8.45 (d, J = 2.4 Hz, 1H), |
| | | 8.38 (d, J = 4.4 Hz, 1H), 8.06 (d, J = 9.6 Hz, 1H), 7.41 (d, J = 8.0 Hz, |
| | | 1H), 7.26-7.23 (m, 1H), 4.09 (q, J = 7.2 Hz, 2H), 3.91-3.80 (m, 2H), |
| | | 3.80-3.73 (m, 2H), 3.02-2.82 (m, 4H), 2.66-2.34 (m, 5H), 2.12-2.04 (m, |
| | | 1H), 1.96-1.66 (m, 4H), 1.55-1.40 (m, 1H), 1.23 (t, J = 7.2 Hz, 3H). |
| | | LCMS m/z [M + H]⁺ 440. |
| A27 | A1 | ¹H NMR (CDCl₃ 400 MHz) δ_{H} = 9.51 (s, 1H), 8.86 (s, 1H), 8.65 (s, 1H), |
| | | 8.42 (s, 1H), 7.55-7.40 (m, 1H), 7.38-7.26 (m, 1H), 4.09 (q, J = 7.2 Hz, |
| | | 2H), 3.95-3.74 (m, 4H), 3.14-2.76 (m, 4H), 2.74-2.20 (m, 5H), 2.18-2.04 |
| | | (m, 1H), 1.99-1.59 (m, 4H), 1.50-1.44 (m, 1H), 1.24 (t, J = 7.2 Hz, 3H). |
| | | LCMS m/z [M + H]⁺ 447. |
| A28 | A1 | ¹H NMR (CDCl₃ 400 MHz) δ_{H} = 9.15 (d, J = 1.6 Hz, 1H), 8.47 (d, J = 2.4 |
| | | Hz, 1H), 8.42 (d, J = 4.4 Hz, 1H), 8.16 (d, J = 7.2 Hz, 2H), 7.47 (d, J = |
| | | 7.6 Hz, 1H), 7.29 (dd, J = 4.8 Hz, 8.4 Hz, 1H), 6.92-6.39 (m, 1H), 4.09 |
| | | (q, J = 6.8 Hz, 2H), 3.95-3.82 (m, 2H), 3.81-3.75 (m, 2H), 3.12-2.98 (m, |
| | | 4H), 2.96-2.54 (m, 5H), 2.20-2.10 (m, 1H), 2.05-1.75 (m, 5H), 1.23 (t, |
| | | J = 6.8 Hz, 3H). LCMS m/z [M + H]⁺ 488. |
| A29 | A2 | ¹H NMR (CDCl₃ 400 MHz) δ_{H} = 9.38 (d, J = 1.6 Hz, 1H), 8.47-8.17 (m, |
| | | 2H), 7.88-7.67 (m, 1H), 7.23-7.09 (m, 1H), 4.10 (q, J = 7.2 Hz, 2H), |
| | | 3.59-3.77 (m, 2H), 3.76-3.72 (m, 2H), 2.90 (br s, 4H), 2.70-2.35 (m, |
| | | 5H), 2.10 (br s, 1H), 2.00-1.76 (m, 3H), 1.73-1.58 (m, 1H), 1.55-1.44 |
| | | (m, 1H), 1.24 (t, J = 7.2 Hz, 3H). LCMS m/z [M + H]⁺ 465. |
| A30 | A2 | ¹H NMR (CDCl₃ 400 MHz) δ_{H} = 9.69-9.58 (m, 1H), 8.32 (d, J = 5.2 Hz, |
| | | 1H), 8.20 (d, J = 2.4 Hz, 1H), 7.45 (d, J = 5.2 Hz, 1H), 7.26-7.35(m, |
| | | 1H), 7.06 (dd, J = 2.4, 10.4 Hz, 1H), 6.48 (d, J = 2.4 Hz, 1H), 4.01 (q, |
| | | J = 7.2 Hz, 2H), 3.81-3.66 (m, 4H), 2.85 (s, 4H), 2.49-2.22 (m, 5H), |
| | | 1.94 (s, 1H), 1.70-1.80 (m, 5H), 1.15 (t, J = 7.2 Hz, 3H). LCMS m/z |
| | | [M + H]⁺ 479. |
| A31 | A2 | ¹H NMR (CDCl₃ 400 MHz) δ_{H} = 9.03 (s, 1H), 8.42 (s, 1H), 8.24 (d, J = |
| | | 1.6 Hz, 1H), 7.95 (s, 1H), 7.11 (dd, J = 1.6 Hz, 10.0 Hz, 1H), 4.09 (q, |
| | | J = 7.2 Hz, 2H), 3.92-3.81 (m, 2H), 3.77 (s, 2H), 2.97 (br s, 4H), 2.77- |
| | | 2.44 (m, 5H), 2.40 (s, 3H), 2.11 (dd, J = 6.8 Hz, 12.4 Hz, 1H), 2.01- |
| | | 1.78 (m, 5H), 1.23 (t, J = 7.2 Hz, 3H). LCMS m/z [M + H]⁺ 454. |
| A32 | A2 | ¹H NMR (CDCl₃ 400 MHz) δ_{H} = 8.17 (d, J = 2.4 Hz, 1H), 7.42 (d, J = 8.0 |
| | | Hz, 1H), 7.33 (s, 1H), 7.29-7.26 (m, 1H), 7.03 (dd, J = 2.4, 10.4 Hz, |
| | | 1H), 6.82 (dd, J = 2.4, 7.6 Hz, 1H), 4.10 (q, J = 7.2 Hz, 2H), 3.89-3.80 |
| | | (m, 2H), 3.77 (s, 2H), 2.91 (s, 4H), 2.47 (s, 5H), 2.08 (dd, J = 7.2, 12.4 |
| | | Hz, 1H), 1.98-1.67 (m, 5H), 1.23 (t, J = 7.2 Hz, 3H). LCMS m/z [M + H]⁺ |
| | | 455. |
| A33 | A2 | ¹H NMR (CDCl₃ 400 MHz) δ_{H} = 8.26-8.18 (m, 1H), 8.08 (s, 1H), 7.95 (s, |
| | | 1H), 7.81 (d, J = 9.2Hz, 1H), 7.70 (d, J = 8.8 Hz, 1H), 7.11-7.04 (m, |
| | | 1H), 4.24 (s, 3H), 4.09 (d, J = 6.8 Hz, 2H), 3.93-3.72 (m, 5H), 3.05-2.85 |
| | | (m, 4H), 2.60-2.44 (m, 3H), 2.13-2.06 (m, 1H), 1.82 (d, J = 17.6 Hz, |
| | | 6H), 1.22 (t, J = 6.8 Hz, 3H). LCMS m/z [M + H]⁺ 493. |
| A34 | A2 | ¹H NMR (CDCl₃ 400 MHz) δ_{H} = 8.26-8.17 (m, 1H), 7.85 (d, J = 7.6 |
| | | Hz, 2H), 7.46-7.39 (m, 2H), 7.08 (d, J = 9.2 Hz, 1H), 4.76 (s, 2H), 4.04- |
| | | 4.13 (m, 2H), 3.90-3.70 (m, 4H), 3.40-3.20 (m, 1H), 3.20-2.80 (m, |
| | | 4H), 2.78-2.42 (m, 4H), 2.20-2.09 (m, 1H), 2.07-1.74 (m, 6H), 1.23 |
| | | (t, J = 7.2 Hz, 3H). LCMS m/z [M + H]⁺ 469. |
| A35 | A2 | LCMS m/z [M + H]⁺ 454. |
| A36 | A2 | LCMS m/z [M + H]⁺ 470. |
| A37 | A2 | LCMS m/z [M + H]⁺ 440. |
| A38 | A2 | LCMS m/z [M + H]⁺ 487. |
| A39 | A2 | LCMS m/z [M + H]⁺ 464. |
| A40 | A2 | LCMS m/z [M + H]⁺ 485. |
| A41 | A2 | LCMS m/z [M + H]⁺ 498. |
| A42 | A2 | LCMS m/z [M + H]⁺ 473. |
| A43 | A2 | LCMS m/z [M + H]⁺ 494. |
| A44 | A2 | LCMS m/z [M + H]⁺ 473. |
| A45 | A2 | LCMS m/z [M + H]⁺ 458. |
| A46 | A2 | LCMS m/z [M + H]⁺ 470. |
| A47 | A2 | LCMS m/z [M + H]⁺ 464. |
| A48 | A2 | LCMS m/z [M + H]⁺ 497. |
| A49 | A2 | LCMS m/z [M + H]⁺ 484. |
| A50 | A2 | LCMS m/z [M + H]⁺ 484. |
| A51 | A2 | LCMS m/z [M + H]⁺ 494. |
| A52 | A2 | LCMS m/z [M + H]⁺ 470. |
| A53 | A2 | LCMS m/z [M + H]⁺ 440. |
| A54 | A2 | LCMS m/z [M + H]⁺ 493. |
| A55 | A2 | LCMS m/z [M + H]⁺ 458. |
| A56 | A2 | LCMS m/z [M + H]⁺ 483. |
| A57 | A2 | LCMS m/z [M + H]⁺ 469. |
| A58 | A2 | LCMS m/z [M + H]⁺ 493. |
| A59 | A2 | LCMS m/z [M + H]⁺ 446. |
| A60 | A2 | LCMS m/z [M + H]⁺ 493. |
| A61 | A2 | LCMS m/z [M + H]⁺ 479. |
| A62 | A2 | LCMS m/z [M + H]⁺ 479. |
| A63 | A2 | LCMS m/z [M + H]⁺ 487. |
| A64 | A2 | LCMS m/z [M + H]⁺ 465. |
| A65 | A2 | LCMS m/z [M + H]⁺ 493. |
| A66 | A2 | ¹H NMR (CDCl₃ 400 MHz) δ_{H} = 8.26 (d, J = 1.6 Hz, 1H), 7.95 (s, 1H), |
| | | 7.94-7.94 (m, 1H), 7.67 (d, J = 8.4 Hz, 1H), 7.56 (d, J = 7.2 Hz, 1H), |
| | | 7.16-7.03 (m, 2H), 4.23 (s, 3H), 4.08 (q, J = 7.2 Hz, 2H), 3.86-3.77 |
| | | (m, 2H), 3.73 (s, 2H), 2.89 (s, 4H), 2.53-2.17 (m, 5H), 2.06-1.99 (m, |
| | | 1H), 1.92-1.73 (m, 4H), 1.22 (t, J = 7.2 Hz, 3H). LCMS m/z [M + H]⁺ |
| | | 493. |
| A67 | A2 | ¹H NMR (CDCl₃ 400 MHz) δ_{H} = 8.19 (br, 1H), 7.52 (s, 1H), 7.13-7.01 |
| | | (m, 1H), 6.60 (s, 1H), 4.09 (q, J = 6.8 Hz, 2H), 3.90 (s, 3H), 3.88-3.80 |
| | | (m, 2H), 3.77 (s, 2H), 2.87 (br, 4H), 2.60-2.35 (m, 5H), 2.08 (dd, J = |
| | | 7.2, 12.4 Hz, 1H), 1.97-1.82 (m, 3H), 1.80 (br, 1H), 1.55-1.43 (m, |
| | | 1H), 1.23 (br, J = 7.2 Hz, 3H). LCMS m/z [M + H]⁺ 443. |
| A68 | A2 | LCMS m/z [M + H]⁺ 493. |
| A69 | A2 | LCMS m/z [M + H]⁺ 470. |
| A70 | A2 | LCMS m/z [M + H]⁺ 470. |
| A71 | A2 | LCMS m/z [M + H]⁺ 485. |
| A72 | A2 | LCMS m/z [M + H]⁺ 499. |
| A73 | A2 | LCMS m/z [M + H]⁺ 484. |
| A74 | A2 | LCMS m/z [M + H]⁺ 457. |
| A75 | A2 | ¹H NMR (CDCl₃ 400 MHz) δ_{H} = 8.83 (s, 1H), 8.34-8.21 (m, 3H), 8.01 (s, |
| | | 1H), 7.11 (d, J = 9.6 Hz, 1H), 4.10 (q, J = 7.2 Hz, 2H), 3.93-3.75 (m, |
| | | 5H), 2.99-2.83 (m, 4H), 2.60-2.43 (m, 4H), 2.24 (s, 3H), 2.10 (dd, J = |
| | | 8.4, 9.6 Hz, 1H), 2.00-1.67 (m, 5H), 1.23 (t, J = 7.2 Hz, 3H). LCMS m/z |
| | | [M + H]⁺ 497. |
| A76 | A2 | LCMS m/z [M + H]⁺ 429. |
| A77 | A2 | LCMS m/z [M + H]⁺ 485. |
| A78 | A2 | LCMS m/z [M + H]⁺ 458. |
| A79 | A2 | LCMS m/z [M + H]⁺ 476. |
| A80 | A2 | LCMS m/z [M + H]⁺ 484. |
| A81 | A2 | ¹H NMR (CDCl₃ 400 MHz) δ_{H} = 8.20 (s, 1H), 7.02 (d, J = 8.4 Hz, 1H), |
| | | 4.09 (q, J = 7.2 Hz, 2H), 3.91-3.80 (m, 2H), 3.77 (s, 2H), 3.00-2.75 (m, |
| | | 4H), 2.55-2.36 (m, 4H), 2.24 (s, 6H), 2.09 (dd, J = 4.8, 12.4 Hz, 2H), |
| | | 2.01-1.75 (m, 6H), 1.23 (t, J = 7.2 Hz, 3H). LCMS m/z [M + H]⁺ 457. |
| A82 | A2 | LCMS m/z [M + H]⁺ 456. |
| A83 | A2 | ¹H NMR (CDCl₃ 400 MHz) δ_{H} = 9.27 (s, 1H), 8.37-8.16 (m, 2H), 7.24- |
| | | 7.19 (m, 1H), 4.09 (q, J = 7.2 Hz, 2H), 3.95-3.81 (m, 2H), 3.79 (s, 2H), |
| | | 2.80-3.10 (m, 4H), 2.40-2.75 (m, 4H), 2.21-2.09 (m, 1H), 2.08-1.66 |
| | | (m, 6H), 1.23 (t, J = 7.2 Hz, 3H). LCMS m/z [M + H]⁺ 483. |
| A84 | A2 | LCMS m/z [M + H]⁺ 469. |
| A85 | A2 | ¹H NMR (CDCl₃ 400 MHz) δ_{H} = 8.20 (d, J = 2.4 Hz, 1H), 8.07 (s, 1H), |
| | | 7.12 (dd, J = 2.4, 10.0 Hz, 1H), 4.09 (q, J = 7.2 Hz, 2H), 3.89-3.74 (m, |
| | | 4H), 2.90 (s, 4H), 2.65-2.42 (m, 5H), 2.05-2.13(m, 1H), 1.96-1.68 (m, |
| | | 6H), 1.22 (t, J = 7.2 Hz, 3H). LCMS m/z [M + H]⁺ 497. |
| A86 | A2 | LCMS m/z [M + H]⁺ 475. |
| A87 | A2 | LCMS m/z [M + H]⁺ 498. |
| A88 | A2 | LCMS m/z [M + H]⁺ 475. |
| A89 | A2 | LCMS m/z [M + H]⁺ 493. |
| A90 | A2 | LCMS m/z [M + H]⁺ 483. |
| A91 | A2 | LCMS m/z [M + H]⁺ 459. |
| A92 | A2 | LCMS m/z [M + H]⁺ 479. |
| A93 | A2 | LCMS m/z [M + H]⁺ 457. |
| A94 | A2 | ¹H NMR (CDCl₃ 400 MHz) δ_{H} = 8.21 (d, J = 2.4 Hz, 1H), 7.44 (d, J = 7.6 |
| | | Hz, 1H), 7.34 (dd, J = 1.6, 7.6 Hz, 1H), 7.21 (d, J = 1.2 Hz, 1H), 7.12- |
| | | 7.07 (m, 1H), 4.08 (q, J = 7.2 Hz, 2H), 3.91-3.70 (m, 8H), 2.84 (t, J = |
| | | 4.4 Hz, 4H), 2.32 (s, 4H), 2.12-2.02 (m, 1H), 1.86 (s, 3H), 1.72-1.63 |
| | | (m, 1H), 1.56-1.43 (m, 1H), 1.22 (t, J = 7.2 Hz, 3H). LCMS m/z [M + H]⁺ |
| | | 494. |
| A95 | A2 | LCMS m/z [M + H]⁺ 496. |
| A96 | A2 | ¹H NMR (CDCl₃ 400 MHz) δ_{H} = 8.72 (s, 1H), 8.29-8.12 (m, 1H), 7.93- |
| | | 7.82 (m, 1H), 7.69-7.59 (m, 3H), 7.13 (d, J = 9.6 Hz, 1H), 4.09 (q, J = |
| | | 6.8 Hz, 2H), 3.90-3.73 (m, 4H), 2.97 (s, 4H), 2.66-2.45 (m, 5H), 2.10 |
| | | (dd, J = 7.2, 11.2 Hz, 1H), 1.97-1.74 (m, 5H), 1.23 (t, J = 6.8 Hz, 3H). |
| | | LCMS m/z [M + H]⁺ 479. |
| A97 | A2 | LCMS m/z [M + H]⁺ 496. |
| A98 | A2 | LCMS m/z [M + H]⁺ 454. |
| A99 | A2 | ¹H NMR (CDCl₃ 400 MHz) δ_{H} = 8.94 (d, J = 2.8 Hz, 1H), 8.37 (d, J = |
| | | 2.80 Hz, 1H), 8.25 (d, J = 2.4 Hz, 1H), 7.21 (d, J = 3.6 Hz, 1H), 7.11 |
| | | (dd, J = 2.8, 10.0 Hz, 1H), 6.51 (d, J = 3.6 Hz, 1H), 4.09 (q, J = 7.2 Hz, |
| | | 2H), 3.93 (s, 3H), 3.89-3.79 (m, 2H), 3.76 (s, 2H), 2.97 (s, 4H), 2.79- |
| | | 2.61 (m, 5H), 2.09 (dd, J = 7.2, 12.8 Hz, 1H), 1.98-1.75 (m, 4H), 1.68- |
| | | 1.59 (m, 1H), 1.23 (t, J = 7.2 Hz, 3H). LCMS m/z [M + H]⁺ 493. |
| A100 | A2 | LCMS m/z [M + H]⁺ 480. |
| A101 | A2 | LCMS m/z [M + H]⁺ 443. |
| A102 | A2 | LCMS m/z [M + H]⁺ 470. |
| A103 | A2 | LCMS m/z [M + H]⁺ 496. |
| A104 | A2 | ¹H NMR (CDCl₃ 400 MHz) δ_{H} = 8.20 (s, 1H), 7.96 (br d, J = 8.4 Hz, 2H), |
| | | 7.82 (br d, J = 8.4 Hz, 2H), 7.10-7.04 (m, 1H), 6.24 (br d, J = 3.6 Hz, |
| | | 1H), 4.09 (q, J = 7.2 Hz, 2H), 3.88-3.80 (m, 2H), 3.79-3.74 (m, 2H), |
| | | 3.04 (br d, J = 4.8 Hz, 3H), 2.88 (br s, 4H), 2.57-2.42 (m, 4H), 2.08 (br |
| | | dd, J = 7.2, 12.0 Hz, 1H), 1.96-1.85 (m, 2H), 1.85-1.73 (m, 2H), 1.63- |
| | | 1.43 (m, 2H), 1.23 (br t, J = 7.2 Hz, 3H). LCMS m/z [M + H]⁺ 455. |
| A105 | A2 | LCMS m/z [M + H]⁺ 487. |
| A106 | A2 | LCMS m/z [M + H]⁺ 488. |
| A107 | A2 | LCMS m/z [M + H]⁺ 484. |
| A108 | A2 | LCMS m/z [M + H]⁺ 472. |
| A109 | A2 | LCMS m/z [M + H]⁺ 454. |
| A110 | A2 | LCMS m/z [M + H]⁺ 470. |
| A111 | A2 | ¹H NMR (CDCl₃ 400 MHz) δ_{H} = 8.62 (d, J = 2.0 Hz, 1H), 8.19 (d, J = 2.0 |
| | | Hz, 1H), 7.92 (s, 1H), 7.06 (dd, J = 2.4, 10.4 Hz, 1H), 4.09 (q, J = 7.2 |
| | | Hz, 2H), 4.01 (s, 3H), 3.89-3.80 (m, 2H), 3.80-3.75 (m, 2H), 2.91 (s, |
| | | 4H), 2.63-2.39 (m, 5H), 2.24 (s, 3H), 2.10 (dd, J = 6.8, 12.4 Hz, 1H), |
| | | 1.98-1.96 (m, 1H), 1.98-1.66 (m, 4H), 1.23 (t, J = 7.2 Hz, 3H). LCMS |
| | | m/z [M + H]⁺ 484. |
| A112 | A2 | ¹H NMR (CDCl₃ 400 MHz) δ_{H} = 8.17 (d, J = 2.0 Hz, 1H), 7.80-7.73 (m, |
| | | 1H), 7.67-7.60 (m, 1H), 7.07-6.97 (m, 2H), 4.09 (q, J = 7.2 Hz, 2H), |
| | | 3.94 (s, 3H), 3.89-3.74 (m, 4H), 2.95-2.83 (m, 4H), 2.64-2.39 (m, |
| | | 5H), 2.15-2.06 (m, 1H), 1.95-1.70 (m, 4H), 1.58-1.45 (m, 1H), 1.23 |
| | | (t, J = 7.2 Hz, 3H). LCMS m/z [M + H]⁺ 487. |
| A113 | A2 | LCMS m/z [M + H]⁺ 444. |
| A114 | A2 | LCMS m/z [M + H]⁺ 472. |
| A115 | A2 | LCMS m/z [M + H]⁺ 473. |
| A116 | A2 | LCMS m/z [M + H]⁺ 484. |
| A117 | A2 | ¹H NMR (CDCl₃ 400 MHz) δ_{H} = 8.15 (br s, 1H), 7.49 (dd, J = 6.0, 8.4 |
| | | Hz, 1H), 7.39 (dd, J = 2.8, 10.0 Hz, 1H), 7.18-7.02 (m, 2H), 4.83 (br s, |
| | | 1H), 4.35-4.28 (m, 2H), 4.08 (q, J = 7.2 Hz, 2H), 3.87-3.78 (m, 2H), |
| | | 3.78-3.72 (m, 2H), 2.92-2.80 (m, 4H), 2.56-2.36 (m, 5H), 2.06 (dd, |
| | | J = 7.2, 12.4 Hz, 1H), 1.77 (br s, 4H), 1.52-1.41 (m, 1H), 1.22 (t, J = 7.2 |
| | | Hz, 3H). LCMS m/z [M + H]⁺ 487. |
| A118 | A2 | LCMS m/z [M + H]⁺ 473. |
| A119 | A2 | LCMS m/z [M + H]⁺ 476. |
| A120 | A2 | LCMS m/z [M + H]⁺ 443. |
| A121 | A2 | LCMS m/z [M + H]⁺ 457. |
| A122 | A2 | LCMS m/z [M + H]⁺ 484. |
| A123 | A2 | LCMS m/z [M + H]⁺ 470. |
| A124 | A2 | LCMS m/z [M + H]⁺ 496. |
| A125 | A2 | LCMS m/z [M + H]⁺ 472. |
| A126 | A2 | LCMS m/z [M + H]⁺ 485. |
| A127 | A2 | LCMS m/z [M + H]⁺ 473. |
| A128 | A2 | LCMS m/z [M + H]⁺ 458. |
| A129 | A2 | LCMS m/z [M + H]⁺ 494. |
| A130 | A2 | LCMS m/z [M + H]⁺ 483. |
| A131 | A2 | LCMS m/z [M + H]⁺ 493. |
| A132 | A2 | LCMS m/z [M + H]⁺ 485. |
| A133 | A2 | LCMS m/z [M + H]⁺ 470. |
| A134 | A2 | LCMS m/z [M + H]⁺ 483. |
| A135 | A2 | LCMS m/z [M + H]⁺ 454. |
| A136 | A2 | LCMS m/z [M + H]⁺ 473. |
| A137 | A2 | LCMS m/z [M + H]⁺ 484. |
| A138 | A2 | LCMS m/z [M + H]⁺ 484. |
| A139 | A2 | LCMS m/z [M + H]⁺ 488. |
| A140 | A2 | LCMS m/z [M + H]⁺ 444. |
| A141 | A2 | LCMS m/z [M + H]⁺ 483. |
| A142 | A2 | ¹H NMR (CDCl₃ 400 MHz) δ_{H} = 8.20 (br s, 1H), 7.11-6.99 (m, 4H), |
| | | 4.09 (q, J = 7.2 Hz, 2H), 3.88-3.74 (m, 4H), 2.88 (br s, 4H), 2.60-2.48 |
| | | (m, 1H), 2.37 (br d, J = 1.2 Hz, 4H), 2.10-2.02 (m, 1H), 1.97-1.63 (m, |
| | | 6H), 1.23 (br t, J = 6.8 Hz, 3H). LCMS m/z [M + H]⁺ 473. |
| A143 | A2 | LCMS m/z [M + H]⁺ 456. |
| A144 | A2 | LCMS m/z [M + H]⁺ 457. |
| A145 | A2 | ¹H NMR (CDCl₃ 400 MHz) δ_{H} = 8.13 (d, J = 2.4 Hz, 1H), 7.59 (d, J = 8.8 |
| | | Hz, 1H), 7.15-6.99 (m, 2H), 6.89 (dd, J = 2.8, 8.8 Hz, 1H), 5.10 (s, 1H), |
| | | 4.31 (s, 2H), 4.08 (q, J = 7.2 Hz, 2H), 3.87 (s, 3H), 3.85-3.78 (m, 2H), |
| | | 3.75 (s, 2H), 2.85 (s, 4H), 2.56-2.20 (m, 5H), 2.06 (dd, J = 7.2, 12.8 Hz, |
| | | 1H), 1.94-1.75 (m, 3H), 1.47 (d, J = 1.6 Hz, 1H), 1.22 (t, J = 7.2 Hz, |
| | | 3H). LCMS m/z [M + H]⁺ 499. |
| A146 | A2 | ¹H NMR (CDCl₃ 400 MHz) δ_{H} = 9.12 (s, 2H), 8.24 (d, J = 2.4 Hz, 1H), |
| | | 7.14 (dd, J = 2.4, 10.0 Hz, 1H), 4.15-4.03 (m, 5H), 3.89-3.80 (m, 2H), |
| | | 3.80-3.75 (m, 2H), 2.91 (br s, 4H), 2.71-2.35 (m, 5H), 2.09 (dd, J = 7.2, |
| | | 12.8 Hz, 1H), 1.96-1.80 (m, 3H), 1.79-1.70 (m, 1H), 1.52 (s, 1H), 1.23 |
| | | (t, J = 7.2 Hz, 3H). LCMS m/z [M + H]⁺ 471. |
| A147 | A2 | LCMS m/z [M + H]⁺ 487. |
| A148 | A2 | LCMS m/z [M + H]⁺ 479. |
| A149 | A2 | ¹H NMR (CDCl₃ 400 MHz) δ_{H} = 8.21-8.10 (m, 1H), 8.08-7.92 (m, 2H), |
| | | 7.14-7.03 (m, 1H), 6.64-6.54 (m, 1H), 5.37-5.25 (m, 1H), 4.09 (q, |
| | | J = 7.2 Hz, 2H), 3.92-3.73 (m, 4H), 2.93 (s, 4H), 2.54 (s, 5H), 2.11 (dd, |
| | | J = 7.2, 12.4 Hz, 1H), 1.97-1.80 (m, 3H), 1.78-1.49 (m, 7H), 1.23 (t, J = |
| | | 7.2 Hz, 3H). LCMS m/z [M + H]⁺ 498. |
| A150 | A2 | LCMS m/z [M + H]⁺ 483. |
| A151 | A2 | ¹H NMR (CDCl₃ 400 MHz) δ_{H} = 8.18 (s, 1H), 7.74-7.68 (m, 1H), 7.61- |
| | | 7.51 (m, 1H), 7.51-7.49 (m, 1H), 7.48 (s, 2H), 7.04 (dd, J = 2.4, 10.0 |
| | | Hz, 1H), 4.09 (q, J = 7.2 Hz, 2H), 3.88-3.79 (m, 2H), 3.77-3.72 (m, |
| | | 2H), 2.91-2.78 (m, 4H), 2.72 (d, J = 4.8 Hz, 4H), 2.52-2.39 (m, 3H), |
| | | 2.11-2.05 (m, 1H), 1.93-1.73 (m, 5H), 1.23 (t, J = 7.2 Hz, 3H). LCMS |
| | | m/z [M + H]⁺ 496. |
| A152 | A2 | LCMS m/z [M + H]⁺ 494. |
| A153 | A2 | LCMS m/z [M + H]⁺ 476. |
| A154 | A2 | LCMS m/z [M + H]⁺ 487. |
| A155 | A2 | LCMS m/z [M + H]⁺ 484. |
| A156 | A2 | ¹H NMR (CDCl₃ 400 MHz) δ_{H} = 8.18 (d, J = 2.4 Hz, 1H), 7.54 (dd, J = |
| | | 2.0, 8.8 Hz, 1H), 7.34 (ddd, J = 2.4, 4.8, 8.4 Hz, 1H), 7.14-7.01 (m, 2H), |
| | | 4.10 (q, J = 7.2 Hz, 2H), 3.96-3.71 (m, 4H), 2.96 (s, 4H), 2.63 (s, 5H), |
| | | 2.17-2.04 (m, 1H), 2.00-1.79 (m, 4H), 1.72-1.60 (m, 1H), 1.23 (t, J = 7.2 |
| | | Hz, 3H). LCMS m/z [M + H]⁺ 473. |
| A157 | A2 | LCMS m/z [M + H]⁺ 457. |
| A158 | A2 | LCMS m/z [M + H]⁺ 499. |
| A159 | A2 | ¹H NMR (CDCl₃ 400 MHz) δ_{H} = 8.26-8.20 (m, 1H), 8.11-8.06 (m, 1H), |
| | | 8.00-7.95 (m, 1H), 7.80-7.72 (m, 2H), 7.12-7.05 (m, 1H), 4.08 (q, |
| | | J = 7.2 Hz, 2H), 3.87-3.74 (m, 4H), 2.96-2.85 (m, 4H), 2.68-2.25 (m, |
| | | 6H), 2.12-2.02 (m, 1H), 1.95-1.77 (m, 4H), 1.53-1.44 (m, 1H), 1.22 |
| | | (t, J = 7.2 Hz, 3H). LCMS m/z [M + H]⁺ 479. |
| A160 | A2 | LCMS m/z [M + H]⁺ 483. |
| A161 | A2 | ¹H NMR (CDCl₃ 400 MHz) δ_{H} = 8.22 (d, J = 1.6 Hz, 1H), 7.80 (t, J = 8.0 |
| | | Hz, 1H), 7.15-7.09 (m, 1H), 6.84 (dd, J = 3.2, 8.4 Hz, 1H), 4.09 (q, J = |
| | | 7.2 Hz, 2H), 3.87-3.79 (m, 2H), 3.76 (s, 2H), 2.83 (br s, 3H), 2.61- |
| | | 2.23 (m, 8H), 2.06 (br dd, J = 7.2, 12.4 Hz, 1H), 1.94-1.67 (m, 4H), |
| | | 1.47 (br d, J = 2.0 Hz, 2H), 1.23 (t, J = 7.2 Hz, 3H). LCMS m/z [M + H]⁺ |
| | | 472. |
| A162 | A2 | LCMS m/z [M + H]⁺ 487. |
| A163 | A2 | LCMS m/z [M + H]⁺ 488. |
| A164 | A2 | LCMS m/z [M + H]⁺ 493. |
| A165 | A2 | LCMS m/z [M + H]⁺ 479. |
| A166 | A2 | ¹H NMR (CDCl₃ 400 MHz) δ_{H} = 8.16 (s, 1H), 8.04 (s, 1H), 7.99 (s, 1H), |
| | | 7.15-7.00 (m, 1H), 4.18-4.01 (m, 2H), 3.97-3.58 (m, 5H), 3.04-2.82 (m, |
| | | 4H), 2.73-2.41 (m, 5H), 2.22 (s, 3H), 2.17-1.87 (m, 5H), 1.23 (t, J = 7.2 |
| | | Hz, 3H). LCMS m/z [M + H]⁺ 470. |
| A167 | A2 | LCMS m/z [M + H]⁺ 473. |
| A168 | A2 | LCMS m/z [M + H]⁺ 473. |
| A169 | A2 | LCMS m/z [M + H]⁺ 483. |
| A170 | A2 | LCMS m/z [M + H]⁺ 469. |
| A171 | A2 | ¹H NMR (CDCl₃ 400 MHz) δ_{H} = 8.27 (s, 1H), 8.21 (s, 1H), 8.17 (s, 1H), |
| | | 7.14 (d, J = 10.0 Hz, 1H), 4.87 (t, J = 4.8 Hz, 1H), 4.75 (t, J = 4.8 Hz, |
| | | 1H), 4.49 (t, J = 4.8 Hz, 1H), 4.43 (t, J = 4.8 Hz, 1H), 4.10 (q, J = 7.2 |
| | | Hz, 2H), 3.93-3.83 (m, 2H), 3.82-3.78 (m, 2H), 2.96 (s, 4H), 2.64 (s, |
| | | 5H), 2.14 (s, 1H), 2.03-1.83 (m, 3H), 1.73 (s, 1H), 1.57-1.50 (m, 1H), |
| | | 1.24 (t, J = 7.2 Hz, 3H) LCMS m/z [M + H]⁺ 475. |
| A172 | A2 | LCMS m/z [M + H]⁺ 493. |
| A173 | A2 | ¹H NMR (CDCl₃ 400 MHz) δ_{H} = 8.22-8.09 (m, 2H), 7.08-7.00 (m, 1H), |
| | | 4.48-4.40 (m, 2H), 4.23 (t, J = 6.4 Hz, 2H), 4.10 (q, J = 7.2 Hz, 2H), |
| | | 3.92-3.81 (m, 2H), 3.78 (s, 2H), 2.96 (s, 4H), 2.60 (s, 4H), 2.40-2.25 |
| | | (m, 2H), 2.15-2.05 (m, 1H), 1.98-1.80 (m, 3H), 1.77-1.65 (m, 2H), |
| | | 1.59-1.49 (m, 1H), 1.24 (t, J = 7.2 Hz, 3H). LCMS m/z [M + H]⁺ 485. |
| A174 | A2 | LCMS m/z [M + H]⁺ 455. |
| A175 | A2 | LCMS m/z [M + H]⁺ 474. |
| A176 | A2 | LCMS m/z [M + H]⁺ 494. |
| A177 | A2 | ¹H NMR (CDCl₃ 400 MHz) δ_{H} = 8.66-8.60 (m, 1H), 8.53-8.46 (m, 1H), |
| | | 8.40-8.35 (m, 1H), 8.28-8.23 (m, 1H), 7.73-7.66 (m, 1H), 7.19-7.12 |
| | | (m, 1H), 4.09 (q, J = 7.2 Hz, 2H), 3.89-3.73 (m, 4H), 2.96 (s, 4H), 2.69- |
| | | 2.36 (m, 5H), 2.15-2.02 (m, 1H), 1.97-1.70 (m, 4H), 1.57-1.41 (m, |
| | | 1H), 1.23 (t, J = 7.2 Hz, 3H). LCMS m/z [M + H]⁺ 480. |
| A178 | A2 | ¹H NMR (CDCl₃ 400 MHz) δ_{H} = 8.36-8.33 (m, 1H), 8.32-8.28 (m, 1H), |
| | | 7.87-7.82 (m, 1H), 7.68-7.61 (m, 1H), 7.25-7.21 (m, 1H), 4.06 (q, |
| | | J = 7.2 Hz, 2H), 3.84-3.71 (m, 4H), 2.94-2.76 (m, 4H), 2.45-2.31 (m, |
| | | 1H), 2.23-2.03 (m, 4H), 1.99-1.81 (m, 2H), 1.79-1.73 (m, 2H), 1.62- |
| | | 1.32 (m, 3H), 1.22 (t, J = 7.2 Hz, 3H). LCMS m/z [M + H]⁺ 480. |
| A179 | A2 | LCMS m/z [M + H]⁺ 458. |
| A180 | A2 | LCMS m/z [M + H]⁺ 488. |
| A181 | A2 | LCMS m/z [M + H]⁺ 455. |
| A182 | A2 | LCMS m/z [M + H]⁺ 464. |
| A183 | A2 | LCMS m/z [M + H]⁺ 481. |
| A184 | A2 | LCMS m/z [M + H]⁺ 459. |
| A185 | A2 | ¹H NMR (CDCl₃ 400 MHz) δ_{H} = 9.24 (s, 2H), 8.27 (d, J = 2.0 Hz, 1H), |
| | | 7.16 (dd, J = 1.6, 10.0 Hz, 1H), 4.09 (q, J = 7.2 Hz, 2H), 3.89-3.81 (m, |
| | | 2H), 3.78 (s, 2H), 2.92 (br s, 4H), 2.79 (s, 3H), 2.52 (br s, 4H), 2.10 (br |
| | | dd, J = 7.2, 12.4 Hz, 1H), 2.02-1.80 (m, 4H), 1.61-1.45 (m, 2H), 1.23 |
| | | (t, J = 7.2 Hz, 3H). LCMS m/z [M + H]⁺ 455. |
| A186 | A2 | ¹H NMR (CDCl₃ 400 MHz) δ_{H} = 9.10 (d, J = 2.0 Hz, 1H), 8.23 (d, J = 2.0 |
| | | Hz, 1H), 8.08 (dd, J = 2.0, 8.0 Hz, 1H), 7.23 (d, J = 8.0 Hz, 1H), 7.10 |
| | | (dd, J = 2.0, 10.0 Hz, 1H), 4.09 (q, J = 7.2 Hz, 2H), 3.92-3.73 (m, 4H), |
| | | 3.05-2.77 (m, 4H), 2.61 (s, 4H), 2.55-2.41 (m, 3H), 2.16-2.03 (m, |
| | | 1H), 1.99-1.63 (m, 5H), 1.56-1.44 (m, 1H), 1.23 (t, J = 7.2 Hz, 3H). |
| | | LCMS m/z [M + H]⁺ 454. |
| A187 | A2 | LCMS m/z [M + H]⁺ 446. |
| A188 | A2 | ¹H NMR (CDCl₃ 400 MHz) δ_{H} = 8.75 (d, J = 2.0 Hz, 1H), 8.23-8.18 (m, |
| | | 1H), 8.11 (dd, J = 2.0, 8.8 Hz, 1H), 7.10 (dd, J = 2.0, 10.0 Hz, 1H), 6.81 |
| | | (d, J = 8.8 Hz, 1H), 4.09 (q, J = 7.2 Hz, 2H), 3.99 (s, 3H), 3.90-3.82 |
| | | (m, 2H), 3.77 (s, 2H), 2.97 (br s, 4H), 2.64 (br s, 4H), 2.13 (br dd, J = |
| | | 7.2, 12.8 Hz, 1H), 2.05-1.54 (m, 6H), 1.23 (t, J = 7.2 Hz, 3H). LCMS |
| | | m/z [M + H]⁺ 470. |
| A189 | A2 | LCMS m/z [M + H]⁺ 506. |
| A190 | A2 | ¹H NMR (CDCl₃ 400 MHz) δ_{H} = 8.26 (s, 1H), 8.15 (d, J = 2.0 Hz, 1H), |
| | | 8.04 (s, 1H), 7.13 (dd, J = 2.4 Hz, 10.0 Hz, 1H), 4.11 (q, J = 7.2 Hz, |
| | | 2H), 3.96 (s, 3H), 3.93-3.88 (m, 1H), 3.87-3.84 (m, 1H), 3.83-3.78 (m, |
| | | 2H), 2.97 (br s, 4H), 2.64 (br s, 5H), 2.15 (br dd, J = 7.2 Hz, 12.8 Hz, |
| | | 1H), 2.01-1.81 (m, 3H), 1.78-1.69 (m, 1H), 1.56 (br s, 1H), 1.25 (t, J = |
| | | 7.2 Hz, 3H). LCMS m/z [M + H]⁺ 443. |
| A191 | A2 | ¹H NMR (CD₃OD 400 MHz) δ_{H} = 8.40 (s, 1H), 8.21(s, 1H), 8.13 (d, J = |
| | | 2.4 Hz, 1H), 7.45-7.38 (dd, J = 2.4 Hz, 10 Hz, 1H), 4.08 (q, J = 7.2 Hz, |
| | | 2H), 3.96-3.82 (m, 4H), 3.00-2.98 (m, 4H), 2.74-2.62 (m, 5H), 2.23-2.16 |
| | | (m, 1H), 2.00-1.88 (m, 3H), 1.78-1.72 (m, 1H), 1.64 (s, 9H), 1.58-1.43 |
| | | (m, 1H), 1.25 (t, J = 7.2 Hz, 3H). LCMS m/z [M + H]⁺ 485. |
| A192 | A2 | ¹H NMR (CDCl₃ 400 MHz) δ_{H} = 8.23-8.20 (m, 1H), 8.08-8.03 (m, 1H), |
| | | 7.87 (s, 1H), 7.86 (s, 1H), 7.09 (dd, J = 2.0 Hz, 10 Hz, 1H), 4.43 (s, 2H), |
| | | 4.09 (q, J = 7.2 Hz, 2H), 3.89-3.79 (m, 2H), 3.79-3.75 (m, 2H), 3.23 (s, |
| | | 3H), 2.88 (br s, 4H), 2.63-2.28 (m, 5H), 2.13-2.03 (m, 1H), 1.97-1.76 |
| | | (m, 3H), 1.68-1.63 (m, 1H), 1.50-1.45 (m, 1H), 1.23 (t, J = 7.2 Hz, 3H). |
| | | LCMS m/z [M + H]⁺ 508. |
| A193 | A2 | ¹H NMR (CDCl₃ 400 MHz) δ_{H} = 8.08 (d, J = 2.0 Hz, 1H), 6.99 (dd, J = |
| | | 2.4 Hz, 10.4 Hz, 1H), 6.36 (br s, 1H), 4.33 (d, J = 2.8 Hz, 2H), 4.10 (q, |
| | | J = 7.2 Hz, 2H), 3.95-3.72 (m, 6H), 3.03 (br s, 4H), 2.70-2.49 (m, 7H), |
| | | 2.14 (dd, J = 7.2 Hz, 12.8 Hz, 1H), 2.02-1.79 (m, 3H), 1.77-1.71 (m, |
| | | 1H), 1.62-1.49 (m, 1H), 1.23 (t, J = 7.2 Hz, 3H). LCMS m/z [M + H]⁺ 445. |
| A194 | A2 | ¹H NMR (CDCl₃ 400 MHz) δ_{H} = 8.54 (s, 1H), 8.38 (s, 1H), 8.20 (s, 1H), |
| | | 7.26-7.18 (m, 2H), 4.13-4.07 (q, J = 6.8 Hz, 2H), 3.91-3.80 (m, 4H), |
| | | 3.00 (br s, 4H), 2.70 (br s, 5H), 2.16 (m, 1H), 1.97-1.86 (m, 5H), 1.24 (t, |
| | | J = 7.2 Hz, 3H). LCMS m/z [M + H]⁺ 479. |
| A195 | A2 | ¹H NMR (CDCl₃ 400 MHz) δ_{H} = 8.28 (s, 1H), 8.20-8.07 (m, 2H), 7.13 |
| | | (dd, J = 2.4 Hz, 10.0 Hz, 1H), 4.62-4.46 (m, 1H), 4.10 (q, J = 7.2 Hz, |
| | | 2H), 3.94-3.76 (m, 4H), 2.97 (br s, 4H), 2.63 (br s, 5H), 2.19-2.09 (m, |
| | | 1H), 2.00-1.79 (m, 3H), 1.79-1.68 (m, 1H), 1.64-1.63 (m, 1H), 1.56 (d, |
| | | J = 6.4 Hz, 6H), 1.24 (t, J = 7.2 Hz, 3H). LCMS m/z [M + H]⁺ 471. |
| A196 | A2 | ¹H NMR (CDCl₃ 400 MHz) δ_{H} = 8.18 (s, 1H), 7.59 (d, J = 1.6 Hz, 1H), |
| | | 7.07-7.04 (m, 2H), 6.70 (s, 1H), 5.71 (s, 2H), 4.09 (q, J = 7.2 Hz, 2H), |
| | | 3.88-3.84 (m, 2H), 3.77 (s, 2H), 3.41 (t, J = 8.8 Hz, 2H), 2.88 (s, 4H), |
| | | 2.54-2.45 (m, 5H), 2.10 (s, 1H), 1.90-1.85 (m, 3H), 1.66 (m, 1H), 1.23 (t, |
| | | J = 7.2 Hz, 3H), 0.77-0.69 (m, 2H), 0.09 (s, 9H). LCMS m/z [M + H]⁺ 559. |
| A197 | A2 | ¹H NMR (CDCl₃ 400 MHz) δ_{H} = 8.07 (d, J = 2.4 Hz, 1H), 6.99 (br d, J = |
| | | 8.4 Hz, 1H), 6.37 (br s, 1H), 4.10 (q, J = 7.2 Hz, 2H), 3.93-3.75 (m, 4H), |
| | | 3.52-2.94 (m, 6H), 2.94-2.36 (m, 10H), 2.20-2.10 (m, 1H), 1.99-1.89 (m, |
| | | 2H), 1.89-1.62 (m, 4H), 1.52-1.47 (m, 1H), 1.24 (t, J = 7.2 Hz, 3H). |
| | | LCMS m/z [M + H]⁺ 458. |
| A198 | A2 | ¹H NMR (CDCl₃ 400 MHz) δ_{H} = 8.26 (d, J = 2.0 Hz, 1H), 7.87 (d, J = 2.8 |
| | | Hz, 1H), 7.32 (t, J = 20.8 Hz, 1H), 7.17-7.14 (m, 2H), 4.10 (q, J = 7.2 |
| | | Hz, 2H), 3.92-3.70 (m, 4H), 3.07-2.86 (m, 4H), 2.77-2.43 (m, 5H), 2.16- |
| | | 2.14 (m, 1H), 2.04-1.68 (m, 5H), 1.23 (t, J = 7.2 Hz, 3H). LCMS m/z |
| | | [M + H]⁺ 479. |
| A199 | A2 | ¹H NMR (CDCl₃ 400 MHz) δ_{H} = 8.22 (s, 1H), 7.38 (s, 1H), 7.08 (dd, J = |
| | | 2.4 Hz, 10.4 Hz, 1H), 6.97 (s, 1H), 4.09 (q, J = 7.2 Hz, 2H), 3.90-3.75 |
| | | (m, 4H), 3.05-2.86 (m, 4H), 2.71-2.40 (m, 5H), 2.12 (dd, J = 6.8 Hz, |
| | | 12.4 Hz, 1H), 1.98-1.80 (m, 3H), 1.71-1.66 (br s, 1H), 1.59-1.48 (m, |
| | | 1H), 1.23 (t, J = 7.2 Hz, 3H). LCMS m/z [M + H]⁺ 446. |
| A200 | A2 | ¹H NMR (CDCl₃ 400 MHz) δ_{H} = 8.21 (d, J = 2.4 Hz, 1H), 7.55 (t, J = 2.0 |
| | | Hz, 1H), 7.06 (dd, J = 2.4 Hz, 10.4 Hz, 1H), 6.82 (d, J = 2.4 Hz, 1H), |
| | | 4.15-4.01 (m, 2H), 3.92-3.76 (m, 4H), 2.97 (s, 4H), 2.57 (s, 5H), 2.20- |
| | | 2.01 (m, 1H), 1.97-1.76 (m, 4H), 1.65 (s, 9H), 1.57-1.43 (m, 1H), 1.23 |
| | | (t, J = 7.2 Hz, 3H). LCMS m/z [M + H]⁺ 485. |
| A201 | A2 | ¹H NMR (CDCl₃ 400 MHz) δ_{H} = 12.70 (s, 1H), 8.19 (d, J = 2.0 Hz, 1H), |
| | | 7.60 (d, J = 9.2 Hz, 1H), 7.07 (dd, J = 2.0 Hz, 10.0 Hz, 1H), 6.51 (d, J = |
| | | 9.2 Hz, 1H), 4.09 (q, J = 7.2 Hz, 2H), 3.90-3.74 (m, 4H), 2.89 (br s, 4H), |
| | | 2.70-2.38 (m, 5H), 2.33 (s, 3H), 2.12-2.07 (m, 1H), 1.99-1.81 (m, 3H), |
| | | 1.76-1.67 (m, 1H), 1.55-1.42 (m, 1H), 1.23 (t, J = 7.2 Hz, 3H). LCMS |
| | | m/z [M + H]⁺ 470. |
| A202 | A2 | ¹H NMR (CDCl₃ 400 MHz) δ_{H} = 8.14 (d, J = 2.0 Hz, 1H), 7.97 (s, 1H), |
| | | 7.91 (s, 1H), 7.06 (dd, J = 2.4 Hz, 10.0 Hz, 1H), 4.09 (q, J = 7.2 Hz, |
| | | 2H), 3.94-3.74 (m, 4H), 3.62 (s, 3H), 2.94 (br s, 4H), 2.77-2.27 (m, 5H), |
| | | 2.21 (s, 3H), 2.11 (br dd, J = 7.2 Hz, 12.8 Hz, 1H), 1.97-1.78 (m, 3H), |
| | | 1.75-1.65 (m, 1H), 1.59-1.42 (m, 1H), 1.23 (t, J = 7.2 Hz, 3H). LCMS |
| | | m/z [M + H]⁺ 484. |
| A203 | A2 | ¹H NMR (CDCl₃ 400 MHz) δ_{H} = 8.30-8.10 (m, 1H), 7.52 (dd, J = 2.0 Hz, |
| | | 6.8 Hz, 1H), 7.36 (br d, J = 4.8 Hz, 1H), 7.15-6.97 (m, 1H), 6.23 (t, J = |
| | | 6.8 Hz, 1H), 4.09 (q, J = 7.2 Hz, 2H), 3.90-3.79 (m, 2H), 3.75 (s, 2H), |
| | | 3.62 (s, 3H), 2.93 (br s, 4H), 2.64-2.23 (m, 5H), 2.08 (br d, J = 12.4 Hz, |
| | | 1H), 1.97-1.76 (m, 3H), 1.72-1.64 (m, 1H), 1.53-1.43 (m, 1H), 1.23 (t, |
| | | J = 7.2 Hz, 3H). LCMS m/z [M + H]⁺ 470. |
| A204 | A2 | ¹H NMR (CDCl₃ 400 MHz) δ_{H} = 8.28 (s, 1H), 8.22 (d, J = 2.0 Hz, 1H), |
| | | 8.07 (s, 1H), 7.34 (d, J = 2.0 Hz, 1H), 4.10 (q, J = 7.2 Hz, 2H), 3.95 (s, |
| | | 3H), 3.92-3.83 (m, 2H), 3.82-3.77 (m, 2H), 3.13-2.87 (m, 4H), 2.86-2.41 |
| | | (m, 5H), 2.17-2.12 (m, 1H), 2.01-1.83 (m, 3H), 1.79-1.70 (m, 1H), 1.59- |
| | | 1.52 (m, 1H), 1.24 (t, J = 7.2 Hz, 3H). LCMS m/z [M + H]⁺ 459. |
| A205 | A2 | ¹H NMR (CDCl₃ 400 MHz) δ_{H} = 12.11-11.91 (m, 1H), 8.18 (s, 1H), 7.09- |
| | | 7.01 (m, 2H), 6.55 (s, 1H), 4.09 (q, J = 7.2 Hz, 2H), 3.90-3.80 (m, 2H), |
| | | 3.78 (s, 2H), 2.98 (br s, 4H), 2.59 (br s, 5H), 2.39 (s, 3H), 2.12 (br dd, |
| | | J = 6.8, 12.0 Hz, 1H), 1.96-1.81 (m, 3H), 1.72-1.67 (m, 1H), 1.54-1.47 (m, |
| | | 1H), 1.23 (t, J = 7.2 Hz, 3H). LCMS m/z [M + H]⁺ 470. |
| A206 | A2 | ¹H NMR (CDCl₃ 400 MHz) δ_{H} = 13.00-12.71 (m, 1H), 8.17 (d, J = 2.0 |
| | | Hz, 1H), 8.09 (dd, J = 1.6 Hz, 11.6 Hz, 1H), 8.03 (s, 1H), 7.12 (dd, J = |
| | | 2.4 Hz, 10.0 Hz, 1H), 4.09 (q, J = 7.2 Hz, 2H), 3.90-3.81 (m, 2H), 3.81- |
| | | 3.76 (m, 2H), 2.95 (br s, 4H), 2.74-2.40 (m, 5H), 2.13 (dd, J = 7.2 Hz, |
| | | 12.8 Hz, 1H), 1.99-1.80 (m, 3H), 1.72-1.66 (m, 1H), 1.56-1.46 (m, 1H), |
| | | 1.23 (t, J = 7.2 Hz, 3H). LCMS m/z [M + H]⁺ 474. |
| A207 | A2 | ¹H NMR (CDCl₃ 400 MHz) δ_{H} = 8.23 (d, J = 2.0 Hz, 1H), 8.14 (dd, J = |
| | | 2.0 Hz, 9.2 Hz, 1H), 8.04 (s, 1H), 7.31 (s, 1H), 6.60 (d, J = 9.6 Hz, 1H), |
| | | 4.10 (q, J = 7.2 Hz, 2H), 3.92-3.76 (m, 4H), 3.62 (s, 3H), 3.07-2.82 (m, |
| | | 4H), 2.77-2.28 (m, 5H), 2.15-2.10 (m, 1H), 2.01-1.79 (m, 3H), 1.76-1.68 |
| | | (m, 1H), 1.57-1.44 (m, 1H), 1.23 (t, J = 7.2 Hz, 3H). LCMS m/z [M + H]⁺ |
| | | 486. |
| A208 | A2 | ¹H NMR (CDCl₃ 400 MHz) δH = 8.32 (d, J = 2.0 Hz, 1H), 7.40 (d, J = |
| | | 2.0 Hz, 1H), 7.33 (d, J = 1.6 Hz, 1H), 7.02 (d, J = 2.0 Hz, 1H), 4.12 (q, |
| | | J = 7.2 Hz, 2H), 4.00 (s, 3H), 3.92-3.82 (m, 2H), 3.81-3.76 (m, 2H), 2.98 |
| | | (s, 4H), 2.78-2.42 (m, 5H), 2.16-2.11 (m, 1H), 1.99-1.81 (m, 3H), 1.79- |
| | | 1.68 (m, 1H), 1.57-1.48 (m, 1H), 1.23 (t, J = 7.2 Hz, 3H). LCMS m/z |
| | | [M + H]⁺ 459. |
| A209 | A2 | ¹H NMR (CDCl₃ 400 MHz) δ_{H} = 8.27 (s, 1H), 7.37-7.26 (m, 3H), 6.70 |
| | | (dd, J = 1.6 Hz, 7.2 Hz, 1H), 4.09 (q, J = 7.2 Hz, 2H), 3.93-3.70 (m, |
| | | 4H), 3.59 (s, 3H), 2.98 (br s, 4H), 2.55 (br s, 5H), 2.20-2.04 (m, 1H), |
| | | 2.02-1.77 (m, 3H), 1.76-1.67 (m, 1H), 1.55-1.44 (m, 1H), 1.23 (t, J = 7.2 |
| | | Hz, 3H). LCMS m/z [M + H]⁺ 486. |
| A210 | A2 | ¹H NMR (CDCl₃ 400 MHz) δ_{H} = 12.41 (br s, 1H), 8.20 (dd, J = 2.0, 9.6 |
| | | Hz, 1H), 8.17 (d, J = 2.4 Hz, 1H), 8.08 (d, J = 2.0 Hz, 1H), 7.10 (dd, J = |
| | | 2.4, 10.0 Hz, 1H), 6.65 (d, J = 9.6 Hz, 1H), 4.10 (q, J = 7.2 Hz, 2H), |
| | | 3.94-3.76 (m, 4H), 2.95 (br s, 4H), 2.74-2.40 (m, 5H), 2.21-2.07 (m, |
| | | 1H), 2.01-1.79 (m, 3H), 1.75-1.70 (m, 1H), 1.56-1.46 (m, 1H), 1.24 (t, |
| | | J = 7.2 Hz, 3H). LCMS m/z [M + H]⁺ 456. |
| A211 | A2 | ¹H NMR (CDCl₃ 400 MHz) δ_{H} = 9.55 (br s, 1H), 8.23 (s, 1H), 7.55 (s, |
| | | 1H), 7.15 (d, J = 8.4 Hz, 1H), 4.10 (q, J = 6.8 Hz, 2H), 3.96-3.70 (m, 4H), |
| | | 2.97 (br s, 4H), 3.13-2.81 (m, 1H), 2.73-2.37 (m, 5H), 2.18-2.07 (m, |
| | | 1H), 2.02-1.80 (m, 3H), 1.77-1.70 (m, 1H), 1.56-1.46 (m, 1H), 1.24 (br |
| | | t, J = 7.2 Hz, 3H). LCMS m/z [M + H]⁺ 473. |
| A212 | A2 | ¹H NMR (CDCl₃ 400 MHz) δ_{H} = 8.92 (d, J = 2.0 Hz, 1H), 8.35 (d, J = 2.0 |
| | | Hz, 1H), 8.25 (d, J = 2.0 Hz, 1H), 7.37 (d, J = 2.0 Hz, 1H), 4.09 (q, J = |
| | | 7.2 Hz, 2H), 3.89-3.81 (m, 2H), 3.81-3.76 (m, 2H), 2.94 (br t, J = 4.8 |
| | | Hz, 4H), 2.66-2.47 (m, 5H), 2.12 (dd, J = 6.8 Hz, 12.4 Hz, 1H), 1.98- |
| | | 1.79 (m, 3H), 1.74-1.69 (m, 1H), 1.58-1.48 (m, 1H), 1.23 (t, J = 7.2 Hz, |
| | | 3H). LCMS m/z [M + H]⁺ 462. |
| A213 | A2 | ¹H NMR (CDCl₃ 400 MHz) δ_{H} = 8.49 (d, J = 1.6 Hz, 1H), 8.36 (d, J = 2.4 |
| | | Hz, 1H), 8.05 (d, J = 1.6 Hz, 1H), 7.45-7.35 (m, 1H), 4.11 (q, J = 7.2 |
| | | Hz, 2H), 3.97-3.72 (m, 4H), 2.99 (s, 4H), 2.79 (s, 5H), 2.18 (dd, J = 7.2 |
| | | Hz, 12.8 Hz, 1H), 2.03-1.83 (m, 3H), 1.77 (d, J = 10.4 Hz, 1H), 1.66- |
| | | 1.60 (m, 1H), 1.24 (t, J = 7.2 Hz, 3H). LCMS m/z [M + H]⁺ 446. |
| A214 | A2 | ¹H NMR (CDCl₃ 400 MHz) δ_{H} = 9.25 (d, J = 14.4 Hz, 2H), 8.21 (d, J = |
| | | 2.4 Hz, 1H), 7.16 (dd, J = 2.4 Hz, 10.0 Hz, 1H), 4.07-4.05 (m, 1H), 4.09 |
| | | (q, J = 7.2 Hz, 1H), 3.91-3.73 (m, 4H), 2.92 (s, 4H), 2.59 (s, 5H), 2.12 |
| | | (dd, J = 6.8 Hz, 12.8 Hz, 1H), 1.99-1.82 (m, 3H), 1.71 (s, 1H), 1.53 (s, |
| | | 1H), 1.23 (t, J = 7.2 Hz, 3H). LCMS m/z [M + H]⁺ 446. |
| A215 | A3 | ¹H NMR (CDCl₃ 400 MHz) δ_{H} = 7.94 (d, J = 4.4 Hz, 1H), 7.08 (d, J = 7.6 |
| | | Hz, 1H), 6.80 (dd, J = 4.8 Hz, 7.2 Hz, 1H), 4.10 (q, J = 7.6 Hz, 2H), |
| | | 3.97-3.85 (m, 2H), 3.83-3.76 (m, 2H), 3.49-3.05 (m, 8H), 2.95-2.40 (m, |
| | | 5H), 2.25-2.16 (m, 1H), 2.04-1.95 (m, 2H), 1.91-1.80 (m, 2H), 1.66-1.54 |
| | | (m, 7H), 1.24 (t, J = 7.2 Hz, 3H). LCMS m/z [M + H]⁺ 428. |
| A216 | A4 | ¹H NMR (CDCl₃ 400 MHz) δ_{H} = 7.91 (d, J = 4.4 Hz, 1H), 7.08 (d, J = 7.6 |
| | | Hz, 1H), 6.81 (dd, J = 4.8 Hz, 7.6 Hz, 1H), 4.15-3.97 (m, 4H), 3.93-3.77 |
| | | (m, 4H), 3.57-3.27 (m, 4H), 3.24-2.95 (m, 4H), 2.88-2.52 (m, 7H), 2.28- |
| | | 2.03 (m, 4H), 1.99-1.79 (m, 4H), 1.78-1.67 (m, 2H), 1.24 (t, J = 7.2 Hz, |
| | | 3H). LCMS m/z [M + H]⁺ 458. |
| A217 | A4 | ¹H NMR (CDCl₃ 400 MHz) δ_{H} = 7.92 (dd, J = 1.2 Hz, 4.8 Hz, 1H), 7.12 |
| | | (dd, J = 5.6 Hz, 8.0 Hz, 1H), 6.85 (dd, J = 4.8 Hz, 7.6 Hz, 1H), 4.10 (q, |
| | | J = 6.8 Hz, 2H), 3.95-3.76 (m, 6H), 3.39-2.91 (m, 6H), 2.86-2.45 (m, |
| | | 6H), 2.21-2.04 (m, 3H), 2.00-1.83 (m, 5H), 1.79-1.70 (m, 1H), 1.56-1.44 |
| | | (m, 1H), 1.24 (t, J = 7.2 Hz, 3H). LCMS m/z [M + H]⁺ 453. |
| A218 | A6 | ¹H NMR (CDCl₃ 400 MHz) δ_{H} = 8.54 (s, 1H), 8.17 (s, 1H), 7.87 (dd, J = |
| | | 4.4 Hz, 8.8 Hz, 1H), 7.42 (dt, J = 3.2 Hz, 8.4 Hz, 1H), 7.04 (dd, J = 2.4 |
| | | Hz, 10.4 Hz, 1H), 4.02 (q, J = 7.2 Hz, 2H), 3.7-3.8 (m, 2H), 3.7-3.7 (m, |
| | | 2H), 2.82 (br s, 4H), 2.4-2.6 (m, 1H), 2.3-2.4 (m, 4H), 2.02 (br dd, J = |
| | | 7.2 Hz, 12.4 Hz, 1H), 1.7-1.9 (m, 3H), 1.6-1.7 (m, 1H), 1.4-1.5 (m, 1H), |
| | | 1.16 (t, J = 6.8 Hz, 3H). LCMS m/z [M + H]⁺ 458. |
| A219 | A6 | ¹H NMR (CDCl₃ 400 MHz) δ_{H} = 8.98 (s, 1H), 8.73 (s, 1H), 8.59 (d, J = |
| | | 2.0 Hz, 1H), 8.29 (s, 1H), 7.25-7.19 (m, 1H), 4.19-4.02 (m, 5H), 3.95 (d, |
| | | J = 8.8 Hz, 1H), 3.34-3.15 (m, 2H), 3.05-2.88 (m, 4H), 2.85-2.67 (m, |
| | | 3H), 2.43-2.31 (m, 1H), 2.13-2.01 (m, 1H), 1.87-1.70 (m, 2H), 1.67-1.61 |
| | | (m, 2H), 1.23 (t, J = 7.2 Hz, 3H). LCMS m/z [M + H]⁺ 485. Peak 1, |
| | | stereochemistry arbitrarily assigned. |
| A220 | A6 | ¹H NMR (CDCl₃ 400 MHz) δ_{H} = 8.98 (s, 1H), 8.73 (s, 1H), 8.59 (d, J = |
| | | 2.0 Hz, 1H), 8.29 (s, 1H), 7.25-7.19 (m, 1H), 4.19-4.01 (m, 5H), 3.94 (d, |
| | | J = 8.8 Hz, 1H), 3.36-3.13 (m, 2H), 3.07-2.85 (m, 4H), 2.84-2.66 (m, |
| | | 3H), 2.42-2.29 (m, 1H), 2.14-1.97 (m, 1H), 1.90-1.72 (m, 2H), 1.67-1.63 |
| | | (m, 2H), 1.22 (t, J = 7.2 Hz, 3H). LCMS m/z [M + H]⁺ 485. Peak 2, |
| | | stereochemistry arbitrarily assigned. |
| A221 | A6 | ¹H NMR (CDCl₃ 400 MHz) δ_{H} = 9.24 (s, 1H), 8.72 (br s, 1H), 8.58-8.52 |
| | | (m, 1H), 8.40 (s, 1H), 7.49-7.36 (m, 1H), 4.14-4.04 (m, 2H), 3.89-3.78 |
| | | (m, 2H), 3.76 (s, 2H), 2.92 (br s, 4H), 2.58-2.52 (m, 1H), 2.48 (br s, 4H), |
| | | 2.12-2.01 (m, 1H), 1.94-1.79 (m, 3H), 1.70-1.62 (m, 1H), 1.52-1.43 (m, |
| | | 1H), 1.24 (t, J = 7.2 Hz, 3H). LCMS m/z [M + H]⁺ 458. |
| A222 | A6 | ¹H NMR (CDCl₃ 400 MHz) δ_{H} = 8.98 (s, 1H), 8.71 (br s, 1H), 8.60 (br s, |
| | | 1H), 8.44-8.20 (m, 1H), 7.23 (br s, 1H), 4.08 (q, J = 7.2 Hz, 2H), 3.95- |
| | | 3.75 (m, 4H), 3.20 (br s, 4H), 2.85-2.53 (m, 3H), 2.36 (br s, 1H), 2.21- |
| | | 1.96 (m, 3H), 1.80 (br s, 4H), 1.72-1.66 (m, 1H), 1.38 (br s, 1H), 1.22 (t, |
| | | J = 7.2 Hz, 3H). LCMS m/z [M + H]⁺ 467. Peak 1, stereochemistry |
| | | arbitrarily assigned. |
| A223 | A6 | ¹H NMR (CDCl₃ 400 MHz) δ_{H} = 8.98 (d, J = 1.6 Hz, 1H), 8.72 (dd, J = |
| | | 1.6 Hz, 2.4 Hz, 1H), 8.58 (d, J = 2.8 Hz, 1H), 8.27 (d, J = 2.0 Hz, 1H), |
| | | 7.21 (dd, J = 2.4 Hz, 10.4 Hz, 1H), 4.15-4.02 (m, 1H), 4.08 (s, 1H), |
| | | 3.91-3.72 (m, 4H), 3.19 (s, 2H), 2.93 (dd, J = 4.0 Hz, 10.0 Hz, 2H), |
| | | 2.78-2.63 (m, 3H), 2.03-1.88 (m, 2H), 1.83-1.73 (m, 4H), 1.72-1.62 (m, |
| | | 2H), 1.60-1.53 (m, 2H), 1.30-1.16 (m, 3H). LCMS m/z [M + H]⁺ 467. Peak |
| | | 2, stereochemistry arbitrarily assigned. |
| A224 | A6 | ¹H NMR (CDCl₃ 400 MHz) δ_{H} = 9.22 (d, J = 1.2 Hz, 1H), 8.76-8.64 (m, |
| | | 1H), 8.54 (d, J = 2.4 Hz, 1H), 8.31 (s, 1H), 7.17 (d, J = 10.0 Hz, 1H), |
| | | 4.09 (q, J = 7.2 Hz, 2H), 3.61 (d, J = 13.2 Hz, 4H), 2.91 (br s, 4H), 2.55 |
| | | (br s, 4H), 2.20 (s, 1H), 1.95 (d, J = 12.8 Hz, 2H), 1.79 (br s, 2H), 1.52- |
| | | 1.37 (m, 2H), 1.35-1.25 (m, 2H), 1.23(t, J = 7.2 Hz,, 3H). LCMS m/z |
| | | [M + H]⁺ 455. |
| A225 | A6 | ¹H NMR (CDCl₃ 400 MHz) δ_{H} = 9.21 (s, 1H), 8.73 (s, 1H), 8.56 (d, J = |
| | | 2.4 Hz, 1H), 8.33 (s, 1H), 7.20-7.16 (m, 1H), 4.14-4.04 (m, 5H), 3.99- |
| | | 3.97 (m, 1H), 3.03-2.82 (m, 7H), 2.64-2.47 (m, 4H), 2.05-2.03 (m, 1H), |
| | | 1.64-1.63 (m, 1H), 1.23 (t, J = 7.2 Hz, 3H). LCMS m/z [M + H]⁺ 459. |
| A226 | A6 | ¹H NMR (CDCl₃ 400 MHz) δ_{H} = 8.78 (s, 1H), 8.46-8.12 (m, 2H), 7.13 (d, |
| | | J = 10.0 Hz, 1H), 4.09 (q, J = 7.2 Hz, 2H), 4.03 (s, 3H), 3.90-3.77 (m, |
| | | 4H), 3.01-2.83 (m, 4H), 2.68-2.43 (m, 5H), 2.14-2.04 (m, 1H), 1.94-1.81 |
| | | (m, 3H), 1.76-1.68 (m, 1H), 1.59-1.52 (m, 1H), 1.26-1.23 (t, J = 7.2 Hz, |
| | | 3H). LCMS m/z [M + H]⁺ 471. |
| A227 | A6 | ¹H NMR (CDCl₃ 400 MHz) δ_{H} = 8.75 (d, J = 4.4 Hz, 1H), 8.24 (d, J = |
| | | 2.0 Hz, 1H), 7.87 (d, J = 8.0 Hz, 1H), 7.76 (dt, J = 1.6 Hz, 7.6 Hz, 1H), |
| | | 7.30-7.26 (m, 1H), 7.08 (dd, J = 2.4 Hz, 10.4 Hz, 1H), 4.09 (q, J = 7.2 |
| | | Hz, 2H), 3.89-3.79 (m, 2H), 3.79-3.73 (m, 2H), 2.98-2.81 (m, 4H), 2.61- |
| | | 2.32 (m, 5H), 2.08 (dd, J = 6.8 Hz, 12.8 Hz, 1H), 1.96-1.75 (m, 3H), |
| | | 1.72-1.68 (m, 1H), 1.55-1.40 (m, 1H), 1.22 (t, J = 7.2 Hz, 3H). LCMS |
| | | m/z [M + H]⁺ 440. |
| A228 | A6 | ¹H NMR (CDCl₃ 400 MHz) δ_{H} = 8.57 (s, 1H), 8.22 (d, J = 2.0 Hz, 1H), |
| | | 7.79 (d, J = 8.0 Hz, 1H), 7.56 (dd, J = 1.6 Hz, 8.0 Hz, 1H), 7.06 (dd, J = |
| | | 2.4 Hz, 10.4 Hz, 1H), 4.09 (q, J = 7.2 Hz, 2H), 3.89-3.73 (m, 4H), 2.99- |
| | | 2.80 (m, 4H), 2.59-2.34 (m, 8H), 2.09 (dd, J = 7.2 Hz, 12.4 Hz, 1H), |
| | | 1.98-1.70 (m, 4H), 1.57-1.41 (m, 1H), 1.23 (t, J = 7.2 Hz, 3H). LCMS |
| | | m/z [M + H]⁺ 454. |
| A229 | A6 | ¹H NMR (CDCl₃ 400 MHz) δ_{H} = 7.56 (d, J = 2.8 Hz, 1H), 6.83 (dd, J = |
| | | 2.8 Hz, 9.6 Hz, 1H), 5.35-5.20 (m, 1H), 4.10 (q, J = 7.2 Hz, 2H), 4.00- |
| | | 3.72 (m, 6H), 3.70-3.58 (m, 2H), 3.30-3.00 (m, 4H), 2.80-2.35 (m, 5H), |
| | | 2.22-2.02 (m, 3H), 2.02-1.74 (m, 6H), 1.61-1.52 (m, 1H), 1.24 (t, J = 7.2 |
| | | Hz, 3H). LCMS m/z [M + H]⁺ 463. |
| A230 | A6 | ¹H NMR (CDCl₃ 400 MHz) δ_{H} = 8.25 (d, J = 1.6 Hz, 1H), 8.18 (s, 1H), |
| | | 7.17 (dd, J = 2.4 Hz, 10.4 Hz, 1H), 4.17 (s, 3H), 4.10 (q, J = 7.2 Hz, |
| | | 2H), 4.13-4.07 (m, 1H), 3.92-3.73 (m, 4H), 2.99 (br s, 4H), 2.62 (br s, |
| | | 5H), 2.19-2.06 (m, 1H), 2.01-1.79 (m, 3H), 1.77-1.67 (m, 1H), 1.53-1.44 |
| | | (m, 1H), 1.24 (t, J = 7.2 Hz, 3H). LCMS m/z [M + H]⁺ 444. |
| A231 | A6 | ¹H NMR (CDCl₃ 400 MHz) δ_{H} = 9.13 (s, 1H), 8.60 (s, 1H), 8.30 (d, J = |
| | | 2.0 Hz, 1H), 7.19-7.08 (m, 1H), 4.09 (q, J = 7.2 Hz, 2H), 3.92-3.74 (m, |
| | | 4H), 3.10-2.80(m, 4H), 2.69-2.39 (m, 8H), 2.09 (dd, J = 7.2 Hz, 12.0 Hz, |
| | | 1H), 1.99-1.79 (m, 3H), 1.74-1.67 (m, 1H), 1.57-1.42 (m, 1H), 1.23 (t, |
| | | J = 7.2 Hz, 3H). LCMS m/z [M + H]⁺ 455. |
| A232 | A6 | ¹H NMR (CDCl₃ 400 MHz) δ_{H} = 8.21 (s, 1H), 8.06 (s, 1H), 7.79 (dt, J = |
| | | 3.2 Hz, 7.2 Hz, 1H), 6.93 (dd, J = 2.0 Hz, 12.4 Hz, 1H), 4.10 (q, J = 7.2 |
| | | Hz, 2H), 3.92-3.76 (m, 4H), 3.34 (s, 4H), 2.64 (s, 5H), 2.18-2.13 (m, |
| | | 1H), 2.01-1.84 (m, 3H), 1.80-1.71 (m, 1H), 1.56-1.49 (m, 1H), 1.24 (t, |
| | | J = 7.2 Hz, 3H). LCMS m/z [M + H]⁺ 476. |
| A233 | A6 | ¹H NMR (CDCl₃ 400 MHz) δ_{H} = 7.56 (d, J = 2.8 Hz, 1H), 6.84 (dd, J = |
| | | 2.8 Hz, 9.6 Hz, 1H), 5.06-4.98 (m, 1H), 4.76 (s, 2H), 4.68 (s, 2H), 4.10 |
| | | (q, J = 7.2 Hz, 2H), 3.92-3.83 (m, 2H), 3.82-3.77 (m, 2H), 3.11 (br s, |
| | | 4H), 2.86-2.79 (m, 2H), 2.65 (br s, 5H), 2.33-2.27 (m, 2H), 2.15 (dd, J = |
| | | 7.2, 12.4 Hz, 1H), 1.99-1.89 (m, 2H), 1.89-1.82 (m, 1H), 1.74 (s, 1H), |
| | | 1.58-1.48 (m, 1H), 1.24 (t, J = 7.2 Hz, 3H). LCMS m/z [M + H]⁺ 475. |
| A234 | A6 | ¹H NMR (CDCl₃ 400 MHz) δ_{H} = 9.00 (s, 1H), 8.25 (s, 1H), 8.14-8.07 (m, |
| | | 1H), 8.03-8.00 (m, 1H), 7.13 (d, J = 10.4 Hz, 1H), 4.08 (q, J = 7.2 Hz, |
| | | 2H), 3.92-3.59 (m, 4H), 2.90 (br s, 4H), 2.62-2.35 (m, 5H), 2.11-2.06 |
| | | (m, 1H), 1.91-1.79 (m, 3H), 1.71-1.62 (m, 1H), 1.50-1.48 (m, 1H), 1.22 |
| | | (t, J = 7.2 Hz, 3H). LCMS m/z [M + H]⁺ 466. |
| A235 | A6 | ¹H NMR (CDCl₃ 400 MHz) δ_{H} = 8.82 (d, J = 1.2 Hz, 1H), 8.49 (d, J = 1.2 |
| | | Hz, 1H), 8.28 (d, J = 2.0 Hz, 1H), 7.46 (t, J = 72 Hz, 1H), 7.16 (dd, J = |
| | | 2.4 Hz, 10.4 Hz, 1H), 4.08 (q, J = 7.2 Hz, 2H), 3.94-3.68 (m, 4H), 2.91 |
| | | (t, J = 4.4 Hz, 4H), 2.68-2.36 (m, 5H), 2.09 (dd, J = 6.8 Hz, 12.8 Hz, |
| | | 1H), 1.97-1.81 (m, 3H), 1.72-1.61 (m, 1H), 1.55-1.43 (m, 1H), 1.22 (t, |
| | | J = 7.2 Hz, 3H). LCMS m/z [M + H]⁺ 507. |
| A236 | A6 | ¹H NMR (CDCl₃ 400 MHz) δ_{H} = 9.14 (s, 1H), 8.26 (s, 1H), 7.25 (s, 1H), |
| | | 4.10 (q, J = 6.8 Hz, 2H), 3.95-3.76 (m, 4H), 3.16 (br s, 4H), 2.80 (br s, |
| | | 5H), 2.20-2.15 (m, 1H), 2.04-1.78 (m, 4H), 1.59-1.44 (m, 1H), 1.24 (t, |
| | | J = 7.2 Hz, 3H). LCMS m/z [M + H]⁺ 447. |
| A237 | A6 | ¹H NMR (CDCl₃ 400 MHz) δ 9.16 (s, 1H), 8.42 (dd, J = 0.8, 4.0 Hz, 1H), |
| | | 7.58 (dd, J = 1.2 Hz, 8.0 Hz, 1H), 7.38 (dd, J = 4.8 Hz, 8.4 Hz, 1H), |
| | | 4.15-4.07 (m, 3H), 4.07-3.98 (m, 3H), 3.94-3.87 (m, 1H), 3.76 (br t, J = |
| | | 7.6 Hz, 1H), 3.24-3.01 (m, 5H), 2.88-2.59 (m, 4H), 2.38 (dd, J = 7.6 Hz, |
| | | 12.8 Hz, 1H), 2.18-2.04 (m, 1H), 1.24 (t, J = 7.2 Hz, 3H). LCMS m/z |
| | | [M + H]⁺ 431. |
| A238 | A6 | ¹H NMR (CDCl₃ 400 MHz) δ_{H} = 8.29-8.23 (m, 2H), 8.16 (d, J = 2.6 Hz, |
| | | 1H), 7.16 (d, J = 10.4 Hz, 1H), 4.08 (q, J = 7.2 Hz, 2H), 3.94 (s, 3H), |
| | | 3.88-3.79 (m, 2H), 3.74 (s, 2H), 2.87 (br s, 4H), 2.60-2.19 (m, 5H), |
| | | 2.08-2.03 (m, 1H), 1.97-1.73 (m, 4H), 1.55-1.39 (m, 1H), 1.22 (t, J = 7.2 |
| | | Hz, 3H). LCMS m/z [M + H]⁺ 471. |
| A239 | A6 | ¹H NMR (CDCl₃ 400 MHz) δ_{H} = 9.16 (s, 1H), 8.61 (s, 1H), 8.38 (s, 1H), |
| | | 7.38 (d, J = 1.6 Hz, 1H), 4.08 (q, J = 7.2 Hz, 2H), 3.89-3.73 (m, 4H), |
| | | 2.92 (br s, 4H), 2.63 (s, 3H), 2.60-2.39 (m, 5H), 2.10-2.06 (m, 1H), |
| | | 1.96-1.76 (m, 4H), 1.58-1.42 (m, 1H), 1.22 (t, J = 7.2 Hz, 3H). LCMS |
| | | m/z [M + H]⁺ 471. |
| A240 | A6 | ¹H NMR (CDCl₃ 400 MHz) δ_{H} = 8.46 (d, J = 2.4 Hz, 1H), 8.26 (d, J = 2.4 |
| | | Hz, 1H), 7.33-7.26 (m, 1H), 7.15 (dd, J = 2.4 Hz, 10.0 Hz, 1H), 4.07 (q, |
| | | J = 7.2 Hz, 2H), 3.87-3.69 (m, 4H), 2.85 (t, J = 4.4 Hz, 4H), 2.56-2.26 |
| | | (m, 5H), 2.04 (dd, J = 7.2 Hz, 12.8 Hz, 1H), 1.92-1.78 (m, 3H), 1.68- |
| | | 1.57 (m, 1H), 1.53-1.39 (m, 1H), 1.21 (t, J = 7.2 Hz, 3H). LCMS m/z |
| | | [M + H]⁺ 476. |
| A241 | A6 | ¹H NMR (CDCl₃ 400 MHz) oH = 8.27 (d, J = 2.0 Hz, 1H), 7.89 (d, J = |
| | | 8.0 Hz, 1H), 7.40 (d, J = 8.8 Hz, 1H), 7.17 (d, J = 9.6 Hz, 1H), 4.09 (q, |
| | | J = 7.2 Hz, 2H), 3.95-3.69 (m, 4H), 3.46-2.84 (m, 4H), 2.78 (s, 3H), 2.76- |
| | | 2.16 (m, 5H), 2.16-2.02 (m, 1H), 2.01-1.79 (m, 3H), 1.77-1.71 (m, 1H), |
| | | 1.60-1.44 (m, 1H), 1.23 (t, J = 7.2 Hz, 3H). LCMS m/z [M + H]⁺ 455. |
| A242 | A6 | ¹H NMR (CDCl₃ 400 MHz) δ_{H} = 9.18 (dd, J = 1.6 Hz, 4.8 Hz, 1H), 8.26 |
| | | (d, J = 2.0 Hz, 1H), 7.98 (dd, J = 1.2 Hz, 8.4 Hz, 1H), 7.63-7.46 (m, |
| | | 1H), 7.54 (dd, J = 5.2 Hz, 8.6 Hz, 1H), 7.15 (dd, J = 2.4 Hz, 10.4 Hz, |
| | | 1H), 4.06 (q, J = 7.2 Hz, 2H), 3.89-3.66 (m, 4H), 3.05-2.75 (m, 4H), |
| | | 2.59-2.29 (m, 5H), 2.05 (dd, J = 6.8 Hz, 12.4 Hz, 1H), 1.92-1.75 (m, |
| | | 3H), 1.70-1.60 (m, 1H), 1.52-1.40 (m, 1H), 1.21 (t, J = 7.2 Hz, 3H). |
| | | LCMS m/z [M + H]⁺ 441. |
| A243 | A6 | ¹H NMR (CDCl₃ 400 MHz) δ_{H} = 8.29 (d, J = 2.4 Hz, 1H), 8.23 (d, J = |
| | | 8.8 Hz, 1H), 7.88 (d, J = 8.8 Hz, 1H), 7.22 (dd, J = 2.4 Hz, 10.4 Hz, |
| | | 1H), 4.08 (q, J = 7.2 Hz, 2H), 3.89-3.71 (m, 4H), 3.10-2.80 (m, 4H), |
| | | 2.74-2.35 (m, 5H), 2.09 (dd, J = 7.2 Hz, 12.4 Hz, 1H), 1.95-1.80 (m, |
| | | 3H), 1.73-1.68 (m, 1H), 1.59-1.43 (m, 1H), 1.22 (t, J = 7.2 Hz, 3H). |
| | | LCMS m/z [M + H]⁺ 509. |
| A244 | A6 | ¹H NMR (CDCl₃ 400 MHz) δ_{H} = 9.27 (s, 1H), 8.74 (s, 1H), 8.55 (d, J = |
| | | 2.4 Hz, 1H), 8.48 (d, J = 4.4 Hz, 1H), 7.53-7.45 (m, 1H), 7.35-7.32 (m, |
| | | 1H), 4.09 (q, J = 7.2 Hz, 2H), 3.92-3.80 (m, 2H), 3.76 (s, 2H), 3.18-2.80 |
| | | (m, 4H), 2.77-2.25 (m, 5H), 2.16-2.05 (m, 1H), 1.96-1.77 (m, 3H), 1.76- |
| | | 1.68 (m, 1H), 1.54-1.42 (m, 1H), 1.23 (t, J = 7.2 Hz, 3H). LCMS m/z |
| | | [M + H]⁺ 423. |
| A245 | A6 | ¹H NMR (CDCl3 400 MHz) δH = 9.20 (d, J = 4.0 Hz, 1H), 8.40 (s, 1H), |
| | | 8.25-7.95 (m, 1H), 7.68-7.36 (m, 2H), 4.09 (q, J = 7.2 Hz, 2H), 3.93- |
| | | 3.70 (m, 4H), 2.95 (br s, 4H), 2.44 (br s, 5H), 2.20-2.12 (m, 1H), 1.98- |
| | | 1.74 (m, 3H), 1.70-1.60 (m, 1H), 1.51-1.33 (m, 1H), 1.23 (t, J = 7.2 Hz, |
| | | 3H). LCMS m/z [M + H]⁺ 457. |
| A246 | A6 | ¹H NMR (CDCl₃ 400 MHz) δ_{H} = 8.05 (d, J = 2.0 Hz, 1H), 7.85 (s, 1H), |
| | | 7.19 (dd, J = 2.4 Hz, 9.6 Hz, 1H), 4.09 (q, J = 7.2 Hz, 2H), 3.89-3.80 |
| | | (m, 2H), 3.79-3.74 (m, 2H), 2.94-2.67 (m, 4H), 2.64-2.37 (m, 8H), 2.10 |
| | | (br dd, J = 6.8 Hz, 12.4 Hz, 1H), 2.00-1.74 (m, 4H), 1.58-1.44 (m, 1H), |
| | | 1.23 (t, J = 7.2 Hz, 3H). LCMS m/z [M + H]⁺ 444. |
| A247 | A6 | ¹H NMR (CDCl₃ 400 MHz) δ_{H} = 8.14 (d, J = 2.0 Hz, 1H), 7.89 (s, 1H), |
| | | 7.42 (d, J = 2.0 Hz, 1H), 4.09 (q, J = 7.2 Hz, 2H), 3.89-3.80 (m, 2H), |
| | | 3.79-3.74 (m, 2H), 2.89-2.73 (m, 4H), 2.62-2.42 (m, 8H), 2.08 (dd, J = |
| | | 6.8, 12.4 Hz, 1H), 1.96-1.79 (m, 3H), 1.78-1.73 (m, 1H), 1.55-1.44 (m, |
| | | 1H), 1.23 (t, J = 7.2 Hz, 3H). LCMS m/z [M + H]⁺ 460. |
| A248 | A6 | ¹H NMR (CDCl₃ 400 MHz) δ_{H} = 8.71 (d, J = 5.2 Hz, 1H), 8.35 (d, J = 2.0 |
| | | Hz, 1H), 7.74 (d, J = 5.2 Hz, 1H), 7.36 (d, J = 2.0 Hz, 1H), 4.09 (q, J = |
| | | 7.2 Hz, 2H), 3.90-3.70 (m, 4H), 2.92 (t, J = 4.4 Hz, 4H), 2.84 (s, 3H), |
| | | 2.59-2.36 (m, 5H), 2.08 (dd, J = 6.8 Hz, 12.4 Hz, 1H), 1.95-1.80 (m, |
| | | 3H), 1.68-1.63 (m, 1H), 1.54-1.41 (m, 1H), 1.22 (t, J = 7.2 Hz, 3H). |
| | | LCMS m/z [M + H]⁺ 471. |
| A249 | A6 | ¹H NMR (CDCl₃ 400 MHz) δ_{H} = 9.16 (s, 1H), 8.34 (d, J = 1.6 Hz, 1H), |
| | | 7.52 (d, J = 1.6 Hz, 1H), 4.10 (q, J = 7.2 Hz, 2H), 3.98-3.84 (m, 2H), |
| | | 3.83-3.75 (m, 2H), 3.54-2.41 (m, 9H), 2.26-2.13 (m, 1H), 2.05-1.83 (m, |
| | | 3H), 1.71-1.63 (m, 1H), 1.56-1.48 (m, 1H), 1.24 (t, J = 7.2 Hz, 3H). |
| | | LCMS m/z [M + H]⁺ 463. |
| A250 | A6 | ¹H NMR(CDCl₃ 400 MHz) δ_{H} = 9.37 (s, 1H), 9.06 (s, 1H), 8.34 (d, J = |
| | | 2.0 Hz, 1H), 7.22 (dd, J = 2.0 Hz, 10.0 Hz, 1H), 4.09 (q, J = 7.2 Hz, |
| | | 2H), 3.89-3.80 (m, 2H), 3.80-3.74 (m, 2H), 2.95 (br s, 4H), 2.70-2.36 |
| | | (m, 5H), 2.12-2.07 (m, 1H), 1.98-1.78 (m, 3H), 1.77-1.69 (m, 1H), 1.58- |
| | | 1.42 (m, 1H), 1.23 (t, J = 7.2 Hz, 3H). LCMS m/z [M + H]⁺ 509. |
| A251 | A6 | ¹H NMR (CDCl3 400 MHz) δ_{H} = 8.35 (d, J = 2.0 Hz, 1H), 7.88 (d, J = |
| | | 8.4 Hz, 1H), 7.40 (d, J = 8.4 Hz, 2H), 4.09 (q, J = 7.2 Hz, 2H), 3.90-3.71 |
| | | (m, 4H), 2.94 (br s, 4H), 2.79 (s, 3H), 2.45 (br s, 5H), 2.13-2.00 (m, 1H), |
| | | 1.96-1.74 (m, 3H), 1.73-1.66 (m, 1H), 1.49 (s, 1H), 1.23 (t, J = 7.2 Hz, |
| | | 3H). LCMS m/z [M + H]⁺ 472. |
| A252 | A6 | ¹H NMR (CDCl₃ 400 MHz) δ_{H} = 8.60 (s, 1H), 8.34 (s, 1H), 7.96 (dd, J = |
| | | 4.0 Hz, 8.8 Hz, 1H), 7.52-7.49 (m, 1H), 7.35 (s, 1H), 4.09 (q, J = 7.2 Hz, |
| | | 2H), 3.94-3.69 (m, 4H), 3.03-2.80 (m, 4H), 2.60-2.35 (m, 5H), 2.18-2.10 |
| | | (m, 1H), 1.99-1.81 (m, 3H), 1.72-1.64 (m, 1H), 1.54-1.35 (m, 1H), 1.23 |
| | | (t, J = 7.2 Hz, 3H). LCMS m/z [M + H]⁺ 474. |
| A253 | A6 | ¹H NMR (MeOD 400 MHz) δ_{H} = 9.26 (s, 1H), 9.01 (s, 1H), 8.29 (d, J = |
| | | 2.4 Hz, 1H), 7.55 (dd, J = 2.4 Hz, 10.4 Hz, 1H), 7.11-6.74 (m, 1H), 4.07 |
| | | (q, J = 6.8 Hz, 2H), 3.91-3.74 (m, 4H), 3.00-2.91 (m, 4H), 2.67-2.57 (m, |
| | | 1H), 2.56-2.46 (m, 4H), 2.14 (dd, J = 7.2 Hz, 12.8 Hz, 1H), 1.98-1.83 |
| | | (m, 3H), 1.73-1.63 (m, 1H), 1.57-1.43 (m, 1H), 1.22 (t, J = 6.8 Hz, 3H). |
| | | LCMS m/z [M + H]⁺ 491. |
| A254 | A6 | ¹H NMR (CDCl₃ 400 MHz) δ_{H} = 8.71 (d, J = 5.2 Hz, 1H), 8.27 (d, J = 2.0 |
| | | Hz, 1H), 7.72 (d, J = 5.2 Hz, 1H), 7.11 (dd, J = 2.4 Hz, 10.4 Hz, 1H), |
| | | 4.09 (q, J = 7.2 Hz, 2H), 3.88-3.80 (m, 2H), 3.80-3.74 (m, 2H), 3.02- |
| | | 2.88 (m, 4H), 2.84 (s, 3H), 2.61-2.39 (m, 5H), 2.09 (br dd, J = 6.8 Hz, |
| | | 12.4 Hz, 1H), 1.98-1.75 (m, 3H), 1.67-1.58 (m, 1H), 1.55-1.43 (m, 1H), |
| | | 1.23 (t, J = 7.2 Hz, 3H). LCMS m/z [M + H]⁺ 455. |
| A255 | A6 | ¹H NMR (CDCl₃ 400 MHz) δ_{H} = 9.27 (s, 1H), 8.82-8.65 (m, 1H), 8.51 (d, |
| | | J = 2.4 Hz, 1H), 8.30 (s, 1H), 7.26-7.20 (m, 1H), 4.09 (q, J = 7.2 Hz, |
| | | 2H), 3.94-3.72 (m, 4H), 2.90 (br s, 4H), 2.71-2.29 (m, 8H), 2.10-2.01 |
| | | (m, 1H), 1.97-1.78 (m, 3H), 1.70-1.60 (m, 1H), 1.55-1.45 (m, 1H), 1.23 |
| | | (t, J = 7.2 Hz, 3H). LCMS m/z [M + H]⁺ 437. |
| A256 | A6 | ¹H NMR (CDCl₃400 MHz) δ_{H} = 8.33 (d, J = 1.6 Hz, 1H), 8.23 (s, 1H), |
| | | 7.40 (d, J = 2.0 Hz, 1H), 4.17 (s, 3H), 4.13-4.06 (m, 2H), 3.92-3.82 (m, |
| | | 2H), 3.82-3.75 (m, 2H), 3.00 (s, 4H), 2.83-2.44 (m, 5H), 2.16-2.12 (m, |
| | | 1H), 1.98-1.80 (m, 3H), 1.73 (s, 1H), 1.62-1.51 (m, 1H), 1.24 (t, J = 7.2 |
| | | Hz, 3H). LCMS m/z [M + H]⁺ 460. |
| A257 | A6 | ¹H NMR (CDCl₃ 400 MHz) δ_{H} = 8.33-8.27 (m, 2H), 7.18 (dd, J = 2.4 Hz, |
| | | 10.0 Hz, 1H), 4.98-4.92 (m, 1H), 4.09 (q, J = 7.2 Hz, 2H), 3.91-3.76 (m, |
| | | 4H), 2.96 (s, 4H), 2.63 (s, 5H), 2.15-2.10 (m, 1H), 2.00-1.79 (m, 4H), |
| | | 1.63 (d, J = 6.8 Hz, 6H), 1.58-1.56 (m, 1H), 1.23 (t, J = 7.2 Hz, 3H). |
| | | LCMS m/z [M + H]⁺ 472. |
| A258 | A6 | ¹H NMR (CDCl₃ 400 MHz) δ_{H} = 8.44-8.30 (m, 2H), 7.43 (s, 1H), 4.99- |
| | | 4.93 (m, 1H), 4.10 (q, J = 7.2 Hz, 2H), 3.94-3.76 (m, 4H), 3.01 (s, 4H), |
| | | 2.87-2.47 (m, 5H), 2.21-2.10 (m, 1H), 2.03-1.80 (m, 4H), 1.65 (s, 3H), |
| | | 1.63 (s, 3H), 1.60-1.57 (m, 1H), 1.24 (t, J = 7.2 Hz, 3H). LCMS m/z |
| | | [M + H]⁺ 488. |
| A259 | A6 | ¹H NMR (CDCl₃ 400 MHz) δ_{H} = 8.69 (d, J = 2.4 Hz, 1H), 8.25 (d, J = 2.0 |
| | | Hz, 1H), 7.90 (d, J = 8.4 Hz, 1H), 7.74 (dd, J = 2.4 Hz, 8.4 Hz, 1H), |
| | | 7.11 (d, J = 10.4 Hz, 1H), 4.09 (q, J = 7.2 Hz, 2H), 3.91-3.75 (m, 4H), |
| | | 3.10-2.80 (m, 4H), 2.70-2.25 (m, 5H), 2.20-2.05 (m, 1H), 1.95-1.78 (m, |
| | | 3H), 1.76-1.66 (m, 1H), 1.58-1.44 (m, 1H), 1.23 (t, J = 7.2 Hz, 3H). |
| | | LCMS m/z [M + H]⁺ 474. |
| A260 | A6 | ¹H NMR (CDCl₃ 400 MHz) δ_{H} = 8.95 (s, 2H), 8.06 (d, J = 2.0 Hz, 1H), |
| | | 7.29 (d, J = 2.4 Hz, 1H), 4.09 (q, J = 7.2 Hz, 2H), 3.92-3.70 (m, 4H), |
| | | 2.84 (br s, 4H), 2.57 (br s, 5H), 2.10 (dd, J = 6.8 Hz, 12.8 Hz, 1H), 1.96- |
| | | 1.79 (m, 3H), 1.72-1.69 (m, 1H), 1.50 (d, J = 8.4 Hz, 1H), 1.23 (t, J = |
| | | 7.2 Hz, 3H). LCMS m/z [M + H]⁺ 430. |
| A261 | A6 | ¹H NMR (CDCl₃ 400 MHz) δ_{H} = 7.54 (d, J = 2.8 Hz, 1H), 6.82 (dd, |
| | | J = 2.8Hz, 10.0 Hz, 1H), 4.10 (q, J = 7.2 Hz, 2H), 3.92-3.68 (m, 8H), 3.12 |
| | | (br s, 4H), 2.60 (br s, 5H), 2.37-2.28 (m, 2H), 2.19-2.11 (m, 1H), 2.00- |
| | | 1.89 (m, 2H), 1.88-1.78 (m, 3H), 1.77-1.69 (m, 1H), 1.65 (s, 3H), 1.61- |
| | | 1.51 (m, 1H), 1.24 (t, J = 7.2 Hz, 3H). LCMS m/z [M + H]⁺ 477. |
| A262 | A6 | ¹H NMR (CDCl₃ 400 MHz) δ_{H} = 8.13 (d, J = 2.0 Hz, 1H), 7.98 (s, 2H), |
| | | 7.41 (d, J = 2.0 Hz, 1H), 4.09 (q, J = 7.2 Hz, 2H), 3.88-3.72 (m, 4H), |
| | | 2.79-2.70 (m, 4H), 2.57-2.51 (m, 1H), 2.47-2.35 (m, 4H), 2.10-2.03 (m, |
| | | 1H), 1.93-1.78 (m, 3H), 1.68-1.60 (m, 1H), 1.54-1.44 (m, 1H), 1.23 (t, |
| | | J = 7.2 Hz, 3H). LCMS m/z [M + H]⁺ 446. |
| A263 | A6 | ¹H NMR (CDCl₃ 400 MHz) δ_{H} = 8.02 (d, J = 2.4 Hz, 1H), 7.22 (d, J = 2.4 |
| | | Hz, 1H), 4.10 (q, J = 7.2 Hz, 2H), 3.94-3.72 (m, 4H), 3.10 (br s, 4H), |
| | | 2.79-2.51 (m, 5H), 2.14 (dd, J = 7.2 Hz, 12.8 Hz, 1H), 2.01-1.79 (m, |
| | | 3H), 1.74 (dd, J = 9.6 Hz, 12.8 Hz, 1H), 1.60-1.49 (m, 1H), 1.24 (t, J = |
| | | 7.2 Hz, 3H). LCMS m/z [M + H]⁺ 459. |
| A264 | A6 | ¹H NMR (CDCl₃ 400 MHz) δ_{H} = 9.17 (s, 1H), 8.44 (s, 1H), 7.60 (d, J = |
| | | 8.0 Hz, 1H), 7.39 (s, 1H), 4.11 (q, J = 7.2 Hz, 2H), 3.93-3.84 (m, 2H), |
| | | 3.83-3.78 (m, 2H), 3.13 (br s, 4H), 2.83-2.65 (m, 5H), 2.25-2.17 (m, |
| | | 1H), 2.01-1.85 (m, 3H), 1.79-1.64 (m, 2H), 1.25 (t, J = 7.2 Hz, 3H). |
| | | LCMS m/z [M + H]⁺ 429. |
| A265 | A6 | ¹H NMR (CDCl₃ 400 MHz) δ_{H} = 8.25 (d, J = 2.0 Hz, 1H), 7.99 (s, 1H), |
| | | 7.11 (dd, J = 2.4 Hz, 10.4 Hz, 1H), 4.10 (q, J = 7.2 Hz, 2H), 3.91-3.71 |
| | | (m, 4H), 2.94 (s, 4H), 2.81 (s, 3H), 2.67-2.45 (m, 5H), 2.12 (dd, J = 6.8 |
| | | Hz, 12.8 Hz, 1H), 1.99-1.79 (m, 3H), 1.70 (dd, J = 9.6 Hz, 12.4 Hz, |
| | | 1H), 1.56-1.45 (m, 1H), 1.24 (t, J = 7.2 Hz, 3H). LCMS m/z [M + H]⁺ 460. |
| A266 | A6 | ¹H NMR (CDCl₃ 400 MHz) δ_{H} = 8.24 (d, J = 2.0 Hz, 1H), 7.59 (d, J = 1.2 |
| | | Hz, 1H), 7.21 (dd, J = 2.4 Hz, 10.0 Hz, 1H), 4.10 (q, J = 7.2 Hz, 2H), |
| | | 3.93-3.76 (m, 4H), 3.05 (d, J = 4.4 Hz, 4H), 2.77-2.61 (m, 5H), 2.52 (d, |
| | | J = 0.8 Hz, 3H), 2.16 (dd, J = 6.8 Hz, 12.8 Hz, 1H), 2.01-1.82 (m, 3H), |
| | | 1.79-1.71 (m, 1H), 1.58-1.49 (m, 1H), 1.24 (t, J = 7.2 Hz, 3H). LCMS |
| | | m/z [M + H]⁺ 460. |
| A267 | A6 | ¹H NMR (CDCl₃ 400 MHz) δ_{H} = 8.28 (d, J = 2.0 Hz, 1H), 7.23 (dd, J = |
| | | 2.8 Hz, 10.0 Hz, 1H), 7.02-6.87 (m, 1H), 4.10 (q, J = 7.2 Hz, 2H), 3.95- |
| | | 3.70 (m, 4H), 3.06 (s, 4H), 2.83-2.60 (m, 5H), 2.55 (s, 3H), 2.16 (dd, J = |
| | | 6.8 Hz, 12.8 Hz, 1H), 2.02-1.80 (m, 3H), 1.78-1.69 (m, 1H), 1.64-1.58 |
| | | (m, 1H), 1.24 (t, J = 7.2 Hz, 3H). LCMS m/z [M + H]⁺ 460. |
| A268 | A6 | ¹H NMR (CDCl₃ 400 MHz) δ_{H} = 8.57 (d, J = 4.4 Hz, 1H), 8.36 (d, J = 1.6 |
| | | Hz, 1H), 7.61-7.46 (m, 1H), 7.41-7.34 (m, 2H), 4.08 (q, J = 7.2 Hz, 2H), |
| | | 3.89-3.69 (m, 4H), 2.87 (s, 4H), 2.32 (s, 5H), 2.04 (dd, J = 6.8 Hz, 12.7 |
| | | Hz, 1H), 1.93-1.74 (m, 3H), 1.65 (s, 1H), 1.54-1.37 (m, 1H), 1.22 (t, J = |
| | | 7.2 Hz, 3H). LCMS m/z [M + H]⁺ 474. |
| A269 | A6 | ¹H NMR (CDCl₃ 400 MHz) δ_{H} = 8.64 (dd, J = 1.6 Hz, 4.8 Hz, 1H), 8.34 |
| | | (d, J = 1.6 Hz, 1H), 7.80 (dd, J = 1.6 Hz, 8.4 Hz, 1H), 7.38 (s, 1H), 7.30 |
| | | (dd, J = 4.8 Hz, 8.4 Hz, 1H), 4.08 (q, J = 7.2 Hz, 2H), 3.90-3.68 (m, |
| | | 4H), 2.88 (s, 4H), 2.52-2.17 (m, 5H), 2.10-1.97 (m, 1H), 1.93-1.73 (m, |
| | | 3H), 1.68-1.63 (m, 1H), 1.46 (s, 1H), 1.22 (t, J = 7.2 Hz, 3H). LCMS m/z |
| | | [M + H]⁺ 490. |
| A270 | A6 | ¹H NMR (CDCl₃ 400 MHz) δ_{H} = 8.72 (d, J = 4.8 Hz, 1H), 8.25 (d, J = 2.4 |
| | | Hz, 1H), 7.91 (d, J = 4.8 Hz, 1H), 7.13 (dd, J = 2.0 Hz, 10.0 Hz, 1H), |
| | | 4.10 (q, J = 7.2 Hz, 2H), 3.92-3.76 (m, 4H), 2.93 (br s, 4H), 2.67-2.44 |
| | | (m, 5H), 2.13 (br dd, J = 6.8 Hz, 12.0 Hz, 1H), 1.98-1.80 (m, 3H), 1.76- |
| | | 1.63 (m, 1H), 1.57-1.47 (m, 1H), 1.24 (t, J = 7.2 Hz, 3H). LCMS m/z |
| | | [M + H]⁺ 446. |
| A271 | A6 | ¹H NMR (CDCl₃ 400 MHz) δ_{H} = 9.26 (s, 2H), 8.34 (d, J = 1.6 Hz, 1H), |
| | | 7.37 (d, J = 1.6 Hz, 1H), 4.09 (q, J = 7.2 Hz, 2H), 3.88-3.80 (m, 2H), |
| | | 3.79-3.76 (m, 2H), 2.90 (t, J = 4.4 Hz, 4H), 2.79 (s, 3H), 2.64-2.43 (m, |
| | | 5H), 2.09 (d, J = 6.8, 12.8 Hz, 1H), 1.96-1.80 (m, 3H), 1.69-1.63 (m, |
| | | 1H), 1.55-1.44 (m, 1H), 1.23 (t, J = 7.2 Hz, 3H). LCMS m/z [M + H]⁺ 471. |
| A272 | A6 | ¹H NMR (CDCl₃ 400 MHz) δ_{H} = 9.28 (d, J = 1.2 Hz, 1H), 8.77 (s, 1H), |
| | | 8.68-8.57 (m, 2H), 7.62 (s, 1H), 4.09 (q, J = 7.2 Hz, 2H), 3.90-3.71 (m, |
| | | 4H), 2.94 (br s, 4H), 2.64-2.40 (m, 5H), 2.13-2.01 (m, 1H), 1.95-1.61 (m, |
| | | 4H), 1.55-1.50 (m, 1H), 1.23 (t, J = 7.2 Hz, 3H). LCMS m/z [M + H]⁺ 448. |
| A273 | A7 | ¹H NMR (CDCl₃ 400 MHz) δ_{H} = 8.17 (d, J = 2.4 Hz, 1H), 7.08 (dd, J = |
| | | 2.4 Hz, 10.0 Hz, 1H), 4.10 (q, J = 7.2 Hz, 2H), 3.93-3.77 (m, 4H), 3.03 |
| | | (br s, 2H), 2.89 (br t, J = 4.8 Hz, 4H), 2.79-2.49 (m, 5H), 2.35 (br s, 3H), |
| | | 2.20-1.82 (m, 10H), 1.78-1.73 (m, 2H), 1.63-1.50 (m, 1H), 1.24 (t, J = |
| | | 7.2 Hz, 3H). LCMS m/z [M + H]⁺ 460. |
| A274 | A7 | ¹H NMR (CDCl₃ 400 MHz) δ_{H} = 8.19 (d, J = 2.4 Hz, 1H), 7.11 (br d, J = |
| | | 8.4 Hz, 1H), 4.14-4.05 (m, 4H), 3.94-3.83 (m, 2H), 3.83-3.77 (m, 2H), |
| | | 3.52 (t, J = 11.6 Hz, 2H), 3.37-3.27 (m, 1H), 2.91 (br s, 4H), 2.66 (br s, |
| | | 5H), 2.21-2.13 (m, 1H), 2.11-1.92 (m, 4H), 1.91-1.82 (m, 1H), 1.81-1.70 |
| | | (m, 1H), 1.63-1.53 (m, 3H), 1.24 (t, J = 7.2 Hz, 3H). LCMS m/z [M + H]⁺ |
| | | 447. |
| A275 | A7 | ¹H NMR (CDCl₃ 400 MHz) δ_{H} = 8.17 (d, J = 2.4 Hz, 1H), 7.11 (dd, J = |
| | | 2.4Hz, 10.0 Hz, 1H), 4.10 (q, J = 7.2 Hz, 2H), 3.94-3.74 (m, 4H), 3.22- |
| | | 3.11 (m, 1H), 2.96-2.82 (m, 4H), 2.78-2.49 (m, 4H), 2.30-2.11 (m, 3H), |
| | | 2.10-1.69 (m, 10H), 1.59-1.44 (m, 2H), 1.24 (t, J = 7.2 Hz, 3H). LCMS |
| | | m/z [M + H]⁺ 481. |
| A276 | A7 | ¹H NMR (CDCl₃ 400 MHz) δ_{H} = 8.04 (s, 1H), 6.82 (dd, J = 2.4 Hz, 12.8 |
| | | Hz, 1H), 4.10 (q, J = 7.2 Hz, 2H), 3.97-3.97 (m, 1H), 3.97 (s, 4H), 3.91- |
| | | 3.74 (m, 4H), 3.19 (s, 4H), 3.03-2.89 (m, 1H), 2.60 (d, J = 3.6 Hz, 5H), |
| | | 2.14 (dd, J = 8.0 Hz, 12.4 Hz, 1H), 2.06-1.78 (m, 9H), 1.76-1.62 (m, |
| | | 3H), 1.24 (t, J = 7.2 Hz, 3H). LCMS m/z [M + H]⁺ 460. |
| A277 | A7 | ¹H NMR (CDCl₃ 400 MHz) δ_{H} = 8.15 (d, J = 2.4 Hz, 1H), 6.99 (dd, J = |
| | | 2.4 Hz, 10.4 Hz, 1H), 4.82 (s, 2H), 4.70 (s, 2H), 4.10 (q, J = 7.2 Hz, |
| | | 2H), 3.94-3.76 (m, 4H), 3.75-3.59 (m, 1H), 3.06-2.57 (m, 9H), 2.55 (d, |
| | | J = 8.4 Hz, 4H), 2.23-2.10 (m, 1H), 2.03-1.83 (m, 3H), 1.80-1.69 (m, 1H), |
| | | 1.60-1.45 (m, 1H), 1.24 (t, J = 7.2 Hz, 3H). LCMS m/z [M + H]⁺ 459. |
| A278 | A7 | ¹H NMR (CDCl₃ 400 MHz) δ_{H} = 8.34 (dd, J = 4.8, 1.2 Hz, 1H), 7.41 (dd, |
| | | J = 8.0, 1.2 Hz, 1H), 7.10 (dd, J = 8.0, 4.8 Hz, 1H), 4.11 (q, J = 7.2 Hz, |
| | | 4H), 3.84-3.94 (m, 2H), 3.78-3.84 (m, 2H), 3.56 (t, J = 11.6 Hz, 2H), |
| | | 3.49-3.39 (m, 1H), 2.92 (br s, 4H), 2.67 (br s, 5H), 2.22-2.03 (m, 3H), |
| | | 2.02-1.84 (m, 3H), 1.81-1.73 (m, 1H), 1.65-1.53 (m, 3H), 1.25 (t, J = 7.2 |
| | | Hz, 3H). LCMS m/z [M + H]⁺ 429. |
| A279 | A7 | ¹H NMR (CDCl₃ 400 MHz) δ_{H} = 8.23 (d, J = 2.0 Hz, 1H), 7.20 (d, J = 2.4 |
| | | Hz, 1H), 4.81 (s, 2H), 4.69 (s, 2H), 4.10 (q, J = 7.2 Hz, 2H), 3.91-3.77 |
| | | (m, 4H), 3.75-3.65 (m, 1H), 2.93-2.80 (m, 4H), 2.72-2.57 (m, 5H), 2.55 |
| | | (d, J = 8.4 Hz, 4H), 2.14 (dd, J = 6.8 Hz, 12.4 Hz, 1H), 2.01-1.84 (m, |
| | | 3H), 1.77-1.71 (m, 1H), 1.63-1.50 (m, 1H), 1.23 (t, J = 7.2 Hz, 3H). |
| | | LCMS m/z [M + H]⁺ 475. |
| A280 | A7 | ¹H NMR (CDCl₃ 400 MHz) δ_{H} = 8.26 (d, J = 1.6 Hz, 1H), 7.30 (s, 1H), |
| | | 4.12 (q, J = 7.2 Hz, 2H), 3.94-3.84 (m, 2H), 3.84-3.74 (m, 2H), 3.10- |
| | | 2.98 (m, 3H), 2.95-2.87 (m, 4H), 2.71-2.58 (m, 5H), 2.34 (s, 3H), 2.20- |
| | | 2.13 (m, 1H), 2.10-1.83 (m, 7H), 1.76-1.65 (m, 3H), 1.61-1.51 (m, 1H), |
| | | 1.25 (t, J = 7.2 Hz, 3H). LCMS m/z [M + H]⁺ 476. |
| A281 | A7 | ¹H NMR (CDCl₃ 400 MHz) δ_{H} = 8.20 (d, J = 2.4 Hz, 1H), 7.09 (d, J = |
| | | 10.0 Hz, 1H), 4.17-4.07 (m, 4H), 4.00-3.77 (m, 7H), 2.92 (d, J = 15.6 |
| | | Hz, 4H), 2.65 (br s, 5H), 2.25 (q, J = 7.2 Hz, 2H), 2.15 (dd, J = 6.8 Hz, |
| | | 12.4 Hz, 1H), 2.00-1.80 (m, 3H), 1.77-1.70 (m, 1H), 1.57-1.45 (m, 1H), |
| | | 1.24 (t, J = 7.2 Hz, 3H). LCMS m/z [M + H]⁺ 433. Peak 1. |
| A282 | A7 | ¹H NMR (CDCl₃ 400 MHz) δ_{H} = 8.19 (d, J = 2.4Hz, 1H), 7.08 (dd, J = |
| | | 2.4 Hz, 10.0 Hz, 1H), 4.19-4.07 (m, 4H), 3.99-3.76 (m, 7H), 2.92 (d, J = |
| | | 15.6 Hz, 4H), 2.66 (br s, 5H), 2.24 (q, J = 7.2 Hz, 2H), 2.15 (dd, J = 7.2 |
| | | Hz, 12.8 Hz, 1H), 2.02-1.82 (m, 3H), 1.78-1.71 (m, 1H), 1.57-1.50 (m, |
| | | 1H), 1.24 (t, J = 7.2 Hz, 3H). LCMS m/z [M + H]⁺ 433. Peak 2. |
| A283 | A8 | LCMS m/z [M + H]⁺ 458. |
| A284 | A8 | ¹H NMR (CDCl₃ 400 MHz) δ_{H} = 8.71 (dd, J = 2.0 Hz, 5.2 Hz, 1H), 8.25 |
| | | (d, J = 2.0 Hz, 1H), 8.03-7.85 (m, 1H), 7.15 (dd, J = 2.4 Hz, 10.4 Hz, |
| | | 1H), 4.09 (q, J = 7.2 Hz, 2H), 3.93-3.72 (m, 4H), 3.10-2.90 (m, 4H), |
| | | 2.69-2.43 (m, 5H), 2.11 (dd, J = 6.8 Hz, 12.4 Hz, 1H), 1.97-1.80 (m, |
| | | 3H), 1.75-1.68 (m, 1H), 1.58-1.47 (m, 1H), 1.23 (t, J = 7.2 Hz, 3H). |
| | | LCMS m/z [M + H]⁺ 459. |
| A285 | A8 | ¹H NMR (CDCl₃ 400 MHz) δ_{H} = 8.53 ( d, J = 8.4 Hz, 2H), 8.21 (s, 1H), |
| | | 7.21 (d, J = 9.2 Hz, 1H), 4.10 (q, J = 7.2 Hz, 2H), 3.94-3.75 (m, 4H), |
| | | 3.14-2.88 (m, 4H), 2.85-2.44 (m, 5H), 2.30-2.10 (m, 1H), 2.05-1.86 (m, |
| | | 3H), 1.80-1.70 (m, 1H), 1.58-1.48 (m, 1H), 1.24 (t, J = 7.2 Hz, 3H). |
| | | LCMS m/z [M + H]⁺ 497. |
| A286 | A8 | ¹H NMR (CDCl₃ 400 MHz) δ_{H} = 8.30-8.17 (m, 1H), 7.95 (d, J = 3.2 Hz, |
| | | 1H), 7.41 (d, J = 3.2 Hz, 1H), 7.24 (dd, J = 2.4 Hz, 10.2 Hz, 1H), 4.10 |
| | | (q, J = 7.2 Hz, 2H), 3.94-3.72 (m, 4H), 3.07 (t, J = 4.4 Hz, 4H), 2.79- |
| | | 2.58 (m, 5H), 2.16 (dd, J = 6.8 Hz, 12.8 Hz, 1H), 2.01-1.81 (m, 3H), |
| | | 1.74 (dd, J = 9.6 Hz, 12.8 Hz, 1H), 1.58-1.50 (m, 1H), 1.24 (t, J = 7.2 |
| | | Hz, 3H). LCMS m/z [M + H]⁺ 446. |
| A287 | A8 | ¹H NMR (CDCl₃ 400 MHz) δ_{H} = 8.67-8.58 (m, 1H), 8.25 (d, J = 2.0 Hz, |
| | | 1H), 7.80 (d, J = 8.0 Hz, 1H), 7.30 (dd, J = 4.8 Hz, 8.0 Hz, 1H), 7.19- |
| | | 7.08 (m, 1H), 4.08 (q, J = 7.2 Hz, 2H), 3.89-3.70 (m, 4H), 2.88 (s, 4H), |
| | | 2.47 (s, 1H), 2.30 (s, 4H), 2.04 (dd, J = 7.2 Hz, 12.4 Hz, 1H), 1.94-1.75 |
| | | (m, 3H), 1.64-1.55 (m, 1H), 1.52-1.35 (m, 1H), 1.22 (t, J = 7.2 Hz, 3H). |
| | | LCMS m/z [M + H]⁺ 474. |
| A288 | A9 | ¹H NMR (CDCl₃ 400 MHz) δ_{H} = 7.62 (d, J = 2.4 Hz, 1H), 7.13 (dd, J = |
| | | 2.4 Hz, 9.6 Hz, 1H), 4.78 (s, 2H), 4.64 (s, 2H), 4.16-4.07 (m, 2H), 3.98- |
| | | 3.75 (m, 4H), 2.98-2.65 (m, 10H), 2.43-1.39 (m, 10H), 1.23 (t, J = 7.2 |
| | | Hz, 3H). LCMS m/z [M + H]⁺ 474. |
| A289 | A9 | ¹H NMR (CDCl₃ 400 MHz) δ_{H} = 7.80 (s, 1H), 6.90 (dd, J = 2.4 Hz, 10.0 |
| | | Hz, 1H), 4.10 (q, J = 7.2 Hz, 2H), 3.96-3.78 (m, 8H), 3.50-3.10 (m, 8H), |
| | | 2.64 (br s, 5H), 2.20-2.15 (m, 1H), 1.98 (s, 2H), 1.88 (dd, J = 6.8 Hz, |
| | | 13.6 Hz, 1H), 1.75-1.66 (m, 1H), 1.39 (s, 1H), 1.24 (t, J = 7.2 Hz, 3H). |
| | | LCMS m/z [M + H]⁺ 448. |
| A290 | A10 | ¹H NMR (CDCl₃ 400 MHz) δ_{H} = 8.20 (s, 1H), 7.99 (s, 1H), 7.30 (s, 1H), |
| | | 7.16 (d, J = 9.2 Hz, 1H), 4.09 (q, J = 7.2 Hz, 2H), 3.94-3.67 (m, 4H), |
| | | 2.93-2.85 (m, 4H), 2.68-2.52 (m, 5H), 2.26 (s, 3H), 2.20-2.12 (m, 1H), |
| | | 1.94-1.79 (m, 3H), 1.75-1.65 (s, 1H), 1.58-1.48 (m, 1H), 1.23 (t, J = 7.2 |
| | | Hz, 3H). LCMS m/z [M + H]⁺ 443. |
| A291 | A10 | ¹H NMR (CD₃OD 400 MHz) δ_{H} = 8.02 (dd, J = 2.4 Hz, 12.8 Hz, 2H), |
| | | 7.64 (d, J = 1.6 Hz, 1H), 7.45 (dd, J = 2.4 Hz, 10.0 Hz, 1H), 6.35 (d, J = |
| | | 2.4 Hz, 1H), 4.07 (q, J = 7.2 Hz, 2H), 3.91-3.71 (m, 4H), 2.75 (t, J = 4.8 |
| | | Hz, 4H), 2.66-2.48 (m, 5H), 2.35 (s, 3H), 2.25-2.10 (m, 1H), 1.99-1.81 |
| | | (m, 3H), 1.68 (dd, J = 9.2 Hz, 12.8 Hz, 1H), 1.55-1.43 (m, 1H), 1.22 (t, |
| | | J = 7.2 Hz, 3H). LCMS m/z [M + H]⁺ 443. |
| A292 | A11 | ¹H NMR (CDCl₃ 400 MHz) δ_{H} = 8.48 (d, J = 3.2 Hz, 1H), 8.34 (d, J = 2.4 |
| | | Hz, 1H), 7.77-7.74 (m, 1H), 7.56-7.44 (m, 2H), 4.08 (q, J = 7.2 Hz, 2H), |
| | | 3.89-3.79 (m, 2H), 3.79-3.71 (m, 2H), 3.22-3.16 (m, 1H), 3.08-3.03 (m, |
| | | 2H), 2.60-2.52 (m, 1H), 2.13-2.08 (m, 1H), 2.00-1.84 (m, 6H), 1.81-1.65 |
| | | (m, 4H), 1.62-1.49 (m, 1H), 1.22 (t, J = 7.2 Hz, 3H). LCMS m/z [M + H]⁺ |
| | | 457. Peak 1. |
| A293 | A11 | ¹H NMR (CDCl₃ 400 MHz) δ_{H} = 8.48 (d, J = 2.8 Hz, 1H), 8.37 (d, J = 2.4 |
| | | Hz, 1H), 7.86-7.73 (m, 1H), 7.56-7.51 (m, 2H), 4.09 (q, J = 7.2 Hz, 2H), |
| | | 3.95-3.81 (m, 2H), 3.76 (s, 2H), 3.48-2.95 (m, 3H), 2.81-2.51 (m, 1H), |
| | | 2.15-2.13 (m, 1H), 2.06-1.86 (m, 6H), 1.73-1.52 (m, 5H), 1.23 (t, J = 7.2 |
| | | Hz, 3H). LCMS m/z [M + H]⁺ 457. Peak 2. |

### Example #20: Biological Assays

### FLIPR Assay

Fluorescence Imaging Plate Reader (FLIPR) assays were performed using the intracellular calcium sensitive dye, Fluo 8, that exhibits increased fluorescence intensity upon calcium binding. Stimulation of a Gq-coupled G-protein coupled receptors (GPCRs), result in an efflux of calcium from the endoplasmic reticulum to the cytoplasm (Berridge, 1993), thus rendering this assay appropriate for the assessment of M1, M3 and M5 receptors. CHO-K1 cells overexpressing the M1, M3, or M5 muscarinic acetylcholine receptor (mAChR) were dispensed at 15,000 cells per well in 30 μl volume to 384-well plates and grown overnight at 37° C. in a 5% CO₂ atmosphere. Fluo-8 Solution, 10 μl per well, was added to each well and incubated for 30 minutes at 37° C. in a 5% CO₂ atmosphere. Compounds diluted in Hanks' balanced salt solution (HBSS) were transferred to the cell assay plate at 10 μl per well, and then read on a FLIPR instrument. EC₅₀ and Eₘₐₓ figures were calculated from the resulting data for each receptor subtype.

Results for select compounds provided herein are shown in the table below.

cAMP Assay

The M2 and M4 mACh receptors are Gi-coupled receptors which, upon activation, produce a decrease in cAMP. Assays were developed that measured the potency and efficacy of compounds at the M2 or M4 mAChR using M2- or M4-overexpressing CHO-K1 cell lines. Time-resolved fluorescence resonance energy transfer (TR-FRET) technology was used, where signal occurs as a result of transfer of energy if the donor molecule is in close proximity to an acceptor molecule when the molecules have bound to the molecule of interest. The cAMP detection is a competition binding assay, where cAMP produced by cells competes for binding with the labeled donor molecule to the anti-cAMP acceptor antibody. Due to the low basal levels of cAMP in the CHO-K1 cell line, forskolin was used to increase cAMP levels to enable assessment of agonist activity at the mAChR of interest.

Results for select compounds provided herein are shown in the table below.

| | M4 | M4 | M1 | M1 | M2 | M2 | M3 | M3 | M5 | M5 |
|---|---|---|---|---|---|---|---|---|---|---|
| | EC₅₀ | Emax | EC₅₀ | Emax | EC₅₀ | Emax | EC₅₀ | Emax | EC₅₀ | Emax |
| | (nM) | (%) | (nM) | (%) | (nM) | (%) | (nM) | (%) | (nM) | (%) |
| A1 | ++++ | * | I.A. | N/A | I.A. | N/A | I.A. | N/A | ++ | *** |
| A2 | ++++ | * | ++++ | *** | I.A. | N/A | I.A. | N/A | I.A. | N/A |
| A3 | N/A | N/A | +++ | *** | N/A | N/A | I.A. | N/A | I.A. | N/A |
| A4 | ++++ | * | N/A | N/A | N/A | N/A | N/A | N/A | N/A | N/A |
| A5 | N/A | N/A | I.A. | N/A | N/A | N/A | I.A. | N/A | I.A. | N/A |
| A6 | ++++ | * | I.A. | N/A | N/A | N/A | I.A. | N/A | I.A. | N/A |
| A7 | ++++ | * | I.A. | N/A | I.A | N/A | I.A. | N/A | I.A. | N/A |
| A8 | ++++ | * | N/A | N/A | + | N/A | N/A | N/A | N/A | N/A |
| A9 | ++++ | * | N/A | N/A | I.A. | N/A | I.A. | N/A | I.A. | N/A |
| A10 | ++++ | * | N/A | N/A | I.A. | N/A | I.A. | N/A | I.A. | N/A |
| A11 | ++++ | * | N/A | N/A | I.A. | N/A | N/A | N/A | N/A | N/A |
| A12 | ++++ | * | N/A | N/A | I.A. | N/A | N/A | N/A | N/A | N/A |
| A13 | ++ | N/A | N/A | N/A | I.A. | N/A | N/A | N/A | N/A | N/A |
| A14 | ++++ | * | N/A | N/A | I.A. | N/A | N/A | N/A | N/A | N/A |
| A15 | ++++ | * | N/A | N/A | I.A. | N/A | I.A. | N/A | I.A. | N/A |
| A16 | ++ | N/A | N/A | N/A | I.A. | N/A | I.A. | N/A | I.A. | N/A |
| A17 | N/A | N/A | N/A | N/A | I.A. | N/A | N/A | N/A | N/A | N/A |
| A18 | ++ | N/A | N/A | N/A | I.A. | N/A | N/A | N/A | N/A | N/A |
| A19 | ++++ | * | N/A | N/A | I.A. | N/A | N/A | N/A | N/A | N/A |
| A20 | ++ | N/A | N/A | N/A | I.A. | N/A | N/A | N/A | N/A | N/A |
| A21 | ++++ | * | N/A | N/A | I.A. | N/A | N/A | N/A | N/A | N/A |
| A22 | ++++ | * | N/A | N/A | I.A. | N/A | N/A | N/A | N/A | N/A |
| A23 | ++++ | * | N/A | N/A | I.A. | N/A | N/A | N/A | N/A | N/A |
| A24 | ++++ | * | N/A | N/A | I.A. | N/A | N/A | N/A | N/A | N/A |
| A25 | ++++ | * | I.A. | N/A | I.A. | N/A | I.A. | N/A | I.A. | N/A |
| A26 | ++++ | * | I.A. | N/A | I.A. | N/A | I.A. | N/A | I.A. | N/A |
| A27 | N/A | N/A | I.A. | N/A | N/A | N/A | I.A. | N/A | I.A. | N/A |
| A28 | ++++ | * | N/A | N/A | N/A | N/A | N/A | N/A | N/A | N/A |
| A29 | ++++ | * | + | N/A | N/A | N/A | I.A. | N/A | I.A. | N/A |
| A30 | ++++ | * | I.A. | N/A | N/A | N/A | I.A. | N/A | I.A. | N/A |
| A31 | ++++ | * | I.A. | N/A | N/A | N/A | I.A. | N/A | I.A. | N/A |
| A32 | ++++ | * | I.A. | N/A | N/A | N/A | I.A. | N/A | I.A. | N/A |
| A33 | ++++ | * | I.A. | N/A | N/A | N/A | I.A. | N/A | I.A. | N/A |
| A34 | ++++ | * | I.A. | N/A | N/A | N/A | I.A. | N/A | I.A. | N/A |
| A35 | ++++ | * | I.A. | N/A | N/A | N/A | I.A. | N/A | I.A. | N/A |
| A36 | ++++ | * | I.A. | N/A | N/A | N/A | I.A. | N/A | I.A. | N/A |
| A37 | ++++ | * | I.A. | N/A | N/A | N/A | I.A. | N/A | I.A. | N/A |
| A38 | ++++ | * | I.A. | N/A | N/A | N/A | I.A. | N/A | I.A. | N/A |
| A39 | ++++ | * | I.A. | N/A | N/A | N/A | I.A. | N/A | I.A. | N/A |
| A40 | ++++ | * | I.A. | N/A | N/A | N/A | I.A. | N/A | I.A. | N/A |
| A41 | ++++ | * | I.A. | N/A | N/A | N/A | I.A. | N/A | I.A. | N/A |
| A42 | ++++ | * | I.A. | N/A | N/A | N/A | I.A. | N/A | I.A. | N/A |
| A43 | +++ | * | I.A. | N/A | N/A | N/A | I.A. | N/A | I.A. | N/A |
| A44 | ++++ | * | I.A. | N/A | N/A | N/A | I.A. | N/A | I.A. | N/A |
| A45 | ++++ | * | I.A. | N/A | N/A | N/A | I.A. | N/A | I.A. | N/A |
| A46 | ++++ | * | I.A. | N/A | N/A | N/A | I.A. | N/A | I.A. | N/A |
| A47 | ++++ | * | I.A. | N/A | N/A | N/A | I.A. | N/A | I.A. | N/A |
| A48 | ++++ | * | I.A. | N/A | N/A | N/A | I.A. | N/A | I.A. | N/A |
| A49 | +++ | * | + | ** | N/A | N/A | I.A. | N/A | + | N/A |
| A50 | +++ | ** | I.A. | N/A | I.A. | N/A | I.A. | N/A | I.A. | N/A |
| A51 | ++++ | * | ++++ | *** | I.A. | N/A | I.A. | N/A | I.A. | N/A |
| A52 | ++++ | * | I.A. | N/A | I.A. | N/A | I.A. | N/A | I.A. | N/A |
| A53 | ++++ | * | +++ | *** | I.A. | N/A | I.A. | N/A | I.A. | N/A |
| A54 | +++ | * | I.A. | N/A | I.A. | N/A | I.A. | N/A | I.A. | N/A |
| A55 | ++++ | * | I.A. | N/A | I.A. | N/A | I.A. | N/A | I.A. | N/A |
| A56 | ++++ | * | I.A. | N/A | I.A. | N/A | I.A. | N/A | I.A. | N/A |
| A57 | ++++ | * | I.A. | N/A | I.A. | N/A | I.A. | N/A | I.A. | N/A |
| A58 | ++++ | * | I.A. | N/A | I.A. | N/A | I.A. | N/A | I.A. | N/A |
| A59 | ++++ | * | ++++ | *** | I.A. | N/A | I.A. | N/A | I.A. | N/A |
| A60 | ++++ | * | ++++ | N/A | I.A. | N/A | I.A. | N/A | I.A. | N/A |
| A61 | ++++ | * | ++++ | N/A | I.A. | N/A | I.A. | N/A | I.A. | N/A |
| A62 | ++++ | * | ++++ | N/A | I.A. | N/A | I.A. | N/A | I.A. | N/A |
| A63 | ++++ | * | ++++ | *** | I.A. | N/A | I.A. | N/A | +++ | *** |
| A64 | +++ | * | I.A. | N/A | I.A. | N/A | I.A. | N/A | I.A. | N/A |
| A65 | +++ | ** | I.A. | N/A | I.A. | N/A | I.A. | N/A | I.A. | N/A |
| A66 | ++ | N/A | I.A. | N/A | I.A. | N/A | I.A. | N/A | I.A. | N/A |
| A67 | ++++ | * | ++ | *** | I.A. | N/A | I.A. | N/A | I.A. | N/A |
| A68 | ++++ | * | I.A. | N/A | I.A. | N/A | I.A. | N/A | I.A. | N/A |
| A69 | +++ | * | I.A. | N/A | I.A. | N/A | I.A. | N/A | I.A. | N/A |
| A70 | ++++ | * | +++ | *** | I.A. | N/A | I.A. | N/A | I.A. | N/A |
| A71 | ++++ | * | I.A. | N/A | I.A. | N/A | I.A. | N/A | ++++ | *** |
| A72 | +++ | ** | I.A. | N/A | I.A. | N/A | I.A. | N/A | I.A. | N/A |
| A73 | ++++ | * | I.A. | N/A | I.A. | N/A | I.A. | N/A | I.A. | N/A |
| A74 | ++++ | * | I.A. | N/A | I.A. | N/A | I.A. | N/A | + | *** |
| A75 | ++++ | * | ++++ | *** | I.A. | N/A | I.A. | N/A | I.A. | N/A |
| A76 | ++++ | * | ++++ | ** | + | N/A | I.A. | N/A | ++ | *** |
| A77 | ++++ | * | I.A. | N/A | I.A. | N/A | I.A. | N/A | I.A. | N/A |
| A78 | ++++ | * | + | N/A | I.A. | N/A | I.A. | N/A | I.A. | N/A |
| A79 | ++++ | * | I.A. | N/A | I.A. | N/A | I.A. | N/A | I.A. | N/A |
| A80 | ++++ | * | ++++ | *** | I.A. | N/A | I.A. | N/A | +++ | *** |
| A81 | ++++ | * | I.A. | N/A | I.A. | N/A | I.A. | N/A | I.A. | N/A |
| A82 | ++++ | * | +++ | *** | I.A. | N/A | I.A. | N/A | I.A. | N/A |
| A83 | ++++ | * | I.A. | N/A | I.A. | N/A | I.A. | N/A | I.A. | N/A |
| A84 | ++++ | * | I.A. | N/A | I.A. | N/A | I.A. | N/A | I.A. | N/A |
| A85 | ++++ | * | I.A. | N/A | I.A. | N/A | I.A. | N/A | I.A. | N/A |
| A86 | ++++ | * | I.A. | N/A | I.A. | N/A | I.A. | N/A | I.A. | N/A |
| A87 | ++++ | * | ++++ | *** | I.A. | N/A | I.A. | N/A | I.A. | N/A |
| A88 | ++++ | * | I.A. | N/A | I.A. | N/A | I.A. | N/A | I.A. | N/A |
| A89 | ++++ | * | I.A. | N/A | I.A. | N/A | I.A. | N/A | I.A. | N/A |
| A90 | ++++ | * | I.A. | N/A | I.A. | N/A | I.A. | N/A | I.A. | N/A |
| A91 | ++++ | * | I.A. | N/A | I.A. | N/A | I.A. | N/A | ++ | *** |
| A92 | ++++ | * | I.A. | N/A | I.A. | N/A | I.A. | N/A | I.A. | N/A |
| A93 | ++++ | * | ++ | *** | I.A. | N/A | I.A. | N/A | I.A. | N/A |
| A94 | +++ | * | I.A. | N/A | I.A. | N/A | I.A. | N/A | I.A. | N/A |
| A95 | ++++ | * | I.A. | N/A | I.A. | N/A | I.A. | N/A | I.A. | N/A |
| A96 | ++++ | * | I.A. | N/A | I.A. | N/A | I.A. | N/A | I.A. | N/A |
| A97 | ++++ | * | I.A. | N/A | I.A. | N/A | I.A. | N/A | I.A. | N/A |
| A98 | ++++ | * | I.A. | N/A | I.A. | N/A | I.A. | N/A | I.A. | N/A |
| A99 | ++++ | * | I.A. | N/A | I.A. | N/A | I.A. | N/A | I.A. | N/A |
| A100 | ++++ | * | +++ | *** | I.A. | N/A | I.A. | N/A | I.A. | N/A |
| A101 | ++++ | * | I.A. | N/A | I.A. | N/A | I.A. | N/A | I.A. | N/A |
| A102 | ++++ | * | I.A. | N/A | I.A. | N/A | I.A. | N/A | I.A. | N/A |
| A103 | ++++ | * | +++ | *** | I.A. | N/A | I.A. | N/A | I.A. | N/A |
| A104 | ++++ | * | + | *** | I.A. | N/A | I.A. | N/A | I.A. | N/A |
| A105 | ++++ | * | I.A. | N/A | I.A. | N/A | I.A. | N/A | I.A. | N/A |
| A106 | ++++ | * | I.A. | N/A | I.A. | N/A | I.A. | N/A | I.A. | N/A |
| A107 | ++++ | * | I.A. | N/A | I.A. | N/A | I.A. | N/A | I.A. | N/A |
| A108 | ++++ | * | I.A. | N/A | I.A. | N/A | I.A. | N/A | I.A. | N/A |
| A109 | ++++ | * | I.A. | N/A | I.A. | N/A | I.A. | N/A | I.A. | N/A |
| A110 | ++++ | * | I.A. | N/A | I.A. | N/A | I.A. | N/A | I.A. | N/A |
| A111 | ++++ | * | I.A. | N/A | I.A. | N/A | I.A. | N/A | I.A. | N/A |
| A112 | ++++ | * | I.A. | N/A | I.A. | N/A | I.A. | N/A | I.A. | N/A |
| A113 | ++++ | * | ++ | *** | I.A. | N/A | I.A. | N/A | I.A. | N/A |
| A114 | ++++ | * | I.A. | N/A | I.A. | N/A | I.A. | N/A | I.A. | N/A |
| A115 | ++++ | * | I.A. | N/A | I.A. | N/A | I.A. | N/A | I.A. | N/A |
| A116 | +++ | * | I.A. | N/A | I.A. | N/A | I.A. | N/A | I.A. | N/A |
| A117 | ++++ | * | I.A. | N/A | I.A. | N/A | I.A. | N/A | I.A. | N/A |
| A118 | ++++ | * | I.A. | N/A | I.A. | N/A | I.A. | N/A | I.A. | N/A |
| A119 | ++++ | * | I.A. | N/A | I.A. | N/A | I.A. | N/A | I.A. | N/A |
| A120 | ++++ | * | I.A. | N/A | I.A. | N/A | I.A. | N/A | I.A. | N/A |
| A121 | ++++ | * | I.A. | N/A | I.A. | N/A | I.A. | N/A | I.A. | N/A |
| A122 | ++++ | * | I.A. | N/A | I.A. | N/A | I.A. | N/A | I.A. | N/A |
| A123 | ++++ | * | I.A. | N/A | I.A. | N/A | I.A. | N/A | I.A. | N/A |
| A124 | ++++ | * | I.A. | N/A | I.A. | N/A | I.A. | N/A | I.A. | N/A |
| A125 | ++++ | * | I.A. | N/A | I.A. | N/A | I.A. | N/A | I.A. | N/A |
| A126 | ++++ | * | I.A. | N/A | I.A. | N/A | I.A. | N/A | I.A. | N/A |
| A127 | ++++ | * | I.A. | N/A | I.A. | N/A | I.A. | N/A | I.A. | N/A |
| A128 | ++++ | * | I.A. | N/A | I.A. | N/A | I.A. | N/A | I.A. | N/A |
| A129 | ++++ | * | I.A. | N/A | I.A. | N/A | I.A. | N/A | I.A. | N/A |
| A130 | ++++ | * | I.A. | N/A | I.A. | N/A | I.A. | N/A | I.A. | N/A |
| A131 | ++++ | * | I.A. | N/A | I.A. | N/A | I.A. | N/A | I.A. | N/A |
| A132 | ++++ | * | I.A. | N/A | I.A. | N/A | I.A. | N/A | I.A. | N/A |
| A133 | +++ | *** | I.A. | N/A | I.A. | N/A | I.A. | N/A | I.A. | N/A |
| A134 | ++++ | * | I.A. | N/A | I.A. | N/A | I.A. | N/A | I.A. | N/A |
| A135 | ++++ | *** | I.A. | N/A | N/A | N/A | I.A. | N/A | I.A. | N/A |
| A136 | ++++ | * | I.A. | N/A | I.A. | N/A | I.A. | N/A | I.A. | N/A |
| A137 | ++++ | * | I.A. | N/A | I.A. | N/A | I.A. | N/A | I.A. | N/A |
| A138 | ++++ | * | I.A. | N/A | I.A. | N/A | I.A. | N/A | I.A. | N/A |
| A139 | ++++ | * | I.A. | **** | N/A | N/A | I.A. | N/A | I.A. | N/A |
| A140 | ++++ | * | +++ | *** | I.A. | N/A | I.A. | N/A | I.A. | N/A |
| A141 | +++ | * | I.A. | N/A | I.A. | N/A | I.A. | N/A | I.A. | N/A |
| A142 | ++++ | * | I.A. | N/A | I.A. | N/A | I.A. | N/A | I.A. | N/A |
| A143 | ++++ | * | I.A. | N/A | I.A. | N/A | I.A. | N/A | I.A. | N/A |
| A144 | ++++ | * | I.A. | N/A | I.A. | N/A | I.A. | N/A | I.A. | N/A |
| A145 | ++++ | * | I.A. | N/A | I.A. | N/A | I.A. | N/A | I.A. | N/A |
| A146 | ++++ | * | +++ | *** | I.A. | N/A | I.A. | N/A | I.A. | N/A |
| A147 | ++++ | * | I.A. | N/A | I.A. | N/A | I.A. | N/A | I.A. | N/A |
| A148 | ++++ | * | I.A. | N/A | I.A. | N/A | I.A. | N/A | I.A. | N/A |
| A149 | ++ | N/A | I.A. | N/A | N/A | N/A | I.A. | N/A | I.A. | N/A |
| A150 | ++++ | ** | I.A. | N/A | N/A | N/A | I.A. | N/A | I.A. | N/A |
| A151 | ++ | N/A | I.A. | N/A | N/A | N/A | I.A. | N/A | I.A. | N/A |
| A152 | ++++ | * | I.A. | N/A | N/A | N/A | I.A. | N/A | I.A. | N/A |
| A153 | ++++ | * | I.A. | N/A | I.A. | N/A | I.A. | N/A | I.A. | N/A |
| A154 | ++++ | * | I.A. | N/A | N/A | N/A | I.A. | N/A | I.A. | N/A |
| A155 | ++++ | * | I.A. | N/A | I.A. | N/A | I.A. | N/A | I.A. | N/A |
| A156 | ++++ | * | I.A. | N/A | I.A. | N/A | I.A. | N/A | I.A. | N/A |
| A157 | ++++ | * | I.A. | N/A | I.A. | N/A | I.A. | N/A | I.A. | N/A |
| A158 | ++++ | * | ++++ | *** | I.A. | N/A | I.A. | N/A | I.A. | N/A |
| A159 | ++++ | * | I.A. | N/A | I.A. | N/A | I.A. | N/A | I.A. | N/A |
| A160 | ++++ | * | I.A. | N/A | I.A. | N/A | I.A. | N/A | I.A. | N/A |
| A161 | ++++ | * | I.A. | N/A | I.A. | N/A | I.A. | N/A | I.A. | N/A |
| A162 | ++++ | * | I.A. | N/A | I.A. | N/A | I.A. | N/A | I.A. | N/A |
| A163 | ++++ | * | I.A. | N/A | I.A. | N/A | I.A. | N/A | I.A. | N/A |
| A164 | +++ | ** | I.A. | N/A | I.A. | N/A | I.A. | N/A | I.A. | N/A |
| A165 | ++++ | * | I.A. | N/A | I.A. | N/A | I.A. | N/A | I.A. | N/A |
| A166 | ++++ | * | I.A. | N/A | I.A. | N/A | I.A. | N/A | ++ | *** |
| A167 | ++++ | * | I.A. | N/A | I.A. | N/A | I.A. | N/A | I.A. | N/A |
| A168 | ++++ | * | I.A. | N/A | I.A. | N/A | I.A. | N/A | I.A. | N/A |
| A169 | ++++ | * | I.A. | N/A | I.A. | N/A | I.A. | N/A | I.A. | N/A |
| A170 | ++++ | * | I.A. | N/A | I.A. | N/A | I.A. | N/A | I.A. | N/A |
| A171 | ++++ | * | I.A. | N/A | I.A. | N/A | I.A. | N/A | I.A. | N/A |
| A172 | ++++ | * | I.A. | N/A | I.A. | N/A | I.A. | N/A | I.A. | N/A |
| A173 | +++ | ** | I.A. | N/A | I.A. | N/A | I.A. | N/A | I.A. | N/A |
| A174 | ++++ | * | I.A. | N/A | I.A. | N/A | I.A. | N/A | I.A. | N/A |
| A175 | ++++ | * | I.A. | N/A | I.A. | N/A | I.A. | N/A | I.A. | N/A |
| A176 | ++++ | * | I.A. | N/A | I.A. | N/A | I.A. | N/A | I.A. | N/A |
| A177 | ++++ | * | I.A. | N/A | I.A. | N/A | I.A. | N/A | I.A. | N/A |
| A178 | +++ | *** | I.A. | N/A | I.A. | N/A | I.A. | N/A | I.A. | N/A |
| A179 | ++++ | * | I.A. | N/A | I.A. | N/A | I.A. | N/A | I.A. | N/A |
| A180 | ++++ | * | I.A. | N/A | I.A. | N/A | I.A. | N/A | I.A. | N/A |
| A181 | ++++ | * | I.A. | N/A | I.A. | N/A | I.A. | N/A | I.A. | N/A |
| A182 | ++++ | * | I.A. | N/A | I.A. | N/A | I.A. | N/A | I.A. | N/A |
| A183 | ++++ | * | I.A. | N/A | I.A. | N/A | I.A. | N/A | I.A. | N/A |
| A184 | ++++ | * | ++++ | *** | I.A. | N/A | I.A. | N/A | I.A. | N/A |
| A185 | ++++ | * | ++++ | *** | I.A. | N/A | I.A. | N/A | I.A. | N/A |
| A186 | ++++ | * | I.A. | N/A | I.A. | N/A | I.A. | N/A | I.A. | N/A |
| A187 | ++++ | * | I.A. | N/A | I.A. | N/A | I.A. | N/A | I.A. | N/A |
| A188 | ++++ | * | I.A. | N/A | I.A. | N/A | I.A. | N/A | I.A. | N/A |
| A189 | ++++ | * | I.A. | N/A | I.A. | N/A | I.A. | N/A | I.A. | N/A |
| A190 | ++++ | * | I.A. | N/A | I.A. | N/A | I.A. | N/A | I.A. | N/A |
| A191 | ++++ | ** | I.A. | N/A | N/A | N/A | I.A. | N/A | I.A. | N/A |
| A192 | ++++ | * | I.A. | N/A | N/A | N/A | I.A. | N/A | I.A. | N/A |
| A193 | ++++ | * | I.A. | N/A | N/A | N/A | I.A. | N/A | I.A. | N/A |
| A194 | ++++ | * | I.A. | N/A | +++ | **** | I.A. | N/A | I.A. | N/A |
| A195 | +++ | ** | I.A. | N/A | + | N/A | I.A. | N/A | I.A. | N/A |
| A196 | N/A | N/A | I.A. | N/A | N/A | N/A | I.A. | N/A | I.A. | N/A |
| A197 | ++++ | * | I.A. | N/A | N/A | N/A | I.A. | N/A | I.A. | N/A |
| A198 | +++ | ** | I.A. | N/A | + | N/A | I.A. | N/A | I.A. | N/A |
| A199 | +++ | *** | I.A. | N/A | + | N/A | I.A. | N/A | I.A. | N/A |
| A200 | ++ | *** | I.A. | N/A | I.A. | N/A | I.A. | N/A | I.A. | N/A |
| A201 | ++++ | * | I.A. | N/A | I.A. | N/A | I.A. | N/A | I.A. | N/A |
| A202 | ++ | *** | I.A. | N/A | I.A. | N/A | I.A. | N/A | I.A. | N/A |
| A203 | ++ | *** | I.A. | N/A | I.A. | N/A | I.A. | N/A | I.A. | N/A |
| A204 | ++++ | * | I.A. | N/A | I.A. | N/A | I.A. | N/A | I.A. | N/A |
| A205 | ++++ | * | I.A. | N/A | I.A. | N/A | I.A. | N/A | I.A. | N/A |
| A206 | ++++ | * | +++ | *** | I.A. | N/A | I.A. | N/A | ++ | *** |
| A207 | ++++ | * | I.A. | N/A | I.A. | N/A | I.A. | N/A | I.A. | N/A |
| A208 | +++ | ** | I.A. | N/A | I.A. | N/A | I.A. | N/A | I.A. | N/A |
| A209 | ++++ | * | I.A. | N/A | I.A. | N/A | I.A. | N/A | I.A. | N/A |
| A210 | ++++ | * | ++++ | ** | I.A. | N/A | I.A. | N/A | ++ | *** |
| A211 | +++ | * | N/A | N/A | I.A. | N/A | N/A | N/A | N/A | N/A |
| A212 | ++++ | * | N/A | N/A | I.A. | N/A | N/A | N/A | N/A | N/A |
| A213 | ++++ | * | N/A | N/A | I.A. | N/A | N/A | N/A | N/A | N/A |
| A214 | ++++ | * | N/A | N/A | I.A. | N/A | N/A | N/A | N/A | N/A |
| A215 | ++++ | * | I.A. | N/A | I.A. | N/A | I.A. | N/A | I.A. | N/A |
| A216 | ++++ | * | I.A. | N/A | N/A | N/A | I.A. | N/A | I.A. | N/A |
| A217 | ++++ | * | I.A. | N/A | N/A | N/A | I.A. | N/A | I.A. | N/A |
| A218 | ++++ | * | I.A. | N/A | I.A. | N/A | I.A. | N/A | I.A. | N/A |
| A219 | + | **** | I.A. | N/A | + | N/A | I.A. | N/A | I.A. | N/A |
| A220 | ++ | N/A | I.A. | N/A | + | N/A | I.A. | N/A | I.A. | N/A |
| A221 | ++++ | * | I.A. | N/A | + | N/A | I.A. | N/A | I.A. | N/A |
| A222 | ++ | N/A | I.A. | N/A | + | N/A | I.A. | N/A | I.A. | N/A |
| A223 | +++ | * | I.A. | N/A | + | N/A | I.A. | N/A | I.A. | N/A |
| A224 | ++ | N/A | I.A. | N/A | + | N/A | I.A. | N/A | I.A. | N/A |
| A225 | N/A | N/A | I.A. | N/A | N/A | N/A | I.A. | N/A | I.A. | N/A |
| A226 | ++++ | * | I.A. | N/A | + | N/A | I.A. | N/A | I.A. | N/A |
| A227 | +++ | * | I.A. | N/A | + | N/A | I.A. | N/A | I.A. | N/A |
| A228 | +++ | * | I.A. | N/A | I.A. | N/A | I.A. | N/A | I.A. | N/A |
| A229 | ++++ | * | I.A. | N/A | I.A. | N/A | I.A. | N/A | I.A. | N/A |
| A230 | ++++ | * | I.A. | N/A | I.A. | N/A | I.A. | N/A | I.A. | N/A |
| A231 | ++++ | ** | I.A. | N/A | N/A | N/A | I.A. | N/A | I.A. | N/A |
| A232 | ++ | N/A | I.A. | N/A | N/A | N/A | I.A. | N/A | I.A. | N/A |
| A233 | ++++ | * | I.A. | N/A | I.A. | N/A | I.A. | N/A | I.A. | N/A |
| A234 | +++ | * | I.A. | N/A | I.A. | N/A | I.A. | N/A | I.A. | N/A |
| A235 | ++++ | * | I.A. | N/A | I.A. | N/A | I.A. | N/A | I.A. | N/A |
| A236 | ++++ | * | N/A | N/A | I.A. | N/A | N/A | N/A | N/A | N/A |
| A237 | + | N/A | I.A. | N/A | N/A | N/A | I.A. | N/A | I.A. | N/A |
| A238 | +++ | *** | N/A | N/A | N/A | N/A | N/A | N/A | N/A | N/A |
| A239 | +++ | * | N/A | N/A | N/A | N/A | N/A | N/A | N/A | N/A |
| A240 | ++++ | * | N/A | N/A | N/A | N/A | N/A | N/A | N/A | N/A |
| A241 | +++ | ** | N/A | N/A | N/A | N/A | N/A | N/A | N/A | N/A |
| A242 | ++++ | * | N/A | N/A | N/A | N/A | N/A | N/A | N/A | N/A |
| A243 | +++ | ** | I.A. | N/A | N/A | N/A | I.A. | N/A | I.A. | N/A |
| A244 | +++ | * | I.A. | N/A | N/A | N/A | I.A. | N/A | I.A. | N/A |
| A245 | ++++ | * | I.A. | N/A | N/A | N/A | I.A. | N/A | I.A. | N/A |
| A246 | ++++ | * | I.A. | N/A | N/A | N/A | I.A. | N/A | I.A. | N/A |
| A247 | ++++ | * | I.A. | N/A | N/A | N/A | I.A. | N/A | I.A. | N/A |
| A248 | ++ | N/A | I.A. | N/A | N/A | N/A | I.A. | N/A | I.A. | N/A |
| A249 | ++++ | * | I.A. | N/A | N/A | N/A | I.A. | N/A | I.A. | N/A |
| A250 | +++ | * | I.A. | N/A | N/A | N/A | I.A. | N/A | I.A. | N/A |
| A251 | ++++ | * | I.A. | N/A | N/A | N/A | I.A. | N/A | I.A. | N/A |
| A252 | ++++ | * | I.A. | N/A | N/A | N/A | I.A. | N/A | I.A. | N/A |
| A253 | ++++ | * | I.A. | N/A | N/A | N/A | I.A. | N/A | I.A. | N/A |
| A254 | ++ | N/A | I.A. | N/A | N/A | N/A | I.A. | N/A | I.A. | N/A |
| A255 | ++++ | * | I.A. | N/A | N/A | N/A | I.A. | N/A | I.A. | N/A |
| A256 | ++++ | * | I.A. | N/A | N/A | N/A | I.A. | N/A | I.A. | N/A |
| A257 | ++ | N/A | I.A. | N/A | N/A | N/A | I.A. | N/A | I.A. | N/A |
| A258 | +++ | ** | I.A. | N/A | N/A | N/A | I.A. | N/A | I.A. | N/A |
| A259 | ++++ | * | I.A. | N/A | N/A | N/A | I.A. | N/A | I.A. | N/A |
| A260 | ++++ | * | N/A | N/A | N/A | N/A | N/A | N/A | N/A | N/A |
| A261 | ++++ | * | N/A | N/A | N/A | N/A | N/A | N/A | N/A | N/A |
| A262 | ++++ | * | N/A | N/A | N/A | N/A | N/A | N/A | N/A | N/A |
| A263 | ++++ | * | N/A | N/A | N/A | N/A | N/A | N/A | N/A | N/A |
| A264 | ++++ | * | N/A | N/A | N/A | N/A | N/A | N/A | N/A | N/A |
| A265 | ++ | N/A | N/A | N/A | N/A | N/A | N/A | N/A | N/A | N/A |
| A266 | ++++ | * | N/A | N/A | N/A | N/A | N/A | N/A | N/A | N/A |
| A267 | +++ | ** | N/A | N/A | N/A | N/A | N/A | N/A | N/A | N/A |
| A268 | ++++ | * | N/A | N/A | N/A | N/A | N/A | N/A | N/A | N/A |
| A269 | ++++ | * | N/A | N/A | N/A | N/A | N/A | N/A | N/A | N/A |
| A270 | ++++ | * | N/A | N/A | N/A | N/A | N/A | N/A | N/A | N/A |
| A271 | ++++ | * | N/A | N/A | N/A | N/A | N/A | N/A | N/A | N/A |
| A272 | +++ | *** | N/A | N/A | N/A | N/A | I.A. | N/A | I.A. | N/A |
| A273 | ++++ | * | I.A. | N/A | + | N/A | I.A. | N/A | I.A. | N/A |
| A274 | +++ | * | I.A. | N/A | N/A | N/A | I.A. | N/A | I.A. | N/A |
| A275 | ++++ | * | N/A | N/A | N/A | N/A | N/A | N/A | N/A | N/A |
| A276 | ++ | N/A | N/A | N/A | N/A | N/A | N/A | N/A | N/A | N/A |
| A277 | ++++ | * | N/A | N/A | N/A | N/A | N/A | N/A | N/A | N/A |
| A278 | ++++ | * | N/A | N/A | N/A | N/A | N/A | N/A | N/A | N/A |
| A279 | ++++ | * | I.A. | N/A | I.A. | N/A | I.A. | N/A | I.A. | N/A |
| A281 | ++++ | * | N/A | N/A | N/A | N/A | N/A | N/A | N/A | N/A |
| A282 | ++++ | * | N/A | N/A | N/A | N/A | N/A | N/A | N/A | N/A |
| A283 | ++++ | * | N/A | N/A | N/A | N/A | I.A. | N/A | I.A. | N/A |
| A284 | ++++ | * | N/A | N/A | N/A | N/A | I.A. | N/A | I.A. | N/A |
| A285 | ++++ | * | N/A | N/A | N/A | N/A | I.A. | N/A | I.A. | N/A |
| A286 | ++++ | * | N/A | N/A | N/A | N/A | N/A | N/A | N/A | N/A |
| A287 | ++++ | * | N/A | N/A | N/A | N/A | N/A | N/A | N/A | N/A |
| A288 | ++++ | *** | N/A | N/A | N/A | N/A | I.A. | N/A | I.A. | N/A |
| A289 | ++++ | * | ++++ | *** | N/A | N/A | I.A. | N/A | I.A. | N/A |
| A290 | ++++ | * | N/A | N/A | N/A | N/A | I.A. | N/A | I.A. | N/A |
| A291 | +++ | ** | N/A | N/A | N/A | N/A | I.A. | N/A | I.A. | N/A |
| A292 | ++ | N/A | N/A | N/A | N/A | N/A | I.A. | N/A | I.A. | N/A |
| A293 | ++++ | * | N/A | N/A | N/A | N/A | I.A. | N/A | I.A. | N/A |

I.A. indicates EC50 >10 μM; N/A indicates not tested or not available; “+” means 100 to 499.9 nM; “++” means 20 to 99.9 nM; and “+++” means <20 nM; “*” means 80 to 100%; “**” means 50 to 79.9%; and “***” means 19 to 49.9%; and “***” means <19%.

1. A compound having a structure of Formula (I): 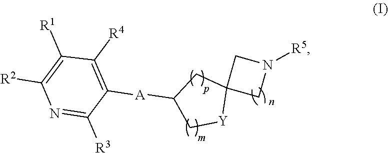
or a pharmaceutically acceptable salt of the compound, wherein:
A is a 6-8 membered heterocycle comprising 1 or 2 ring nitrogen atoms and optionally substituted with 1 to 3 substituents independently selected from halogen, OH, and C₁₋₃alkyl;
Y is a bond, O, S, CH₂, CHF, CF₂, or C(OH)H;
m is 1 or 2;
n is 1 or 2;
p is 1 or 2;
R¹ is H, halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle or —[O]₀₋₁-5-10 membered heteroaryl, wherein the heterocycle and heteroaryl each comprises 1, 2, or 3 ring heteroatoms selected from N, O, and S, and when R¹ is C₁₋₆alkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, —[O]₀₋₁-5-10 membered heteroaryl, —NH—C₃₋₆cycloalkyl, —NH—C₆₋₁₀aryl, —NH-4-8 membered heterocycle, —NH-5-10 membered heteroaryl, —N(C₁₋₆alkyl)-C₃₋₆cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, R¹ is optionally substituted with 1, 2, or 3 substituents independently selected from halogen, CN, OH, ═O, SO₂, and C₁₋₃alkyl,
R² is H, halogen, CN, OH, —N(R⁶)(R⁷), C₁₋₆alkyl, C₁₋₆haloalkyl, C₁₋₆alkoxy, C₁₋₆haloalkoxy, C₂₋₆heteroalkyl, C₃₋₆cycloalkyl, or 4-8 membered heterocycle comprising 1, 2, or 3 ring heteroatoms selected from N, O, and S;
R³ is halogen, CN, OH, —N(R⁶)(R⁷), C₁₋₆alkyl, C₂₋₆heteroalkyl, C₂₋₆alkenyl, C₂₋₆alkynyl, C₁₋₆alkoxy, —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, —NH—C₃₋₆cycloalkyl, —NH—C₆₋₁₀aryl, —NH-4-8 membered heterocycle, —NH-5-10 membered heteroaryl, —N(C₁₋₆alkyl)-C₃₋₆cycloalkyl, —N(C₁₋₆alkyl)-C₆₋₁₀aryl, —N(C₁₋₆alkyl)-4-8 membered heterocycle, or —N(C₁₋₆alkyl)-5-10 membered heteroaryl, wherein the heterocycle and heteroaryl each comprises 1, 2, or 3 ring heteroatoms selected from N, O, and S, and the C₃₋₆cycloalkyl, —C₆₋₁₀aryl, 4-8 membered heterocycle, or 5-10 membered heteroaryl is substituted with 0, 1, 2, or 3 R³a substituents;
each R^{3a} is independently selected from halogen, CN, OH, ═O, ═N(C₁₋₃alkly), SO₂, C₁₋₆alkyl, C₂₋₁₀alkene, C₁₋₆hydroxyalkyl, C₁₋₆haloalkyl, C₁₋₆alkoxy, C₁₋₆haloalkoxy, C₁₋₆alkylene-O—C₁₋₆alkyl, C₀₋₆alkylene-NH₂, C₀₋₆alkylene-NH(C₁₋₆alkyl), C₀₋₆alkylene-N(C₁₋₆alkyl)₂, —S—C₁₋₆alkyl, C₀₋₆alkylene-SO₂C₁₋₆alkyl, C₀₋₆alkylene-C(O)NH₂, C₀₋₆alkylene-C(O)NH(C₁₋₆alkyl), C₀₋₆alkylene-C(O)N(C₁₋₆alkyl)₂, C₀₋₆alkylene-NHC(O)C₁₋₆alkyl, C₀₋₆alkylene-COOH, C₀₋₆alkylene-C₃₋₆cycloalkyl, C₁₋₆alkylene-O—C₁₋₆alklyeneSi(C₁₋₃alkyl)₃, and C₀₋₆alkylene-3-6 membered heterocycle comprising 1, 2, or 3 heteroatoms selected from N, O, and S;
R⁴ is H, halogen, CN, or OH;
R⁵ is —CO₂—Z, or a bioisostere thereof;
each R⁶ and R⁷ is independently H, C₁₋₆alkyl, C(O)—C₁₋₆alkyl, spiro or bicyclic C₈₋₁₄cycloalkyl, 8-14 membered heterocycle comprising 1, 2, or 3 ring heteroatoms selected from N, O, and S, and when R⁶ or R⁷ is other than H, it can be optionally substituted with 1, 2, or 3 substituents independently selected from the group consisting of halogen, CN, =O, SO₂, OH, C₀₋₆alkylene-NH₂, C₀₋₆alkylene-NH(C₁₋₆alkyl), C₀₋₆alkylene-N(C₁₋₆alkyl)₂, C₀₋₆alkylene-SO₂C₁₋₆alkyl, C₁₋₆alkyl and C₁₋₆alkoxy, or
R⁶ and R⁷, together with the nitrogen to which they are attached, form a 4-10 membered heterocycle comprising 0-2 additional ring heteroatoms independently selected from N, O, and S; and
Z is C₁₋₇alkyl, C₁₋₇haloalkyl, C₃₋₆cycloalkyl, or C₂₋₆alkyne, and Z is optionally substituted with C₁₋₆alkoxy or C₃₋₆cycloalkyl;
with the proviso that when R¹, R², and R⁴ are each H, Y is CH₂, m, n, and p are each 1, A is 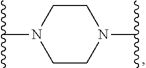 [SMILES: *N1CCN(*)CC1]
and R³ is trifluoroethoxy, trifluoromethoxy, difluoromethoxy, methoxy, or 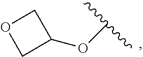 [SMILES: *OC1COC1]
then R⁵ is not CO₂CH₂CH₃.

2-3. (canceled)

4. The compound or salt of claim 1, having a structure of Formula (Ib):

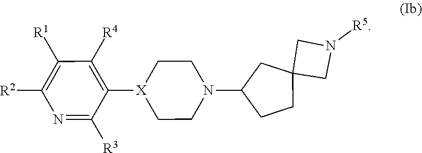

5-10. (canceled)

11. The compound or salt of claim 4, having a structure of Formula (Id):

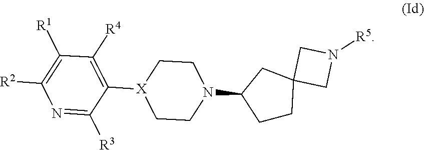

12. (canceled)

13. The compound or salt of claim 1, having a structure of Formula (Ie), or a pharmaceutically acceptable salt thereof:

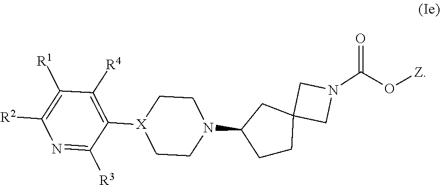

14. (canceled)

15. The compound or salt of claim 1, wherein R^{s} is CO₂CH₂CH₃.

16-17. (canceled)

18. The compound or salt of claim 1, wherein R¹ is H or halogen.

19-24. (canceled)

25. The compound or salt of claim 1, wherein at least one of R¹, R², R³, and R⁴ is halogen.

26-29. (canceled)

30. The compound or salt of claim 1, wherein R³ is 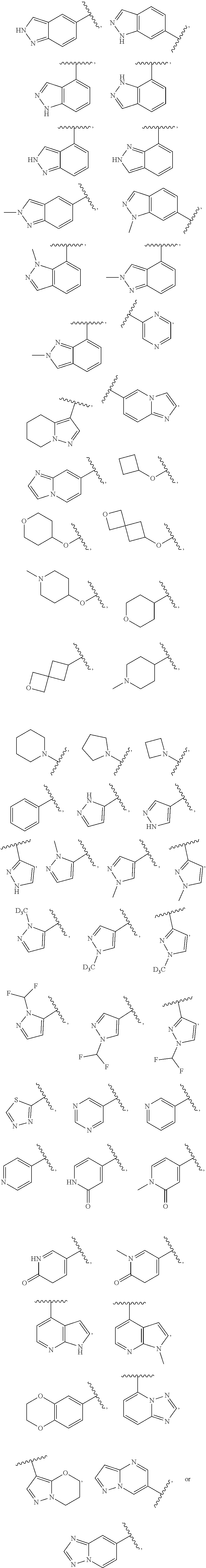
and is optionally substituted with 1, 2, or 3 R^{3a}.

31. The compound or salt of claim 1, wherein R³ is unsubstituted.

32-36. (canceled)

37. The compound or salt of claim 1, having a structure of Formula (If), or a pharmaceutically acceptable salt thereof: 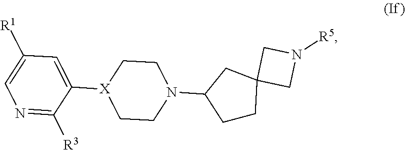
wherein
R¹ is halogen;
R³ is —[O]₀₋₁—C₃₋₆cycloalkyl, —[O]₀₋₁—C₆₋₁₀aryl, —[O]₀₋₁-4-8 membered heterocycle, or —[O]₀₋₁-5-10 membered heteroaryl, wherein the heterocycle and heteroaryl each comprises 1, 2, or 3 ring heteroatoms selected from N, O, and S, and R³ is substituted with 0, 1, 2, or 3 R^{3a} substituents;
R⁵ is CO₂Z. or a bioisostere thereof; and
Z is C₁₋₇alkyl, C₁₋₇haloalkyl, C₃₋₆cycloalkyl, or C₂₋₆alkyne, and is optionally substituted with C₁₋₆alkoxy or C₃₋₆cycloalkyl.

38. The compound or salt of claim 37, wherein R¹ is Cl or F.

39. The compound or salt of claim 37, wherein R³ is 5-6-membered heterocycle comprising 1 ring heteroatom selected from S and O, or 5-6-membered heteroaryl comprising 2 or 3 ring heteroatoms independently selected from N and S, and R³ is substituted with 0, 1 or 2 R^{3a} substituents independently selected from halogen, CN, OH, and C₁₋₆alkyl.

40. The compound or salt of claim 37, wherein R⁵ is CO₂C₁₋₇alkyl.

41. The compound or salt of claim 40, wherein R⁵ is CO₂Et.

42. (canceled)

43. A pharmaceutical formulation comprising a therapeutically effective amount of the compound or salt of claim 1, and a pharmaceutically acceptable excipient.

44. A method for treating an M4-mediated (or M4-associated) disease or disorder in a patient comprising administering to the patient a therapeutically effective amount of the compound or salt of claim 1.

45-46. (canceled)

47. The compound of claim 1, wherein the compound has the structure: 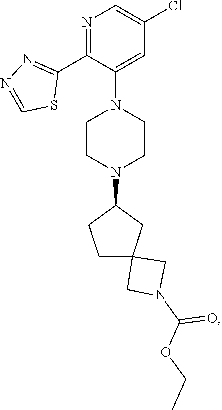 [SMILES: CCOC(=O)N1CC2(CC[C@@H](N3CCN(c4cc(Cl)cnc4-c4nncs4)CC3)C2)C1]
or a pharmaceutically acceptable salt thereof.

48. The compound of claim 47, wherein the compound has the structure:

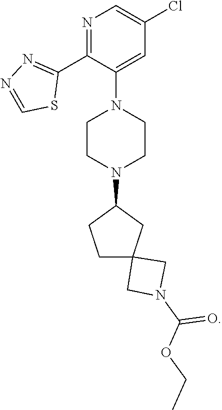 [SMILES: CCOC(=O)N1CC2(CC[C@@H](N3CCN(c4cc(Cl)cnc4-c4nncs4)CC3)C2)C1]

49. The compound of claim 47, wherein the compound is a pharmaceutically acceptable salt of the compound having the structure: 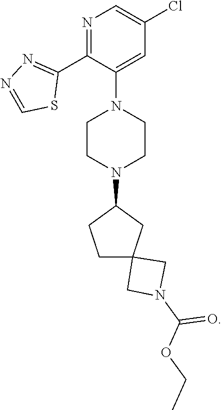 [SMILES: CCOC(=O)N1CC2(CC[C@@H](N3CCN(c4cc(Cl)cnc4-c4nncs4)CC3)C2)C1]
